(19) United States
(12) Patent Application Publication
(10) Pub. No.: US 2026/0034111 A1
(43) Pub. Date: Feb. 5, 2026

(54) NOVEL HETEROCYCLES AS sPLA2-X INHIBITORS

(71) Applicant: Cayman Chemical Company Incorporated, Ann Arbor, MI (US)

(72) Inventors: Zahra Assar-Nossoni, Northville, MI (US); Stephen Douglas Barrett, Hartland, MI (US); Fred Lawrence Ciske, Dexter, MI (US); Andrei Mikhailovich Kornilov, Ypsilanti, MI (US); James Bernard Kramer, Sylvania, OH (US); Kirk Lang Olson, Canton, MI (US); Sreenu Jennepalli, Ann Arbor, MI (US)

(73) Assignee: Cayman Chemical Company Incorporated, Ann Arbor, MI (US)

(21) Appl. No.: 18/997,666

(22) PCT Filed: Jul. 25, 2023
(86) PCT No.: PCT/US2023/070910

§ 371 Date: Jan. 22, 2025

Related U.S. Application Data
(60) Provisional application No. 63/392,092, filed on Jul. 25, 2022.
(60) Provisional application No. 63/493,905, filed on Apr. 3, 2023.

(51) Int. Cl. A61K 31/4439; A61K 31/404; A61K 31/4427; A61K 31/4436; A61K 31/444; C07D 209/42; C07D 401/04; C07D 409/04; C07D 421/04; C07D 471/04

(52) U.S. Cl. CPC A61K 31/4439; A61K 31/404; A61K 31/4427; A61K 31/4436; A61K 31/444; C07D 209/42; C07D 401/04; C07D 409/04; C07D 421/04; C07D 471/04

(57) ABSTRACT

The present invention relates to compounds, pharmaceutically acceptable salts of the compounds, and pharmaceutical compositions of the compounds, or salts thereof, that can inhibit secreted phospholipases A2 (sPLA2-X) enzymes, wherein the compound is a compound of Formula (I), wherein Ring B, R₆, Z₁, Z₂, Z₃, and Z₄ are described herein. The invention also relates to the use of the compounds, salts, or compositions described herein in methods of inhibiting sPLA2-X enzymes in a sample. The invention also relates to the use of the compounds, salts, or compositions in methods of treating or lessening the severity of an sPLA2-X mediated disease in a subject.

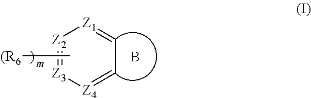

### CROSS REFERENCE TO RELATED APPLICATIONS

This application claims the benefit of, and priority to, U.S. Provisional Application Ser. No. 63/392,092, filed on Jul. 25, 2022, and U.S. Provisional Application Ser. No. 63/493,905, filed on Apr. 3, 2023, the disclosures of which are incorporated by reference herein in their entireties.

### TECHNICAL FIELD OF THE INVENTION

The present invention relates to compounds, pharmaceutically acceptable salts of the compounds, and pharmaceutical compositions of the compounds, or salts thereof, that can inhibit secreted phospholipase A2 Group X (sPLA2-X) enzymatic activity. The invention also relates to the use of the compounds, salts, or compositions described herein in methods of inhibiting sPLA2-X enzymatic activity in a sample. The invention also relates to the use of compounds, salts, or compositions in methods of treating or lessening the severity of an sPLA2-X mediated disease in a subject.

### BACKGROUND

Phospholipases A2 (PLA2s) are a superfamily of key enzymes involved in a multitude of (patho) physiological and cellular processes (Balsinde et al., (1999) Annu. Rev. Pharmacol. Toxicol. 39: 175-189 and Yuan and Tsai (1999) Biochim Biophys Acta 1441: 215). Phospholipases A2 (PLA2s) constitute one of the largest families of lipolytic enzymes and are defined by their ability to catalyze the hydrolysis of the ester bond at the sn-2 position of glycerophospholipids, yielding free fatty acids and lysophospholipids, from which secondary messengers may be generated. In vivo, the sn-2 position of phospholipids frequently contains polyunsaturated fatty acids that may be metabolized to form various eicosanoids and related bioactive lipids. Lysophospholipids also have various important roles in biological processes. The PLA2 superfamily currently consists of sixteen groups and many subgroups which differ in primary sequence, structure and catalytic mechanism. There are six main types or classes of PLA2: the secreted PLA2 (sPLA2), the cytosolic PLA2 (cPLA2), the Ca²⁺-independent PLA2 (iPLA2), lysosomal PLA2 (LPLA2), adipose PLA2 (AdPLA2), and the lipoprotein associated PLA2 (LpPLA2).

The various PLA2 types have been implicated in diverse kinds of lipid signaling and inflammatory diseases. Rheumatoid arthritis, lung inflammation, neurological disorders, such as multiple sclerosis, cardiovascular diseases, including atherosclerosis, and cancer are included among the diseases where PLA2 enzymes are involved.

Cancer remains one of the deadliest threats to human health. Worldwide, an estimated 19.3 million new cancer cases (18.1 million excluding non-melanoma skin cancer) and almost 10 million cancer deaths (9.9 million excluding non-melanoma skin cancer) occurred in 2020. These statistics are predicted to increase further by 2025. An effective treatment strategy is needed.

Inhibition of PLA2 has found some practical use in the treatment of cancer. For example, US Patent Application Publication No. 2019/0142835 ('835 publication) is directed to the treatment of cancer by administering an anti-cancer agent in combination with a PLA2 inhibitor. Cancer therapies also increase autophagy, though the mechanisms responsible for this are unclear. The '835 publication similarly found that survival of treated cancer cells with an anti-cancer agent was critically dependent on phospholipase A2 (PLA2) to mobilize lysophospholipids and free fatty acids to support fatty acid oxidation and oxidative phosphorylation.

Notwithstanding existing cancer therapies and promising therapies in development, the high rates of relapse and refractory disease among the lymphoma and other cancer populations drive the continual need for new approaches to effective cancer treatments for patients.

### BRIEF DESCRIPTION OF THE FIGURE

FIG. 1 is affinity sensogram providing the kinetics and steady state affinity (1:1 fit) of Compound 5 for sPLA2-X.

### SUMMARY OF THE INVENTION

In one aspect, the invention includes a compound of Formula I

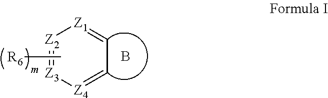

or a pharmaceutically acceptable salt thereof, wherein
Z₁-Z₄ are each independently selected from N and CH, wherein at least two of Z₁-Z₄ are CH;
each R₆ is independently selected from CN, halo, NH₂, OH, C₁₋₆ alkyl, C₁₋₆ haloalkyl, —OC₁₋₆ alkyl, and —OC₁₋₆ haloalkyl;
Ring B is selected from B1, B2, B3, B4, and B5

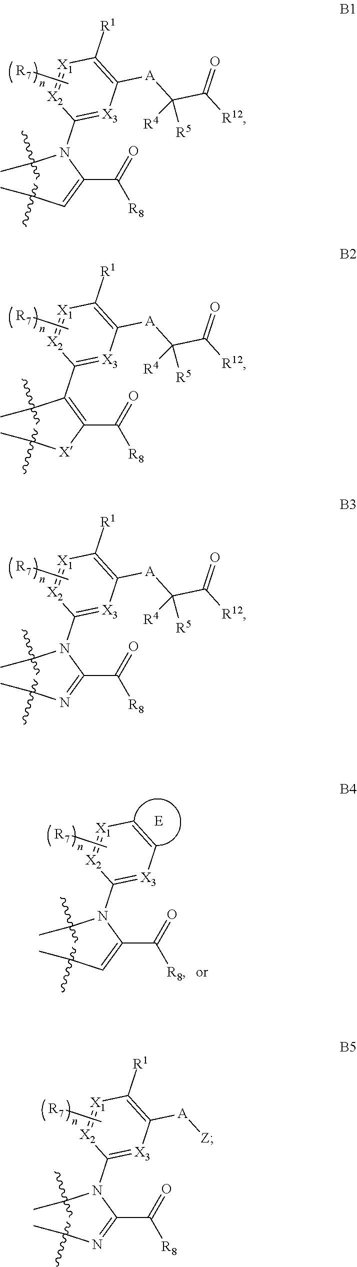

wherein,
X′ is O, S, Se, or NR₉, wherein R₉ is selected from H, C₁₋₆ alkyl, C₃₋₆ cyclyl, 3-6 membered heterocyclyl, and 5-6 membered heteroaryl, wherein said alkyl, cyclyl, heterocyclyl, or heteroaryl are each independently and optionally substituted with one or more R₁₀ substituents;
X₁-X₃ are each independently N or CH;
A is —O—, —S—, —S(O₂)—, or C(R₂)R₃
R₁ is H or R₇;
R₂ and R₃ are each independently selected from hydrogen CN, OH, NH₂, halo, and C₁₋₆ alkyl, or R₂ and R₃, together with the carbon atom to which they are attached, form a 3-6 membered cyclyl or a 3-6 membered heterocyclyl, wherein each alkyl, cyclyl, and heterocyclyl is independently and optionally substituted with one or more R₁₀ substituents;
R₄ and R₅ are each independently selected from hydrogen, CN, OH, NH₂, halo, C₁₋₆ alkyl, and OC₁₋₆ alkyl, wherein each alkyl is independently and optionally substituted with one or more R₁₀ substituents;
   or, R₄ and R₅, together with the carbon atom to which they are attached, form an oxo, a 3-6 membered cyclyl or a 3-6 membered heterocyclyl, wherein each cyclyl and heterocyclyl are optionally substituted with one or more R₁₀ substituents;
or, R₃ and R₄ form a double bond, or, together with the carbon atom to which they are attached, form a 3-6 membered cyclyl or a 3-6 membered heterocyclyl, wherein each cyclyl and heterocyclyl are optionally substituted with one or more R₁₀ substituents;
each R₇ is independently R₁₀;
R₈ and R₁₂ are each independently selected from OH, OC₁₋₆ alkyl, O-phenyl, NH₂, NH(C₁₋₆ alkyl), and N(C₁₋₆ alkyl)₂, wherein each alkyl and phenyl are optionally and independently substituted with one to three R₁₀ substituents;
Ring E is a 5-6 membered fused cyclic moiety selected from cyclyl, heterocyclyl, and heteroaryl, each of which is optionally and independently substituted with one to three R₁₀ substituents;
L is a bond or a bivalent moiety selected from C₁₋₆ alkyl, C₃₋₆ cyclyl, and a 3-6 membered heterocyclyl, each of which is optionally and independently substituted with one to three R₁₁ substituents;
Z is R₁₀;
each R₁₀ is selected from oxo, CN, OR₁₁, N(R₁₁)₂, halo, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, S(O)₂N(R₁₁)₂, C₁₋₆ alkyl, a phenyl, a 3-6 membered cyclyl, a 3-6 membered heterocyclyl, or a 5 or 6 membered heteroaryl, wherein each alkyl, phenyl, cyclyl, heterocyclyl, or heteroaryl are each independently and optionally substituted with one or more R₁₁ substituents;
each R₁₁ is independently selected from hydrogen, oxo, CN, OH, NH₂, halo, OC₁₋₆ alkyl, OC₁₋₆ haloalkyl, C₁₋₆ alkyl, and C₁₋₆ haloalkyl;
n is an integer selected from 0, 1, 2, and 3; and m is an integer selected from 1, 2, 3, and 4;
provided that the compound of Formula I is not any of the following compounds:
a compound having the formula

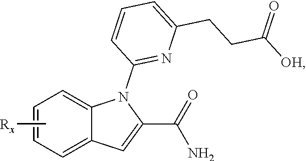

wherein R_{X} is a single substituent independently selected from hydrogen, 4-methyl, 5-methyl, 6-methyl, 7-methyl, 6-fluoro, 6-chloro, 6-CN, 6-methoxy, 6-CF₃, 6-OCF₃, 6-OCF₂H, 6-OCH₂CF₃, 6-CH₂OH, 6-ethyl, and 6-cyclopropyl; or
a compound selected from

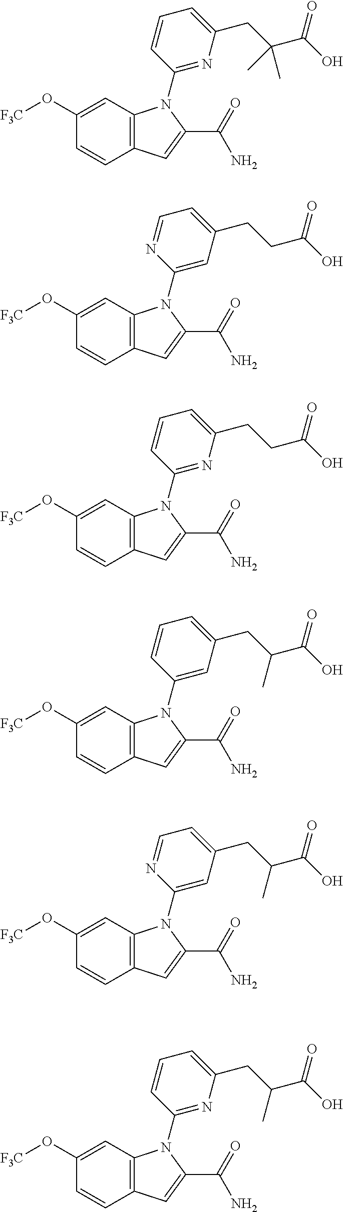 [SMILES: CC(C)(Cc1cccc(-n2c(C(N)=O)cc3ccc(OC(F)(F)F)cc32)n1)C(=O)O ; NC(=O)c1cc2ccc(OC(F)(F)F)cc2n1-c1cc(CCC(=O)O)ccn1 ; NC(=O)c1cc2ccc(OC(F)(F)F)cc2n1-c1cccc(CCC(=O)O)n1 ; CC(Cc1cccc(-n2c(C(N)=O)cc3ccc(OC(F)(F)F)cc32)c1)C(=O)O ; CC(Cc1ccnc(-n2c(C(N)=O)cc3ccc(OC(F)(F)F)cc32)c1)C(=O)O ; CC(Cc1cccc(-n2c(C(N)=O)cc3ccc(OC(F)(F)F)cc32)n1)C(=O)O]

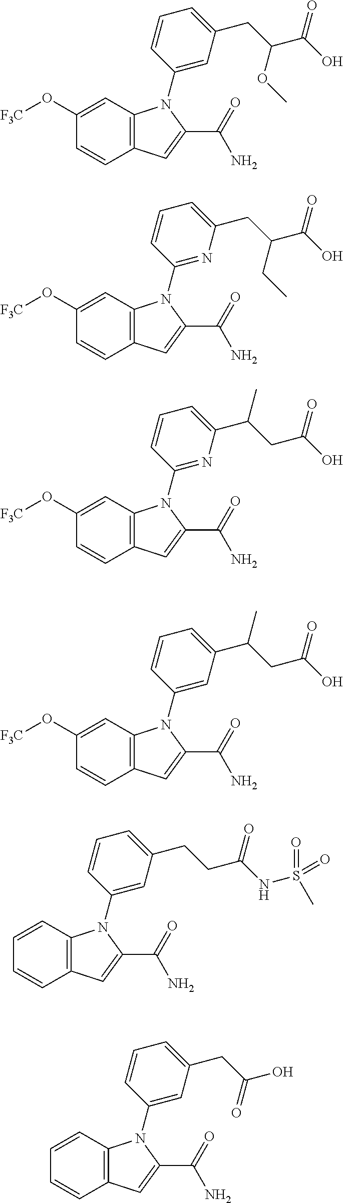

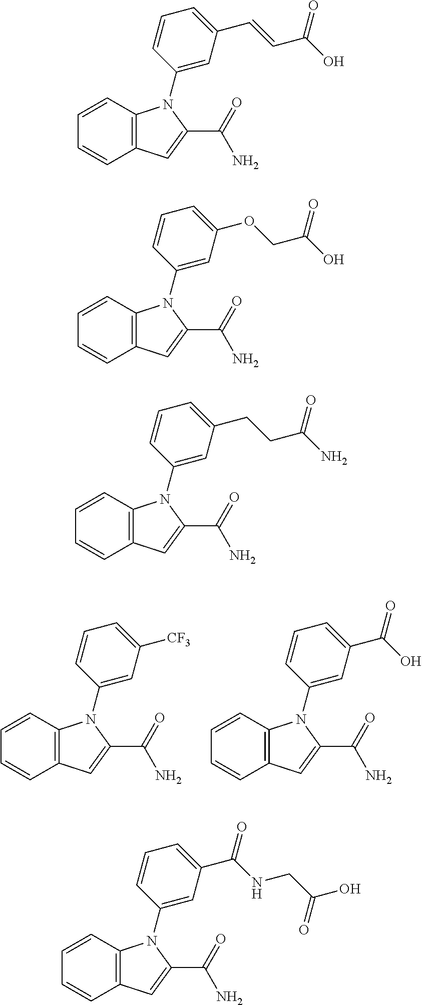 [SMILES: NC(=O)c1cc2ccccc2n1-c1cccc(/C=C/C(=O)O)c1 ; NC(=O)c1cc2ccccc2n1-c1cccc(OCC(=O)O)c1 ; NC(=O)CCc1cccc(-n2c(C(N)=O)cc3ccccc32)c1 ; NC(=O)c1cc2ccccc2n1-c1cccc(C(F)(F)F)c1 ; NC(=O)c1cc2ccccc2n1-c1cccc(C(=O)O)c1 ; NC(=O)c1cc2ccccc2n1-c1cccc(C(=O)NCC(=O)O)c1]

In another aspect, the invention includes a method of inhibiting an sPLA2-X enzyme, said method comprising contacting the enzyme with a compound or pharmaceutically acceptable salt thereof or pharmaceutical composition described herein.

In another aspect, the invention includes a method of treating or lessening the severity of a disease mediated by an sPLA2-X enzyme in a subject, said method comprising administering to the subject a therapeutically effective amount of a compound or pharmaceutically acceptable salt or a pharmaceutical composition described herein.

### DETAILED DESCRIPTION OF THE INVENTION

### Abbreviations

In describing the invention, chemical elements are identified in accordance with the Periodic Table of Elements. Abbreviations and symbols utilized herein are in accordance with the common usage of such abbreviations and symbols by those skilled in the chemical arts. The following abbreviations are used herein:
ACN acetonitrile
AcOEt ethyl acetate
AcOH acetic acid
APCI atmospheric pressure chemical ionization
Boc tert-butyloxycarbonyl
Br₂ Bromine
Cu(OAc)₂ copper (II) acetate
DBU 1,8-diazabicyclo[5.4.0]undec-7-ene
DCM dichloromethane
DIPEA diisopropylethylamine
DMAP 4-dimethylamino pyridine
DMF dimethyl formamide
DMSO-d₆ deuterated dimethylsulfoxide
DMSO dimethylsulfoxide
EtOH ethanol
Et₂NH diethylamine
g gram(s)
Hep heptane
Hex hexane
h hours
H₂O water
HPLC high pressure liquid chromatography
I₂ Iodine
i-PrOH isopropanol
KI potassium iodide
KHSO₄ potassium hydrogen sulfate
K₂CO₃ potassium carbonate
LDA lithium diisopropyl amide
LiOH lithium hydroxide
M molar
MeOH methanol
MαDG methyl α-D-glucopyranoside
MgSO₄ magnesium sulfate
min minutes
mg milligram(s)
mmol millimolar
mol mole
N₂ Nitrogen
NaCl sodium chloride
NaH sodium hydride
NaHCO₃ sodium bicarbonate
Na₂SO₄ sodium sulfate
NaO^{t}Bu sodium tert-butoxide
NaBH(OAc)₃ sodium triacetoxyborohydride
NH₄Cl ammonium chloride
NMP N-methyl-2-pyrrolidone
NMR Nuclear Magnetic Resonance spectroscopy
Pd₂(dba)₃ Tris(dibenzylideneacetone)dipalladium (0)
R_{f} retention factor
RT room temperature
Rt retention time
RuPhos 2-Dicyclohexylphosphino-2′,6′-diisopropoxybiphenyl
TBTU 2-(1H-Benzotriazole-1-yl)-1,1,3,3-tetramethylaminium tetrafluoroborate
TEA triethylamine
TFA trifluoracetic acid
THE tetrahydrofuran

### Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such asp,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A cycloalkyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S-], sulfoxy, urea, thiourea, sulfamoyl, sulfamide, oxo, or carbamoyl.

As used herein, a “cyclyl” group includes all cycloalkyl moieties and further includes non-aromatic mono or multicyclic carbocycles with one or more degrees of unsaturation. Examples of cyclyl groups include, but are not limited to cyclohexene, cyclohexa-1,3-diene, 4,5,6,7-tetrahydro-2H-indene, cyclohexa-1,4-diene, cyclopentene, cyclopentadiene, cyclobutene, and cyclopropane.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0]nonyl. A monocyclic heterocycloalkyl group can be fused with a phenyl moiety to form structures, such as tetrahydroisoquinoline, that would be categorized as heteroaryls.

A heterocycloalkyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

As used herein, a “heterocyclyl” group includes all heterocycloalkyl moieties and further includes non-aromatic mono or multicyclic heterocycles with one or more degrees of unsaturation. Examples of heterocyclyl groups include, but are not limited to 3,4-dihydro-2H-pyran, 2H-pyran, dihydropyridine, 1,2,3,4-tetrahydropyridine, 4,5,6,7-tetrahydroisobenzofuran, 2,3-dihydro-1H-pyrrole, 1H-azirine, and 1,2-dihydroazete.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophene-yl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, an “alkoxy” group refers to an alkyl-O— group where “alkyl” has been defined previously.

As used herein, a “haloalkyl” group refers to an alkyl group substituted with 1-3 halogen. For instance, the term haloalkyl includes the group —CF₃.

As used herein, a “carbonyl” refers to —C(O)—.

As used herein, a “carboxyl” refers to —C(O)OH.

As used herein, an “oxo” refers to ═O.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl, is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

The phrase “optionally substituted” is used herein interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R¹, X, L, X¹, X², X³, X⁴, X⁵, X⁶ and other variables contained in Formula (I), (II), and (II-A) described herein encompass specific groups, such as alkyl and aryl. Unless otherwise noted, each of the specific groups for the variables R¹, X, L, X¹, X², X³, X⁴, X⁵, X⁶ and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

As used herein, the phrase “stable or chemically feasible” refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless otherwise stated, structures depicted herein also are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

The term “pharmaceutically acceptable salt” as used herein, refers to any salt (e.g., obtained by reaction with an acid or a base) of a compound of the present invention that is physiologically tolerated in the target patient (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

As used herein, the term “prodrug” is intended to encompass therapeutically inactive compounds that, under physiological conditions, are converted into the therapeutically active agents of the present invention. One method for making a prodrug is to design selected moieties that are hydrolyzed or cleaved at a targeted in vivo site of action under physiological conditions to reveal the desired molecule which then produces its therapeutic effect. In certain embodiments, the prodrug is converted by an enzymatic activity of the subject. In some embodiments, the compound of the invention comprises a carboxylic acid moiety that is converted to an alkyl, aryl, or heteroaryl ester or amide, wherein the ester or amide is cleaved under physiological conditions to release the base carboxylic acid compound.

The term “sPLA2-X” refers to an enzyme (also known as group X secretory phospholipase A2, phosphatidylcholine 2-acylhydrolase 10, PLA2G10 or GXPLA2) which is a calcium-dependent enzyme that hydrolyzes glycerophospholipids to produce free fatty acids and lysophospholipids. In various embodiments, an exemplary human sPLA2-X enzyme that is inhibited by the compounds or pharmaceutically acceptable salts thereof, described herein, include human group X secretory phospholipase A2 having a pre-protein sequence defined by NCBI reference sequence No. NP_003552.1, (also UniProt/Swiss-Protein No. 015496). The mature sPLA2-X enzyme being amino acids 43-165 of said referenced NP_003552.1 sPLA2-X amino acid sequence.

In an alternate embodiment, the present invention provides prodrugs of compounds of Formula I.

### Embodiments of the Invention

In one aspect, the invention includes a compound of Formula I

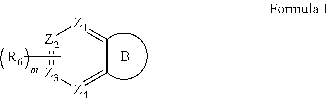

or a pharmaceutically acceptable salt thereof, wherein
Z₁-Z₄ are each independently selected from N and CH, wherein at least two of Z₁-Z₄ are CH;
each R₆ is independently selected from CN, halo, NH₂, OH, C₁₋₆ alkyl, C₁₋₆ haloalkyl, —OC₁₋₆ alkyl, and —OC₁₋₆ haloalkyl;
Ring B is selected from B1, B2, B3, B4, and B5

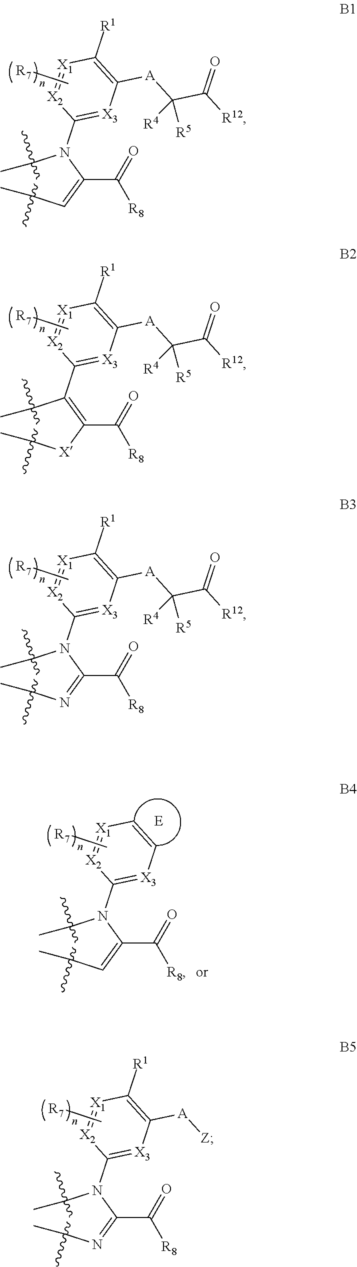

wherein,
X′ is O, S, Se, or NR₉, wherein R₉ is selected from H, C₁₋₆ alkyl, C₃₋₆ cyclyl, 3-6 membered heterocyclyl, and 5-6 membered heteroaryl, wherein said alkyl, cyclyl, heterocyclyl, or heteroaryl are each independently and optionally substituted with one or more R₁₀ substituents;
X₁-X₃ are each independently N or CH;
A is —O—, —S—, —S(O₂)—, or C(R₂)R₃
R₁ is H or R₇;
R₂ and R₃ are each independently selected from hydrogen CN, OH, NH₂, halo, and C₁₋₆ alkyl, or R₂ and R₃, together with the carbon atom to which they are attached, form a 3-6 membered cyclyl or a 3-6 membered heterocyclyl, wherein each alkyl, cyclyl, and heterocyclyl is independently and optionally substituted with one or more R₁₀ substituents;
R₄ and R₅ are each independently selected from hydrogen, CN, OH, NH₂, halo, C₁₋₆ alkyl, and OC₁₋₆ alkyl, wherein each alkyl is independently and optionally substituted with one or more R₁₀ substituents;
   or, R₄ and R₅, together with the carbon atom to which they are attached, form an oxo, a 3-6 membered cyclyl or a 3-6 membered heterocyclyl, wherein each cyclyl and heterocyclyl are optionally substituted with one or more R₁₀ substituents;
or, R₃ and R₄ form a double bond, or, together with the carbon atom to which they are attached, form a 3-6 membered cyclyl or a 3-6 membered heterocyclyl, wherein each cyclyl and heterocyclyl are optionally substituted with one or more R₁₀ substituents;
each R₇ is independently R₁₀;
R₈ and R₁₂ are each independently selected from OH, OC₁₋₆ alkyl, O-phenyl, NH₂, NH(C₁₋₆ alkyl), and N(C₁₋₆ alkyl)₂, wherein each alkyl and phenyl are optionally and independently substituted with one to three R₁₀ substituents;
Ring E is a 5-6 membered fused cyclic moiety selected from cyclyl, heterocyclyl, and heteroaryl, each of which is optionally and independently substituted with one to three R₁₀ substituents;
L is a bond or a bivalent moiety selected from C₁₋₆ alkyl, C₃₋₆ cyclyl, and a 3-6 membered heterocyclyl, each of which is optionally and independently substituted with one to three R₁₁ substituents;
Z is R₁₀;
each R₁₀ is selected from oxo, CN, OR₁₁, N(R₁₁)₂, halo, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, S(O)₂N(R₁₁)₂, C₁₋₆ alkyl, a phenyl, a 3-6 membered cyclyl, a 3-6 membered heterocyclyl, or a 5 or 6 membered heteroaryl, wherein each alkyl, phenyl, cyclyl, heterocyclyl, or heteroaryl are each independently and optionally substituted with one or more Ru substituents;
each R₁₁ is independently selected from hydrogen, oxo, CN, OH, NH₂, halo, OC₁₋₆ alkyl, OC₁₋₆ haloalkyl, C₁₋₆ alkyl, and C₁₋₆ haloalkyl;
n is an integer selected from 0, 1, 2, and 3; and
m is an integer selected from 1, 2, 3, and 4;
provided that the compound of Formula I is not any of the following compounds:
a compound having the formula

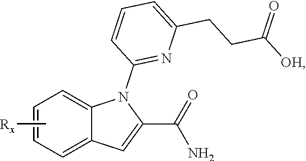

wherein R_{X} is a single substituent independently selected from hydrogen, 4-methyl, 5-methyl, 6-methyl, 7-methyl, 6-fluoro, 6-chloro, 6-CN, 6-methoxy, 6-CF₃, 6-OCF₃, 6-OCF₂H, 6-OCH₂CF₃, 6-CH₂OH, 6-ethyl, and 6-cyclopropyl; or
a compound selected from

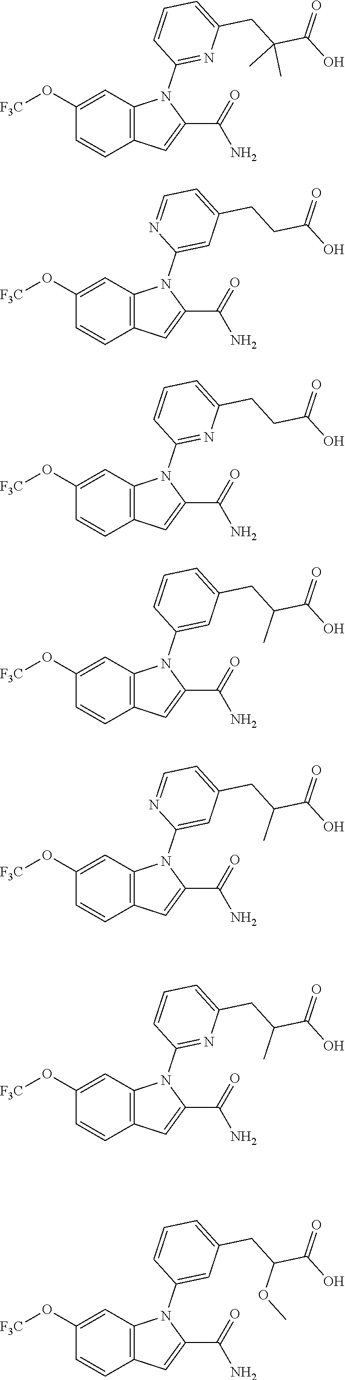

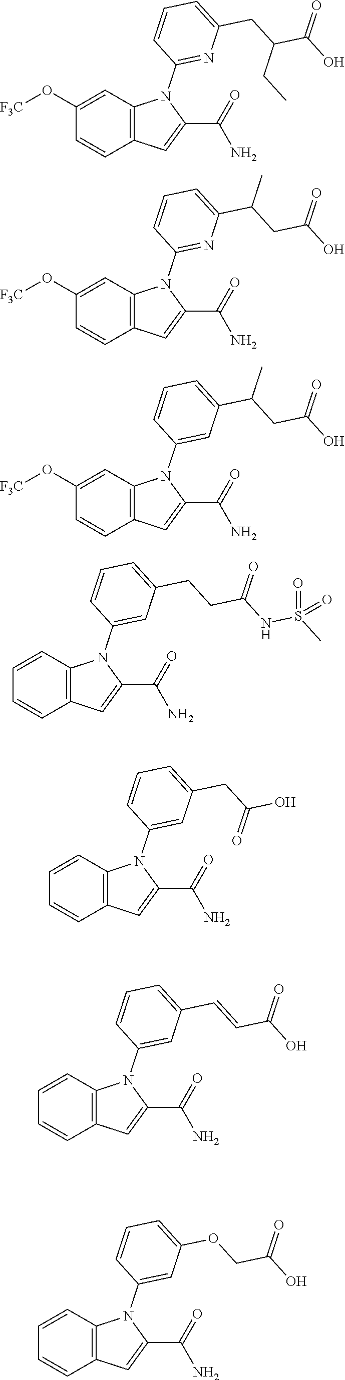

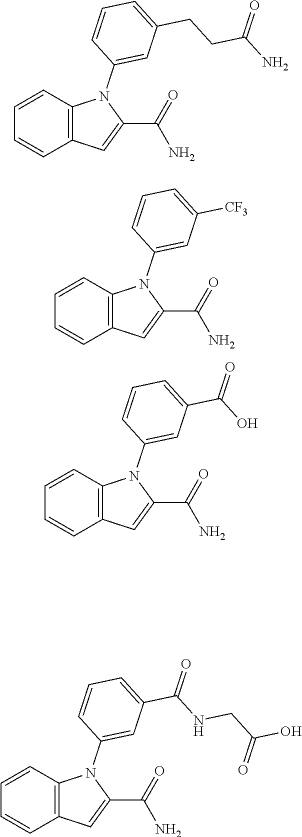

In one embodiment, at least three of Z₁-Z₄ are CH.

In a further embodiment, each of Z₁-Z₄ are CH.

In one embodiment, each R₆ is independently selected from halo, C₁₋₆ alkyl, C₁₋₆ haloalkyl, —OC₁₋₆ alkyl, and —OC₁₋₆ haloalkyl.

In another embodiment, each R₆ is independently selected from —OC₁₋₆ alkyl, and —OC₁₋₆ haloalkyl.

In a further embodiment, each R₆ is —OC₁₋₆ haloalkyl.

In one embodiment, m is an integer selected from 1, 2, and 3.

In another embodiment, m is an integer selected from 1, and 2.

In a further embodiment, m is 1.

In one embodiment, X′ is O, S, Se, or NR₉, wherein R₉ is selected from H, C₁₋₆ alkyl, optionally substituted with one or more R₁₀ substituents.

In another embodiment, X′ is O.

In another embodiment, X′ is S or Se.

In another embodiment, X′ is NR₉, wherein R₉ is selected from H, methyl, ethyl, or isopropyl.

In one embodiment, two of X₁-X₃ are CH, and one of X₁-X₃ is N.

In another embodiment, two of X₁ and X₂ are CH, and X₃ is N.

In a further embodiment, X₁-X₃ are all CH.

In one embodiment, X′ is NH.

In another embodiment, A is —O—, S(O)₂, or —S—.

In another embodiment, A is C(R₂)R₃ and R₂ and R₃ are both hydrogen.

In one embodiment, A is C(R₂)R₃ and R₂ and R₃ are each independently selected from CN, OH, NH₂, halo, and C₁₋₆ alkyl, wherein each alkyl is independently and optionally substituted with one or more R₁₀ substituents.

In another embodiment, R₂ and R₃ are the same and selected from halo and C₁₋₆ alkyl.

In a further embodiment, R₂ and R₃ are both chloro, fluoro, methyl, or ethyl.

In yet a further embodiment, R₂ and R₃ are both fluoro or both methyl.

In one embodiment, R₂ is hydrogen, and R₃ is NH₂.

In one embodiment, A is C(R₂)R₃ and R₂ and R₃, together with the carbon atom to which they are attached, form a 3-6 membered cyclyl or a 3-6 membered heterocyclyl, wherein each cyclyl, and heterocyclyl is independently and optionally substituted with one or more R₁₀ substituents.

In another embodiment, R₂ and R₃, together with the carbon atom to which they are attached, form a 3-6 membered cyclyl, independently and optionally substituted with one or more R₁₀ substituents.

In a further embodiment, R₂ and R₃, together with the carbon atom to which they are attached, form a cyclopropyl or cyclobutyl ring.

In one embodiment, R₁ is H.

In one embodiment, R₄ and R₅ are both hydrogen.

In another embodiment, R₄ and R₅ are each independently selected from hydrogen, CN, OH, NH₂, halo, and C₁₋₆ alkyl, wherein each alkyl is independently and optionally substituted with one or more R₁₀ substituents.

In a further embodiment, R₄ and R₅ are the same and selected from halo, NH₂, and C₁₋₆ alkyl.

In yet a further embodiment, R₄ and R₅ are both chloro, fluoro, methyl, or ethyl.

In one embodiment, R₄ and R₅ are both fluoro or both methyl.

In one embodiment, one of R₄ and R₅ is hydrogen and the other of R₄ and R₅ is NH₂.

In another embodiment, R₄ and R₅, together with the carbon atom to which they are attached, form an oxo, a 3-6 membered cyclyl or a 3-6 membered heterocyclyl, wherein each cyclyl and heterocyclyl are optionally substituted with one or more R₁₀ substituents.

In one embodiment, R₄ and R₅, together with the carbon atom to which they are attached, form a 3-6 membered cyclyl or a 3-6 membered heterocyclyl, wherein each cyclyl and heterocyclyl are optionally substituted with one or more R₁₀ substituents.

In another embodiment, R₄ and R₅, together with the carbon atom to which they are attached, form a 3-6 membered cyclyl.

In a further embodiment, R₄ and R₅, together with the carbon atom to which they are attached, form a cyclopropyl or a cyclobutyl.

In one embodiment, A is C(R₂)R₃ and R₃ and R₄ form a double bond, or, together with the carbon atom to which they are attached, form a 3-6 membered cyclyl or a 3-6 membered heterocyclyl, wherein each cyclyl and heterocyclyl are optionally substituted with one or more R₁₀ substituents.

In one embodiment, R₃ and R₄, together with the carbon atom to which they are attached, form a 3-6 membered cyclyl or a 3-6 membered heterocyclyl, wherein each cyclyl and heterocyclyl are optionally substituted with one or more R₁₀ substituents.

In another embodiment, R₃ and R₄, together with the carbon atom to which they are attached, form a 3-6 membered cyclyl, optionally substituted with one or more C₁₋₆ alkyl substituents.

In a further embodiment, R₃ and R₄, together with the carbon atom to which they are attached, form a cyclopropyl or a cyclobutyl, optionally substituted with one or more methyl groups.

In one embodiment, Ring E is a 5-6 membered fused cyclic moiety selected from heterocyclyl and heteroaryl, each of which is optionally and independently substituted with one to three R₁₀ substituents.

In one embodiment, Ring E is a 5 membered fused cyclic moiety selected from heterocyclyl and heteroaryl, each of which is optionally and independently substituted with one to three substituents selected from C(O)R₁₁, C(O)OR₁₁, C(O)N(R₁₁)₂, (C₁₋₆ alkyl)-C(O)R₁₁, (C₁₋₆ alkyl)-C(O)OR₁₁, and (C₁₋₆ alkyl)-C(O)N(R₁₁)₂, and wherein R₁₁ is hydrogen or C₁₋₆ alkyl.

In a further embodiment, Ring E is selected from

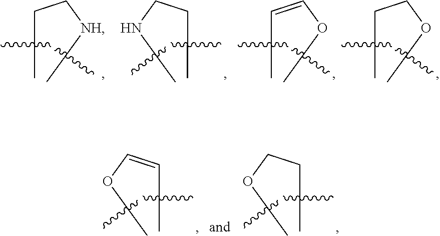 [SMILES: *CCN* ; *CCN* ; */C=C\O* ; *CCO* ; */C=C\O* ; *CCO*]

each of which is optionally and independently substituted with one to three substituents selected from C(O)OH and (C₁₋₆ alkyl)-C(O)OH.

In one embodiment, L is a bivalent moiety selected from methylene, ethylene, cyclopropylene, cyclobutylene, aziridine, and azetidine, each of which is optionally and independently substituted with one to three R₁₁ substituents.

In a further embodiment, L is methylene,

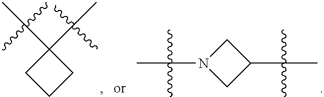 [SMILES: *C1(*)CCC1 ; *C1CN(*)C1]

In one embodiment, Z is selected from CN, OR₁₁, N(R₁₁)₂, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, and S(O)₂N(R₁₁)₂, and wherein R₁₁ is selected from hydrogen and C₁₋₆ alkyl.

In another embodiment, Z is selected from OR₁₁, N(R₁₁)₂, N(R₁₁)C(O)R₁₁, N(R₁₁)C(O)OR₁₁, N(R₁₁)—(CH₂)—C(O)OR₁₁, and OSO₂C₁₋₆ alkyl.

In a further embodiment, Z is NHC(O)O-tert-butyl, NHCH₂C(O)OH, NHC(O)CH₃, NHC(O)CF₃, NHS(O)₂CH₃, NH₂, OS(O)₂CH₃, and OH.

In one embodiment, each R₇ is independently selected from CN, OC₁₋₆ alkyl, halo, and C₁₋₆ alkyl.

In another embodiment, each R₇ is independently selected from halo and C₁₋₆ alkyl.

In one embodiment, n is 0 or 1.

In a further embodiment, n is 0.

In one embodiment, Rs is NH₂, NHCH₃, or OH.

In one embodiment, R₁₂ is selected from OH, OC₁₋₆ alkyl, O-phenyl, NH₂, NH(C₁₋₆ alkyl), and N(C₁₋₆ alkyl)₂, wherein each alkyl and phenyl are optionally and independently substituted with one to three R₁₀ substituents.

In another embodiment, R₁₂ is selected from OH, OC₁₋₆ alkyl, and O-phenyl, wherein each alkyl and phenyl are optionally and independently substituted with one to three R₁₀ substituents.

In a further embodiment, R₁₂ is selected from OH, OC₁₋₆ alkyl, and O-phenyl, wherein each alkyl and phenyl are optionally and independently substituted with one to three R₁₀ substituents.

In one embodiment, R₁₂ is O-Me, O-Et, O-Pr, O-i-Pr, O-tert-Bu, O-phenyl, or OH.

In one embodiment, R₁₂ is OCH₃ or OH.

In one aspect, the compound is a compound of Formula II.

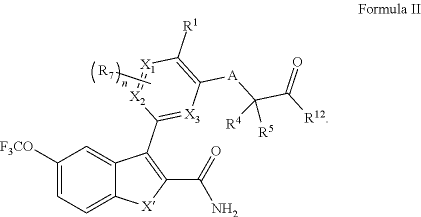

In one embodiment of this aspect, the chemical moiety,

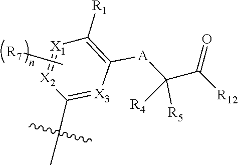

is selected from the group consisting of

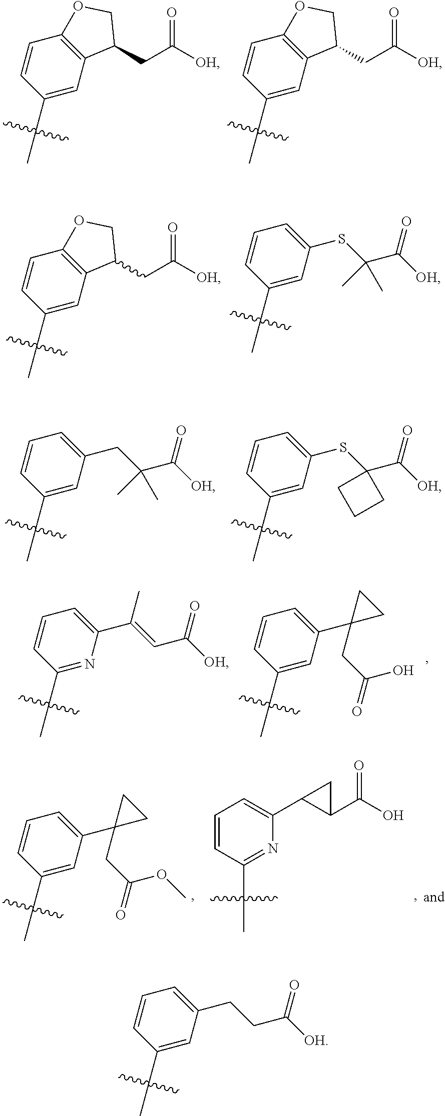

In one embodiment, the compound is a compound of Formula IIIa:

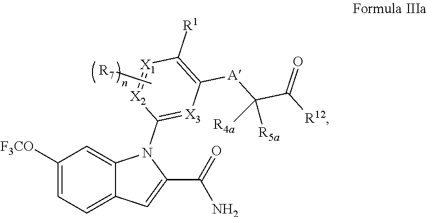

wherein,
A′ is —O— or —S—;
R₁ is H or R₇;
R₄ₐ and R₅ₐ are each independently selected from hydrogen, CN, OH, NH₂, halo, and C₁₋₆ alkyl, wherein each alkyl is independently and optionally substituted with one or more R₁₀ substituents; and
or, R₄ₐ and R₅ₐ, together with the carbon atom to which they are attached, form an oxo, a 3-6 membered cyclyl or a 3-6 membered heterocyclyl, wherein each cyclyl and heterocyclyl are optionally substituted with one or more R₁₀ substituents.

In one embodiment, A′ is —O—.

In another embodiment, A′ is —S— or —S(O)₂—.

In another embodiment, R₄ₐ and R₅ₐ are each independently selected from hydrogen and C₁₋₆ alkyl, wherein each alkyl is independently and optionally substituted with one or more R₁₀ substituents.

In a further embodiment, R₄ₐ and R₅ₐ are both hydrogen.

In one embodiment, R₄ₐ and R₅ₐ are selected from hydrogen, methyl, ethyl, propyl, isopropyl, and tert-butyl.

In a further embodiment, R₄ₐ and R₅ₐ are both methyl.

In one embodiment, R₄ₐ and R₅ₐ, together with the carbon atom to which they are attached, form an oxo, a 3-6 membered cyclyl or a 3-6 membered heterocyclyl, wherein each cyclyl and heterocyclyl are optionally substituted with one or more R₁₀ substituents.

In another embodiment, R₄ₐ and R₅ₐ, together with the carbon atom to which they are attached, form a 3-6 membered cyclyl or a 3-6 membered heterocyclyl, wherein each cyclyl and heterocyclyl are optionally substituted with one or more R₁₀ substituents.

In a further embodiment, R₄ₐ and R₅ₐ, together with the carbon atom to which they are attached, form a cyclopropyl or cyclobutyl.

In one embodiment, the chemical moiety,

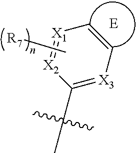

is selected from the group consisting of

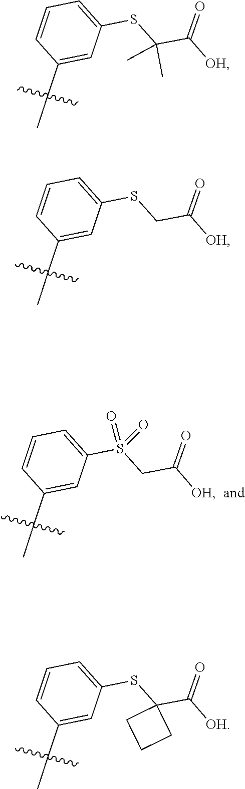

In another aspect, the compound is a compound of Formula IIIb:

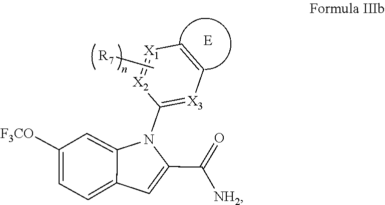

wherein,
Ring E is a 5-6 membered fused cyclic moiety selected from cyclyl, heterocyclyl, and heteroaryl, each of which is optionally and independently substituted with one to three R₁₀ substituents, wherein
each R₁₀ is selected from CN, OR₁, N(R₁)₂, halo, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, and SO₂C₁₋₆ alkyl, S(O)₂N(R₁₁)₂; and
each R₁₁ is independently selected from hydrogen, OH, NH₂, OC₁₋₆ alkyl, OC₁₋₆ haloalkyl, C₁₋₆ alkyl, and C₁₋₆ haloalkyl.

In one embodiment, Ring E is a 5 membered fused cyclic moiety selected from heterocyclyl and heteroaryl, each of which is optionally and independently substituted with one to three substituents selected from C(O)R₁₁, C(O)OR₁₁, C(O)N(R₁₁)₂, (C₁₋₆ alkyl)-C(O)R₁₁, (C₁₋₆ alkyl)-C(O)OR₁₁, and (C₁₋₆ alkyl)-C(O)N(R₁₁)₂, and wherein R₁₁ is hydrogen or C₁₋₆ alkyl.

In a further embodiment, Ring E is selected from

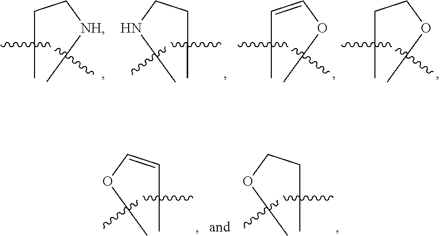 [SMILES: *CCN* ; *CCN* ; */C=C\O* ; *CCO* ; */C=C\O* ; *CCO*]

each of which is optionally and independently substituted with one to three substituents selected from C(O)OH and (C₁₋₆ alkyl)-C(O)OH.

In one embodiment, the chemical moiety,

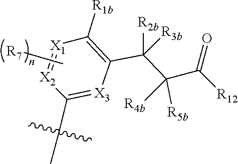

is selected from the group consisting of

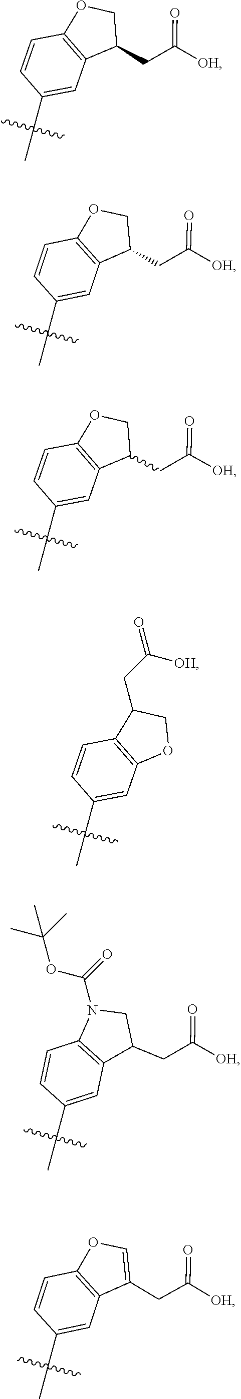 [SMILES: *c1ccc2c(c1)[C@H](CC(=O)O)CO2 ; *c1ccc2c(c1)[C@@H](CC(=O)O)CO2 ; *c1ccc2c(c1)C(CC(=O)O)CO2 ; *c1ccc2c(c1)OCC2CC(=O)O ; *c1ccc2c(c1)C(CC(=O)O)CN2C(=O)OC(C)(C)C ; *c1ccc2occ(CC(=O)O)c2c1]

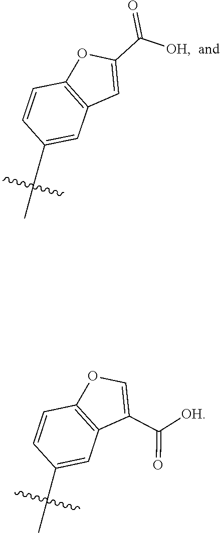

In one aspect, the compound is a compound of Formula IIIc:

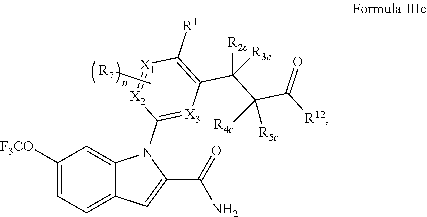

wherein,
R_{3c} and R_{4c} form a double bond;
R_{2c} is selected from hydrogen CN, OH, NH₂, halo, and C₁₋₆ alkyl, wherein each alkyl is independently and optionally substituted with one or more R₁₀ substituents;
   or R₁ and R_{2c}, together with the atoms to which they are attached, form a phenyl, a 5-6 membered cyclyl, a 5-6 membered heterocyclyl, or a 5 or 6 membered heteroaryl, each optionally substituted with one or more R₁₀ substituents; and R_{5c} is selected from hydrogen, CN, OH, NH₂, halo, and C₁₋₆ alkyl, wherein each alkyl is independently and optionally substituted with one or more R₁₀ substituents.

In one embodiment of this aspect, R_{2c} is selected from hydrogen, methyl, ethyl, and isopropyl.

In another embodiment, R_{2c} is methyl.

In a further embodiment, R_{5c} is hydrogen or methyl.

In one embodiment, the chemical moiety,

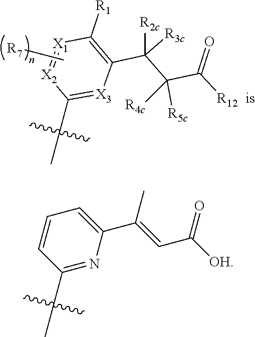

In another aspect, the compound is a compound of Formula IIId:

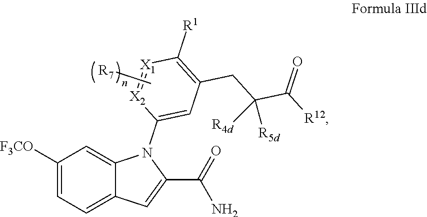

wherein,
R_{4d} and R_{5d} are each independently selected from hydrogen, CN, OH, NH₂, halo, and C₁₋₆ alkyl, wherein each alkyl is independently and optionally substituted with one or more R₁₀ substituents, and wherein R_{4d} and R_{5d} are not both hydrogen;
   or, R_{4d} and R_{5d}, together with the carbon atom to which they are attached, form an oxo, a 3-6 membered cyclyl or a 3-6 membered heterocyclyl, wherein each cyclyl and heterocyclyl are optionally substituted with one or more R₁₀ substituents.

In one embodiment, one of R_{4d} and R_{5d} is hydrogen and the other is NH₂.

In another embodiment, R_{4d} and R_{5d} are each independently selected from hydrogen, halo, and C₁₋₆ alkyl.

In a further embodiment, R_{4d} and R_{5d} are each independently selected from methyl, ethyl, and isopropyl.

In yet a further embodiment, R_{4d} and R_{5d} are both methyl.

In one embodiment, R_{4d} and R_{5d}, together with the carbon atom to which they are attached, form a 3-6 membered cyclyl or a 3-6 membered heterocyclyl, wherein each cyclyl and heterocyclyl are optionally substituted with one or more R₁₀ substituents.

In another embodiment, R_{4d} and R_{5d}, together with the carbon atom to which they are attached, form a 3-6 membered cyclyl.

In a further embodiment, R_{4d} and R_{5d}, together with the carbon atom to which they are attached, form cyclopropyl or cyclobutyl.

In one embodiment, the chemical moiety,

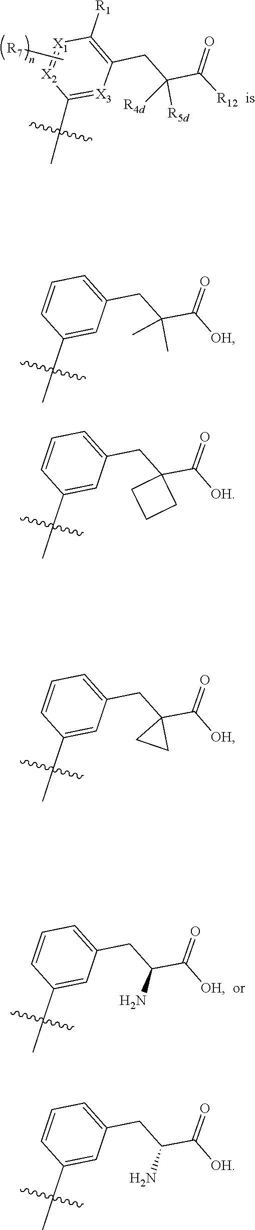

In another aspect, the compound is a compound of Formula IIIe:

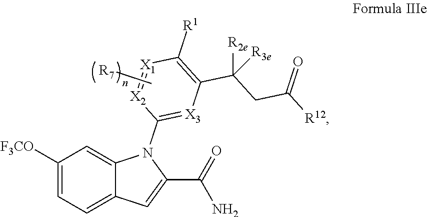

wherein,
R₂ₑ and R₃ₑ are each independently selected from hydrogen CN, OH, NH₂, halo, and C₁₋₆ alkyl, or R₂ₑ and R₃ₑ, together with the carbon atom to which they are attached, form a 3-6 membered cyclyl or a 3-6 membered heterocyclyl, wherein each alkyl, cyclyl, and heterocyclyl is independently and optionally substituted with one or more R₁₀ substituents, and wherein R₂ₑ and R₃ₑ are not both hydrogen.

In one embodiment of this aspect, R₂ₑ and R₃ₑ are each independently selected from hydrogen, NH₂, halo, and C₁₋₆ alkyl.

In one embodiment, R₂ₑ and R₃ₑ are each independently selected from methyl, ethyl, and isopropyl.

In one embodiment, R₂ₑ and R₃ₑ are both methyl.

In a further embodiment, R₂ₑ and R₃ₑ are both halo.

In one embodiment, R₂ₑ and R₃ₑ, together with the carbon atom to which they are attached, form a 3-6 membered cyclyl or a 3-6 membered heterocyclyl.

In a further embodiment, R₂ₑ and R₃ₑ, together with the carbon atom to which they are attached, form a 3-6 membered cyclyl.

In another embodiment, the moiety

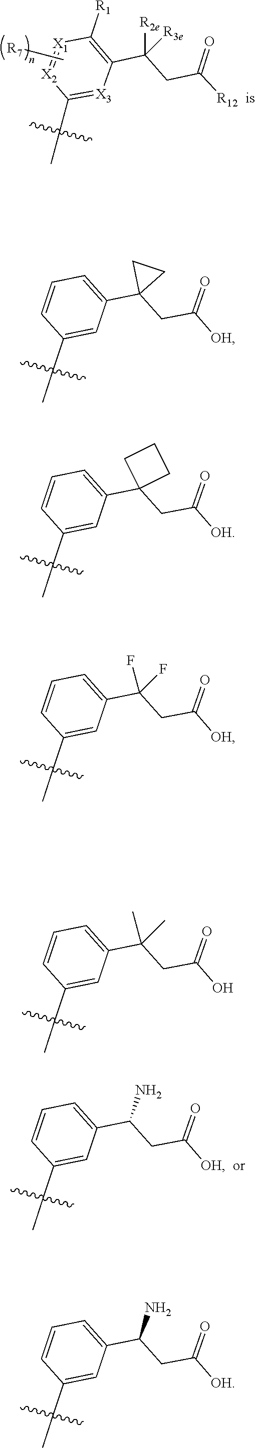

In another aspect, the compound is a compound of Formula IIIf-1-IIIf-7:

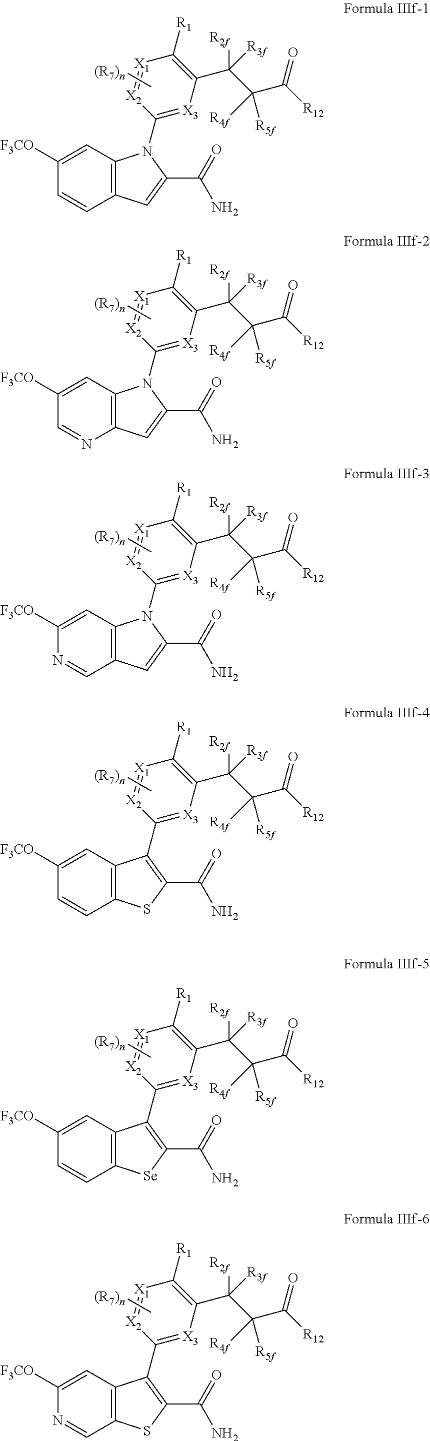

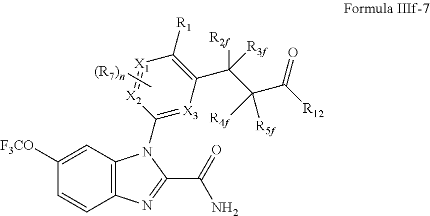

wherein,
R_{2f} is selected from hydrogen CN, OH, NH₂, halo, and C₁₋₆ alkyl;
R_{5f} is selected from hydrogen, CN, OH, NH₂, halo, C₁₋₆ alkyl, and OC₁₋₆ alkyl; and
R_{3f} and R_{4f}, together with the carbon atom to which they are attached, form a 3-6 membered cyclyl or a 3-6 membered heterocyclyl, wherein each cyclyl and heterocyclyl are optionally substituted with one or more R₁₀ substituents.

In one embodiment, R_{3f} and R_{4f}, together with the carbon atom to which they are attached, form a 3-6 membered cyclyl optionally substituted with one or more C₁₋₆ alkyl substituents.

In another embodiment, R_{3f} and R_{4f}, together with the carbon atom to which they are attached, form a cyclopropyl or cyclobutyl moiety, which is optionally substituted with one or more C₁₋₆ alkyl substituents.

In another embodiment, the moiety

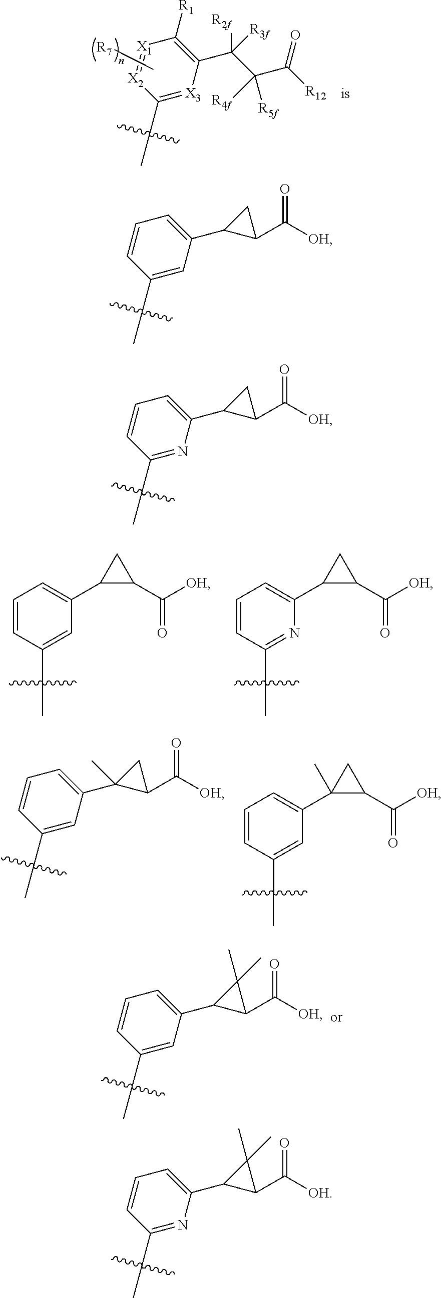

In one embodiment, the compound of Formula I is a compound of Formula IV

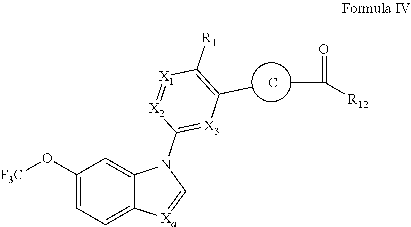

wherein
X₁, X₂, X₃, R₁, R₁₀, and R₁₂ are defined herein;
Xₐ is N or CH; and
Ring C is a phenyl, 3-6 membered cyclyl, 3-6 membered heterocyclyl, or five or six membered heteroaryl, which is bivalent and optionally substituted with one or more Rio substituents.

In one embodiment, the compound of Formula IV is a compound of Formula IVa

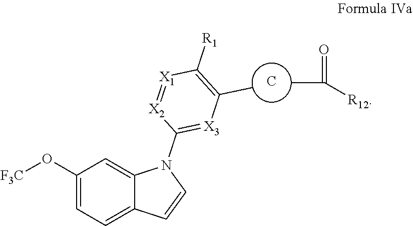

In one embodiment, the compound of Formula IV is a compound of Formula IVb

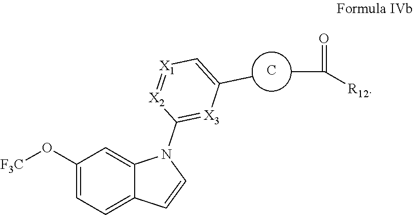

In one embodiment, the compound of Formula IV is a compound of Formula IVc

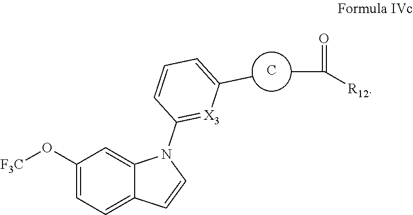

In some embodiments of Formulas IV, IVa, IVb, or IVc, each R₁₂ is independently selected from OH, OC₁₋₆ alkyl, NH₂, NH(C₁₋₆ alkyl), and N(C₁₋₆ alkyl)₂. In another embodiment of Formulas IV, IVa, IVb, or IVc, each R₁₂ is selected from OH or NH₂. In a further embodiment of Formulas IV, IVa, IVb, or IVc, each R₁₂ is OH.

In some embodiments of Formulas IV and IVa, R₁ is H or R₁₀;

In some embodiments of Formulas IV, IVa, IVb, or IVc, each R₁₀ is selected from oxo, CN, OR₁₁, N(R₁₁)₂, halo, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, S(O)₂N(R₁₁)₂, C₁₋₆ alkyl, a phenyl, a 3-6 membered cyclyl, a 3-6 membered heterocyclyl, or a 5 or 6 membered heteroaryl, wherein R₁₁ is defined herein.

In some embodiments of Formulas IV, IVa, IVb, or IVc, each R₁₀ is selected from oxo, CN, OR₁₁, N(R₁₁)₂, halo, C(O)H, C(O)R₁₁, C(O)OR₁₁, C(O)N(R₁₁)₂, C₁₋₆ alkyl, a phenyl, a 3-6 membered cyclyl, a 3-6 membered heterocyclyl, or a 5 or 6 membered heteroaryl.

In some embodiments of Formulas IV, IVa, IVb, or IVc, each R₁₀ is selected from CN, OR₁₁, N(R₁₁)₂, halo, and C₁₋₆ alkyl.

In some embodiments of Formulas IV, IVa, IVb, or IVc, each R₁₀ is selected from halo and C₁₋₆ alkyl.

In some embodiments of Formulas IV, IVa, IVb, or IVc, each Ring C is a phenyl, 3-6 membered cyclyl, 3-6 membered heterocyclyl, or five or six membered heteroaryl, which is bivalent and optionally substituted with one or more R₁₀ substituents. In other embodiments of Formulas IV, IVa, IVb, or IVc, each Ring C is an optionally substituted 3-6 membered cyclyl or an optionally substituted 3-6 membered heterocyclyl. In a further embodiment of Formulas IV, IVa, IVb, or IVc, each Ring C is an optionally substituted 3-6 membered cyclyl. In still a further embodiment of Formulas IV, IVa, IVb, or IVc, each Ring C is an optionally substituted cyclopropyl. In some embodiments of Formulas IV, IVa, IVb, or IVc, Ring C is unsubstituted.

In one embodiment, the compound of Formula I is a compound of Formula V

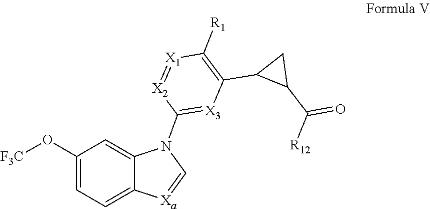

wherein
X₁, X₂, X₃, R₁, and R₁₂ are defined herein; and
Xₐ is N or CH.

In one embodiment, the compound of Formula V is a compound of Formula Va

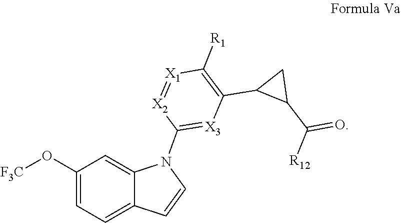

In one embodiment, the compound of Formula V is a compound of Formula Vb

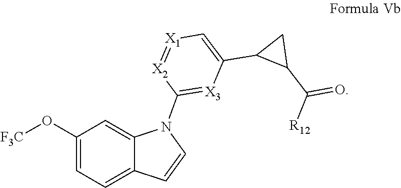

In one embodiment, the compound of Formula V is a compound of Formula Vc

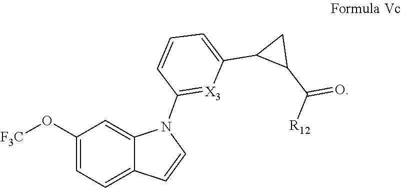

In some embodiments of Formulas V, Va, Vb, or Vc, each R₁₂ is independently selected from OH, OC₁₋₆ alkyl, NH₂, NH(C₁₋₆ alkyl), and N(C₁₋₆ alkyl)₂. In another embodiment of Formulas V, Va, Vb, or Vc, each R₁₂ is selected from OH or NH₂. In a further embodiment of Formulas V, Va, Vb, or Vc, each R₁₂ is OH.

In some embodiments of Formulas V and Va, R₁ is H or R₁₀;

In some embodiments of Formulas V and Va, each R₁₀ is selected from oxo, CN, OR₁₁, N(R₁₁)₂, halo, C(O)H, C(O)R₁₁, C(O)OR₁₁, C(O)N(R₁₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(R₁₁)—(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, S(O)₂N(R₁₁)₂, C₁₋₆ alkyl, a phenyl, a 3-6 membered cyclyl, a 3-6 membered heterocyclyl, or a 5 or 6 membered heteroaryl, wherein R₁₁ is defined herein.

In some embodiments of Formulas V and Va, each R₁₀ is selected from oxo, CN, OR₁₁, N(R₁₁)₂, halo, C(O)H, C(O)R₁₁, C(O)OR₁₁, C(O)N(R₁₁)₂, C₁₋₆ alkyl, a phenyl, a 3-6 membered cyclyl, a 3-6 membered heterocyclyl, or a 5 or 6 membered heteroaryl.

In some embodiments of Formulas V and Va, each R₁₀ is selected from CN, OR₁₁, N(R₁₁)₂, halo, and C₁₋₆ alkyl.

In some embodiments of Formulas V and Va, each R₁₀ is selected from halo and C₁₋₆ alkyl.

In another aspect, the invention includes a process for producing Compound 28a

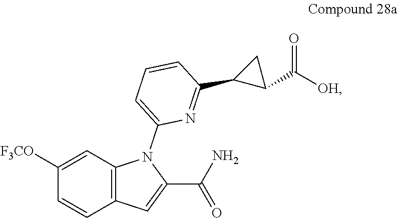 [SMILES: NC(=O)c1cc2ccc(OC(F)(F)F)cc2n1-c1cccc([C@H]2C[C@@H]2C(=O)O)n1]

the process comprising contacting a compound of Formula X

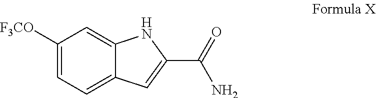 [SMILES: NC(=O)c1cc2ccc(OC(F)(F)F)cc2[nH]1]

with a compound of Formula XI

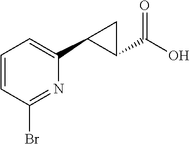 [SMILES: O=C(O)[C@H]1C[C@@H]1c1cccc(Br)n1]

in the presence of a copper catalyst, a solvent, and a base to provide Compound 28a.

In one embodiment of this aspect, the copper catalyst is a copper I catalyst. In a further embodiment, the copper catalyst is a copper halide. In still a further embodiment, the copper catalyst is copper (I) iodide.

In another embodiment, the base is an organic base. In a further embodiment the organic base is selected from trimethylamine, DBU, DIEA, and sodium hydride. In a further embodiment, the base is DBU.

In another embodiment, the solvent is selected from DMSO, dimethylacetamide, dimethylformamide, and THF. In a further embodiment, the solvent is DMSO.

In another aspect, the process further comprises contacting a compound of Formula XII

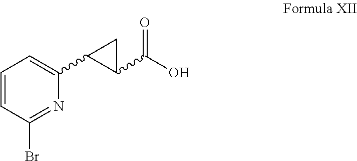 [SMILES: O=C(O)C1CC1c1cccc(Br)n1]

with (R)-1-phenylethanylamine,

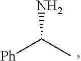

under peptide coupling conditions, and then recrystallized to provide a compound of Formula XIII

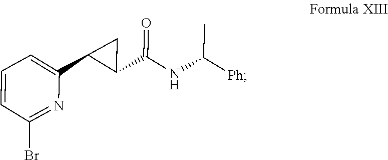 [SMILES: C[C@@H](NC(=O)[C@H]1C[C@@H]1c1cccc(Br)n1)c1ccccc1]

and
hydrolyzing the compound of Formula XIII with an aqueous acid to provide the compound of Formula XI.

In one embodiment of this aspect, the peptide coupling conditions include DMAP and EDC-HCl in an organic solvent. In a further embodiment, the organic solvent is selected from dichloromethane, chloroform, THF, DMF, ethyl acetate, hexane, heptane, or a combination thereof. In a further embodiment, the solvent is dichloromethane.

In another embodiment, the compound of Formula III is recrystallized from a solvent selected from water, dichloromethane, chloroform, THF, DMF, ethyl acetate, hexane, heptane, or a combination thereof. In a further embodiment, the compound of Formula III is recrystallized from a solvent mixture comprising heptane and ethyl acetate. In still a further embodiment, the compound of Formula XIII is recrystallized from a solvent mixture comprising about 70% heptane and about 30% ethyl acetate.

In one embodiment, the compound of Formula XIII is hydrolyzed by contacting the crystalline compound of Formula XIII to concentrated HCl. In a further embodiment, the mixture is stirred at increased temperature, e.g. about 80° C.

In another aspect, the process further comprises step 1) contacting a compound of Formula XIV

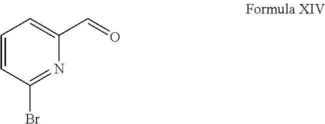

with triethyl phosphonium acetate in the presence of LiCl, a solvent, and a base to provide a compound of Formula XV

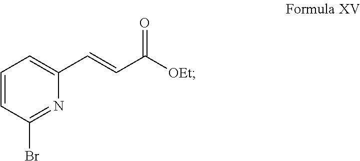 [SMILES: CCOC(=O)/C=C/c1cccc(Br)n1]

step 2) contacting the compound of Formula XV with trimethylsulfoxonium iodide in the presence of a base and a solvent to provide a compound of Formula XVI

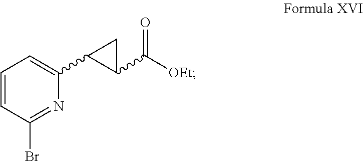 [SMILES: CCOC(=O)C1CC1c1cccc(Br)n1]

and
step 3) hydrolyzing the compound of Formula XVI to provide a compound of Formula XII.

In one embodiment of this aspect, the base in step 1) is an organic base. In another embodiment, the base in step 1) is selected from triethylamine and DIEA. In a further embodiment, the base in step 1) is trimethylamine.

In one embodiment of this aspect, the solvent in step 1) is a polar aprotic solvent. In another embodiment, the solvent in step 1) is selected from THF, DMF, and DMSO. In a further embodiment, the solvent in step 1) is THF.

In one embodiment of this aspect, the base in step 2) is an alkoxide base. In another embodiment, the base in step 2) is selected from methoxide and tert-butoxide. In a further embodiment, the base in step 2) is potassium tert-butoxide.

In one embodiment of this aspect, the solvent in step 2) is a polar aprotic solvent. In another embodiment, the solvent in step 2) is selected from THF, DMF, and DMSO. In a further embodiment, the solvent in step 2) is DMSO.

In another embodiment, the compound of Formula XVI is hydrolyzed using aqueous hydroxide. In a further embodiment, the hydrolysis in step 3) is performed in a solvent that is a mixture of DMSO and water. In still further embodiment, the hydrolysis in step 3) is performed in a solvent that is a mixture of from about 10% to about 20% DMSO.

In another aspect, the invention also includes a compound selected from:

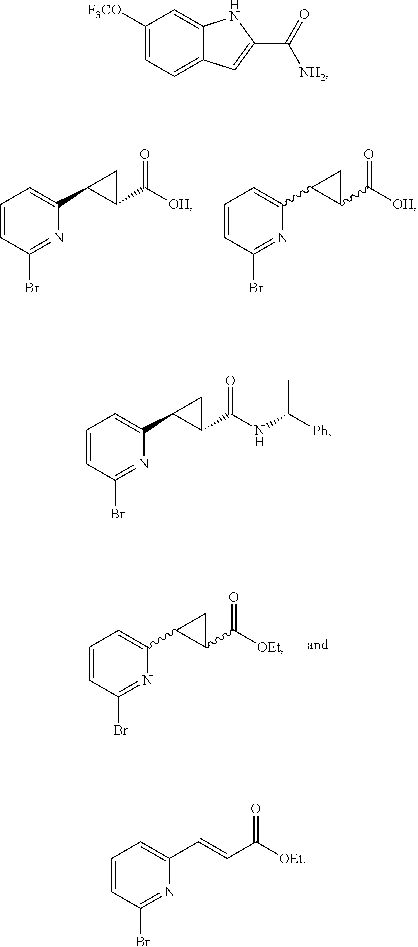 [SMILES: NC(=O)c1cc2ccc(OC(F)(F)F)cc2[nH]1 ; O=C(O)[C@H]1C[C@@H]1c1cccc(Br)n1 ; O=C(O)C1CC1c1cccc(Br)n1 ; C[C@@H](NC(=O)[C@H]1C[C@@H]1c1cccc(Br)n1)c1ccccc1 ; CCOC(=O)C1CC1c1cccc(Br)n1 ; CCOC(=O)/C=C/c1cccc(Br)n1]

In a further aspect, the invention includes the compound

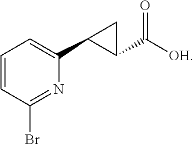 [SMILES: O=C(O)[C@H]1C[C@@H]1c1cccc(Br)n1]

In one embodiment, the compound is selected from the compounds listed in Table 1 below, or a pharmaceutically acceptable salt thereof.

TABLE 1

| Compound | Name | Structure |
|---|---|---|
| 1 | 2-(5-(2-carbamoyl-6- (trifluoromethoxy)-1H-indol-1-yl)-2,3- dihydrobenzofuran-3-yl)acetic acid | 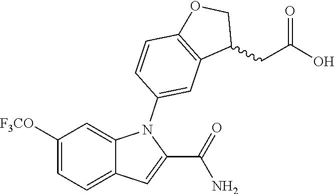 [SMILES: NC(=O)c1cc2ccc(OC(F)(F)F)cc2n1-c1ccc2c(c1)C(CC(=O)O)CO2] |
| 1a | 2-(5-(2-carbamoyl-6- (trifluoromethoxy)-1H-indol-1-yl)-2,3- dihydrobenzofuran-3-yl)acetic acid | 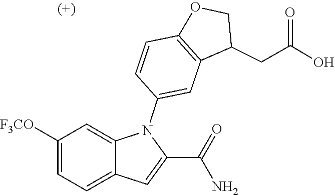 [SMILES: NC(=O)c1cc2ccc(OC(F)(F)F)cc2n1-c1ccc2c(c1)C(CC(=O)O)CO2] |
| 1b | 2-(5-(2-carbamoyl-6- (trifluoromethoxy)-1H-indol-1-yl)-2,3- dihydrobenzofuran-3-yl)acetic acid | 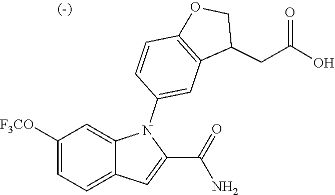 [SMILES: NC(=O)c1cc2ccc(OC(F)(F)F)cc2n1-c1ccc2c(c1)C(CC(=O)O)CO2] |
| 2 | 2-((3-(2-carbamoyl-6- (trifluoromethoxy)-1H-indol-1- yl)phenyl)thio)-2-methylpropanoic acid | 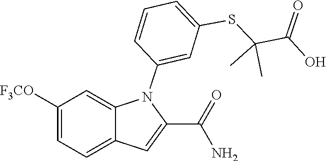 [SMILES: CC(C)(Sc1cccc(-n2c(C(N)=O)cc3ccc(OC(F)(F)F)cc32)c1)C(=O)O] |
| 3 | 3-(3-(2-carbamoyl-6- (trifluoromethoxy)-1H-indol-1- yl)phenyl)-2,2-dimethylpropanoic acid | 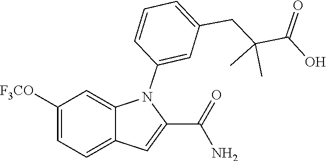 [SMILES: CC(C)(Cc1cccc(-n2c(C(N)=O)cc3ccc(OC(F)(F)F)cc32)c1)C(=O)O] |
| 4 | 1-((3-(2-carbamoyl-6- (trifluoromethoxy)-1H-indol-1- yl)phenyl)thio)cyclobutane-1- carboxylic acid | 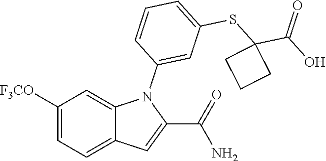 [SMILES: NC(=O)c1cc2ccc(OC(F)(F)F)cc2n1-c1cccc(SC2(C(=O)O)CCC2)c1] |
| 5 | 3-(3-(2-carbamoyl-5- (trifluoromethoxy)benzo[b] thiophen- 3-yl)phenyl)propanoic acid | 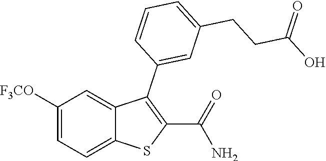 [SMILES: NC(=O)c1sc2ccc(OC(F)(F)F)cc2c1-c1cccc(CCC(=O)O)c1] |
| 6 | (E)-1-(6-(4-methoxy-4-oxobut-2-en-2- yl)pyridin-2-yl)-6-(trifluoromethoxy)- 1H-indole-2-carboxylic acid | 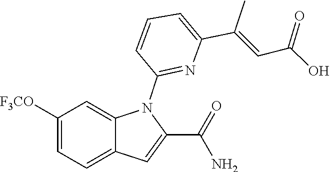 [SMILES: C/C(=C\C(=O)O)c1cccc(-n2c(C(N)=O)cc3ccc(OC(F)(F)F)cc32)n1] |
| 7 | 2-(5-(2-carbamoyl-5- (trifluoromethoxy)benzo[b] thiophen- 3-yl)-2,3-dihydrobenzofuran-3- yl)acetic acid | 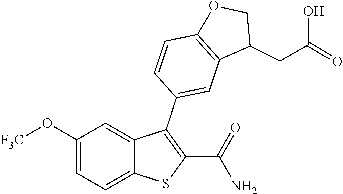 [SMILES: NC(=O)c1sc2ccc(OC(F)(F)F)cc2c1-c1ccc2c(c1)C(CC(=O)O)CO2] |
| 9 | 2-(6-(2-carbamoyl-6- (trifluoromethoxy)-1H-indol-1-yl)-2,3- dihydrobenzofuran-3-yl)acetic acid | 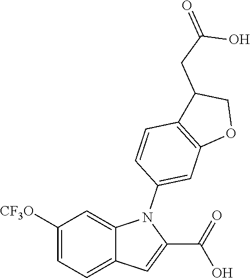 |
| 10 | 1-(3-(2-carbamoyl-6- (trifluoromethoxy)-1H-indol-1- yl)benzyl)cyclobutane-1-carboxylic acid | 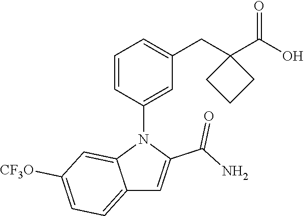 |
| 11 | 2-(1-(tert-butoxycarbonyl)-5-(2- carbamoyl-6-(trifluoromethoxy)-1H- indol-1-yl)indolin-3-yl)acetic acid | 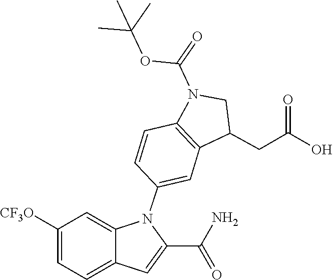 |
| 12 | 2-(5-(2-carbamoyl-6- (trifluoromethoxy)-1H-indol-1- yl)benzofuran-3-yl)acetic acid | 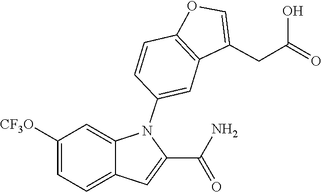 |
| 13 | 3-(3-(2-carbamoylbenzo[b] thiophen-3- yl)phenyl)propanoic acid | 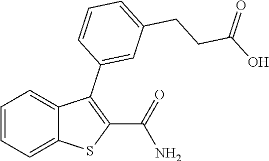 [SMILES: NC(=O)c1sc2ccccc2c1-c1cccc(CCC(=O)O)c1] |
| 14 | 3-(3-(2-(methylcarbamoyl)-6- (trifluoromethoxy)-1H-indol-1- yl)phenyl)propanoic acid | 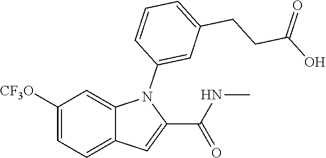 |
| 15 | trans-(rac)-2-(3-(2-carbamoyl-6- (trifluoromethoxy)-1H-indol-1- yl)phenyl)cyclopropane-1-carboxylic acid | 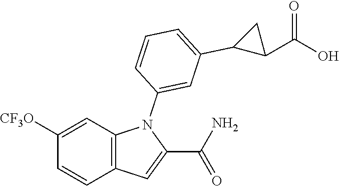 |
| 15a | trans-(+)-2-(3-(2-carbamoyl-6- (trifluoromethoxy)-1H-indol-1- yl)phenyl)cyclopropane-1-carboxylic acid | 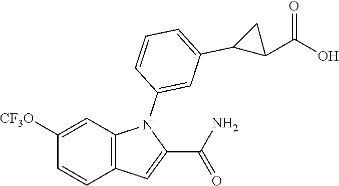 |
| 15b | trans-(−)-2-(3-(2-carbamoyl-6- (trifluoromethoxy)-1H-indol-1- yl)phenyl)cyclopropane-1-carboxylic acid | 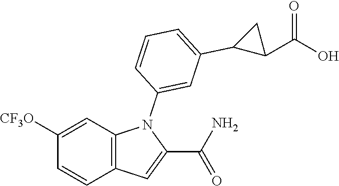 |
| 16 | 5-(2-carbamoyl-6-(trifluoromethoxy)- 1H-indol-1-yl)benzofuran-2-carboxylic acid | 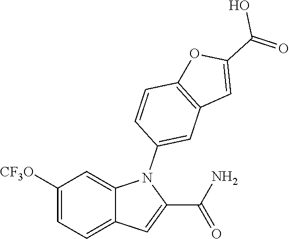 |
| 17 | 3-(3-(2-carbamoyl-5- (trifluoromethoxy)benzofuran-3- yl)phenyl)propanoic acid | 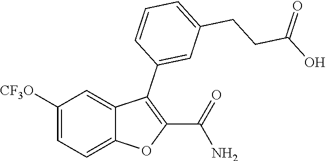 |
| 18 | 3-(6-(2-carbamoyl-6- (trifluoromethoxy)-1H-indol-1- yl)pyridin-2-yl)propanoic acid | 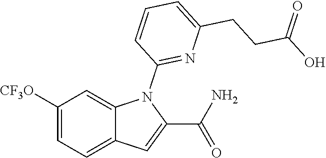 |
| 19 | 1-(3-(2-carbamoyl-6- (trifluoromethoxy)-1H-indol-1- yl)benzyl)cyclopropane-1-carboxylic acid | 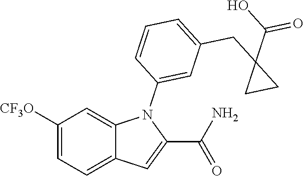 |
| 20 | 5-(2-carbamoyl-6-(trifluoromethoxy)- 1H-indol-1-yl)benzofuran-3-carboxylic acid | 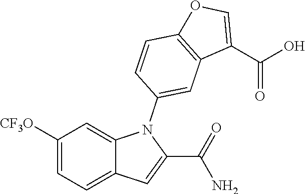 |
| 21 | 3-(3-(2-carbamoyl-1H-indol-1- yl)phenyl)propanoic acid | 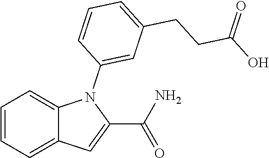 [SMILES: NC(=O)c1cc2ccccc2n1-c1cccc(CCC(=O)O)c1] |
| 22 | 3-(3-(2-carbamoyl-6- (trifluoromethoxy)-1H-indol-1- yl)phenyl)-3-methylbutanoic acid | 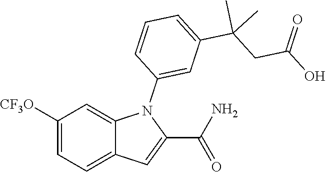 |
| 23 | (R)-2-amino-3-(3-(2-carbamoyl-6- (trifluoromethoxy)-1H-indol-1- yl)phenyl)propanoic acid TFA salt | 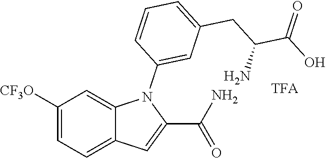 |
| 24 | (S)-2-amino-3-(3-(2-carbamoyl-6- (trifluoromethoxy)-1H-indol-1- yl)phenyl)propanoic acid TFA salt | 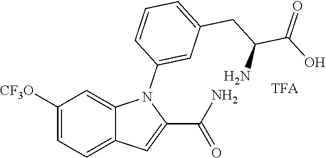 |
| 25 | 3-(3-(2-carbamoylbenzofuran-3- yl)phenyl)propanoic acid | 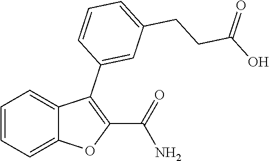 [SMILES: NC(=O)c1oc2ccccc2c1-c1cccc(CCC(=O)O)c1] |
| 26 | tert-butyl ((6-(2-carbamoyl-6- (trifluoromethoxy)-1H-indol-1- yl)pyridin-2-yl)methyl)carbamate | 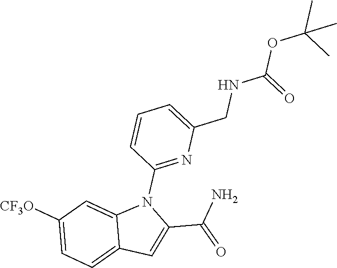 |
| 27 | 1-(6-(aminomethyl)pyridin-2-yl)-6- (trifluoromethoxy)-1H-indole-2- carboxamide 2,2,2-trifluoroacetate | 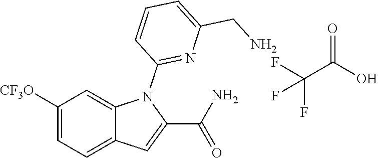 |
| 28 | trans-(rac)-2-(6-(2-carbamoyl-6- (trifluoromethoxy)-1H-indol-1- yl)pyridin-2-yl)cyclopropane-1- carboxylic acid | 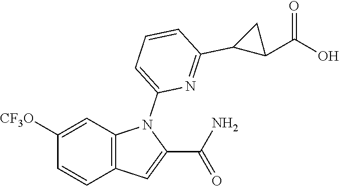 |
| 28a | trans-(−)-2-(6-(2-carbamoyl-6- (trifluoromethoxy)-1H-indol-1- yl)pyridin-2-yl)cyclopropane-1- carboxylic acid | 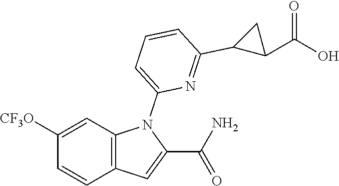 |
| 28b | trans-(+)-2-(6-(2-carbamoyl-6- (trifluoromethoxy)-1H-indol-1- yl)pyridin-2-yl)cyclopropane-1- carboxylic acid | 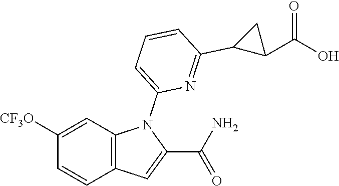 |
| 28c | cis-(rac)-2-(6-(2-carbamoyl-6- (trifluoromethoxy)-1H-indol-1- yl)pyridin-2-yl)cyclopropane-1- carboxylic acid | 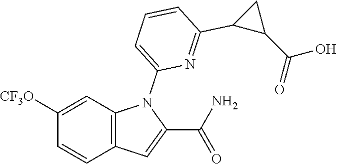 |
| 28d | cis-(−)-2-(6-(2-carbamoyl-6- (trifluoromethoxy)-1H-indol-1- yl)pyridin-2-yl)cyclopropane-1- carboxylic acid | 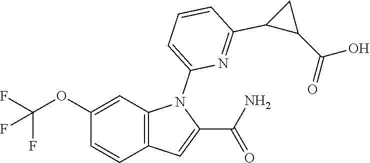 [SMILES: NC(=O)c1cc2ccc(OC(F)(F)F)cc2n1-c1cccc(C2CC2C(=O)O)n1] |
| 28e | cis-(+)-2-(6-(2-carbamoyl-6- (trifluoromethoxy)-1H-indol-1- yl)pyridin-2-yl)cyclopropane-1- carboxylic acid | 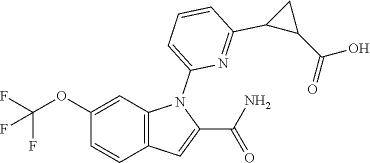 [SMILES: NC(=O)c1cc2ccc(OC(F)(F)F)cc2n1-c1cccc(C2CC2C(=O)O)n1] |
| 29 | 3-(3-(2-carbamoyl-5- (trifluoromethoxy)benzo[b]selenophen- 3-yl)phenyl)propanoic acid | 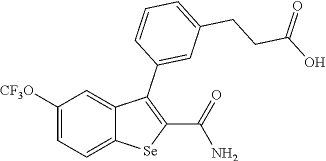 |
| 30 | 2-(1-(3-(2-carbamoyl-6- (trifluoromethoxy)-1H-indol-1- yl)phenyl)cyclobutyl)acetic acid | 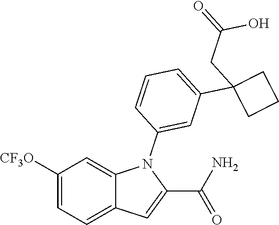 |
| 31 | 2-(1-(3-(2-carbamoyl-6- (trifluoromethoxy)-1H-indol-1- yl)phenyl)cyclopropyl)acetic acid | 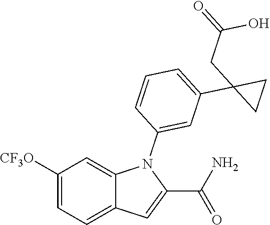 |
| 32 | 1-(6-(acetamidomethyl)pyridin-2-yl)-6- (trifluoromethoxy)-1H-indole-2- carboxamide | 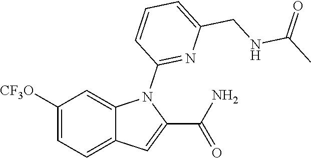 |
| 33 | 1-(6- (methylsulfonamidomethyl)pyridin-2- yl)-6-(trifluoromethoxy)-1H-indole-2- carboxamide | 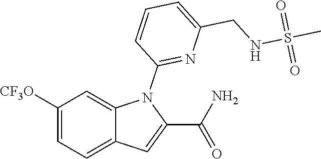 |
| 34 | 2-(1-(3-(2-carbamoyl-5- (trifluoromethoxy)benzo[ b] selenophen- 3-yl)phenyl)cyclopropyl)acetic acid | 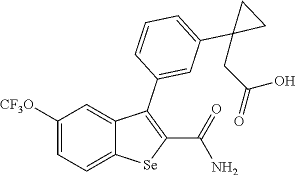 |
| 35 | methyl 2-(1-(3-(2-carbamoyl-5- (trifluoromethoxy)benzo[ b] thiophen-3- yl)phenyl)cyclopropyl)acetate | 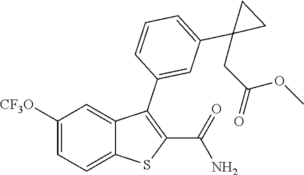 |
| 36 | 2-(1-(3-(2-carbamoyl-5- (trifluoromethoxy)benzo[ b] thiophen-3- yl)phenyl)cyclopropyl)acetic acid | 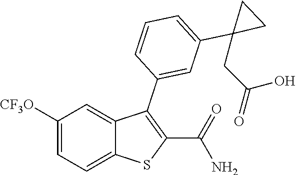 |
| 37 | 1-(6-((2,2,2- trifluoroacetamido)methyl)pyridin-2- yl)-6-(trifluoromethoxy)-1H-indole-2- carboxamide | 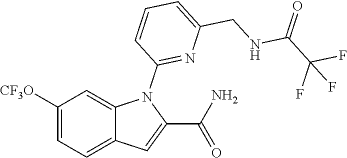 |
| 38a | cis-2-(3-(2-carbamoyl-6- (trifluoromethoxy)-1H-indol-1- yl)phenyl)-2-methylcyclopropane-1- carboxylic acid | 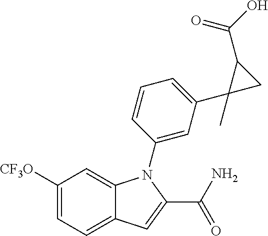 |
| 38b | trans-2-(3-(2-carbamoyl-6- (trifluoromethoxy)-1H-indol-1- yl)phenyl)-2-methylcyclopropane-1- carboxylic acid | 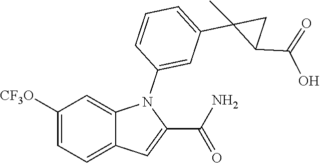 |
| 39 | 1-(6-(1-carboxycyclopropyl)pyridin-2- yl)-6-(trifluoromethoxy)-1H-indole-2- carboxylic acid | 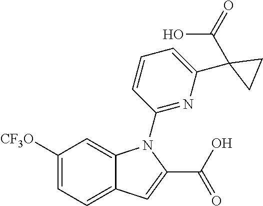 |
| 40 | (S)-3-amino-3-(3-(2-carbamoyl-6- (trifluoromethoxy)-1H-indol-1- yl)phenyl)propanoic acid | 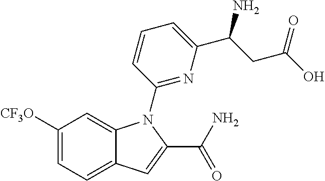 |
| 41 | (S)-1-(3-(1-amino-2- carboxyethyl)phenyl)-6- (trifluoromethoxy)-1H-indole-2- carboxylic acid | 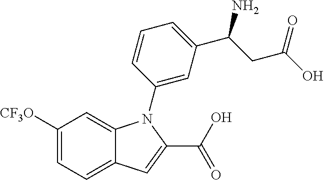 |
| 42 | (R)-3-amino-3-(3-(2-carbamoyl-6- (trifluoromethoxy)-1H-indol-1- yl)phenyl)propanoic acid | 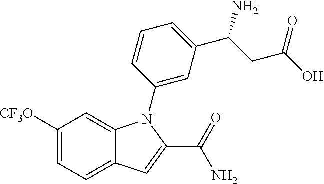 |
| 43 | (R)-1-(3-(1-amino-2- carboxyethyl)phenyl)-6- (trifluoromethoxy)-1H-indole-2- carboxylic acid | 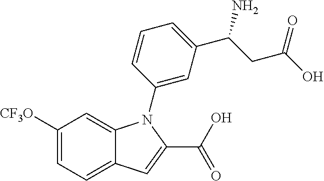 |
| 44 | 2-((6-(2-carbamoyl-6- (trifluoromethoxy)-1H-indol-1- yl)pyridin-2-yl)thio)acetic acid | 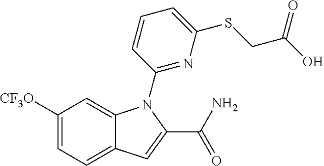 |
| 45 | 2-((6-(2-carbamoyl-6- (trifluoromethoxy)-1H-indol-1- yl)pyridin-2-yl)sulfonyl)acetic acid | 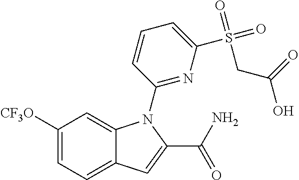 |
| 46 | 1-(3-(1,1-difluoro-2- hydroxyethyl)phenyl)-6- (trifluoromethoxy)-1H-indole-2- carboxamide | 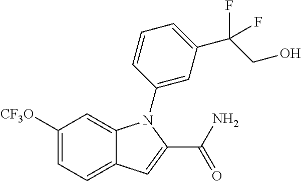 |
| 47 | 2-(3-(2-carbamoyl-6- (trifluoromethoxy)-1H-indol-1- yl)phenyl)-2,2-difluoroethyl methanesulfonate | 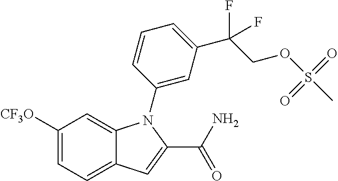 |
| 48 | tert-butyl (1-(6-(2-carbamoyl-6- (trifluoromethoxy)-1H-indol-1- yl)pyridin-2-yl)cyclobutyl)carbamate | 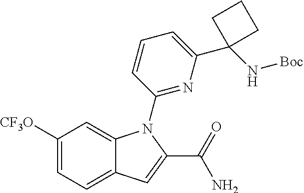 |
| 49 | 1-(6-(1-aminocyclobutyl)pyridin-2-yl)- 6-(trifluoromethoxy)-1H-indole-2- carboxamide TFA salt | 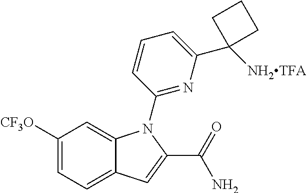 |
| 50 | (1-(6-(2-carbamoyl-6- (trifluoromethoxy)-1H-indol-1- yl)pyridin-2-yl)cyclobutyl)glycine | 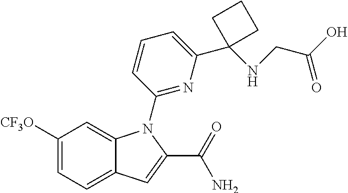 |
| 51 | trans-2-(6-(2-carbamoyl-6-methoxy- 1H-pyrrolo[3,2-b]pyridin-1-yl)pyridin- 2-yl)cyclopropane-1-carboxylic acid | 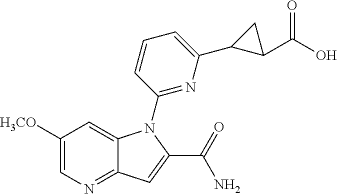 [SMILES: COc1cnc2cc(C(N)=O)n(-c3cccc(C4CC4C(=O)O)n3)c2c1] |
| 52 | 1-(6-(2-carbamoyl-6- (trifluoromethoxy)-1H-indol-1- yl)pyridin-2-yl)azetidine-3-carboxylic acid | 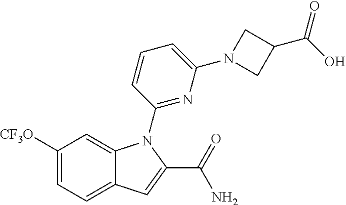 |
| 53 | 3-(5-(2-carbamoyl-6- (trifluoromethoxy)-1H-indol-1- yl)pyridin-3-yl)propanoic acid | 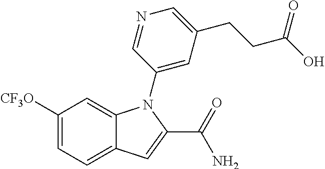 |
| 54 | trans-(rac)-2-(6-(2-carbamoyl-5- (trifluoromethoxy)benzo[b]thiophen-3- yl)pyridin-2-yl)cyclopropane-1- carboxylic acid | 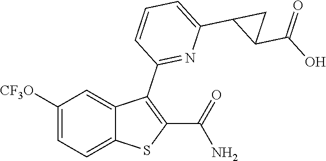 |
| 55 | trans-(rac)-2-(6-(2-carbamoyl-5- (trifluoromethoxy)benzo[b]selenophen- 3-yl)pyridin-2-yl)cyclopropane-1- carboxylic acid | 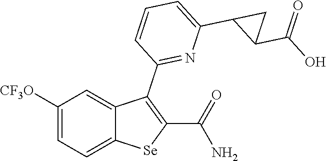 |
| 56 | trans-2-(6-(2-carbamoyl-5- (trifluoromethoxy)benzo[b]thiophen-3- yl)pyridin-2-yl)cyclopropane-1- carboxylic acid | 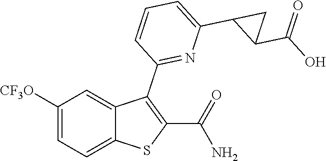 |
| 57 | trans-2-(6-(2-carbamoyl-5- (trifluoromethoxy)benzo[b]selenophen- 3-yl)pyridin-2-yl)cyclopropane-1- carboxylic acid | 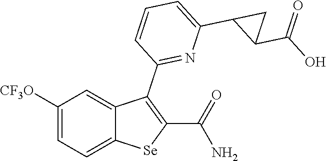 |
| 58 | trans-2-(6-(2-carbamoyl-6- (trifluoromethoxy)-1H- benzo[d]imidazol-1-yl)pyridin-2- yl)cyclopropane-1-carboxylic acid | 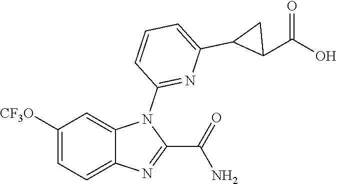 |
| 59 | 2-(1-(6-(2-carbamoyl-6- (trifluoromethoxy)-1H-indol-1- yl)pyridin-2-yl)cyclopropyl)acetic acid | 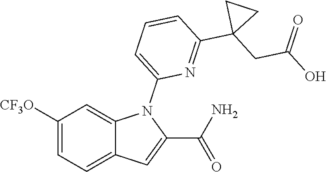 |
| 60 | trans-(rac)-3-(6-(2-carbamoyl-6- (trifluoromethoxy)-1H-indol-1- yl)pyridin-2-yl)-2,2- dimethylcyclopropane-1-carboxylic acid | 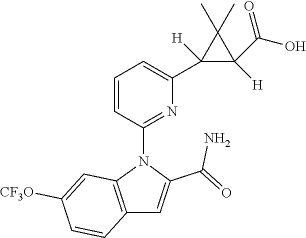 |
| 61 | trans-(rac)-3-(3-(2-carbamoyl-6- (trifluoromethoxy)-1H-indol-1- yl)phenyl)-2,2-dimethylcyclopropane- 1-carboxylic acid | 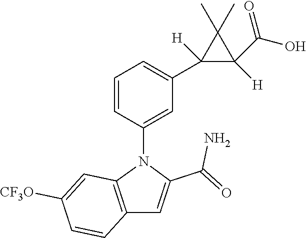 |
| 62 | 3-(6-(2-carbamoyl-6- (trifluoromethoxy)-1H-indol-1- yl)pyridin-2-yl)-3-methylbutanoic acid | 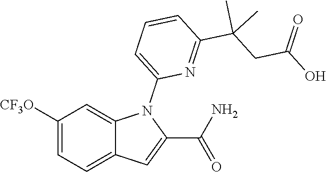 |
| 63 | 3-(6-(2-carbamoyl-6- (trifluoromethoxy)-1H-indol-1- yl)pyridin-2-yl)-3,3-difluoropropanoic acid | 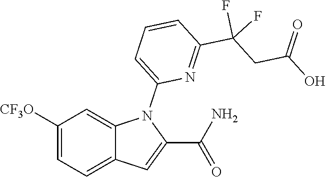 |
| 64 | 3-(3-(2-carbamoyl-6- (trifluoromethoxy)-1H-indol-1- yl)phenyl)-3,3-difluoropropanoic acid | 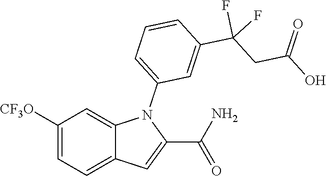 |
| 65 | trans-2-(6-(2-carbamoylthieno[2,3- c]pyridin-3-yl)pyridin-2- yl)cyclopropane-1-carboxylic acid | 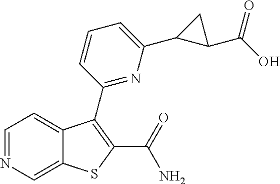 [SMILES: NC(=O)c1sc2cnccc2c1-c1cccc(C2CC2C(=O)O)n1] |
| 66 | trans-2-(6-(2-carbamoyl-6-methoxy- 1H-pyrrolo[3,2-c] pyridin-1-yl)pyridin- 2-yl)cyclopropane-1-carboxylic acid | 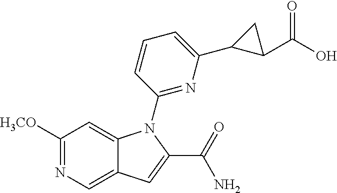 [SMILES: COc1cc2c(cn1)cc(C(N)=O)n2-c1cccc(C2CC2C(=O)O)n1] |
| 67 | 2-(1-(6-(2-carbamoyl-6- (trifluoromethoxy)-1H-indol-1- yl)pyridin-2-yl)cyclobutyl)acetic acid | 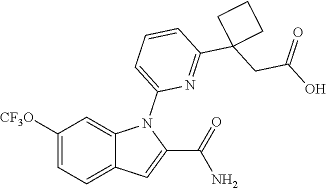 |
| 68 | 1-(6-(3,3-difluoroazetidin-1-yl)pyridin- 2-yl)-6-(trifluoromethoxy)-1H-indole- 2-carboxamide | 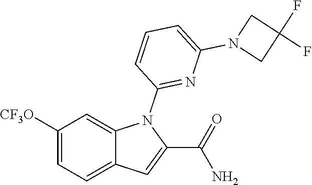 |
| 69 | 1-(6-(3,3-difluoroazetidin-1-yl)pyridin- 2-yl)-6-(trifluoromethoxy)-1H-indole- 2-carboxamide | 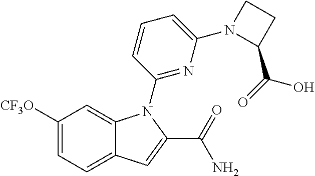 |
| 70 | 1-(6-(3,3-difluoroazetidin-1-yl)pyridin- 2-yl)-6-(trifluoromethoxy)-1H-indole- 2-carboxamide | 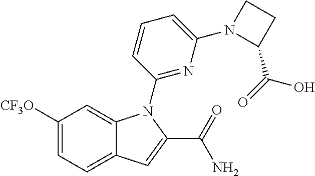 |

In one aspect, the invention includes a pharmaceutical composition comprising a compound or pharmaceutically acceptable salt thereof as described herein, and a pharmaceutically acceptable carrier.

In another aspect, the invention includes a method of inhibiting an sPLA2-X enzyme, said method comprising contacting the enzyme with a compound or pharmaceutically acceptable salt thereof as described herein, or a pharmaceutical composition as described herein.

In one embodiment of this aspect, the compound or pharmaceutically acceptable salt thereof is a selective inhibitor of sPLA2-X over other sPLA2 enzymes.

In one embodiment, the concentration at 50% inhibition (IC₅₀) of the compound or pharmaceutically acceptable salt thereof is at least 5-fold greater for other sPLA2 enzymes compared to the sPLA2-X enzyme.

In another embodiment, the concentration at 50% inhibition (IC₅₀) of the compound or pharmaceutically acceptable salt thereof is at least 10-fold greater for other sPLA2 enzymes compared to the sPLA2-X enzyme.

In another embodiment, the concentration at 50% inhibition (IC₅₀) of the compound or pharmaceutically acceptable salt thereof is at least 20-fold greater for other sPLA2 enzymes compared to the sPLA2-X enzyme.

In another embodiment, the concentration at 50% inhibition (IC₅₀) of the compound or pharmaceutically acceptable salt thereof is at least 50-fold greater for other sPLA2 enzymes compared to the sPLA2-X enzyme.

In another embodiment, the concentration at 50% inhibition (IC₅₀) of the compound or pharmaceutically acceptable salt thereof is at least 100-fold greater for other sPLA2 enzymes compared to the sPLA2-X enzyme.

In another embodiment, the concentration at 50% inhibition (IC₅₀) of the compound or pharmaceutically acceptable salt thereof is at least 500-fold greater for other sPLA2 enzymes compared to the sPLA2-X enzyme.

In another embodiment, the concentration at 50% inhibition (IC₅₀) of the compound or pharmaceutically acceptable salt thereof is at least 1000-fold greater for other sPLA2 enzymes compared to the sPLA2-X enzyme.

In one embodiment, the other sPLA2 enzymes are selected from sPLA2-IIE, sPLA2-IIA and sPLA2-V enzymes.

In one embodiment, the other sPLA2 enzymes are selected from sPLA2-IIA and sPLA2-V enzymes.

In another aspect, the invention includes a method of treating or lessening the severity of a disease mediated by an sPLA2-X enzyme in a subject, said method comprising administering to the subject a therapeutically effective amount of a compound or pharmaceutically acceptable salt thereof as described herein, or a pharmaceutical composition as described herein.

In one embodiment of this aspect, the disease is selected from cancer, atherosclerosis, or cardiovascular disease.

In another embodiment, the cancer is selected from multiple myeloma (MM), diffuse large B cell lymphoma (DLBCL), B cell lymphoma or non-small cell lung cancer.

In a further embodiment, the multiple myeloma (MM) and/or diffuse large B cell lymphoma (DLBCL) is recurring, refractory, or relapsing DLBCL and/or MM.

In another embodiment, the cancer is selected from B cell lymphoma or non-small cell lung cancer.

### Pharmaceutical Compositions

The compounds described herein can be formulated into pharmaceutical compositions that further comprise a pharmaceutically acceptable carrier, diluent, adjuvant or vehicle. In one embodiment, the present invention provides a pharmaceutical composition comprising a compound of the invention described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

According to another embodiment, the invention provides a composition comprising a compound of this invention or a pharmaceutically acceptable salt thereof and a pharmaceutically acceptable carrier, adjuvant, or vehicle. Pharmaceutical compositions of this invention comprise a therapeutically effective amount of a compound of Formula I, wherein a “therapeutically effective amount” is an amount that is (a) effective to inhibit one or more secreted PLA2 enzyme subtypes, for example, subtype-X in a biological sample or in a patient, or (b) effective in treating and/or ameliorating a disease or disorder that is mediated by one or more secreted PLA2 enzyme subtypes, for example, subtype-X.

The term “patient,” or “subject” used interchangeably herein, means an animal, preferably a mammal, and most preferably a human.

It also will be appreciated that certain of the compounds of the present invention can exist in free form for treatment, or where appropriate, as a pharmaceutically acceptable derivative (e.g., a salt) thereof. According to the present invention, a pharmaceutically acceptable derivative includes, but is not limited to, pharmaceutically acceptable prodrugs, salts, esters, salts of such esters, or any other adduct or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A pharmaceutically acceptable carrier may contain inert ingredients that do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents. Preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid may be used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, or preferentially, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds also can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also may comprise, as is normal practice, additional substances other than inert diluents, e.g., tableting lubricants and other tableting aids such as magnesium stearate and microcrystalline cellulose. In the case of capsules, tablets and pills, the dosage forms also may comprise buffering agents. They may optionally contain opacifying agents and can be of a composition such that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, ear drops, and eye drops also are contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers also can be used to increase the flux of the compound across the skin. The rate can be controlled by either providing a rate controlling membrane or by dispersing the compound in a polymer matrix or gel.

The compounds of the invention preferably are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The amount of the compounds of the present invention that may be combined with the carrier materials to produce a composition in a single dosage form will vary depending upon the host treated, the particular mode of administration, and other factors. Preferably, the compositions should be formulated so that a dosage of between 0.01-100 mg/kg body weight/day of the inhibitor can be administered to a patient receiving these compositions.

Depending upon the particular condition, or disease, to be treated or prevented, additional therapeutic agents, which are normally administered to treat or prevent that condition, also may be present in the compositions of this invention. As used herein, additional therapeutic agents that are normally administered to treat or prevent a particular disease, or condition, are known as “appropriate for the disease, or condition, being treated.”

Some embodiments of the present invention provide methods for administering an effective amount of a compound, or a pharmaceutically acceptable salt thereof of the invention and at least one additional therapeutic agent (including, but not limited to, chemotherapeutic antineoplastics, apoptosis-inhibiting agents, antimicrobials, antivirals, antifungals, and anti-inflammatory agents) and/or therapeutic technique (e.g., surgical intervention, and/or radiotherapies). In a particular embodiment, the additional therapeutic agent(s) is an anticancer agent.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB inhibitors; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide); and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

For example, chemotherapeutic agents or other anti-proliferative agents may be combined with the compounds of this invention to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxetan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy—Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of the present invention (or a pharmaceutically acceptable salt thereof) is administered to a patient in need thereof.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, RI 15777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

### Methods of Treatment

The compounds of the invention are inhibitors (e.g., inhibitors) of the activity or function of secreted phospholipase A2-X (sPLA2-X) enzyme.

Accordingly, the present invention contemplates that exposure of patients (e.g., humans) suffering from a condition characterized by aberrant sPLA2-X protein activity (e.g., cancer (e.g., B cell lymphoma and non-small cell lung cancer) and atherosclerosis) to therapeutically effective amounts of sPLA2-X inhibitors (e.g. inhibitors) of the present invention. Accordingly, the present invention contemplates that exposure of patients (e.g., humans) in need thereof, suffering from a condition characterized by aberrant sPLA2-X protein activity (e.g., cancer (e.g., B cell lymphoma and non-small cell lung cancer) and atherosclerosis) to therapeutically effective amounts of an sPLA2-X inhibitor, or a combination of sPLA2-X inhibitors (e.g. inhibitors) of the present invention, wherein the sPLA2-X inhibitor can be any compound of Formulas I, II, IIIa, IIIb, IIIc, IIId, IIIe, IIIf-1-IIIf-7, IV, Iva, IVb, IVc, V, Va, Vb and Vc, and/or those specifically recited in Table 1, or a pharmaceutically acceptable salt thereof, as disclosed herein, that are operable to inhibit sPLA2-X protein activity in therapeutically effective amounts useful to treat diseases and/or disorders that are associated with aberrant sPLA2-X protein activity, for example, cancer (e.g., B cell lymphoma and non-small cell lung cancer) and atherosclerosis, among others exemplified below. The present invention contemplates that inhibitors of sPLA2-X satisfy an unmet need for the treatment of multiple conditions characterized with aberrant sPLA2-X, either when administered as monotherapy, or when administered in a temporal relationship with additional agent(s), such as other cell death-inducing or cell cycle disrupting therapeutic drugs (e.g., cancer therapeutic drugs or radiation therapies) (combination therapies), so as to render a greater proportion of the cells (e.g., cancer cells) or supportive cells susceptible to executing the apoptosis program compared to the corresponding proportion of cells in a patient treated only with the therapeutic drug or radiation therapy alone.

In some embodiments, the present disclosure provides a method of treating or lessening the severity of a disease mediated by an sPLA2-X enzyme in a subject, said method comprising administering to the subject a therapeutically effective amount of a compound or pharmaceutically acceptable salt thereof according to any one of Formulas IV, IVa, IVb, IVc, V, Va, Vb, or Vc. As described herein, exemplary diseases mediated or associated with an aberrant sPLA2-X enzyme activity includes, but is not limited to, cancer, atherosclerosis, and cardiovascular disease. In certain embodiments, the cancer includes one or more of multiple myeloma (MM), diffuse large B cell lymphoma (DLBCL), B cell lymphoma or non-small cell lung cancer. In some illustrative embodiments, the multiple myeloma (MN) and/or diffuse large B cell lymphoma (DLBCL) is recurring, refractory, or relapsing DLBCL and/or MM. In further therapeutic methods contemplated herein, the sPLA2-X inhibitors (e.g. inhibitors) of the present invention can be used to treat cancer, which may include B cell lymphoma or non-small cell lung cancer, in addition to the other cancers illustrated herein.

In certain embodiments of the invention, wherein the condition being treated is cancer, characterized by the presence of pathophysiological sPLA2-X protein activity, combination treatment of patients with a therapeutically effective amount of a compound of the present invention and a course of an anticancer agent produces a greater anti-cancer response and clinical benefit in such patients compared to those treated with the compound or anticancer drugs/radiation alone. Since the doses for all approved anticancer drugs and radiation treatments are known, the present invention contemplates the various combinations of them with the present compounds. Administration of the combination therapies may be performed in any order provided that the order of administration is contemplated with an appropriate risk/benefit ratio, and optionally as clinically tested in one or more clinical trials.

The invention also provides pharmaceutical compositions comprising the compounds of the invention, or the pharmaceutically acceptable salts thereof, in a pharmaceutically acceptable carrier.

The invention also provides kits comprising a compound of the invention and instructions for administering the compound to a patient. The kits may optionally contain other therapeutic agents, e.g., anticancer agents or apoptosis-inhibiting agents.

Moreover, the present invention provides methods for inhibiting (e.g. inhibiting) sPLA2-X protein activity in cells through exposing such cells to one or more of the compounds of the present invention, or their pharmaceutically acceptable salts thereof.

In some embodiments, the compositions and methods of the present invention are used to treat diseased cells, tissues, organs, or pathological conditions and/or disease states in a patient (e.g., a mammalia patient including, but not limited to, humans and veterinary animals). In this regard, various diseases and pathologies are amenable to treatment or prophylaxis using the present methods and compositions. A non-limiting exemplary list of these diseases and conditions includes, but is not limited to, colorectal cancer, non-small cell lung carcinoma, head or neck carcinoma, glioblastoma multiform cancer, pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, lymphoma, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

In other embodiments, the disorder is any disorder having cells having aberrant sPLA2-X protein activity (e.g., proliferative diseases and cardiovascular diseases)).

The present invention provides methods for administering a compound of the invention with radiation therapy. The invention is not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tirapazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the patient to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the patient's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to a patient under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention or their pharmaceutically acceptable salts thereof are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 100 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 1,000 mg, conveniently about 0.25 to 500 mg of the compound or its solvates. In some embodiments, unit, daily or therapeutically effective doses can include, a dose from about 10 mg to about 1,000 mg, or from about 20 mg to about 900 mg, or from about 50 mg to about 800 mg, or from about 60 mg to about 700 mg, or from about 70 mg to about 600 mg, or from about 80 mg to about 500 mg, or from about 90 mg to about 400 mg, or from about 100 mg to about 300 mg. Unit, daily or therapeutically effective doses can include any integer or numerical range within these stated ranges described herein.

In a topical formulation, the compound may be present at a concentration of about 0.01 to 100 mg per gram of carrier. In one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary patients (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired. Dosages are also informed by clinical studies in which a population of subjects with and without the treatable diseases contemplated herein are dosed with varying amounts of the compositions of the present invention. Results from these clinical trials may also determine the therapeutically effective doses and dosing regimens.

### Experimental Section

### Synthesis of the Compounds of the Invention

### Synthetic Methods

The general procedures used to synthesize the compounds are described in reaction Schemes 1-X and are illustrated in the examples below. The following examples are put forth so the invention can be well understood and are not intended to limit the scope of the invention, nor are they intended to represent that they are all of the experiments that could be performed. Synthesized compounds were analyzed and characterized by use of the following equipment: Liquid chromatography-mass spectra (LC/MS) were obtained using an Agilent LC/MSD G1946D or an Agilent 1100 Series LC/MSD Trap G1311A or G2435A. Quantifications were obtained on a Cary 50 Bio UV-visible spectrophotometer. 1H, 13C, and 19F nuclear magnetic resonance (NMR) spectra were obtained using a JEOL ECZ400S NMR spectrometer at 400, 100, and 376 MHz, respectively. High-performance liquid chromatography (HPLC) analytical separations were performed on an Agilent 1100 or Agilent 1200 HPLC analytical system and followed by an Agilent Technologies G1315B Diode Array Detector set at or near the UVmax @210 nm. HPLC preparatory separations were performed on a Gilson preparative HPLC system or an Agilent 1100 preparative HPLC system and followed by an Agilent Technologies G1315B Diode Array Detector set at or near the UVmax @210 nm. Analytical chiral HPLC separations were performed on an Agilent 1100 analytical system and followed by an Agilent Technologies G1315B Diode Array Detector set at or near the UVmax @210 nm. The separations are accomplished with a Gemini 3μ or 5μ C18 50×2.5 mm or 250×4.6 mm solid-phase column eluting with acetic acid-methanol-water gradient or ammonium acetate-acetonitrile-water gradient. Flash chromatography is performed using CombiFlash NextGen 300+ using RediSep Silica columns. All final compounds gave satisfactory purity (≥95%) by HPLC and by ¹H NMR spectroscopy. Thin-layer chromatography (TLC) analyses are performed on Uniplate 250μ silica gel plates (Analtech, Inc. Catalog no. 02521) and are typically developed for visualization by UV/Vis, using 50 vol % concentrated sulfuric acid in water spray, Iodine stain, or Hanessian's stain.

### General Synthetic Scheme:

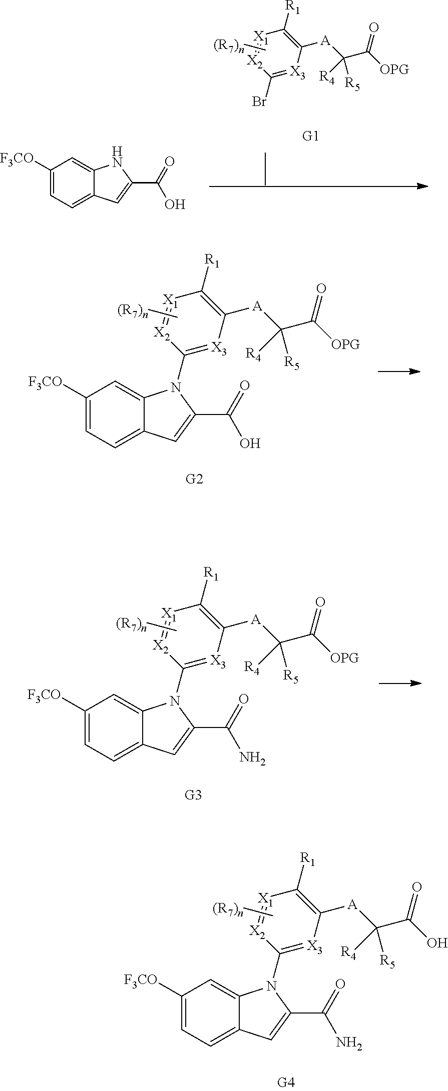

The compounds of the invention can generally be synthesized according to the general synthetic scheme above, where X₁, X₂, X₃, A, R₁, R₄, R₅, R₇, and n are defined herein and PG is a protecting group. Referring to the scheme, 6-(trifluoromethoxy)-2-carboxy indole can be coupled with a reactant of the Formula G1 in the presence of an aryl bromide, a copper reagent, such as Cu(OAc)₂, methyl α-D-glucopyranoside, KI, DBU, and a solvent such as DMSO. The resulting carboxylic acid product of Formula G2 can be converted to an amide of Formula G3 under peptide coupling conditions, such as TBTU, NH₄Cl, and DIPEA in a solvent such as DMF. The compound of Formula G3 can be converted into the carboxylic acid compound of Formula G4 under hydrolysis conditions, such as 1M LiOH in MeOH.

### Synthesis of Trifluoromethoxy Cores:

### Example 1: Preparation of tert-butyl 5-(trifluoromethoxy)benzofuran-2-carboxylate

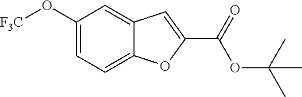 [SMILES: CC(C)(C)OC(=O)c1cc2cc(OC(F)(F)F)ccc2o1]

To a mixture consisting of 2-hydroxy-5(trifluoromethoxy)benzaldehyde (1.03 g, 5.00 mmol) in DMSO (8 mL) was added cesium carbonate (5.05 g, 15.5 mmol) at room temperature and under N₂. The reaction mixture was heated to 100° C. where it was then treated with tert-butyl bromoacetate (1.02 g, 5.23 mmol) and let stir for 2 hours. The reaction mixture was subsequently partitioned between ethyl acetate (40 mL) and water (80 mL), and the phases separated. The aqueous phase was partitioned once more with ethyl acetate (100 mL) and the phases separated. The combined organic phase was sequentially washed with potassium carbonate (2×25 mL), 50% brine (2×25 mL), and brine (25 mL). The organic layer was then dried over anhydrous sodium sulfate, filtered, and concentrated under reduced pressure to give 1.16 g as a crude yellow oil. The product was purified by flash silica column chromatography on a Cole-Palmer digital gear pump system using a 24 g RediSep Gold R_{f} flash silica cartridge. The product eluted after 400 mL of 1% ethyl acetate in heptane affording the title compound as a clear colorless oil (1.04 g, 68%); ¹H-NMR (400 MHz; CDCl₃) δ 7.56 (d, 1H, J=8.9 Hz), 7.51 (d, 1H, J=0.9 Hz), 7.28 (dd, 1H, J=1.7, 9.1 Hz), 7.25 (s, 1H), 1.6-1.7 (s, 9H); ¹⁹F-NMR (376 MHz; CDCl₃) δ −58.1 (s, 3F); MS (FIA MS−) m/z 245 (M-tBu).

### Example 2: Preparation of methyl 5-(trifluormethoxy)benzo[b]thiophene-2-carboxylate

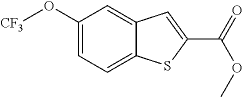 [SMILES: COC(=O)c1cc2cc(OC(F)(F)F)ccc2s1]

To a mixture consisting of 2-fluoro-5-(trifluoromethoxy)benzaldehyde (1.04 g, 5.00 mmol) in DMF (50 mL) was added potassium carbonate (1.38 g, 10.00 mmol) followed by methyl thioglycolate (583 mg, 5.49 mmol). The reaction mixture was heated to 60° C. under an N₂ atmosphere and let stir overnight. The reaction mixture was subsequently partitioned between H₂O (100 mL) and ethyl acetate (500 mL). The aqueous was reextracted twice with ethyl acetate (200 mL). The combined organic phase was washed with 50% brine (3×100 mL) and brine (100 mL), then dried over anhydrous sodium sulfate, filtered, and concentrated under reduced pressure to give 1.21 g of a crude yellow solid. The product was purified by flash silica column chromatography utilizing a Cole-Palmer digital gear pump system and a 24 g RediSep Gold Rf flash silica cartridge. The product eluted with 20% DCM in heptane affording the title compound as a white crystalline solid (981 mg, 71%); ¹H-NMR (400 MHz; CDCl₃) δ 8.03 (s, 1H), 7.86 (d, 1H, J=8.9 Hz), 7.71 (s, 1H), 7.3-7.4 (d, 1H), 3.95 (s, 3H); ¹⁹F-NMR (CDCl₃, 376 MHz) δ −57.8 (s, 3F); MS (FIA MS−) m/z 276.0 (M−1); HPLC UV purity Rt=9.901 min, 100%.

### Example 3: Preparation of 6-(Trifluoromethoxy)-1H-Indole-2-Carbocylic Acid

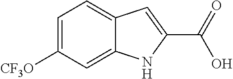

To a mixture consisting of 2-bromo-5-(trifluoromethoxy)aniline (63.0 g, 246 mmol), suspended in dimethylacetamide (540 mL), was added 2-oxopropanoic acid (65.0 g, 738 mmol), acetic acid (21.1 mL, 369 mmol), and magnesium sulfate (14.8 g, 123 mmol). Nitrogen was bubbled through the suspension for 20 minutes while stirring and subsequently treated with potassium phosphate (67.9 g, 319.8 mmol) followed by bis(tri-t-butylphospine)palladium(0) (18.9 g, 36.9 mmol). Nitrogen was bubbled through solution again for 20 minutes and the reaction mixture was heated at 140° C. for 2 hours. The reaction mixture was cooled and passed through a pad a celite. The filtrate was basified using 2N sodium hydroxide (320 mL) and extracted with diethyl ether (2×2 50 mL). The aqueous phase was then acidified with 3N hydrochloric acid (250 mL) and extracted with ethyl acetate (1×2 L). The organic phase was washed with 50% brine (4×200 mL) and brine (2×200 mL), then dried over anhydrous sodium sulfate, filtered, and concentrated under reduced pressure to give 74 g of crude material. The crude material was dissolved in a hot solution of dichloromethane (50 mL) and heptane (100 mL). The solution was then cooled to room temperature resulting in precipitation and the mixture was filtered through a medium fritted filter. The filtrate was concentrated, and the precipitation procedure was repeated. To eliminate residual dimethylacetamide the solid (51 g) was passed through a 2 kg column of 40-63 m (230-400 mesh) silica gel. The product eluted in 20% ethyl acetate in heptane to give the product as a white powder (30 g, 50%); ¹H-NMR (400 MHz; CDCl₃)) δ 9.06 (br s, 1H), 7.71 (d, 1H, J=8.7 Hz), 7.3-7.4 (m, 1H), 7.31 (s, 1H), 7.06 (br d, 1H, J=8.7 Hz); ¹⁹F-NMR (CDCl₃, 376 MHz) δ −57.8 (s, 3F); MS (FIA-MS−) m/z 244.0 (M−1).

### Preparation of Aryl Bromides:

### Example 4: Preparation of ethyl 3-(3-bromophenyl)-2,2-dimethylpropanoate

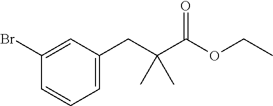 [SMILES: CCOC(=O)C(C)(C)Cc1cccc(Br)c1]

To an oven-dried flask containing ethyl isobutyrate (Alfa Aesar, 2.0 mL, 15 mmol) was added anhydrous THF (50 mL). The flask was cooled to −78° C. and placed under N₂ atmosphere. Next a lithium diisopropylamide solution (Aldrich, 2.0 M THF/heptane/ethylbenzene, 7.5 mL, 15 mmol) was added portion-wise to the cooled solution. After stirring for 30 minutes at −78° C., 3-bromobenzyl bromide (CombiBlocks, 2.5 g, 10 mmol) was added dropwise as a solution in 3 mL of THF. The resulting mixture was stirred for 1 hour at −78° C., then allowed to warm to room temperature. After 3 hours the reaction was complete by TLC (10/90 EA/Hep). The crude reaction mixture was quenched with the slow addition of saturated ammonium chloride solution (25 mL). The phases were separated, and the organic phase was partitioned with H₂O (50 mL), followed by brine (50 mL). The organic layer was concentrated under reduced pressure to afford the crude products as an orange oil (3.02 g). The crude oil was purified by flash silica column chromatography on a CombiFlash NextGen 300+ purification system. Elution through a 120 g RediSep Gold Rf flash silica cartridge with 0-10% ethyl acetate in heptane afforded the title compound as a colorless oil (1.70 g, 59% yield); Rf 0.70 with 90:10 v/v heptane-ethyl acetate (UV 254 nM); ¹H-NMR (400 MHz; CDCl₃) δ 7.35 (d, 1H, J=7.5 Hz), 7.29 (t, 1H, J=1.6 Hz), 7.13 (t, 1H, J=7.7 Hz), 7.05 (d, 1H, J=7.8 Hz) 4.13 (q, 2H, J=7.1 Hz), 2.82 (s, 2H), 1.25 (t, 3H, J=7.1 Hz), 1.19 (s, 6H); MS (APCI⁺) m/z 285.0, 287.0 (M+1, Br isotope), HPLC UV purity Rt=10.737 min, 99.79% pure.

### Example 5: Preparation of methyl (E/Z)-3-(6-bromopyridin-2-yl)but-2-enoate

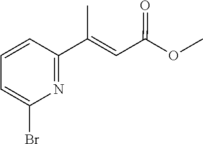

To an oven-dried reaction flask charged with sodium hydride (Aldrich, 60% dispersion in mineral oil, 2.88 g, 72.0 mmol) was added anhydrous THF (90 mL). The reaction flask was cooled to 0° C. and placed under N₂ atmosphere. Next trimethyl phophonoacetate (CombiBlock, 13.11 g, 72.0 mmol) was added dropwise as a solution in THF (30 mL). Stir for 1 h at 0° C. then add 2-acetyl-6-bromopyridine (Combiblock, 12.0 g, 60.0 mmol) dropwise as a solution in THF (25 mL). The mixture was stirred for 1 h at 0° C. then allowed to stir overnight. Next the crude reaction mixture was concentrated under reduced pressure to afford a crude pink solid. The crude solid was portioned between water (200 mL) and EA (250 mL). The organic layer was washed a second time with water (200 mL) and then brine (200 mL). The organic layer was dried over anhydrous MgSO₄, filtered and concentrated under reduced pressure to afford the title compound as a red oil (15.04 g, 97.9%); Rf 0.51 and 0.44 (E and Z isomers) with 75:25 v/v heptane-ethyl acetate (UV 254 nM); ¹H-NMR (400 MHz; CDCl₃) δ 7.5-7.6 (m, 2H), 7.4-7.5 (m, 2H), 7.23 (d, 1H, J=7.2 Hz), 6.76 (d, 1H, J=1.4 Hz), 6.00 (d, 1H, J=1.4), 3.76 (s, 2H), 3.61 (s, 3H), 2.56 (s, 2H), 2.20 (s, 3H); MS (APCI⁺) m/z 256.0, 258.0 (M+1, Br isotope).

### Example 6: Preparation of ethyl 2-((3-bromophenyl)thio)-2-methylpropanoate

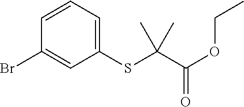 [SMILES: CCOC(=O)C(C)(C)Sc1cccc(Br)c1]

To a mixture consisting of ethyl 2-bromo-2-methylpropanoate (0.516 g, 2.64 mmol) in DMF (10 mL) was added 3-bromobenzenethiol (0.500 g, 2.64 mmol) and cesium carbonate (1.72 g, 5.28 mmol). The reaction mixture was stirred at 55° C. under N₂ overnight. The reaction was partitioned between a saturated solution of ammonium chloride (200 mL) and ethyl acetate (200 mL). The phases were separated, and the organic phase was partitioned again with water, followed by brine. The organic layer was separated and dried over magnesium sulfate, filtered and concentrated under reduced pressure to afford the crude product. The crude material was purified by flash silica column chromatography on a CombeFlash NextGen 300+ purification system. Elution through a 40 g RediSep Gold Rf flash silica cartridge with 5% ethyl acetate in heptane for 5 column volumes followed by a gradient to 10% ethyl acetate in heptane afforded the title compound as a clear colorless oil (0.621 g, 78%); Rf 0.37 with 95:5 v/v heptane-ethyl acetate (UV 254 nM and CAM stain); ¹H-NMR (400 MHz; CDCl₃) δ 7.62 (t, 1H, J=1.7 Hz), 7.49 (d, 1H, J=8.2 Hz), 7.39 (td, 1H, J=1.2, 7.7 Hz), 7.19 (t, 1H, J=7.8 Hz), 4.11 (q, 2H, J=7.3 Hz), 1.48 (s, 6H), 1.21 (t, 3H, J=7.1 Hz); MS (APCI⁺) m/z 229.0, 231.0 (M+1, Br isotope).

### Example 7: Preparation of ethyl 1-((3-bromophenyl)thio)cyclobutane-1-carboxylate

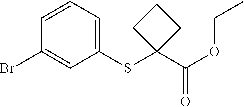 [SMILES: CCOC(=O)C1(Sc2cccc(Br)c2)CCC1]

To a mixture consisting of ethyl 1-bromocyclobutane-1-carboxylate (0.547 g, 2.64 mmol) in DMF (10 mL) was added 3-bromobenzenethiol (0.500 g, 2.64 mmol) and cesium carbonate (1.72 g, 5.28 mmol). The reaction mixture was stirred at 55° C. under N₂ overnight. The reaction was partitioned between a saturated solution of ammonium chloride (200 mL) and ethyl acetate (200 mL). The phases were separated, and the organic phase was partitioned again with water, followed by brine. The organic layer was separated and dried over magnesium sulfate, filtered and concentrated under reduced pressure to afford the crude product. The crude material was purified by flash silica column chromatography on a CombiFlash NextGen 300+ purification system. Elution through a 40 g RediSep Gold Rf flash silica cartridge with 5% ethyl acetate in heptane for 5 column volumes followed by a gradient to 10% ethyl acetate in heptane afforded the title compound as a clear colorless oil (0.646 g, 78%); Rf 0.35 with 95:5 v/v heptane-ethyl acetate (UV 254 nM and CAM stain); ¹H-NMR (400 MHz; CDCl₃) δ 7.52 (t, 1H, J=1.7 Hz), 7.38 (d, 1H, J=7.9 Hz), 7.3-7.3 (m, 1H), 7.14 (t, 1H, J=8.5 Hz), 4.14 (q, 2H, J=7.1 Hz), 2.6-2.7 (m, 2H), 2.1-2.3 (m, 3H), 1.8-2.0 (m, 1H), 1.20 (t, 3H, J=7.1 Hz); MS (APCI⁺) m/z 240.9, 242.9 (M+1, Br isotope).

### Example 8: Preparation of ethyl 2-((3-bromophenyl)thio)-2,2-difluoroacetate

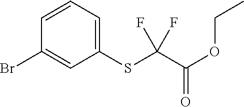 [SMILES: CCOC(=O)C(F)(F)Sc1cccc(Br)c1]

To a mixture consisting of ethyl 2-bromo-2,2-difluoroacetate (0.547 g, 2.64 mmol) in DMF (10 mL) was added 3-bromobenzenethiol (0.500 g, 2.64 mmol) and cesium carbonate (1.72 g, 5.28 mmol). The reaction mixture was stirred at 55° C. under N₂ overnight. The reaction was partitioned between a saturated solution of ammonium chloride (200 mL) and ethyl acetate (200 mL). The phases were separated, and the organic phase was partitioned again with water, followed by brine. The organic layer was separated and dried over magnesium sulfate, filtered and concentrated under reduced pressure to afford the crude product. The crude material was purified by flash silica column chromatography on a CombiFlash NextGen 300+ purification system. Elution through a 40 g RediSep Gold Rf flash silica cartridge with 2% ethyl acetate in heptane for 5 column volumes followed by a gradient to 10% ethyl acetate in heptane afforded the title compound as a clear colorless oil (0.600 g, 73%); Rf 0.43 with 95.5 v/v heptane-ethyl acetate (UV 254 nM and CAM stain); ¹H-NMR (400 MHz; CDCl₃) δ 7.77 (t, 1H, J=1.7 Hz), 7.6-7.6 (m, 1H), 7.55 (d, 1H, J=7.8 Hz), 7.27 (t, 1H, J=8.9 Hz), 4.28 (q, 2H, J=7.3 Hz), 1.28 (t, 3H, J=7.1 Hz); ¹⁹F-NMR (376 MHz; CDCl₃) 81.6; MS (APCI⁺) m/z 309.0, 311.0 (M−1, Br isotope).

### Preparation of Target Compounds

### Example 9: Synthesis of 2-(5-(2-carbamoyl-6-(trifluoromethoxy)-1H-indol-1-yl)-2,3-dihydrobenzofuran-3-yl)acetic acid (Compound 1)

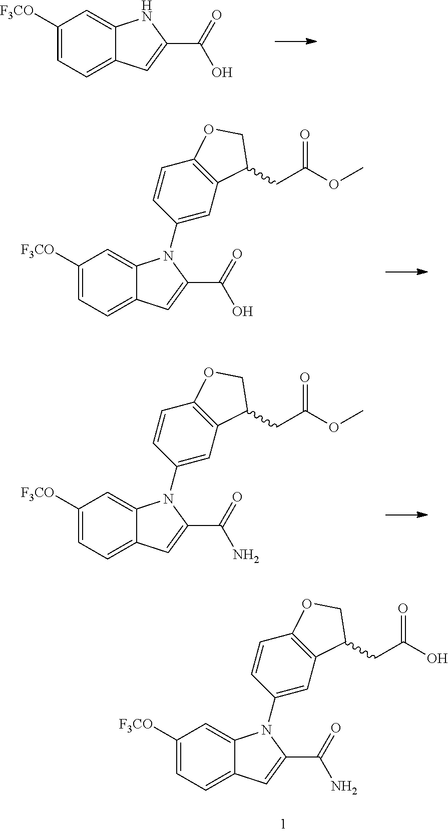 [SMILES: O=C(O)c1cc2ccc(OC(F)(F)F)cc2[nH]1 ; COC(=O)CC1COc2ccc(-n3c(C(=O)O)cc4ccc(OC(F)(F)F)cc43)cc21 ; COC(=O)CC1COc2ccc(-n3c(C(N)=O)cc4ccc(OC(F)(F)F)cc43)cc21 ; NC(=O)c1cc2ccc(OC(F)(F)F)cc2n1-c1ccc2c(c1)C(CC(=O)O)CO2]

Step A: Preparation of 1-(3-(2-methoxy-2-oxoethyl)-2,3-dihydrobenzofuran-5-yl)-6-(trifluoromethoxy)-1H-indole-2-carboxylic acid

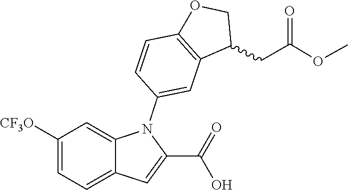

To a mixture consisting of 6-(trifluoromethoxy)-1H-indole-2-carboxylic acid (ChemShuttle, 0.400 g, 1.63 mmol), 5-bromo-2,3-dihydrobenzofuran-3-acetate (Accela, 0.442 g, 1.63 mmol), copper (II) acetate (CombiBlocks, 0.295 g, 1.63 mmol), methyl-α-D-glucopyranoside (CombiBlocks, 0.316 g, 1.63 mmol), potassium iodide (VWR, 0.541 g, 3.26 mmol) in DMSO (24 mL) was added DBU (Oakwood Chemicals, 0.73 mL, 4.89 mmol). The reaction mixture was stirred overnight at 115° C. under N₂ atmosphere. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 1.002 g. The crude oil was purified by flash silica column chromatography on a CombiFlash NextGen 300+ purification system. Elution through a 40 g RediSep Gold Rf flash silica cartridge with 10-100% ethyl acetate in hexanes containing 1% acetic acid afforded the title compound as a white solid (0.147 g, 21% yield); R_{f} 0.18 with 1:30:70 v/v acetic acid-ethyl acetate-hexane (UV 254 nM); ¹H-NMR (400 MHz; DMSO-d6) δ 12.7-13.0 (bs, 1H), 7.87 (d, 1H, J=8.7 Hz), 7.42 (s, 1H), 7.1-7.2 (m, 2H), 6.90 (d, 2H, J=8.3 Hz), 4.83 (t, 1H, J=9.2 Hz), 4.3-4.4 (m, 1H), 3.8-4.0 (m, 1H), 3.60 (s, 3H), 2.9-3.0 (m, 2H) 2.5-2.6 (m, 1H); ¹⁹F-NMR (376 MHz; DMSO-d6) δ −56.66; MS (APCI−) m/z 434 (M−1); HPLC UV purity Rt=9.275 min, 97.93%.

### Step B: Preparation of methyl 2-(5-(2-carbamoyl-6-(trifluoromethoxy)-1H-indol-1-yl)-2,3-dihydrobenzofuran-3-yl)acetate

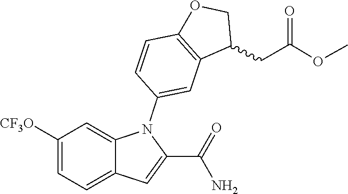

To a mixture consisting of 1-(3-(2-methoxy-2-oxoethyl)-2,3-dihydrobenzofuran-5-yl)-6-(trifluoromethoxy)-1H-indole-2-carboxylic acid (0.30 g, 0.69 mmol) in DMF (1 mL) was added ammonium chloride (Chem-Impex, 0.11 g, 0.21 mmol). Next was added TBTU (Oakwood, 0.033 g, 0. 104 mmol), followed by the addition of DIPEA (0.1 mL, 0.621 mmol). The reaction mixture was stirred overnight at room temperature under N₂ atmosphere. The reaction mixture was subsequently partitioned between ethyl acetate (30 mL) and H₂O (30 mL). The phases were separated, and the organic phase was partitioned with 1M KHSO₄ (30 mL), followed by brine (30 mL). The organic layer was concentrated under reduced pressure to afford the crude product as a colorless oil (0.026 g, 88%). The crude oil was used in the next w/o further purification. R_{f} 0.10 with 1:30:70 v/v acetic acid-ethyl acetate-hexane (UV 254 nM); MS (APCI⁺) m/z 435.0 (M+1); HPLC UV purity Rt=8.452 min, 94.83%.

### Step C: Preparation of 2-(5-(2-carbamoyl-6-(trifluoromethoxy)-1H-indol-1-yl)-2,3-dihydrobenzofuran-3-yl)acetic acid (Compound 1)

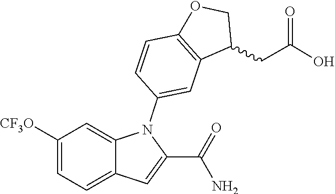

To a mixture consisting of methyl 2-(5-(2-carbamoyl-6-(trifluoromethoxy)-1H-indol-1-yl)-2,3-dihydrobenzofuran-3-yl)acetate (0.026 mg, 0.06 mmol) in methanol (1.5 mL) was added 1M lithium hydroxide (0.239 mL, 0.239 mmol). The reaction mixture was stirred at room temperature for 1.5 h under N₂ atmosphere. The reaction mixture was subsequently partitioned between ethyl acetate (10 mL) and 1M HCl (10 mL). The phases were separated, and the organic phase was partitioned with H₂O (5 mL), followed by brine (5 mL). The organic layer was concentrated under reduced pressure to afford the crude product as a pink solid (0.025 g, 100%). R_{f} 0.48 with 1:80:20 v/v acetic acid-ethyl acetate-hexane (UV 254 nM; ¹H-NMR (400 MHz; CDCl₃) δ 12.34 (br s, 1H), 7.91 (br s, 1H), 7.78 (d, 1H, J=8.7 Hz), 7.32 (br s, 1H), 7.10 (br d, 1H, J=9.6 Hz), 7.01 (dd, 1H, J=2.1, 8.3 Hz), 6.88 (s, 1H). 6.83 (d, 1H, J=8.5 Hz), 3.7-3.9 (m, 1H), 2.81 (dd, 1H, J=5.2, 16.8 Hz), 2.5-2.7 (m, 1H); MS (APCI⁺) m/z 421 (M+1). HPLC UV purity, Rt=7.334 min, 94.3%; melting point=222-223° C.

### Example 10: Separation, hydrolysis, and isolation of (+) and (−) 2-(5-(2-carbamoyl-6-(trifluoromethoxy)-1H-indol-1-yl)-2,3-dihydrobenzofuran-3-yl)acetic acid (Compounds 1a and 1b)

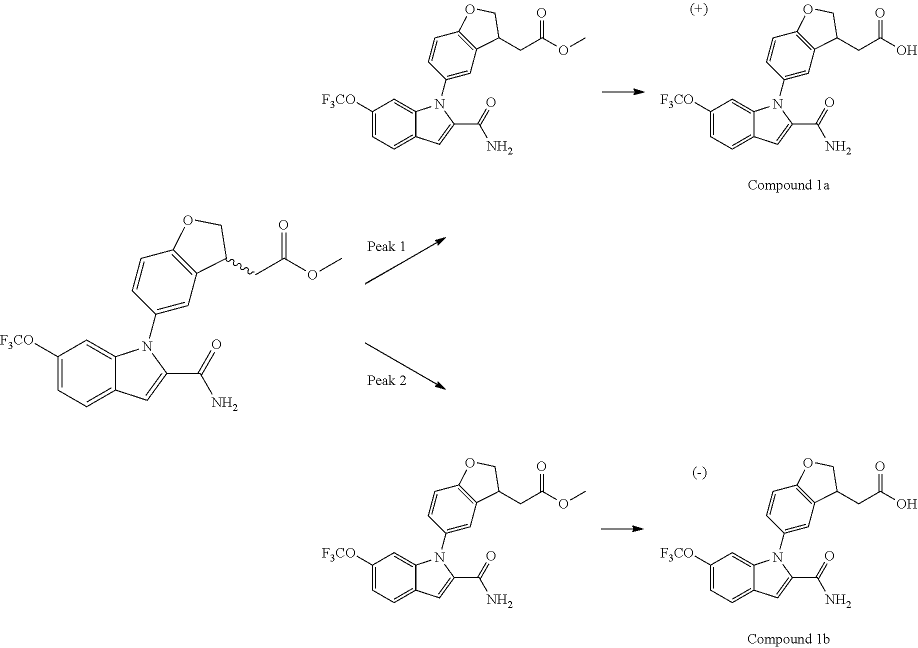 [SMILES: COC(=O)CC1COc2ccc(-n3c(C(N)=O)cc4ccc(OC(F)(F)F)cc43)cc21 ; NC(=O)c1cc2ccc(OC(F)(F)F)cc2n1-c1ccc2c(c1)C(CC(=O)O)CO2 ; COC(=O)CC1COc2ccc(-n3c(C(N)=O)cc4ccc(OC(F)(F)F)cc43)cc21 ; COC(=O)CC1COc2ccc(-n3c(C(N)=O)cc4ccc(OC(F)(F)F)cc43)cc21 ; NC(=O)c1cc2ccc(OC(F)(F)F)cc2n1-c1ccc2c(c1)C(CC(=O)O)CO2]

### Step A: Chiral separation of methyl 2-(5-(2-carbamoyl-6-(trifluoromethoxy)-1H-indol-1-yl)-2,3-dihydrobenzofuran-3-yl)acetate

The enantiomers of methyl 2-(5-(2-carbamoyl-6-(trifluoromethoxy)-1H-indol-1-yl)-2,3-dihydrobenzofuran-3-yl)acetate (0.147 g) was separated by Chiral HPLC on a Chiralpak IA column (250 mm×20 mm, 5 μm particle) using 90:10:0.1 Heptane:IPA:TEA at a flow rate of 20 mL/min with UV detection at 292 nm. Peak 1 eluted at 20.02 minutes and Peak 2 eluted at 25.04 minutes. Enantiomer 1 (peak 1) was concentrated under reduced pressure to afford a white solid (56.1 mg, 38% recovery); R_{f} 0.14 with 1:30:70 v/v acetic acid-ethyl acetate-hexane (UV 254 nM); ¹H-NMR (400 MHz; DMSO-d6) δ 7.91 (br s, 1H), 7.78 (d, 1H, J=8.7 Hz), 7.33 (br s, 1H), 7.19 (d, 1H, J=1.4 Hz), 7.10 (br d, 1H, J=8.7 Hz), 7.01 (dd, 1H, J=2.1, 8.3 Hz), 6.8-6.9 (m, 2H), 4.77 (t, 1H, J=9.2 Hz), 4.30 (dd, 1H, J=7.1, 8.9 Hz), 3.8-3.9 (m, 1H), 3.56 (s, 3H), 3.04 (q, 1H, J=7.1 Hz), 2.87 (dd, 1H, J=5.5, 16.7 Hz), 2.5-2.6 (m, 1H); ¹⁹F-NMR (376 MHz; DMSO-d6) δ −56.71; MS (APCI+) m/z 435 (M+1); HPLC UV purity Rt=8.497 min, 98.22%; Chiral HPLC 98.9% area at 232 nM and 99.2% at 292 nM

Enantiomer 2 (peak 2) was concentrated under reduced pressure to afford a white solid (55.0 mg, 37% recovery); R_{f} 0.14 with 1:30:70 v/v acetic acid-ethyl acetate-hexane (UV 254 nM); ¹H-NMR (400 MHz; DMSO-d6) δ 7.91 (br s, 1H), 7.78 (d, 1H, J=8.7 Hz), 7.33 (br s, 1H), 7.19-7.21 (m, 2H), 7.10 (br d, 1H, J=8.7 Hz), 7.01 (dd, 1H, J=2.1, 8.5 Hz), 6.8-6.9 (m, 2H), 4.77 (t, 1H, J=9.2 Hz), 4.30 (dd, 1H, J=7.1, 8.9 Hz), 3.8-3.9 (m, 1H), 3.56 (s, 3H), 3.04 (q, 1H, J=7.1 Hz), 2.87 (dd, 1H, J=5.5, 16.7 Hz), 2.5-2.8 (m, 1H); ¹⁹F-NMR (376 MHz; DMSO-d6) δ −56.71; MS (APCI+) m/z 435 (M+1); HPLC UV purity Rt=8.499 min, 99.62%; Chiral HPLC 98.9% area at 232 nM and 99.0% at 292 nM

### Step B: Preparation of (+)-2-(5-(2-carbamoyl-6-(trifluoromethoxy)-1H-indol-1-yl)-2,3-dihydrobenzofuran-3-yl)acetic acid (Compound 1a) and (−)-2-(5-(2-carbamoyl-6-(trifluoromethoxy)-1H-indol-1-yl)-2,3-dihydrobenzofuran-3-yl)acetic acid (Compound 1b)

To the individually separated enantiomers of methyl 2-(5-(2-carbamoyl-6-(trifluoromethoxy)-1H-indol-1-yl)-2,3-dihydrobenzofuran-3-yl)acetate (0.052 g, 012 mmol) in methanol (3.0 mL) was added 1M lithium hydroxide (0.50 mL, 0.50 mmol). The reaction mixture was stirred at room temperature overnight under N₂ atmosphere. The reaction mixture was subsequently partitioned between ethyl acetate (25 mL) and 1M HCl (20 mL). The phases were separated, and the organic phase was partitioned with H₂O (25 mL), followed by brine (25 mL). The organic layer was concentrated under reduced pressure to afford the crude products as a white solid.

Enantiomer 1 (peak 1) was concentrated under reduced pressure to afford a white solid (0.048 g, 95.8% yield); R_{f} 0.06 with 1:30:70 v/v acetic acid-ethyl acetate-hexane (UV 254 nM); ¹H-NMR (400 MHz; DMSO-d6) δ 12.31 (br s, 1H), 7.90 (br s, 1H), 7.78 (d, 1H, J=8.7 Hz), 7.32 (br s, 1H), 7.19-7.2 (m, 2H), 7.10 (br d, 1H, J=8.7 Hz), 7.01 (dd, 1H, J=2.2, 8.4 Hz), 6.88 (s, 1H), 6.83 (d, 1H, J=8.3 Hz), 4.78 (t, 1H, J=9.2 Hz), 4.27 (dd, 1H, J=7.8, 8.8 Hz), 3.8-3.9 (m, 1H), 2.80 (dd, 1H, J=5.3, 16.7 Hz), 2.5-2.6 (m, 1H); ¹⁹F-NMR (376 MHz; DMSO-d6) δ −56.69; MS (APCI+) m/z 421 (M+1), MS (APCI−) m/z 419 (M−1); HPLC UV purity Rt=7.420 min, 98.05%; melting point=236.7° C.; optical rotation [α]²⁵_{D}=+16.3 (c=1, 1.5 mL IPA, 1.0 mL of CHCl₃);

Enantiomer 2 (peak 2) was concentrated under reduced pressure to afford a white solid (0.045 g, 87.9% yield); R_{f} 0.06 with 1:30:70 v/v acetic acid-ethyl acetate-hexane (UV 254 nM); ¹H-NMR (400 MHz; DMSO-d6) δ 12.33 (br s, 1H), 7.90 (br s, 1H), 7.78 (d, 1H, J=8.7 Hz), 7.32 (br s, 1H), 7.19-7.2 (m, 2H), 7.10 (br d, 1H, J=8.7 Hz), 7.01 (dd, 1H, J=2.2, 8.4 Hz), 6.88 (s, 1H), 6.83 (d, 1H, J=8.5 Hz), 4.78 (t, 1H, J=9.2 Hz), 4.27 (dd, 1H, J=7.6, 8.7 Hz), 3.8-3.9 (m, 1H), 2.80 (dd, 1H, J=5.3, 16.7 Hz), 2.5-2.6 (m, 1H); ¹⁹F-NMR (376 MHz; DMSO-d6) δ −56.69; MS (APCI+) m/z 421 (M+1), MS (APCI−) m/z 419 (M−1); HPLC UV purity Rt=7.421 min, 99.44%; melting point=237.7° C.; optical rotation [α]²⁵_{D}=−10.9 (c=1, 1.5 mL IPA, 0.5 mL of CHCl₃)

### Example 11: Synthesis of 3-(3-(2-carbamoyl-6-(trifluoromethoxy)-1H-indol-1-yl)phenyl)-2,2-dimethylpropanoic acid (Compound 3)

### Step A: Preparation of 1-(3-(3-ethoxy-2,2-dimethyl-3-oxopropyl)phenyl)-6-(trifluoromethoxy)-1H-indole-2-carboxylic acid

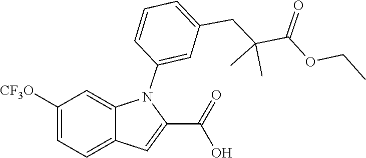

To a mixture consisting of 6-(trifluoromethoxy)-1H-indole-2-carboxylic acid (ChemShuttle, 0.200 g, 0.82 mmol), ethyl 3-(3-bromophenyl)-2,2-dimethylpropanoate (0.279 g, 0.98 mmol), copper (II) acetate (CombiBlocks, 0.148 g, 0.82 mmol), methyl-α-D-glucopyranoside (CombiBlocks, 0.158 g, 0.82 mmol), potassium iodide (VWR, 0.271 g, 1.63 mmol) in DMSO (5 mL) was added DBU (Oakwood Chemicals, 0.36 mL, 2.45 mmol). The reaction mixture was placed under N₂ atmosphere, and the reaction mixture was heated at 115° C. for 1 hour in the microwave. The reaction mixture was subsequently partitioned between ethyl acetate (40 mL) and 1M KHSO₄ (40 mL). An emulsion formed which was broken up by the addition of 5 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude brown oil, 0.400 g. The crude oil was purified by flash silica column chromatography on a CombiFlash NextGen 300+ purification system. Elution through a 40 g RediSep Gold Rf flash silica cartridge with 10-20% ethyl acetate in hexanes containing 1% acetic acid afforded the title compound as a mixture with starting material as a colorless oil (0.181 g). The crude reaction mixture was used as is in the next step.

### Step B: Preparation of ethyl 3-(3-(2-carbamoyl-6-(trifluoromethoxy)-1H-indol-1-yl)phenyl)-2,2-dimethylpropanoate

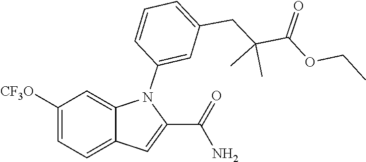

To a mixture consisting of crude 1-(3-(3-ethoxy-2,2-dimethyl-3-oxopropyl)phenyl)-6-(trifluoromethoxy)-1H-indole-2-carboxylic acid (0.180 g, 0.43 mmol) in DMF (5 mL) was added solid ammonium chloride (Chem-Impex, 0.065 g, 1.21 mmol) and 2-(1H-benzotriazole-1-yl)-1,1,3,3-tetramethylaminium tetrafluoroborate (Oakwood, 0.194 g, 0.61 mmol) followed by diisopropylethylamine (0.63 mL, 3.63 mmol). The reaction mixture was stirred overnight at room temperature under N₂ atmosphere. The reaction mixture was subsequently partitioned between ethyl acetate (50 mL) and H₂O (50 mL). The phases were separated, and the organic phase was partitioned with 1M KHSO₄ (50 mL), followed by brine (50 mL). The organic layer was concentrated under reduced pressure to afford the crude product as a colorless oil (0.163 g). The crude oil was purified by flash silica column chromatography on a CombiFlash NextGen 300+ purification system. Elution through a 12 g RediSep Gold Rf flash silica cartridge with 10-30% ethyl acetate in hexanes containing 1% acetic acid afforded the title compound as a colorless oil (0.114 g, 96% yield); R_{f} 0.18 with 1:30:70 v/v acetic acid-ethyl acetate-hexane (UV 254 nM); MS (APCI+) m/z 449.2 (M+1), (APCI−) m/z 447.20 (M−1); HPLC UV purity Rt=10.022 min, 42.5%. The product is a mixture of desired product and impurity: 6-(trifluoromethoxy)-1H-indole-2-carboxamide. The crude reaction mixture was used as is in the next step.

### Step C: Preparation of 3-(3-(2-carbamoyl-6-(trifluoromethoxy)-1H-indol-1-yl)phenyl)-2,2-dimethylpropanoic acid (Compound 3)

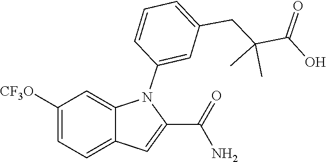

To a mixture consisting of ethyl 3-(3-(2-carbamoyl-6-(trifluoromethoxy)-1H-indol-1-yl)phenyl)-2,2-dimethylpropanoate (0.114 g, 0.25 mmol) in methanol (3 mL) was added 1M lithium hydroxide (1.0 mL, 1.0 mmol). The reaction mixture was stirred overnight at room temperature under N₂ atmosphere. The reaction mixture was subsequently partitioned between ethyl acetate (50 mL) and 1M HCl (25 mL). The phases were separated, and the organic phase was partitioned with H₂O (25 mL), followed by brine (25 mL). The organic layer was concentrated under reduced pressure to afford the crude product as a colorless oil (0.120 g). The crude oil was purified by flash silica column chromatography on a CombiFlash NextGen 300+ purification system. Elution through a 12 g RediSep Gold Rf flash silica cartridge with 10-50% ethyl acetate in hexanes containing 1% acetic acid afforded the title compound as a white solid (0.016 g); Rf 0.48 with 1:70:30 v/v acetic acid-ethyl acetate-hexane (UV 254 nM); ¹H-NMR (400 MHz; DMSO-d6) δ 12.21 (br s, 1H), 7.96 (br s, 1H), 7.80 (d, 1H, J=8.7 Hz), 7.3-7.4 (m, 2H), 7.24 (s, 1H), 7.20 (d, 1H, J=7.8 Hz), 7.1-7.2 (m, 3H), 6.89 (s, 1H), 2.83 (s, 2H), 1.06 (s, 6H); ¹⁹F-NMR (376 MHz; DMSO-d6) 56.78; MS (APCI⁺) m/z 421 (M+1), 419 (M−1). HPLC UV purity, Rt=6.19 min, 98.37%; melting point=88-89° C.

### Example 12: Synthesis of (E)-3-(6-(2-carbamoyl-6-(trifluoromethoxy)-1H-indol-1-yl)pyridin-2-yl)but-2-enoic acid (Compound 6)

### Step A: Preparation of (E)-1-(6-(4-methoxy-4-oxobut-2-en-2-yl)pyridin-2-yl)-6-(trifluoromethoxy)-1H-indole-2-carboxylic acid

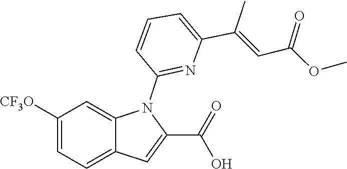

To a mixture consisting of 6-(trifluoromethoxy)-1H-indole-2-carboxylic acid (ChemShuttle, 0.200 g, 0.82 mmol), methyl (E/Z)-3-(6-bromopyridin-2-yl)but-2-enoate (0.209 g, 0.82 mmol), copper (II) acetate (CombiBlocks, 0.148 g, 0.82 mmol), methyl-α-D-glucopyranoside (CombiBlocks, 0.158 g, 0.82 mmol), potassium iodide (VWR, 0.271 g, 1.63 mmol) in DMSO (5 mL) was added DBU (Oakwood Chemicals, 0.36 mL, 2.45 mmol). The reaction mixture was stirred overnight at 115° C. under N₂ atmosphere. The reaction mixture was subsequently partitioned between ethyl acetate (60 mL) and 1M KHSO₄ (50 mL). An emulsion formed which was broken up by the addition of 25 mL of brine. The phases were separated, and the organic phase was partitioned with H₂O (60 mL), followed by brine (60 mL). The organic layer was separated and dried over anhydrous magnesium sulfate, filtered, and concentrated under reduced pressure to give a crude brown oil, 0.400 g. The crude oil was purified by flash silica column chromatography on a CombiFlash NextGen 300+ purification system. Elution through a 24 g RediSep Gold Rf flash silica cartridge with 10-50% ethyl acetate in hexanes containing 1% acetic acid afforded the title compound as a white solid (0.061 g, 18% yield); R_{f} 0.09 with 1:20:80 v/v acetic acid-ethyl acetate-hexane (UV 254 nM); ¹H-NMR (400 MHz; CDCl₃) δ 12.7-13.0 (bs, 1H), 7.92 (t, 1H, J=8.7 Hz), 7.73 (d, 1H, J=8.7 Hz), 7.63 (d, 1H, J=7.8 Hz), 7.55 (s, 1H), 7.3-7.4 (m, 2H), 7.0-7.2 (m, 1H), 6.75 (d, 1H, J=1.1 Hz), 3.74 (s, 3H), 2.62 (d, 3H, J=1.1 Hz); ¹⁹F-NMR (376 MHz; DMSO-d6) δ check; MS (APCI+) m/z 421 (M+1), (APCI−) m/z 419 (M−1); HPLC UV purity Rt=9.396 min, 99.09%.

### Step B: Preparation of methyl (E)-3-(6-(2-carbamoyl-6-(trifluoromethoxy)-1H-indol-1-yl)pyridin-2-yl)but-2-enoate

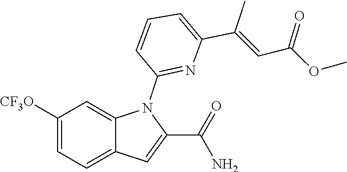

To a mixture consisting of (E)-1-(6-(4-methoxy-4-oxobut-2-en-2-yl)pyridin-2-yl)-6-(trifluoromethoxy)-1H-indole-2-carboxylic acid (0.057 g, 0.14 mmol) in DMF (2 mL) was added solid ammonium chloride (Chem-Impex, 0.022 g, 0.41 mmol) and 2-(1H-benzotriazole-1-yl)-1,1,3,3-tetramethylaminium tetrafluoroborate (Oakwood, 0.066 g, 0.20 mmol) followed by diisopropylethylamine (0.21 mL, 1.22 mmol). The reaction mixture was stirred for 3 hours at room temperature under N₂ atmosphere. The reaction mixture was subsequently partitioned between ethyl acetate (25 mL) and H₂O (25 mL). The phases were separated, and the organic phase was partitioned with 1M KHSO₄ (20 mL), followed by brine (30 mL). The organic layer was concentrated under reduced pressure to afford the crude product as a yellow oil (0.056 g, 98% yield). R_{f} 0.07 with 1:30:70 v/v acetic acid-ethyl acetate-hexane (UV 254 nM); ¹H-NMR (400 MHz; CDCl₃) δ 8.18 (br s, 1H), 8.02 (t, 1H, J=8.7 Hz), 7.8-7.9 (m, 2H), 7.51 (br s, 1H), 7.4-7.5 (m, 2H), 7.25 (s, 1H), 7.18 (br d, 1H, J=8.5 Hz), 6.77 (d, 1H, J=1.4 Hz), 3.66 (s, 3H), 2.6-2.7 (m, 1H), 2.53 (d, 3H, J=1.4 Hz); ¹⁹F-NMR (376 MHz; DMSO-d6) δ −56.76: MS (APCI+) m/z 420.0 (M+1); HPLC UV purity Rt=8.544 min, 88.1%.

### Step C: Preparation of (E)-3-(6-(2-carbamoyl-6-(trifluoromethoxy)-1H-indol-1-yl)pyridin-2-yl)but-2-enoic acid (Compound 6)

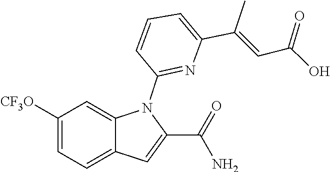

To a mixture consisting of (E)-1-(6-(4-methoxy-4-oxobut-2-en-2-yl)pyridin-2-yl)-6-(trifluoromethoxy)-1H-indole-2-carboxylic acid (0.056 g, 0.13 mmol) in methanol (3 mL) was added 1M lithium hydroxide (0.5 mL, 0.5 mmol). The reaction mixture was stirred overnight at room temperature under N₂ atmosphere. The reaction mixture was subsequently partitioned between ethyl acetate (25 mL) and 1M HCl (10 mL). The phases were separated, and the organic phase was partitioned with H₂O (25 mL), followed by brine (25 mL). The organic layer was concentrated under reduced pressure to afford the crude product as a white solid (0.060 g). The crude solid was purified by flash silica column chromatography on a CombiFlash NextGen 300+ purification system. Elution through a 4 g RediSep Gold Rf flash silica cartridge with 20-80% ethyl acetate in hexanes containing 1% acetic acid afforded the title compound as a white solid (0.014 g, 26% yield); Rf 0.31 with 1:80:20 v/v acetic acid-ethyl acetate-hexane (UV 254 nM); ¹H-NMR (400 MHz; DMSO-d6) δ 12.45 (br s, 1H), 8.21 (br s, 1H), 8.05 (t, 1H, J=7.9 Hz), 7.87 (d, 1H, J=8.7 Hz), 7.80 (d, 1H, J=7.8 Hz), 7.54 (br s, 1H), 7.48 (s, 1H), 7.43 (d, 1H, J=7.8 Hz), 7.29 (s, 2H), 7.21 (br d, 1H, J=8.5 Hz), 6.7-6.8 (m, 1H) 2.53 (d, 3H, J=0.9 Hz); ¹⁹F-NMR (376 MHz; DMSO-d6) 56.78; MS (APCI⁺) m/z 406 (M+1), 404 (M−1). HPLC UV purity, Rt=7.102 min, 99.00%; melting point=232-234° C. Product confirmed by NOE to be (E) configuration.

### Example 13: Synthesis of 2-((3-(2-carbamoyl-6-(trifluoromethoxy)-1H-indol-1-yl)phenyl)thio)-2-methylpropanoic acid (Compound 2)

### Step A: Preparation of 1-(3-((1-ethoxy-2-methyl-1-oxopropan-2-yl)thio)phenyl)-6-(trifluoromethoxy)-1H-indole-2-carboxylic acid

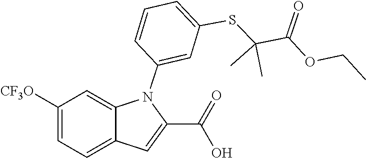

To a mixture consisting of 6-(trifluoromethoxy)-1H-indole-2-carboxylic acid (ChemShuttle, 0.200 g, 0.82 mmol), ethyl 2-((3-bromophenyl)thio)-2-methylpropanoate (0.248 g, 0.82 mmol), copper (II) acetate (CombiBlocks, 0.296 g, 1.64 mmol), methyl-α-D-glucopyranoside (CombiBlocks, 0.158 g, 0.82 mmol), potassium iodide (VWR, 0.272 g, 1.64 mmol) in DMSO (12 mL) was added DBU (Oakwood Chemicals, 0.36 mL, 2.45 mmol). The reaction mixture was stirred overnight at 115° C. under N₂ atmosphere. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.503 g. The crude oil was purified by flash silica column chromatography on a CombiFlash NextGen 300+ purification system. Elution through a 24 g RediSep Gold Rf flash silica cartridge with 5-15% ethyl acetate in hexanes containing 1% acetic acid afforded the title compound as a mixture with starting material. Collected the mixture as a white solid (0.082 g, 21% yield); Rf 0.38 with 1:30:70 v/v acetic acid-ethyl acetate-hexane (UV 254 nM); MS (APCI−) m/z 466 (M−1). The crude isolated reaction mixture was used in the next step.

### Step B: Preparation of ethyl 2-((3-(2-carbamoyl-6-(trifluoromethoxy)-1H-indol-1-yl)phenyl)thio)-2-methylpropanoate

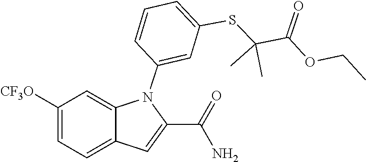

To a mixture consisting of ethyl 2-((3-(2-carbamoyl-6-(trifluoromethoxy)-1H-indol-1-yl)phenyl)thio)-2-methylpropanoate (0.082 g, 0.17 mmol) in DMF (2 mL) was added solid ammonium chloride (Chem-Impex, 0.028 g, 0.53 mmol) and 2-(1H-benzotriazole-1-yl)-1,1,3,3-tetramethylaminium tetrafluoroborate (Oakwood, 0.263 g, 0.84 mmol) followed by diisopropylethylamine (0.27 mL, 1.58 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (25 mL), followed by brine (25 mL). The organic layer was concentrated under reduced pressure to afford the crude product as a colorless oil (0.073 g, 90.9% yield). The crude product was obtained as a mixture with a known impurity. The crude product was used without further purification. Rf 0.11 with 1:20:80 v/v acetic acid-ethyl acetate-hexane (UV 254 nM); LCMS, 2 peaks, Rt=7.55 min, m/z 467.10 (M+1);

### Step C: Preparation of 2-((3-(2-carbamoyl-6-(trifluoromethoxy)-1H-indol-1-yl)phenyl)thio)-2-methylpropanoic acid (Compound 2)

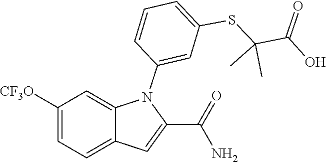

To a mixture consisting of ethyl 2-((3-(2-carbamoyl-6-(trifluoromethoxy)-1H-indol-1-yl)phenyl)thio)-2-methylpropanoate (0.073 g, 0.16 mmol) in methanol (3 mL) was added 1M lithium hydroxide (0.63 mL, 0.63 mmol). The reaction mixture was stirred overnight at room temperature under N₂ atmosphere. The reaction mixture was subsequently partitioned between ethyl acetate (25 mL) and 1M HCl (10 mL). The phases were separated, and the organic phase was partitioned with H₂O (25 mL), followed by brine (25 mL). The organic layer was concentrated under reduced pressure to afford the crude product as a colorless oil (0.073 g). The crude product was purified by flash silica column chromatography on a CombiFlash NextGen 300+ purification system. Elution through a 12 g RediSep Gold Rf flash silica cartridge with 10-80% ethyl acetate in hexanes containing 1% acetic acid afforded the title compound as a white solid (0.036 g, 53% yield); R_{f} 0.09 with 1:30:70 v/v acetic acid-ethyl acetate-hexane (UV 254 nM); ¹H-NMR (400 MHz; DMSO-d6) δ 12.66 (br s, 1H), 8.02 (br s, 1H), 7.82 (d, 1H, J=8.5 Hz), 7.4-7.5 (m, 2H), 7.3-7.4 (m, 3H), 7.29 (s, 1H), 7.13 (br d, 1H, J=8.7 Hz), 6.93 (br s, 1H), 1.38 (s, 6H); ¹⁹F-NMR (376 MHz; DMSO-d6) 56.74; MS (APCI⁺) m/z 439 (M+1), 437 (M−1). HPLC UV purity, Rt=8.376 min, 91.66%.

### Example 14: Synthesis of 1-((3-(2-carbamoyl-6-(trifluoromethoxy)-1H-indol-1-yl)phenyl)thio)cyclobutane-1-carboxylic acid (Compound 4)

### Step A: Preparation of 1-(3-((1-(ethoxycarbonyl)cyclobutyl)thio)phenyl)-6-(trifluoromethoxy)-1H-indole-2-carboxylic acid

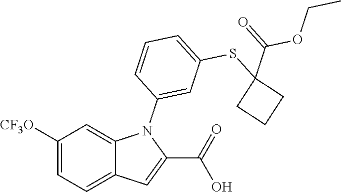

To a mixture consisting of 6-(trifluoromethoxy)-1H-indole-2-carboxylic acid (ChemShuttle, 0.200 g, 0.82 mmol), ethyl 1-((3-bromophenyl)thio)cyclobutane-1-carboxylate (0.258 g, 0.82 mmol), copper (II) acetate (CombiBlocks, 0.296 g, 1.64 mmol), methyl-α-D-glucopyranoside (CombiBlocks, 0.158 g, 0.82 mmol), potassium iodide (VWR, 0.272 g, 1.64 mmol) in DMSO (12 mL) was added DBU (Oakwood Chemicals, 0.36 mL, 2.45 mmol). The reaction mixture was stirred overnight at 115° C. under N₂ atmosphere. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.475 g. The crude oil was purified by flash silica column chromatography on a CombiFlash NextGen 300+ purification system. Elution through a 24 g RediSep Gold Rf flash silica cartridge with 5-20% ethyl acetate in hexanes containing 1% acetic acid afforded the title compound as a mixture with starting material. Collected the mixture as a white solid (0.135 g); R_{f} 0.41 with 1:30:70 v/v acetic acid-ethyl acetate-hexane (UV 254 nM); MS (APCI−) m/z 478 (M−1). The crude isolated reaction mixture was used in the next step.

### Step B: Preparation of ethyl 1-((3-(2-carbamoyl-6-(trifluoromethoxy)-1H-indol-1-yl)phenyl)thio)cyclobutane-1-carboxylate

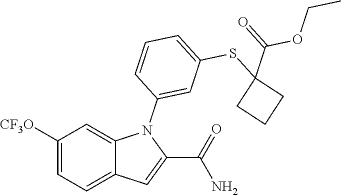

To a mixture consisting of 1-(3-((1-(ethoxycarbonyl)cyclobutyl)thio)phenyl)-6-(trifluoromethoxy)-1H-indole-2-carboxylic acid (0.125 g, 0.26 mmol) in DMF (3 mL) was added solid ammonium chloride (Chem-Impex, 0.042 g, 0.78 mmol) and 2-(1H-benzotriazole-1-yl)-1,1,3,3-tetramethylaminium tetrafluoroborate (Oakwood, 0.125 g, 0.39 mmol) followed by diisopropylethylamine (0.41 mL, 2.34 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (25 mL), followed by brine (25 mL). The organic layer was concentrated under reduced pressure to afford the crude product as a colorless oil (0.109 g, 87.9% yield). The crude product was obtained as a mixture with a known impurity. The crude product was used without further purification. R_{f} 0.11 with 1:20:80 v/v acetic acid-ethyl acetate-hexane (UV 254 nM); LCMS, 2 peaks, Rt=7.60 min, m/z 479.10 (M+1);

### Step C: Preparation of 1-((3-(2-carbamoyl-6-(trifluoromethoxy)-1H-indol-1-yl)phenyl)thio)cyclobutane-1-carboxylic acid (Compound 4)

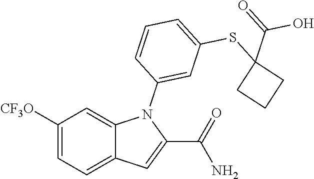

To a mixture consisting of ethyl 1-((3-(2-carbamoyl-6-(trifluoromethoxy)-1H-indol-1-yl)phenyl)thio)cyclobutane-1-carboxylate (0.109 g, 0.23 mmol) in methanol (3 mL) was added 1M lithium hydroxide (0.91 mL, 0.91 mmol). The reaction mixture was stirred overnight at room temperature under N₂ atmosphere. The reaction mixture was subsequently partitioned between ethyl acetate (25 mL) and 1M HCl (15 mL). The phases were separated, and the organic phase was partitioned with H₂O (25 mL), followed by brine (25 mL). The organic layer was concentrated under reduced pressure to afford the crude product as a colorless oil (0.100 g). The crude product was purified by flash silica column chromatography on a CombiFlash NextGen 300+ purification system. Elution through a 12 g RediSep Gold Rf flash silica cartridge with 10-30% ethyl acetate in hexanes containing 1% acetic acid afforded the title compound as a white solid (0.036 g, 33% yield); R_{f} 0.08 with 1:30:70 v/v acetic acid-ethyl acetate-hexane (UV 254 nM); ¹H-NMR (400 MHz; DMSO-d6) δ 12.7-1.2.8 (br s, 1H), 8.02 (br s, 1H), 7.81 (d, 1H, J=8.7 Hz), 7.4-7.5 (m, 1H), 7.3-7.4 (m, 1H), 7.2-7.3 (m, 3H), 7.13 (br d, 1H, J=8.3 Hz), 6.96 (s, 1H), 2.5-2.7 (m, 5H), 2.29 (s, 1H). 2.1-2.2 (m, 3H); ¹⁹F-NMR (376 MHz; DMSO-d6) 56.74; MS (APCI⁺) m/z 439 (M+1), 437 (M−1). HPLC UV purity, Rt=8.55 min, 84.81%.

### Example 15: Synthesis of 3-(3-(2-carbamoyl-5-(trifluoromethoxy)benzo[b]

thiophen-3-yl)phenyl)propanoic acid (Compound 5)

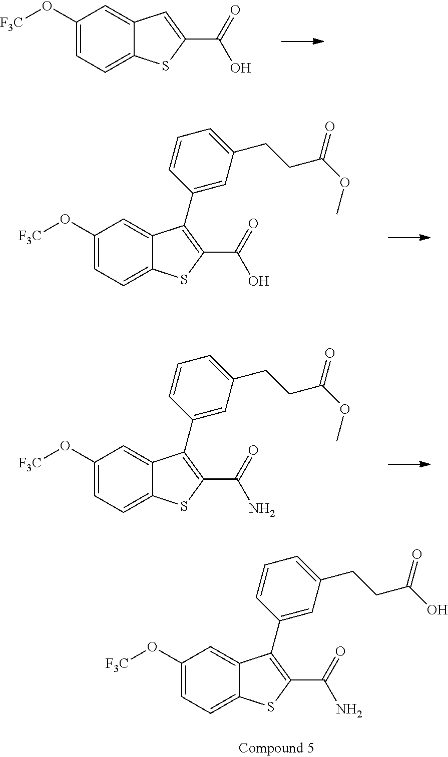 [SMILES: O=C(O)c1cc2cc(OC(F)(F)F)ccc2s1 ; COC(=O)CCc1cccc(-c2c(C(=O)O)sc3ccc(OC(F)(F)F)cc23)c1 ; COC(=O)CCc1cccc(-c2c(C(N)=O)sc3ccc(OC(F)(F)F)cc23)c1 ; NC(=O)c1sc2ccc(OC(F)(F)F)cc2c1-c1cccc(CCC(=O)O)c1]

### Step A: Preparation of 3-(3-(3-methoxy-3-oxopropyl)phenyl)-5-(trifluoromethoxy)benzo[b]thiophene-2-carboxylic acid

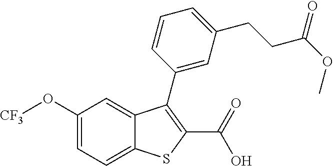 [SMILES: COC(=O)CCc1cccc(-c2c(C(=O)O)sc3ccc(OC(F)(F)F)cc23)c1]

To a 24 mL septa-cap vial was added methyl 3-bromobenzenepropanoate (Combi-Blocks, 0.243 g, 1.0 mmol) and 5-(trifluoromethoxy)benzo[b]thiophene-2-carboxylic acid (0.262 g, 1.0 mmol); prepared by the method of Zhao, L. et. al., Eur J Med Chem 2022, 228, 113987. Next was added Bis(dichloro(η⁶-p-cymene)ruthenium) (Strem, 0.025 g, 4 mol %), trimethylphosphonium tetrafluoroborate (Strem, 0.013 g, 8 mol %), K₂CO₃ (VWR, 0.152 g, 1.1 mmol) and NMP (5 mL). The mixture was degassed by bubbling in nitrogen gas for 3 min with stirring then heated to 105° C. overnight. The reaction mixture was subsequently cooled to room temperature then partitioned between ethyl acetate (30 mL) and 1M KHSO₄ (30 mL). The phases were separated, and the organic phase was partitioned with H₂O (75 mL), followed by brine (75 mL). The organic layer was separated and dried over anhydrous sodium sulfate, filtered, and concentrated under reduced pressure to give a crude oil which was purified by flash silica column chromatography on a CombiFlash NextGen 300+ purification system. Elution through a 40 g RediSep Gold Rf flash silica cartridge with 10-100% ethyl acetate containing 1% acetic acid afforded the title compound as an amorphous solid (0.090 g, 21% yield); R_{f} 0.28 with 1:30:70 v/v acetic acid-ethyl acetate-hexane (UV 254 nM); ¹H-NMR (400 MHz; CD₃OD) δ 8.05 (d, 1H, J=8.9 Hz), 7.4-7.5 (m, 2H), 7.33 (d, 1H, J=7.8 Hz), 7.28 (s, 1H), 7.2-7.3 (m, 2H), 3.64 (s, 3H), 3.3-3.3 (m, 1H), 3.01 (t, 2H, J=7.6 Hz), 2.70 (t, 2H, J=7.5 Hz); MS (APCI⁺) m/z 442.1 (M+NH₄⁺).

### Step B: Preparation of methyl 3-(3-(2-carbamoyl-5-(trifluoromethoxy)benzo[b]thiophen-3-yl)phenyl)propanoate

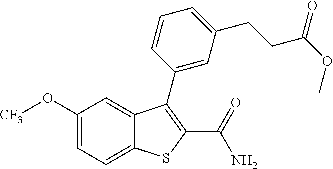 [SMILES: COC(=O)CCc1cccc(-c2c(C(N)=O)sc3ccc(OC(F)(F)F)cc23)c1]

To a mixture consisting of 3-(3-(3-methoxy-3-oxopropyl)phenyl)-5-(trifluoromethoxy)benzo[b]thiophene-2-carboxylic acid (0.090 g, 0.21 mmol) in DMF (1.5 mL) was added ammonium chloride (Chem-Impex, 0.034 g, 0.63 mmol). Next was added TBTU (Oakwood, 0.101 g, 0.31 mmol) followed by the addition of DIPEA (0.33 mL, 1.9 mmol). The reaction mixture was stirred overnight at room temperature under N₂ atmosphere. The reaction mixture was subsequently diluted with water (40 mL) then extracted with 3×20 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 10-100% ethyl acetate in hexanes containing 1% acetic acid afforded the title compound as a white semi-solid (0.076 g, 84% yield); R_{f} 0.47 with 1:40:60 v/v acetic acid-ethyl acetate-hexane (UV 254 nM); ¹H-NMR (400 MHz; CD₃OD) δ 8.06 (d, 1H, J=8.9 Hz), 7.5-7.6 (m, 1H), 7.4-7.5 (m, 2H), 7.35 (s, 1H), 7.31 (d, 1H, J=7.6 Hz), 7.26 (s, 1H), 3.63 (s, 3H), 3.03 (t, 2H, J=7.3 Hz), 2.72 (t, 2H, J=7.3 Hz); MS (APCI⁺) m/z 424.0 (M+1).

### Step C: Preparation of methyl 3-(3-(2-carbamoyl-5-(trifluoromethoxy)benzo[b]thiophen-3-yl)phenyl)propanoic acid (Compound 5)

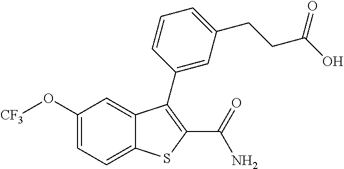 [SMILES: NC(=O)c1sc2ccc(OC(F)(F)F)cc2c1-c1cccc(CCC(=O)O)c1]

To a mixture consisting of methyl 3-(3-(2-carbamoyl-5-(trifluoromethoxy)benzo[b]thiophen-3-yl)phenyl)propanoate (0.071 g, 0.17 mmol) in MeOH (2 mL) was added dropwise, a 1M aqueous solution of LiOH (0.51 mL, 3 eq). The reaction mixture was stirred overnight at room temperature. The clear solution was subsequently diluted with water (10 mL) and subjected to reduced pressure on a rotary evaporator to remove MeOH. Additional water was added (10 mL) and the solution was acidified with 1N HCl to precipitate product. The suspension was filtered and solids washed with excess water then dried under high vac overnight to afford the title compound as a white solid (0.045 g, 65% yield); R_{f} 0.27 with 1/40/60 acetic acid:ethyl acetate:heptane (UV 254 nM); ¹H-NMR (400 MHz; DMSO-d₆) δ 12.17 (br s, 1H), 8.25 (d, 1H, J=8.7 Hz), 7.75 (br s, 1H), 7.4-7.6 (m, 2H), 7.39 (br d, 1H, J=7.6 Hz), 7.34 (s, 1H), 7.3-7.3 (m, 2H), 6.82 (br s, 1H), 2.91 (br t, 2H, J=7.6 Hz), 2.59 (t, 2H, J=7.7 Hz); MS (APCI⁺) m/z 409.1, 410.1 (M+1). HPLC UV purity, Rt=7.98 min, 99.4%. Melting point 185.0-186.0° C.

### Example 16: Synthesis of 2-(5-(2-carbamoyl-5-(trifluoromethoxy)benzo[b]thiophen-3-yl)-2,3-dihydrobenzofuran-3-yl)acetic acid (Compound 7)

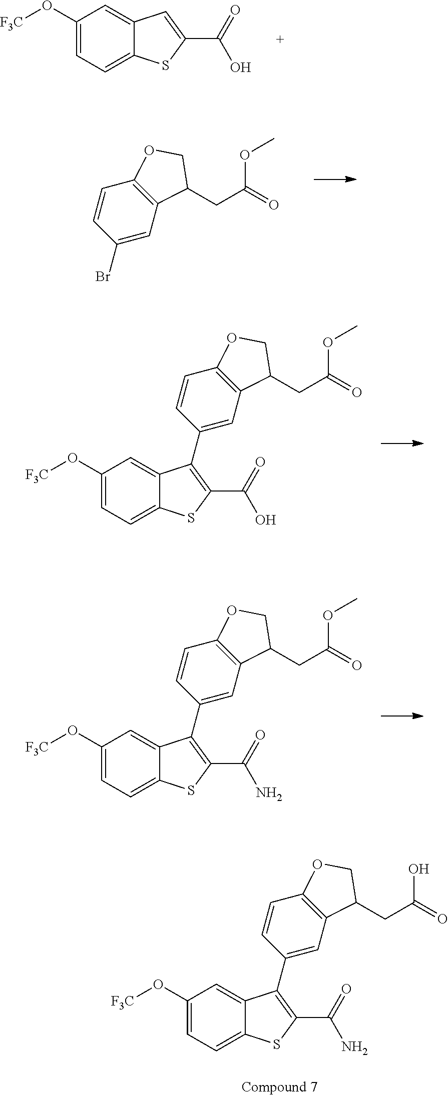 [SMILES: O=C(O)c1cc2cc(OC(F)(F)F)ccc2s1 ; COC(=O)CC1COc2ccc(Br)cc21 ; COC(=O)CC1COc2ccc(-c3c(C(=O)O)sc4ccc(OC(F)(F)F)cc34)cc21 ; COC(=O)CC1COc2ccc(-c3c(C(N)=O)sc4ccc(OC(F)(F)F)cc34)cc21 ; NC(=O)c1sc2ccc(OC(F)(F)F)cc2c1-c1ccc2c(c1)C(CC(=O)O)CO2]

### Step A: Preparation of 3-(3-(2-methoxy-2-oxoethyl)-2,3-dihydrobenzofuran-5-yl)-5-(trifluoromethoxy)benzo[b]thiophene-2-carboxylic acid

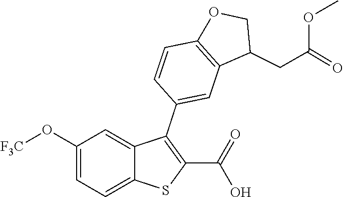 [SMILES: COC(=O)CC1COc2ccc(-c3c(C(=O)O)sc4ccc(OC(F)(F)F)cc34)cc21]

To a 24 mL septa-cap vial was added methyl 5-bromo-2,3-dihydrobenzofuran-3-acetate (Accela, 0.298 g, 1.1 mmol) and 5-(trifluoromethoxy)benzo[b]thiophene-2-carboxylic acid (0.262 g, 1.0 mmol); prepared by the method of Zhao, L. et. al., Eur J Med Chem 2022, 228, 113987. Next was added Bis(dichloro(η⁶-p-cymene)ruthenium) (Strem, 0.025 g, 4 mol %), trimethylphosphonium tetrafluoroborate (Strem, 0.015 g, 8 mol %), K₂CO₃ (VWR, 0.153 g, 1.1 mmol) and NMP (5 mL). The mixture was degassed by bubbling in nitrogen gas for 3 min with stirring then heated to 105° C. overnight. The reaction mixture was subsequently cooled to room temperature then partitioned between ethyl acetate (30 mL) and 1M KHSO₄ (30 mL). The phases were separated, and the organic phase was partitioned with H₂O (75 mL), followed by brine (75 mL). The organic layer was separated and dried over anhydrous sodium sulfate, filtered, and concentrated under reduced pressure to give a crude oil which was purified by flash silica column chromatography on a CombiFlash NextGen 300+ purification system. Elution through a 40 g RediSep Gold Rf flash silica cartridge with 10-100% ethyl acetate containing 1% acetic acid afforded the title compound as an amorphous solid (0.156 g, 34% yield); R_{f} 0.27 with 1:30:70 v/v acetic acid-ethyl acetate-hexane (UV 254 nM); ¹H-NMR (400 MHz; CDCl₃) δ 7.90 (d, 1H, J=8.9 Hz), 7.4-7.4 (m, 2H), 7.2-7.2 (m, 2H), 6.93 (d, 1H, J=8.0 Hz), 4.89 (t, 1H, J=9.2 Hz), 4.39 (dd, 1H, J=6.3, 9.3 Hz), 3.9-4.0 (m, 1H), 3.71 (s, 3H), 2.84 (dd, 1H, J=5.6, 16.6 Hz), 2.69 (dd, 1H, J=9.1, 16.6 Hz); MS (APCI⁺) m/z 435.0 (M+1−H₂O), (APCI⁻) m/z 451.0 (M−1).

### Step B: Preparation of methyl 2-(5-(2-carbamoyl-5-(trifluoromethoxy)benzo[b]thiophen-3-yl)-2,3-dihydrobenzofuran-3-yl)acetate

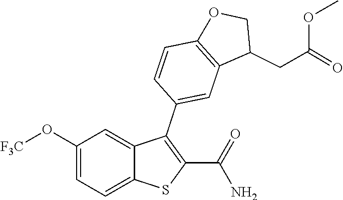 [SMILES: COC(=O)CC1COc2ccc(-c3c(C(N)=O)sc4ccc(OC(F)(F)F)cc34)cc21]

To a mixture consisting of 3-(3-(3-methoxy-3-oxopropyl)phenyl)-5-(trifluoromethoxy)benzo[b]thiophene-2-carboxylic acid (0.090 g, 0.21 mmol) in DMF (2.0 mL) was added ammonium chloride (Chem-Impex, 0.052 g, 0.97 mmol). Next was added TBTU (Oakwood, 0.156 g, 0.49 mmol) followed by the addition of DIPEA (0.51 mL, 2.9 mmol). The reaction mixture was stirred overnight at room temperature under N₂ atmosphere. The reaction mixture was subsequently diluted with water (40 mL) then extracted with 3×20 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 10-100% ethyl acetate in hexanes containing 1% acetic acid afforded the title compound as a white semi-solid (0.119 g, 81% yield); R_{f} 0.26 with 1:30:70 v/v acetic acid-ethyl acetate-hexane (UV 254 nM); ¹H-NMR (400 MHz; CD₃OD) δ 7.88 (d, 1H, J=8.7 Hz), 7.32 (d, 1H, J=8.9 Hz), 7.2-7.2 (m, 3H), 6.99 (d, 1H, J=8.7 Hz), 5.88 (br s, 1H), 5.65 (br s, 1H), 4.88 (t, 1H, J=9.2 Hz), 4.39 (dd, 1H, J=6.3, 9.3 Hz), 3.9-4.0 (m, 1H), 3.69 (s, 3H), 2.80 (dd, 1H, J=5.8, 16.4 Hz), 2.67 (dd, 1H, J=8.6, 16.4 Hz); MS (APCI⁺) m/z 452.0 (M+1).

### Step C: Preparation of 2-(5-(2-carbamoyl-5-(trifluoromethoxy)benzo[b]thiophen-3-yl)-2,3-dihydrobenzofuran-3-yl)acetic acid (Compound 7)

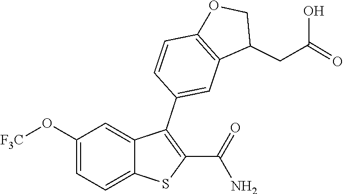 [SMILES: NC(=O)c1sc2ccc(OC(F)(F)F)cc2c1-c1ccc2c(c1)C(CC(=O)O)CO2]

To a mixture consisting of methyl 2-(5-(2-carbamoyl-5-(trifluoromethoxy)benzo[b]thiophen-3-yl)-2,3-dihydrobenzofuran-3-yl)acetate (0.114 g, 0.25 mmol) in MeOH (2 mL) was added dropwise, a 1M aqueous solution of LiOH (0.76 mL, 3 eq). The reaction mixture was stirred overnight at room temperature. The reaction mixture was subsequently partitioned between ethyl acetate (25 mL) and 1M HCl (15 mL). The phases were separated, and the organic phase was partitioned with H₂O (25 mL), followed by brine (25 mL). The organic layer was concentrated under reduced pressure to afford the desired product as a white solid (0.104 g, 95% yield); Rf 0.10 with 1/30/70 acetic acid:ethyl acetate:heptane (UV 254 nM); ¹H-NMR (400 MHz; 7DMSO-d₆) δ 12.34 (br s, 1H), 8.18 (d, 1H, J=8.9 Hz), 7.72 (br s, 1H), 7.47 (br d, 1H, J=8.7 Hz), 7.33 (s, 1H), 7.27 (s, 1H), 7.18 (dd, 1H, J=1.6, 8.9 Hz), 6.92 (d, 1H, J=8.3 Hz), 6.65 (br s, 1H), 4.78 (t, 1H, J=9.2 Hz), 4.27 (dd, 1H, J=7.3, 8.9 Hz), 3.8-3.9 (m, 1H), 2.80 (dd, 1H, J=5.3, 16.7 Hz), 2.55-2.62 (m 1H); ¹⁹F-NMR (376 MHz; DMSO-d6) δ −56.79; MS (APCI⁺) m/z 438.0 (M+1), (APCI−) m/z 436.0 (M-1); HPLC UV purity, Rt=7.975 min, 99.09%; Melting point 235.4-235.7° C.

### Example 17: Synthesis of ethyl 3-(6-(2-carbamoyl-6-(trifluoromethoxy)-1H-indole-1-yl)pyridin-2-yl)-2,2-dimethylpropanoate (Compound 8)

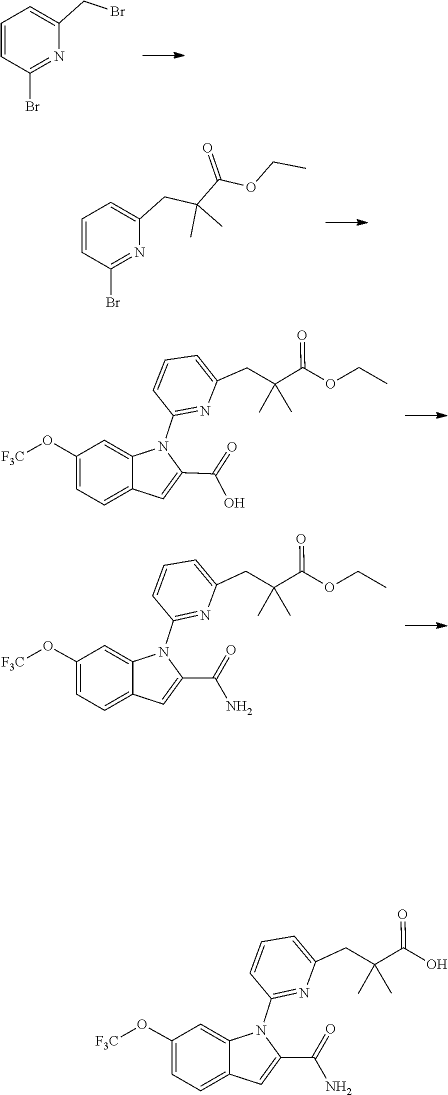 [SMILES: BrCc1cccc(Br)n1 ; CCOC(=O)C(C)(C)Cc1cccc(Br)n1 ; CCOC(=O)C(C)(C)Cc1cccc(-n2c(C(=O)O)cc3ccc(OC(F)(F)F)cc32)n1 ; CCOC(=O)C(C)(C)Cc1cccc(-n2c(C(N)=O)cc3ccc(OC(F)(F)F)cc32)n1 ; CC(C)(Cc1cccc(-n2c(C(N)=O)cc3ccc(OC(F)(F)F)cc32)n1)C(=O)O]

### Step A: Preparation of ethyl 3-(3-bromophenyl)-2,2-dimethylpropanoate

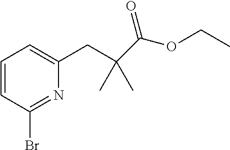 [SMILES: CCOC(=O)C(C)(C)Cc1cccc(Br)n1]

To a 20 mL DMSO solution of ethyl isobutyrate (805 uL, 5.98 mmol) cooled to −78° C. was added 2M LDA (3.0 mL, 5.98 mmol) dropwise under N₂ atmosphere. After stirring 30 minutes, a 3 mL DMSO solution of 1-bromo-3-(bromomethyl)benzene (996.5 mg, 3.97 mmol) was added dropwise and allowed to stir for 1 hour. After which, the reaction was allowed to warm to room temperature and stirred an additional 3 hours. The reaction mixture was partitioned between 25 mL ethyl acetate and 15 mL saturated NH₄Cl. The organic layer was sequentially washed with H₂O (15 mL) and brine (15 mL), then dried over anhydrous sodium sulfate. The crude reaction mixture was purified by flash silica column chromatography on a CombiFlash NextGen 300+ purification system. Elution through a 80 g RediSep Gold Rf flash silica cartridge with 0-10% ethyl acetate in hexanes afforded the title compound as a clear and colorless oil (0.7752 g, 68% yield); ¹H-NMR (400 MHz; CDCl₃-d) 6 7.40 (t, 1H, J=7.7 Hz), 7.29 (d, 1H, J=8.0 Hz), 7.04 (d, 1H, J=7.3 Hz), 4.14 (q, 2H, J=7.1 Hz), 3.00 (s, 2H), 1.2-1.3 (m, 9H); MS (FIA MS+) m/z 287 (M+1).

### Step B: Preparation of 1-(6-(3-ethoxy-2,2-dimethyl-3-oxopropyl)pyridin-2-yl)-6-(trifluoromethoxy)-1H-indole-2-carboxylix acid

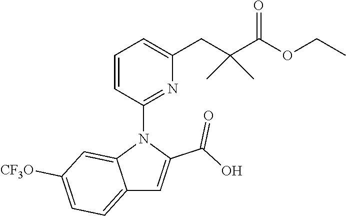

To an oven-dried round bottom flask flushed with N₂ was added 6-(trifluoromethoxy)-1H-indole-2-carboxylic acid (196.3 mg, 0.80 mmol), copper (II) acetate (148.9 mg, 0.82 mmol), methyl-α-D-glucopyranoside (159.7 mg, 0.82 mmol), potassium iodide (272.8 mg, 1.64 mmol), followed by a 6 mL DMSO solution of ethyl 3-(3-bromophenyl)-2,2-dimethylpropanoate (281.9 mg, 0.98 mmol). Lastly added was DBU (366 uL, 2.45 mmol). The reaction mixture was stirred at 115° C. under N₂ atmosphere for 3 hours. The reaction mixture was subsequently partitioned between ethyl acetate (20 mL) and 1M KHSO₄ (15 mL). An emulsion formed which was broken up by the addition of 5 mL of brine. The phases were separated, and the organic phase was partitioned with H₂O (15 mL), followed by brine (15 mL). The organic layer was separated and dried over anhydrous sodium sulfate, filtered, and concentrated under reduced pressure to give a crude oil. The crude oil was purified by flash silica column chromatography on a CombiFlash NextGen 300+ purification system. Elution through a 24 g RediSep Gold Rf flash silica cartridge with 5% ethyl acetate in hexanes containing 1% acetic acid afforded the title compound as a white solid (108.5 mg, 12% yield); ¹H-NMR (400 MHz; DMSO-d6) δ 13.04 (br s, 1H), 7.8-7.9 (m, 2H), 7.39 (s, 1H), 7.32 (d, 1H, J=8.0 Hz), 7.26 (d, 1H, J=7.6 Hz), 7.16 (br d, 1H, J=8.7 Hz), 7.12 (s, 1H), 3.99 (d, 1H, J=7.3 Hz), 3.8-3.9 (m, 1H), 3.85 (d, 1H, J=7.1 Hz), 2.99 (s, 2H), 1.95 (s, 1H), 1.17 (br d, 1H, J=16.7 Hz), 1.13 (s, 6H), 0.93 (t, 3H, J=7.1 Hz), 0.81 (br s, 1H); MS (APCI−) m/z 451 (M+1); HPLC UV purity Rt=10.041 min, 98.29%.

### Step C: Preparation of ethyl 3-(6-(2-carbamoyl-6-(trifluoromethoxy)-1H-indole-1-yl)pyridin-2-yl)-2,2-dimethylpropanoate

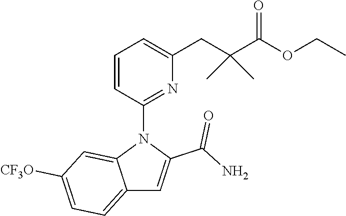

To a 2 mL DMF solution of 1-(6-(3-ethoxy-2,2-dimethyl-3-oxopropyl)pyridin-2-yl)-6-(trifluoromethoxy)-1H-indole-2-carboxylix acid (39.6 mg, 0.08 mmol) was added solid ammonium chloride (14.3 mg, 0.26 mmol) and 2-(1H-benzotriazole-1-yl)-1,1,3,3-tetramethylaminium tetrafluoroborate (42.5, 0.13 mmol) followed by diisopropylethylamine (0.138 mL, 0.79 mmol). The reaction mixture was stirred overnight at room temperature under N₂ atmosphere. The reaction mixture was subsequently partitioned between ethyl acetate (25 mL) and H₂O (20 mL). The phases were separated, and the organic phase was partitioned with 1M KHSO₄ (20 mL), followed by brine (20 mL). The organic layer was concentrated under reduced pressure to afford the crude product as a golden oil (54.2 mg), which was subsequently used in the next reaction without further purification. ¹H-NMR (DMSO-d6, 400 MHz) δ 8.07 (br s, 1H), 7.9-8.0 (m, 1H), 7.84 (t, 1H, J=7.8 Hz), 7.80 (d, 1H, J=8.5 Hz), 7.68 (br d, 1H, J=8.3 Hz), 7.49 (br t, 1H, J=7.6 Hz), 7.3-7.4 (m, 2H), 7.1-7.3 (m, 5H), 3.90 (q, 2H, J=7.1 Hz), 3.0-3.0 (m, 2H), 2.85 (s, 2H), 2.6-2.7 (m, 2H), 1.14 (s, 6H), 0.98 (t, 3H, J=7.1 Hz); MS (APCI+) m/z 450 (M+1).

### Step D: Preparation of 3-(6-(2-carbamoyl-6-(trifluoromethoxy)-1H-indole-1-yl)pyridin-2-yl)-2,2-dimethylpropanoic acid (Compound 8)

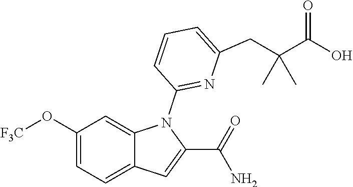 [SMILES: CC(C)(Cc1cccc(-n2c(C(N)=O)cc3ccc(OC(F)(F)F)cc32)n1)C(=O)O]

To a 3 mL methanol solution of ethyl 3-(6-(2-carbamoyl-6-(trifluoromethoxy)-1H-indole-1-yl)pyridin-2-yl)-2,2-dimethylpropanoate (49.8 mg) was added 1M lithium hydroxide (0.443 mL, 0.443 mmol). The reaction mixture was stirred at room temperature for 48 h under N₂ atmosphere. The reaction mixture was subsequently partitioned between ethyl acetate (5 mL) and 1M HCl (5 mL). The phases were separated, and the organic phase was partitioned with H₂O (5 mL), followed by brine (5 mL). The organic layer was concentrated under reduced pressure to afford the crude product. The crude mixture was purified by flash silica column chromatography on a CombiFlash NextGen 300+ purification system. Elution through a 4 g RediSep Gold Rf flash silica cartridge with 50-80% ethyl acetate in hexanes containing 1% acetic acid afforded the title compound as a white film (13.2 mg, 29% yield); ¹H-NMR (DMSO-d6, 400 MHz) δ 12.1-12.4 (m, 1H), 8.0-8.2 (m, 1H), 7.8-7.9 (m, 2H), 7.40 (br s, 1H), 7.2-7.3 (m, 2H), 7.19 (d, 1H, J=7.8 Hz), 2.96 (s, 2H), 1.1-1.1 (m, 1H), 1.12 (s, 5H); ¹⁹F-NMR (376 MHz; DMSO-d6) δ −56.76; MS (APCI⁺) m/z 422 (M+1). HPLC UV purity, Rt=7.372 min, 85.77%.

### Example 18: Synthesis of 2-(6-(2-carbamoyl-6-(trifluoromethoxy)-1H-indol-1-yl)-2,3-dihydrobenzofuran-3-yl)acetic acid (Compound 9)

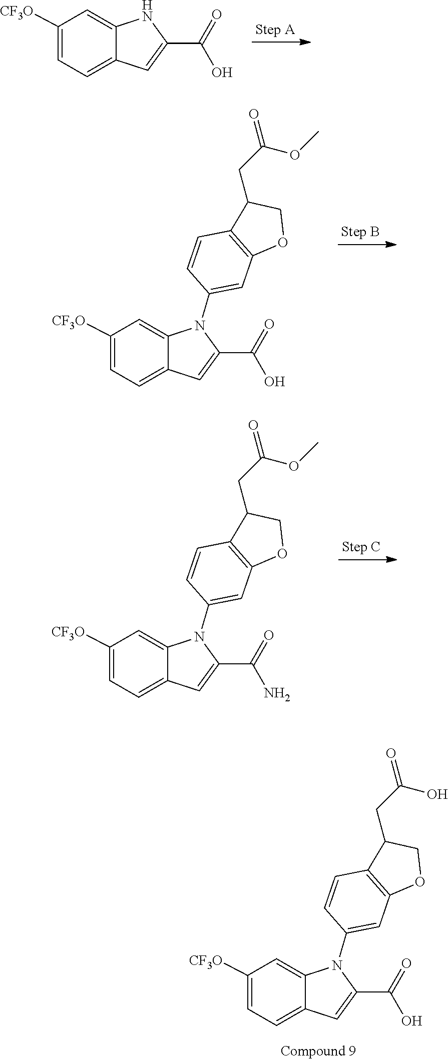

### Step A: Preparation of 1-(3-(carboxymethyl)-2,3-dihydrobenzofuran-6-yl)-6-(trifluoromethoxy)-1H-indole-2-carboxylic acid

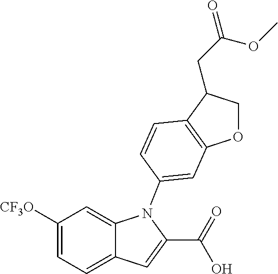

To a mixture consisting of 6-(trifluoromethoxy)-1H-indole-2-carboxylic acid (ChemShuttle, 0.245 g, 1.00 mmol), methyl 6-bromo-2,3-dihydrobenzofuran-3-acetate (Accela, 0.271 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was stirred overnight at 115° C. under N₂ atmosphere. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.606 g. The crude oil was purified by flash silica column chromatography on a CombiFlash NextGen 300+ purification system. Elution through a 24 g RediSep Gold Rf flash silica cartridge with 10-100% ethyl acetate in hexanes containing 1% acetic acid afforded the title compound as a white solid (0.119 g, 27% yield). R_{f} 0.12 with 1:20:80 v/v acetic acid-ethyl acetate-hexane (UV 254 nM); ¹H-NMR (400 MHz; DMSO-d6) δ 12.93 (br s, 1H), 7.82 (d, 1H, J=8.7 Hz), 7.32-7.37 (m, 2H), 7.1-7.2 (br d, 1H, J=8.7 Hz), 6.88 (s, 1H), 6.79-6.84 (m, 2H), 4.80 (t, 1H, J=9.2 Hz), 4.30 (dd, 1H, J=7.5, 9.1 Hz), 3.8-4.0 (m, 2H), 3.63 (s, 3H), 2.94 (dd, 1H, J=5.4, 16.6 Hz), 2.73 (dd, 1H, J=8.9, 16.7 Hz); ¹⁹F-NMR (376 MHz; DMSO-d6) δ −56.67; MS (APCI−) m/z 434 (M−1); HPLC UV purity Rt=9.508 min, 96.01%.

### Step B: Preparation of methyl 2-(6-(2-carbamoyl-6-(trifluoromethoxy)-1H-indol-1-yl)-2,3-dihydrobenzofuran-3-yl)acetate

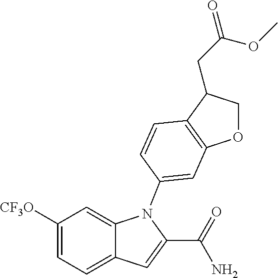

To a mixture consisting of 1-(3-(carboxymethyl)-2,3-dihydrobenzofuran-6-yl)-6-(trifluoromethoxy)-1H-indole-2-carboxylic acid (0.095 g, 0.218 mmol) in DMF (3 mL) was added ammonium chloride (Chem-Impex, 0.035 g, 0.65 mmol). Next add TBTU (Oakwood, 0.105 g, 0.328 mmol), followed by the addition of DIPEA (0.35 mL, 1.96 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (25 mL), followed by brine (25 mL). The organic layer was concentrated under reduced pressure to afford the crude product as a yellow solid (0.078 g, 84% yield). The crude solid was used in the next reaction w/o further purification. R_{f} 0.11 with 1:30:70 v/v acetic acid-ethyl acetate-hexane (UV 254 nM); ¹H-NMR (400 MHz; DMSO-d6) δ 7.95 (br s, 1H), 7.78 (d, 1H, J=8.7 Hz), 7.32 (br d, 2H, J=8.0 Hz), 7.20 (s, 1H), 7.11 (d, 1H, J=8.9 Hz), 6.94 (s, 1H), 6.76 (dd, 1H, J=1.7, 7.9 Hz), 6.70 (d, 1H, J=1.8 Hz), 4.79 (t, 1H, J=9.2 Hz), 4.29 (dd, 1H, J=7.3, 8.9 Hz), 3.8-3.9 (m, 1H), 3.63 (s, 3H), 2.93 (dd, 1H, J=5.4, 16.6 Hz) 2.6-2.8 (m, 1H); ¹⁹F-NMR (376 MHz; DMSO-d6) δ −56.70; MS (APCI⁺) m/z 435.0 (M+1), 433 (M−1); LC/MS Rt=6.798 min, 100%.

### Step C: Preparation of 2-(6-(2-carbamoyl-6-(trifluoromethoxy)-1H-indol-1-yl)-2,3-dihydrobenzofuran-3-yl)acetic acid (Compound 9)

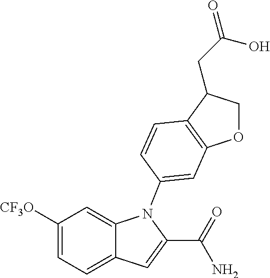

To a mixture consisting of methyl 2-(6-(2-carbamoyl-6-(trifluoromethoxy)-1H-indol-1-yl)-2,3-dihydrobenzofuran-3-yl)acetate (0.078 g, 0.18 mmol) in methanol (5 mL) was added 1M lithium hydroxide (0.72 mL, 0.72 mmol). The reaction mixture was stirred at room temperature overnight under N₂ atmosphere. The reaction mixture was subsequently partitioned between ethyl acetate (25 mL) and 1M HCl (10 mL). The phases were separated, and the organic phase was partitioned with water (20 mL), followed by brine (20 mL). The organic layer was concentrated under reduced pressure to afford the crude product as a yellow solid (0.075 g, 98% yield). R_{f} 0.20 with 1:50:50 v/v acetic acid-ethyl acetate-hexane (UV 254 nM; ¹H-NMR (400 MHz; CDCl₃) δ 12.39 (br s, 1H), 7.95 (br s, 1H), 7.78 (d, 1H, J=8.5 Hz), 7.34 (d, 2H, J=7.6 Hz), 7.2-7.3 (m, 1H), 7.20 (s, 1H), 7.1 (m, 1H), 6.95 (s, 1H), 6.76 (dd, 1H, J=1.8, 7.8 Hz), 6.69 (d, 1H, J=1.87 Hz), 4.79 (t, 1H, J=9.3 Hz), 4.2-4.3 (m, 1H), 3.8-3.9 (m, 1H), 2.8-2.9 (m, 1H), 2.5-2.7 (m, 1H); ¹⁹F-NMR (376 MHz; DMSO-d6) δ −56.69; MS (APCI⁺) m/z 421.0 (M+1), 419.0 (M−1); HPLC UV purity, Rt=7.402 min, 96.0%; melting point=180-181° C.

### Example 19: Synthesis of 1-(3-(2-carbamoyl-6-(trifluoromethoxy)-1H-indol-1-yl)benzyl)cyclobutane-1-carboxylic acid (Compound 10)

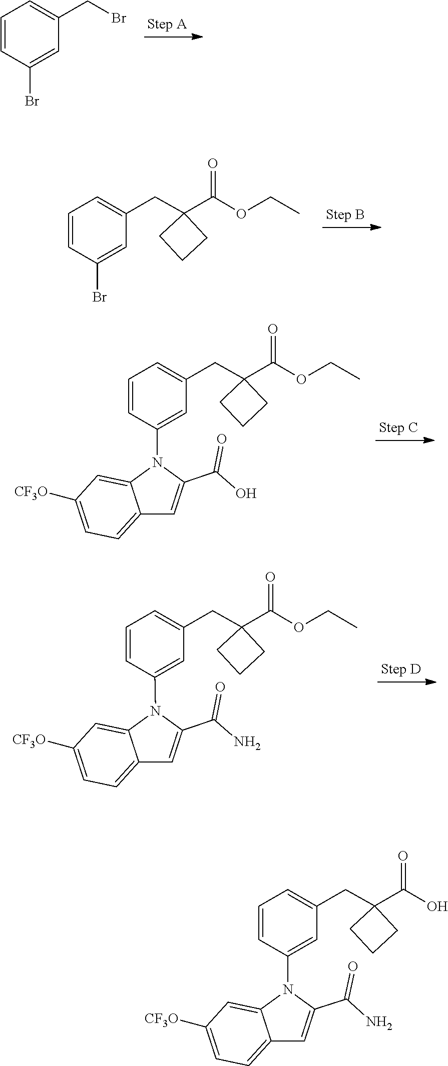

### Step A: Preparation of ethyl 1-(3-bromophenyl)cyclobutene-1-carboxylate

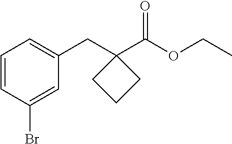 [SMILES: CCOC(=O)C1(Cc2cccc(Br)c2)CCC1]

To a 20 mL DMSO solution of ethyl cyclobutanecarboxylate (830 QL, 6.00 mmol) cooled to −78° C. was added 2M LDA (3.0 mL, 6.00 mmol) dropwise under N₂ atmosphere. After stirring 30 minutes, a 3 mL DMSO solution of 1-bromo-3-(bromomethyl)benzene (0.997 g, 4.00 mmol) was added dropwise and allowed to stir for 1 hour. After which, the reaction was allowed to warm to room temperature and stirred overnight. The reaction mixture was partitioned between 25 mL ethyl acetate and 15 mL saturated NH₄Cl. The organic layer was sequentially washed with water (15 mL) and brine (15 mL), then dried over anhydrous sodium sulfate. The crude reaction mixture was purified by flash silica column chromatography on a CombiFlash NextGen 300+ purification system. Elution through an 80 g RediSep Gold Rf flash silica cartridge with 0-10% ethyl acetate in hexanes afforded the title compound as a clear and colorless oil (0.904 g, 76% yield). R_{f} 0.73 with 1:20:80 v/v acetic acid-ethyl acetate-hexane (UV 254 nM); 1H NMR (CHLOROFORM-d, 400 MHz) δ 7.32 (d, 1H, J=7.8 Hz), 7.3-7.3 (m, 1H), 7.11 (t, 1H, J=7.8 Hz), 7.04 (d, 1H, J=7.6 Hz), 4.11 (q, 2H, J=7.1 Hz), 3.04 (s, 2H), 2.4-2.5 (m, 2H), 2.0-2.1 (m, 2H), 1.8-1.9 (m, 2H), 1.20 (t, 3H, J=7.1 Hz); MS (FIA MS+) m/z 297 (M+1).

### Step B: Preparation of 1-(3-((1-(ethoxycarbonyl)cyclobutyl)methyl)phenyl)-6-(trifluoromethoxy)-1H-indole-2-carboxylic acid

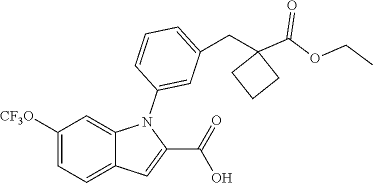

To a mixture consisting of 6-(trifluoromethoxy)-1H-indole-2-carboxylic acid (ChemShuttle, 0.245 g, 1.00 mmol), ethyl 1-(3-bromophenyl)cyclobutene-1-carboxylate (0.297 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was stirred overnight at 115° C. under N₂ atmosphere. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.524 g. The crude oil was purified by flash silica column chromatography on a CombiFlash NextGen 300+ purification system. Elution through a 24 g RediSep Gold Rf flash silica cartridge with 10-100% ethyl acetate in hexanes containing 1% acetic acid afforded the title compound as a white solid (0.250 g, mixture of desired product and starting material). R_{f} 0.29 with 1:20:80 v/v acetic acid-ethyl acetate-hexane (UV 254 nM); MS (APCI−) m/z 460.2 (M−1); HPLC UV purity Rt=11.002 min, 87.8%.

### Step C: Preparation of ethyl 1-(3-(2-carbamoyl-6-(trifluoromethoxy)-1H-indol-1-yl)benzyl)cyclobutane-1-carboxylate

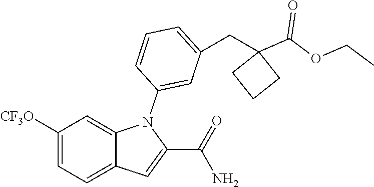

To a mixture consisting of 1-(3-((1-(ethoxycarbonyl)cyclobutyl)methyl)phenyl)-6-(trifluoromethoxy)-1H-indole-2-carboxylic acid (0.250 g, 0.542 mmol) in DMF (3 mL) was added ammonium chloride (Chem-Impex, 0.087 g, 1.63 mmol). Next add TBTU (Oakwood, 0.261 g, 0.812 mmol), followed by the addition of DIPEA (0.85 mL, 4.88 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (25 mL), followed by brine (25 mL). The organic layer was concentrated under reduced pressure to afford the crude product as a yellow oil (0.195 g, mixture of products). The crude solid was used in the next reaction w/o further purification. Rf 0.21 with 1:30:70 v/v acetic acid-ethyl acetate-hexane (UV 254 nM); MS (APCI⁺) m/z 461.0 (M+1), 459 (M−1); LC/MS Rt=7.608 min, multiple peaks).

### Step D: Preparation of 1-(3-(2-carbamoyl-6-(trifluoromethoxy)-1H-indol-1-yl)benzyl)cyclobutane-1-carboxylic acid (Compound 10)

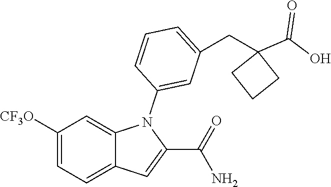

To a mixture consisting of ethyl 1-(3-(2-carbamoyl-6-(trifluoromethoxy)-1H-indol-1-yl)benzyl)cyclobutane-1-carboxylate (0.195 g, 0.42 mmol) in methanol (5 mL) was added 1M lithium hydroxide (1.70 mL, 1.70 mmol). The reaction mixture was stirred at room temperature overnight under N₂ atmosphere. The reaction mixture was subsequently partitioned between ethyl acetate (25 mL) and 1M HCl (10 mL). The phases were separated, and the organic phase was partitioned with water (20 mL), followed by brine (20 mL). The organic layer was concentrated under reduced pressure to afford the crude product as a yellow oil (0.195 g). The crude product was purified by flash silica column chromatography on a CombiFlash NextGen 300+ purification system. Elution through a 12 g RediSep Gold Rf flash silica cartridge with 10-100% ethyl acetate in hexanes containing 1% acetic acid afforded the title compound as a yellow solid (0.032 g, 17% yield). R_{f} 0.37 with 1:50:50 v/v acetic acid-ethyl acetate-hexane (UV 254 nM); ¹H-NMR (400 MHz; DMSO-d6) δ 12.28 (br s, 1H), 8.00 (br s, 1H), 7.84 (d, 1H, J=8.7 Hz), 7.40-7.44 (m, 2H), 7.28 (s, 1H), 7.24 (d, 1H, J=7.6 Hz), 7.17 (br d, 2H, J=4.1 Hz), 7.14 (s, 1H), 6.94 (s, 1H),), 3.10 (s, 2H), 2.2-2.4 (m, 2H), 1.9-2.2 (m, 2H), 1.7-1.9 (m, 2H); ¹⁹F-NMR (376 MHz; DMSO-d6) δ −56.76; MS (APCI⁺) m/z 433.0 (M+1), 431.0 (M−1); HPLC UV purity, Rt=8.428 min, 98.9%; melting point=178-179° C.

### Example 20: Synthesis of 2-(1-(tert-butoxycarbonyl)-5-(2-carbamoyl-6-(trifluoromethoxy)-1H-indol-1-yl)indolin-3-yl)acetic acid (Compound 11)

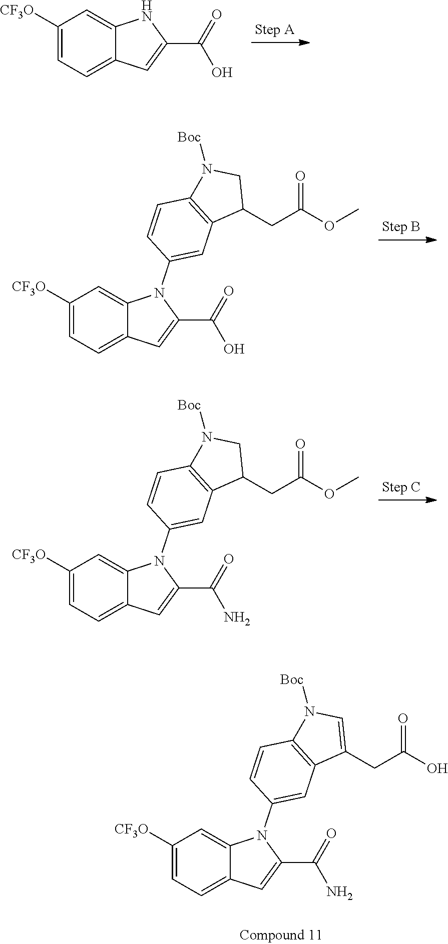

### Step A: Preparation of 1-(1-(tert-butoxycarbonyl)-3-(2-methoxy-2-oxoethyl)indolin-5-yl)-6-(trifluoromethoxy)-1H-indole-2-carboxylic acid

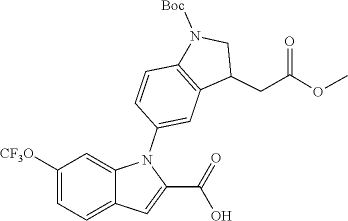

To a mixture consisting of 6-(trifluoromethoxy)-1H-indole-2-carboxylic acid (ChemShuttle, 0.245 g, 1.00 mmol), tert-butyl 5-bromo-3-(2-methoxy-2-oxoethyl)-2,3-dihydro-1H-indole-1-carboxylate (Enamine, 0.269 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 150° C. under N₂ atmosphere for 1 hour and then allowed to cool to room temperature. The reaction mixture was subsequently partitioned between ethyl acetate (50 mL) and water (50 mL×2). The phases were separated, and the organic phase was partitioned with 1M KOH (50 mL×2), followed by brine (50 mL). The organic layer was separated and dried over anhydrous magnesium sulfate, filtered, and concentrated under reduced pressure to give a crude red oil, 0.710 g. The crude oil was purified by flash silica column chromatography on a CombiFlash NextGen 300+ purification system. Elution through a 24 g RediSep Gold Rf flash silica cartridge with 10-30% ethyl acetate in hexanes containing 1% acetic acid afforded the title compound as an orange solid (0.084 g, 21.2% yield). R_{f} 0.24 with 1:30:70 v/v acetic acid-ethyl acetate-hexane (UV 254 nM); LC/MS Rt=6.316 min, (APCI−) m/z 533.10 (M−1).

### Step B: Preparation of tert-butyl 5-(2-carbamoyl-6-(trifluoromethoxy)-1H-indol-1-yl)-3-(2-methoxy-2-oxoethyl)indoline-1-carboxylate

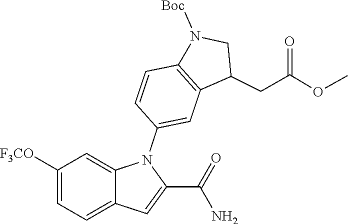 [SMILES: COC(=O)CC1CN(C(=O)OC(C)(C)C)c2ccc(-n3c(C(N)=O)cc4ccc(OC(F)(F)F)cc43)cc21]

To a mixture consisting of 1-(1-(tert-butoxycarbonyl)-3-(2-methoxy-2-oxoethyl)indolin-5-yl)-6-(trifluoromethoxy)-1H-indole-2-carboxylic acid (0.084 g, 0.157 mmol) in DMF (5 mL) was added ammonium chloride (Chem-Impex, 0.025 g, 0.471 mmol). Next add TBTU (Oakwood, 0.076 g, 0.235 mmol), followed by the addition of DIPEA (0.24 mL, 1.41 mmol). The reaction mixture was stirred overnight at room temperature under N₂ atmosphere. The reaction mixture was subsequently partitioned between ethyl acetate (50 mL) and H₂O (25 mL). The phases were separated, and the organic phase was partitioned with 1M KOH (50 mL), followed by brine (50 mL). The organic layer was concentrated under reduced pressure to afford the crude product as an orange solid (0.076 g, 91% yield). The crude solid was used in the next reaction w/o further purification. R_{f} 0.14 with 1:30:70 v/v acetic acid-ethyl acetate-hexane (UV 254 nM); LC/MS Rt=7.536 min, (APCI−) m/z 532.10 (M−1).

### Step C: Preparation of 2-(1-(tert-butoxycarbonyl)-5-(2-carbamoyl-6-(trifluoromethoxy)-1H-indol-1-yl)indolin-3-yl)acetic acid

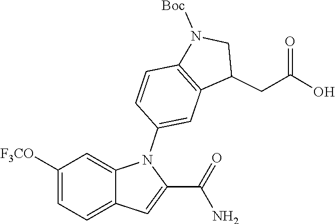 [SMILES: CC(C)(C)OC(=O)N1CC(CC(=O)O)c2cc(-n3c(C(N)=O)cc4ccc(OC(F)(F)F)cc43)ccc21]

To a mixture consisting of tert-butyl 5-(2-carbamoyl-6-(trifluoromethoxy)-1H-indol-1-yl)-3-(2-methoxy-2-oxoethyl)indoline-1-carboxylate (0.102 g, 0.19 mmol) in methanol (5 mL) was added 1M lithium hydroxide (1.00 mL, 1.00 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (15 mL), followed by brine (15 mL). The organic layer was concentrated under reduced pressure to afford the title product as a white solid (0.087 g). The crude product was purified by flash silica column chromatography on a CombiFlash NextGen 300+ purification system. Elution through a 12 g RediSep Gold Rf flash silica cartridge with 10-50% ethyl acetate in hexanes containing 1% acetic acid afforded the title compound as an orange solid (0.052 g, 52% yield). R_{f} 0.54 with 1:70:30 v/v acetic acid-ethyl acetate-hexane (UV 254 nM); ¹H-NMR (400 MHz; DMSO-d6) δ 12.34 (br s, 1H), 7.96 (br s, 1H), 7.82 (d, 1H, J=8.5 Hz), 7.37 (br s, 1H), 7.2-7.3 (m, 2H), 7.1-7.2 (m, 2H), 6.93 (s, 1H), 4.22 (m, 1H), 3.6-3.7 (m, 2H), 2.7-2.9 (m, 1H), 2.5-2.7 (m, 2H), 1.53 (br s, 9H); ¹⁹F-NMR (376 MHz; DMSO-d6) δ −56.70; LC/MS Rt=5.848 min, (APCI−) m/z 518.10 (M−1); HPLC UV purity, Rt=8.919 min, 98.2%.

### Example 21: Synthesis of 2-(5-(2-carbamoyl-6-(trifluoromethoxy)-1H-indol-1-yl)benzofuran-3-yl)acetic acid (Compound 12)

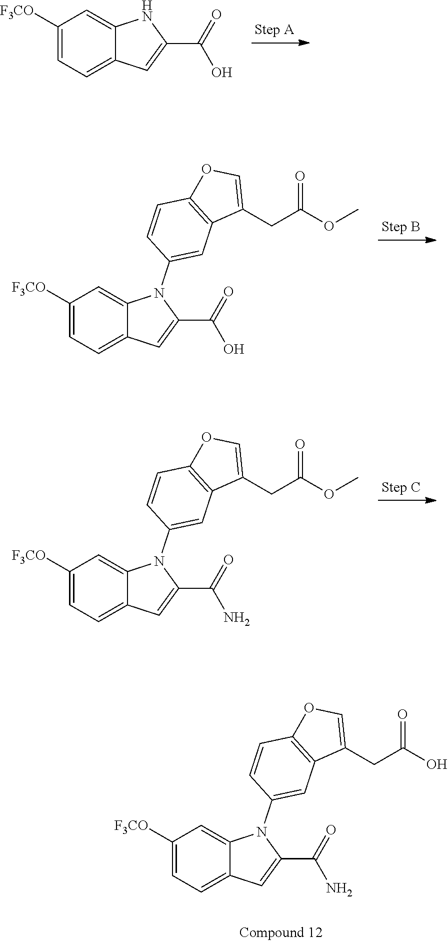 [SMILES: O=C(O)c1cc2ccc(OC(F)(F)F)cc2[nH]1 ; COC(=O)Cc1coc2ccc(-n3c(C(=O)O)cc4ccc(OC(F)(F)F)cc43)cc12 ; COC(=O)Cc1coc2ccc(-n3c(C(N)=O)cc4ccc(OC(F)(F)F)cc43)cc12 ; NC(=O)c1cc2ccc(OC(F)(F)F)cc2n1-c1ccc2occ(CC(=O)O)c2c1]

### Step A: Preparation of 1-(3-(2-methoxy-2-oxoethyl)benzofuran-5-yl)-6-(trifluoromethoxy)-1H-indole-2-carboxylic acid

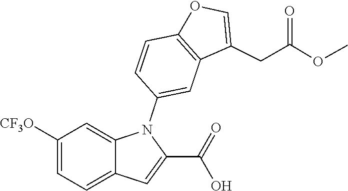

To a mixture consisting of 6-(trifluoromethoxy)-1H-indole-2-carboxylic acid (ChemShuttle, 0.245 g, 1.00 mmol), methyl 2-(5-bromo-1-benzofuran-3-yl) acetate (Accela, 0.269 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 115° C. under N₂ atmosphere for 10 hours and then stirred overnight at room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.630 g. The crude oil was purified by flash silica column chromatography on a CombiFlash NextGen 300+ purification system. Elution through a 24 g RediSep Gold Rf flash silica cartridge with 10-30% ethyl acetate in hexanes containing 1% acetic acid afforded the title compound as an orange solid (0.068 g, 16% yield). R_{f} 0.09 with 1:20:80 v/v acetic acid-ethyl acetate-hexane (UV 254 nM); ¹H-NMR (400 MHz; DMSO-d6) δ 12.96 (bs, 1H), 8.06 (s, 1H), 7.91 (d, 1H, J=8.7 Hz), 7.83 (br d, 1H, J=8.9 Hz), 7.72 (d, 1H, J=8.7 Hz), 7.68 (d, 1H, J=2.1 Hz), 7.48 (s, 1H), 7.32 (dd, 1H, J=2.2, 8.6 Hz), 7.2-7.2 (m, 1H), 6.91 (s, 1H), 3.85 (s, 2H), 3.6-3.7 (m, 1H), 3.61 (s, 3H); ¹⁹F-NMR (376 MHz; DMSO-d6) δ −56.71; MS (APCI−) m/z 432 (M−1); HPLC UV purity Rt=10.972 min, 94.58%.

### Step B: Preparation of methyl 2-(5-(2-carbamoyl-6-(trifluoromethoxy)-1H-indol-1-yl)benzofuran-3-yl)acetate

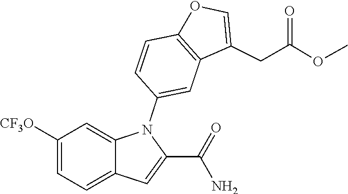

To a mixture consisting of 1-(3-(2-methoxy-2-oxoethyl)benzofuran-5-yl)-6-(trifluoromethoxy)-1H-indole-2-carboxylic acid (0.065 g, 0.148 mmol) in DMF (3 mL) was added ammonium chloride (Chem-Impex, 0.024 g, 0.444 mmol). Next add TBTU (Oakwood, 0.071 g, 0.222 mmol), followed by the addition of DIPEA (0.23 mL, 1.33 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (20 mL), followed by brine (20 mL). The organic layer was concentrated under reduced pressure to afford the crude product as an orange solid (0.068 g, quantitative yield). The crude solid was used in the next reaction w/o further purification. Rf 0.09 with 1:30:70 v/v acetic acid-ethyl acetate-hexane (UV 254 nM); ¹H-NMR (400 MHz; DMSO-d6) δ 8.05 (s, 1H), 8.02 (br s, 1H), 7.86 (d, 1H, J=8.7 Hz), 7.70 (d, 1H, J=8.5 Hz), 7.62 (d, 1H, J=2.1 Hz), 7.39 (br s, 1H), 7.32 (s, 1H), 7.24 (dd, 1H, J=2.1, 8.7 Hz), 7.20 (d, 1H, J=8.7 Hz), 6.95 (s, 1H), 3.85 (s, 2H), 3.62 (s, 3H); ¹⁹F-NMR (376 MHz; DMSO-d6) δ −56.74; MS (APCI+) m/z 433.1 (M+1).

### Step C: Preparation of 2-(5-(2-carbamoyl-6-(trifluoromethoxy)-1H-indol-1-yl)benzofuran-3-yl)acetic acid (Compound 12)

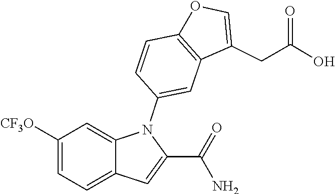

To a mixture consisting of methyl 2-(5-(2-carbamoyl-6-(trifluoromethoxy)-1H-indol-1-yl)benzofuran-3-yl)acetate (0.064 g, 0.15 mmol) in methanol (3 mL) was added 1M lithium hydroxide (0.60 mL, 0.60 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (15 mL), followed by brine (15 mL). The organic layer was concentrated under reduced pressure to afford the title product as an orange solid (0.049 g, 80% yield). R_{f} 0.04 with 1:50:50 v/v acetic acid-ethyl acetate-hexane (UV 254 nM); ¹H-NMR (400 MHz; DMSO-d6) δ 12.48 (br s, 1H), 8.03 (br s, 2H), 7.90 (br d, 1H, J=8.7 Hz), 7.85 (d, 1H, J=8.7 Hz), 7.69 (d, 1H, J=8.7 Hz), 7.61 (d, 1H, J=2.1 Hz), 7.37 (br s, 1H), 7.32 (s, 1H), 7.22 (dd, 1H, J=2.1, 8.7 Hz), 7.1-7.2 (m, 1H), 6.94 (s, 1H), 3.73 (s, 2H); ¹⁹F-NMR (376 MHz; DMSO-d6) δ −56.71; MS (APCI⁺) m/z 419.0 (M+1), 417.0 (M−1); HPLC UV purity, Rt=7.623 min, 93.8%; melting point=238° C.

### Example 22: Synthesis of 3-(3-(2-carbamoylbenzo[b]thiophen-3-yl)phenyl)propanoic acid (Compound 13)

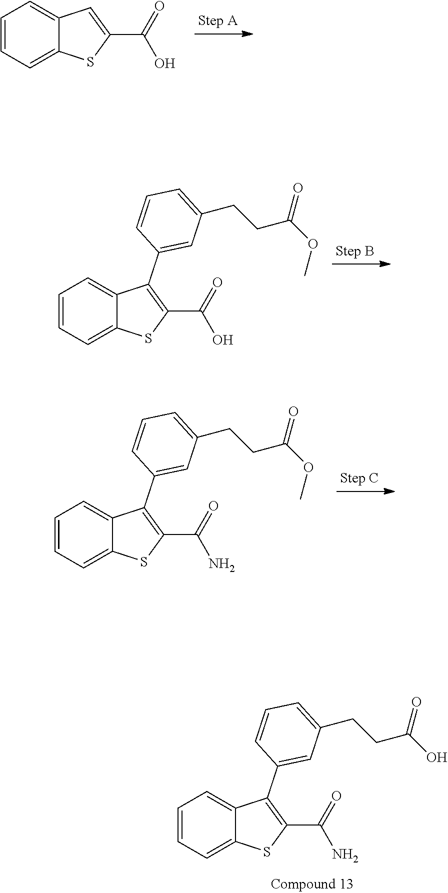 [SMILES: O=C(O)c1cc2ccccc2s1 ; COC(=O)CCc1cccc(-c2c(C(=O)O)sc3ccccc23)c1 ; COC(=O)CCc1cccc(-c2c(C(N)=O)sc3ccccc23)c1 ; NC(=O)c1sc2ccccc2c1-c1cccc(CCC(=O)O)c1]

### Step A: Preparation of 3-(3-(3-methoxy-3-oxopropyl)phenyl)benzo[b]thiophene-2-carboxylic acid

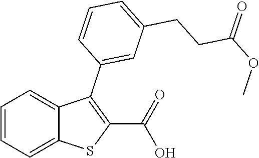 [SMILES: COC(=O)CCc1cccc(-c2c(C(=O)O)sc3ccccc23)c1]

To a 24 mL septa-cap vial was added methyl 3-bromobenzenepropanoate (Combi-Blocks, 0.486 g, 2.0 mmol) and benzo[b]thiophene-2-carboxylic acid (VWR, 0.357 g, 2.0 mmol). Next was added trimethylphosphonium tetrafluoroborate (Strem, 0.027 g, 8 mol %), K₂CO₃ (VWR, 0.305 g, 2.2 mmol) and NMP (8 mL). The mixture was degassed by bubbling in nitrogen gas for 3 min with stirring then Bis(dichloro(η⁶-p-cymene)ruthenium) (Strem, 0.049 g, 4 mol %) added and the mixture heated to 105° C. overnight. The reaction mixture was subsequently cooled to room temperature then diluted with water and extracted with ethyl acetate (3×120 mL). The combined organic layers were dried over anhydrous sodium sulfate, filtered, and concentrated under reduced pressure to give a crude oil which was purified by flash silica column chromatography on a CombiFlash NextGen 300+ purification system. Elution through a 40 g RediSep Gold Rf flash silica cartridge with 10-60% ethyl acetate in heptane containing 1% acetic acid afforded the title compound as a white solid (0.171 g, 25% yield). Rf 0.30 with 1:30:70 v/v acetic acid-ethyl acetate-hexane (UV 254 nM); ¹H-NMR (400 MHz; CDCl₃) δ 7.90 (d, 1H, J=8.0 Hz), 7.5-7.6 (m, 1H), 7.3-7.4 (m, 2H), 7.31 (br d, 1H, J=7.8 Hz), 7.3-7.3 (m, 1H), 7.26 (s, 1H), 3.68 (s, 2H), 3.6-3.7 (m, 1H), 3.04 (t, 2H, J=7.8 Hz), 2.70 (t, 2H, J=7.8 Hz); FIA-MS (APCI Neg Scan) m/z 339.0 (M−1); melting point=134-135.5° C.

### Step B: Preparation of methyl 3-(3-(2-carbamoylbenzo[b]thiophen-3-yl)phenyl)propanoate

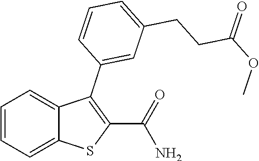 [SMILES: COC(=O)CCc1cccc(-c2c(C(N)=O)sc3ccccc23)c1]

To a mixture consisting of 3-(3-(3-methoxy-3-oxopropyl)phenyl)benzo[b]thiophene-2-carboxylic acid (0.166 g, 0.49 mmol) in DMF (2.0 mL) was added ammonium chloride (Chem-Impex, 0.078 g, 1.46 mmol). Next was added TBTU (Oakwood, 0.235 g, 0.73 mmol) followed by the addition of DIPEA (0.77 mL, 4.4 mmol). The reaction mixture was stirred overnight at room temperature under N₂ atmosphere. The reaction mixture was subsequently diluted with water (150 mL) then extracted with 3×70 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 24 g RediSep Gold Rf flash silica cartridge with 10-100% ethyl acetate in heptane containing 1% acetic acid afforded the title compound as a white semi-solid (0.168 g, 99% yield). R_{f} 0.29 with 1:40:60 v/v acetic acid-ethyl acetate-hexane (UV 254 nM); ¹H-NMR (400 MHz; CDCl₃) δ 7.91 (d, 1H, J=8.3 Hz), 7.4-7.5 (m, 2H), 7.3-7.4 (m, 5H), 6.22 (br s, 1H), 5.60 (br s, 1H), 3.67 (s, 3H), 3.06 (t, 2H, J=7.6 Hz), 2.71 (t, 2H, J=7.6 Hz); MS (APCI⁺) m/z 340.1.0 (M+1).

### Step C: Preparation of 3-(3-(2-carbamoylbenzo[b]thiophen-3-yl)phenyl)propanoic acid (Compound 13)

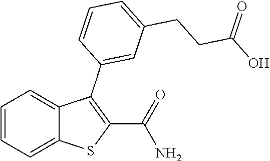 [SMILES: NC(=O)c1sc2ccccc2c1-c1cccc(CCC(=O)O)c1]

To a mixture consisting of methyl 3-(3-(2-carbamoylbenzo[b]thiophen-3-yl)phenyl)propanoate (0.071 g, 0.17 mmol) in MeOH (2 mL) was added dropwise, a 1M aqueous solution of LiOH (0.51 mL, 3 eq). The reaction mixture was stirred overnight at room temperature. The clear solution was subsequently diluted with water (10 mL) and subjected to reduced pressure on a rotary evaporator to remove MeOH. Additional water was added (10 mL) and the solution was acidified with 1N HCl to precipitate product. The suspension was filtered, and solids washed with excess water then dried under high vac overnight to afford the title compound as a white solid (0.045 g, 65% yield). R_{f} 0.27 with 1:40:60 v/v acetic acid-ethyl acetate-heptane (UV 254 nM); ¹H-NMR (400 MHz; DMSO-d₆) δ 12.18 (br s, 1H), 8.06 (d, 1H, J=8.0 Hz), 7.66 (br s, 1H), 7.4-7.5 (m, 5H), 7.33 (s, 1H), 7.28 (br d, 1H, J=7.6 Hz), 6.63 (br s, 1H), 2.91 (br t, 2H, J=7.6 Hz), 2.60 (br t, 2H, J=7.6 Hz); MS (APCI⁺) m/z 326.2, (M+1), m/z 324.0, (M−1); HPLC UV purity, Rt=6.77 min, 99.7%; melting point=198.0-200.0° C.

### Example 23: Synthesis of 3-(3-(2-(methylcarbamoyl)-6-(trifluoromethoxy)-1H-indol-1-yl)phenyl)propanoic acid (Compound 14)

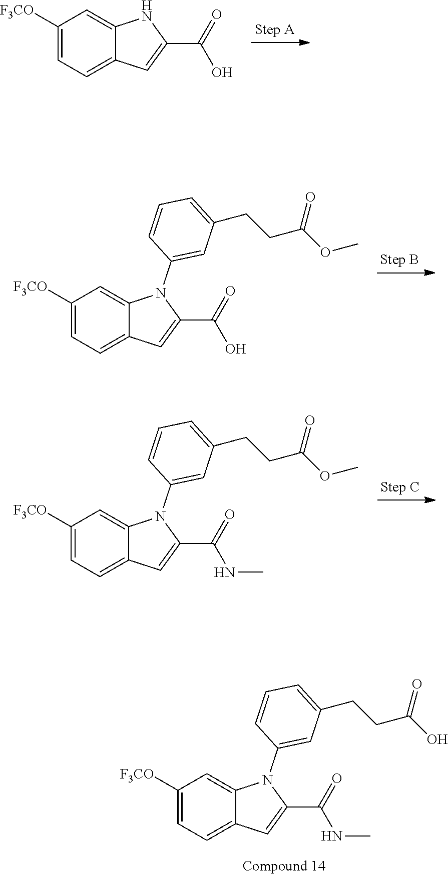 [SMILES: O=C(O)c1cc2ccc(OC(F)(F)F)cc2[nH]1 ; COC(=O)CCc1cccc(-n2c(C(=O)O)cc3ccc(OC(F)(F)F)cc32)c1 ; CNC(=O)c1cc2ccc(OC(F)(F)F)cc2n1-c1cccc(CCC(=O)OC)c1 ; CNC(=O)c1cc2ccc(OC(F)(F)F)cc2n1-c1cccc(CCC(=O)O)c1]

### Step A: Preparation of 1-(3-(3-methoxy-3-oxopropyl)phenyl)-6-(trifluoromethoxy)-1H-indole-2-carboxylic acid

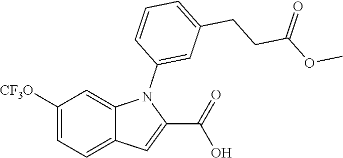

To a mixture consisting of 6-(trifluoromethoxy)-1H-indole-2-carboxylic acid (ChemShuttle, 0.245 g, 1.00 mmol), methyl 3-(3-bromophenyl)propanoate (CombiBlocks, 0.243 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 115° C. under N₂ atmosphere for 10 hours and then stirred overnight at room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.530 g. The crude oil was purified by flash silica column chromatography on a CombiFlash NextGen 300+ purification system. Elution through a 12 g RediSep Gold Rf flash silica cartridge with 5-30% ethyl acetate in hexanes containing 1% acetic acid afforded the title compound as a yellow oil (0.069 g, 17% yield). R_{f} 0.26 with 1:30:70 v/v acetic acid-ethyl acetate-hexane (UV 254 nM); MS (APCI−) m/z 406.0 (M−1); HPLC UV purity Rt=9.48 min, 99.4%.

### Step B: Preparation of methyl 3-(3-(2-(methylcarbamoyl)-6-(trifluoromethoxy)-1H-indol-1-yl)phenyl)propanoate

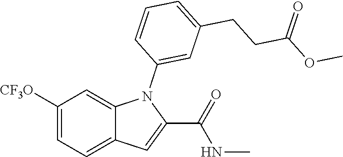

To a mixture consisting of 1-(3-(3-methoxy-3-oxopropyl)phenyl)-6-(trifluoromethoxy)-1H-indole-2-carboxylic acid (0.068 g, 0.167 mmol) in DCM (2 mL) was added TBTU (Oakwood, 0.064 g, 0.204 mmol), followed by the addition of DIPEA (0.07 mL, 0.40 mmol). The reaction mixture was stirred for 15 minutes at room temperature under N₂ atmosphere. Next methylamine (2.0M solution in THF, 0.2 mL, 0.4 mmol) was added to the reaction mixture. After 4 hours of stirring at room temperature the reaction was complete. The reaction mixture was subsequently partitioned between DCM (15 mL) and saturated aqueous NaHCO₃ (15 mL). The phases were separated, and the aqueous layer was back extracted with DCM (10 mL). Separate layers and the combined organic layer were washed with brine (15 mL). The organic layer was concentrated under reduced pressure to afford the crude product as a yellow oil (0.079 g, quantitative yield). The crude solid was used in the next reaction w/o further purification. R_{f} 0.14 with 1:30:70 v/v acetic acid-ethyl acetate-hexane (UV 254 nM); ¹H-NMR (400 MHz; DMSO-d6) δ 8.54 (m, 1H), 7.84 (d, 1H, J=8.5 Hz), 7.4-7.5 (m, 1H), 7.32 (d, 1H, J=8.7 Hz), 7.2-7.2 (m, 2H), 7.1-7.2 (m, 2H), 6.9-7.0 (m, 1H), 3.57 (s, 3H), 2.9-3.0 (m, 2H), 2.9-3.0 (m, 5H); ¹⁹F-NMR (376 MHz; DMSO-d6) δ −56.76; MS (APCI+) m/z 421.0 (M+1); HPLC UV purity Rt=8.98 min, 83.3.%.

### Step C: Preparation of 3-(3-(2-(methylcarbamoyl)-6-(trifluoromethoxy)-1H-indol-1-yl)phenyl)propanoic acid (Compound 13)

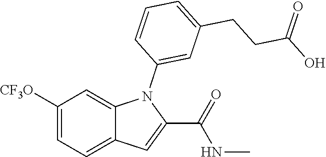

To a mixture consisting of methyl 3-(3-(2-(methylcarbamoyl)-6-(trifluoromethoxy)-1H-indol-1-yl)phenyl)propanoate (0.074 g, 0.15 mmol) in methanol (2 mL) was added 1M lithium hydroxide (0.70 mL, 0.70 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (10 mL), followed by brine (10 mL). The organic layer was concentrated under reduced pressure to afford the title product as a yellow solid (0.057 g, 80% yield). R_{f} 0.19 with 1:50:50 v/v acetic acid-ethyl acetate-hexane (UV 254 nM); ¹H-NMR (400 MHz; DMSO-d6) δ 12.16 (s, 1H), 8.54 (br d, 1H, J=4.6 Hz), 7.84 (d, 1H, J=8.5 Hz), 7.43 (t, 1H, J=7.7 Hz), 7.32 (d, 1H, J=7.6 Hz), 7.2-7.2 (m, 2H), 7.14 (br t, 2H, J=8.6 Hz), 6.98 (s, 1H), 2.90 (t, 2H, J=7.6 Hz), 2.69 (d, 3H, J=4.6 Hz), 2.5-2.6 (m, 2H); ¹⁹F-NMR (376 MHz; DMSO-d6) δ −56.72; MS (APCI⁺) m/z 407.0 (M+1), 405.0 (M−1); HPLC UV purity, Rt=7.71 min, 98.4%; melting point=150-151° C.

### Example 24: Synthesis of 3-(3-(2-(methylcarbamoyl)-6-(trifluoromethoxy)-1H-indol-1-yl)phenyl)propanoic acid (Compound 15)

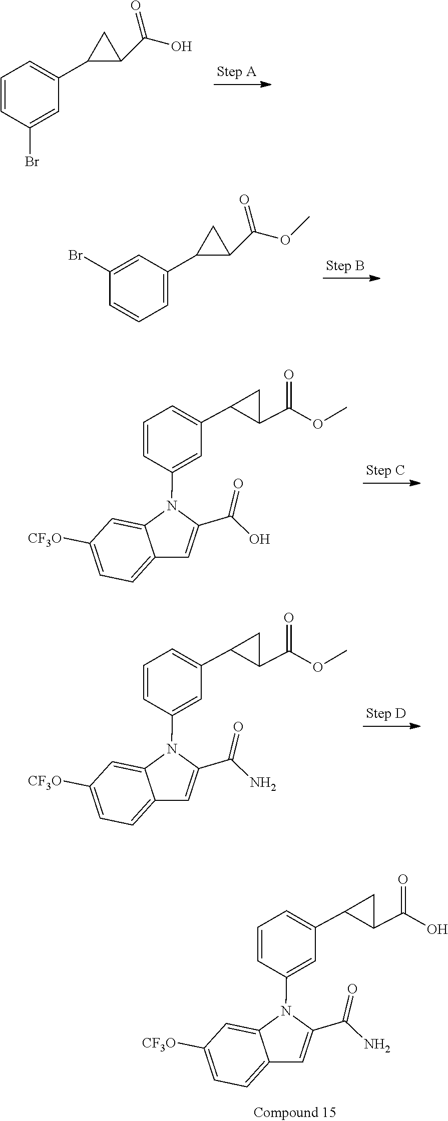

### Step A: Preparation of trans-methyl-2-(3-bromophenyl)cyclopropane-1-carboxylate

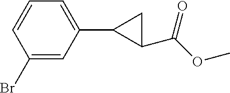

To a mixture consisting of trans-2-(3-bromo-phenyl)-cyclopropanecarboxylic acid (J&W Pharmlab, 0.930 g, 3.86 mmol) in methanol (30 mL) was added acetyl chloride (Aldrich, 1.10 mL, 15.43 mmol) dropwise at 0° C. The reaction was allowed to warm to room temperature over 6 hours in which the reaction showed completion by TLC. The reaction mixture was subsequently azeotroped with Toluene (2×100 mL) under reduced pressure to afford the desired product as a colorless oil (0.974 g, 98.9% yield). R_{f} 0.71 with 1:30:70 v/v acetic acid-ethyl acetate-hexane (UV 254 nM); ¹H-NMR (400 MHz; CDCl₃) δ 7.34 (d, 1H, J=7.6 Hz), 7.2-7.3 (m, 1H), 7.15 (t, 1H, J=7.9 Hz), 7.04 (d, 1H, J=7.8 Hz), 3.73 (s, 3H), 2.50 (ddd, 1H, J=4.1, 6.3, 9.3 Hz), 1.91 (ddd, 1H, J=4.4, 5.2, 8.5), 1.61 (td, 2H, J=5.2, 14.6 Hz), 1.3-1.4 (m, 1H); MS (APCI+) m/z 257 (M+1).

### Step B: Preparation of 1-(3-(trans-2-(methoxycarbonyl)cyclopropyl)phenyl)-6-(trifluoromethoxy)-1H-indole-2-carboxylic acid

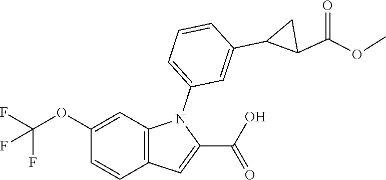 [SMILES: COC(=O)C1CC1c1cccc(-n2c(C(=O)O)cc3ccc(OC(F)(F)F)cc32)c1]

To a mixture consisting of 6-(trifluoromethoxy)-1H-indole-2-carboxylic acid (ChemShuttle, 0.245 g, 1.00 mmol), trans-methyl-2-(3-bromophenyl)cyclopropane-1-carboxylate (0.255 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 115° C. under N₂ atmosphere for 10 hours and then stirred overnight at room temperature. The reaction mixture was subsequently partitioned between ethyl acetate (75 mL) and 1M KHSO₄ (75 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.537 g. The crude oil was purified by flash silica column chromatography on a CombiFlash NextGen 300+ purification system. Elution through a 12 g RediSep Gold Rf flash silica cartridge with 5-30% ethyl acetate in hexanes containing 1% acetic acid afforded the title compound as a yellow solid (0.081 g, 19% yield). R_{f} 0.33 with 1:30:70 v/v acetic acid-ethyl acetate-hexane (UV 254 nM); ¹H-NMR (400 MHz; DMSO-d6) δ 12.94 (br s, 1H), 7.85 (d, 1H, J=8.5 Hz), 7.3-7.5 (m, 2H), 7.28 (d, 1H, J=8.0 Hz), 7.2-7.2 (m, 2H), 7.14 (dd, 1H, J=0.9, 8.7 Hz), 6.86 (s, 1H), 3.60 (s, 3H), 2.5-2.5 (m, 1H), 1.9-2.0 (m, 1H), 1.4-1.5 (m, 2H); ¹⁹F-NMR (376 MHz; DMSO-d6) δ −56.68; MS (APCI−) m/z 418.0 (M−1); HPLC UV purity Rt=9.688 min, 98.9%.

### Step C: Preparation of methyl trans-2-(3-(2-carbamoyl-6-(trifluoromethoxy)-1H-indol-1-yl)phenyl)cyclopropane-1-carboxylate

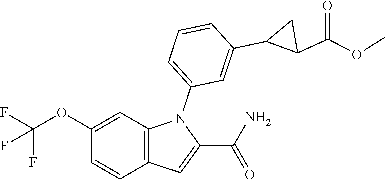 [SMILES: COC(=O)C1CC1c1cccc(-n2c(C(N)=O)cc3ccc(OC(F)(F)F)cc32)c1]

To a mixture consisting of 1-(3-(trans-2-(methoxycarbonyl)cyclopropyl)phenyl)-6-(trifluoromethoxy)-1H-indole-2-carboxylic acid (0.071 g, 0.171 mmol) in DMF (2 mL) was added ammonium chloride (Chem-Impex, 0.027 g, 0.513 mmol). Next add TBTU (Oakwood, 0.082 g, 0.256 mmol), followed by the addition of DIPEA (0.27 mL, 1.54 mmol). The reaction mixture was stirred for 4 hours at room temperature under N₂ atmosphere. The reaction mixture was subsequently partitioned between ethyl acetate (25 mL) and H₂O (25 mL). The phases were separated, and the organic phase was partitioned with 1M KHSO₄ (20 mL), followed by brine (20 mL). The organic layer was concentrated under reduced pressure to afford the crude product as an orange solid (0.041 g, 58% yield). The crude solid was used in the next reaction w/o further purification. R_{f} 0.12 with 1:30:70 v/v acetic acid-ethyl acetate-hexane (UV 254 nM); ¹H-NMR (400 MHz; DMSO-d6) δ 7.97 (br s, 1H), 7.80 (d, 1H, J=8.7 Hz), 7.3-7.4 (m, 2H), 7.2-7.3 (m, 2H), 7.13 (br s, 2H), 7.11 (br s, 1H), 6.90 (s, 1H), 3.60 (s, 3H), 2.5-2.6 (m, 1H), 1.9-2.0 (m, 1H), 1.4-1.5 (m, 2H); ¹⁹F-NMR (376 MHz; DMSO-d6) δ −56.70; MS (APCI+) m/z 419.0 (M+1); HPLC UV purity Rt=8.83 min, 97.3%.

### Step D: Preparation of trans-2-(3-(2-carbamoyl-6-(trifluoromethoxy)-1H-indol-1-yl)phenyl)cyclopropane-1-carboxylic acid (Compound 15)

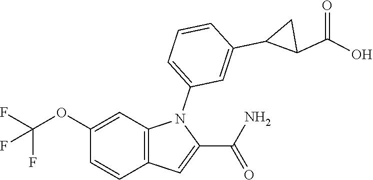 [SMILES: NC(=O)c1cc2ccc(OC(F)(F)F)cc2n1-c1cccc(C2CC2C(=O)O)c1]

To a mixture consisting of methyl trans-2-(3-(2-carbamoyl-6-(trifluoromethoxy)-1H-indol-1-yl)phenyl)cyclopropane-1-carboxylate (0.041 g, 0.099 mmol) in methanol (2 mL) was added 1M lithium hydroxide (0.40 mL, 0.40 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with water (10 mL), followed by brine (10 mL). The organic layer was concentrated under reduced pressure to afford the title product as a white solid (0.038 g, 96% yield). R_{f} 0.20 with 1:50:50 v/v acetic acid-ethyl acetate-hexane (UV 254 nM); ¹H-NMR (400 MHz; DMSO-d6) δ 12.35 (s, 1H), 8.01 (br s, 1H), 7.83 (d, 1H, J=8.7 Hz), 7.40-7.45 (m, 2H), 7.28 (s, 1H), 7.25 (d, 1H, J=7.8 Hz), 7.1-7.2 (m, 3H), 6.94 (s, 1H), 2.4-2.5 (m, 1H), 1.8-1.9 (m, 1H), 1.46 (td, 1H, J=4.7, 9.2 Hz), 1.38 (ddd, 1H, J=4.4, 6.4, 8.3 Hz); ¹⁹F-NMR (376 MHz; DMSO-d6) δ −56.71; MS (APCI−) m/z 403.0 (M−1); HPLC UV purity, Rt=7.53 min, 96.0%; Chiral HPLC purity, Rt=16.9 min, 45.1%, Rt=20.6 min, 49.9%; melting point=182-183° C.

### Example 25: Synthesis of trans-(+)- and trans-(−)-2-(3-(2-carbamoyl-6-(trifluoromethoxy)-1H-indol-1-yl)phenyl)cyclopropane-1-carboxylic acid (Compound 15a and Compound 15b)

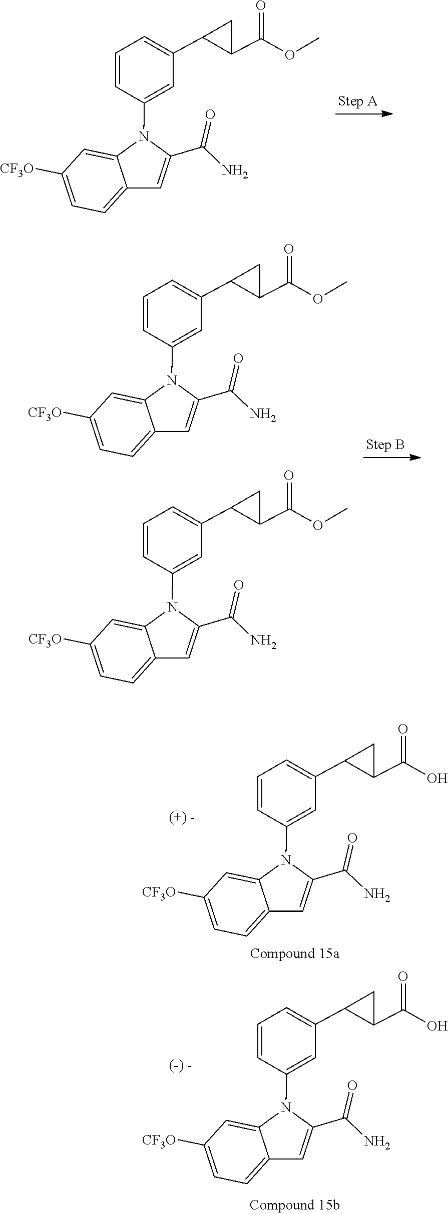

### Step A: Chiral separation of methyl trans-2-(3-(2-carbamoyl-6-(trifluoromethoxy)-1H-indol-1-yl)phenyl)cyclopropane-1-carboxylate

The diastereomers of methyl trans-2-(3-(2-carbamoyl-6-(trifluoromethoxy)-1H-indol-1-yl)phenyl)cyclopropane-1-carboxylate (0.132 g) was separated by Chiral HPLC on a (R, R) Whelk-01 column (250 mm×4.6 mm) using 75:25:0.1 Heptane:IPA:TEA at a flow rate of 21.2 mL/min with UV detection at 290 nm. Peak 1 eluted at 30.95 minutes and Peak 2 eluted at 36.28 minutes.

Diastereomer 1 (peak 1) was concentrated under reduced pressure to afford a white solid (0.40 mg, 30% recovery); R_{f} 0.43 with 1:60:40 v/v acetic acid-ethyl acetate-hexane (UV 254 nM); MS (APCI+) m/z 419.0 (M+1), (APCI−) m/z 417.0 (M−1); HPLC UV purity Rt=8.825 min, 100%.

Diastereomer 2 (peak 2) was concentrated under reduced pressure to afford a white solid (64.7 mg, 49% recovery); R_{f} 0.43 with 1:60:40 v/v acetic acid-ethyl acetate-hexane (UV 254 nM); MS (APCI+) m/z 419.0 (M+1), (APCI−) m/z 417.0 (M−1); HPLC UV purity Rt=8.814 min, 99.64%.

### Step B: Preparation of trans-(+) and trans-(−)-2-(3-(2-carbamoyl-6-(trifluoromethoxy)-1H-indol-1-yl)phenyl)cyclopropane-1-carboxylic acid

To the individually separated diastereomers of methyl trans-2-(3-(2-carbamoyl-6-(trifluoromethoxy)-1H-indol-1-yl)phenyl)cyclopropane-1-carboxylate (Peak 1, 0.040 g, 0.095 mmol), (Peak 2, 0.065 g, 0.155 mmol) in methanol (3.0 mL) was added 1M lithium hydroxide (0.75 mL, 0.75 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with H₂O (10 mL), followed by brine (10 mL). The organic layer was concentrated under reduced pressure to afford the crude products as a white solid.

Diastereomer 1 (peak 1, 15a) was concentrated under reduced pressure to afford a white solid (0.019 g, 52% yield). R_{f} 0.27 with 1:60:40 v/v acetic acid-ethyl acetate-hexane (UV 254 nM); ¹H-NMR (400 MHz; DMSO-d6) δ 12.35 (s, 1H), 8.01 (br s, 1H), 7.83 (d, 1H, J=8.7 Hz), 7.40-7.45 (m, 2H), 7.28 (s, 1H), 7.25 (d, 1H, J=8.0 Hz), 7.1-7.2 (m, 3H), 6.94 (s, 1H), 2.4-2.5 (m, 1H), 1.8-1.9 (m, 1H), 1.46 (td, 1H, J=4.7, 9.2 Hz), 1.38 (ddd, 1H, J=4.4, 6.4, 8.3 Hz); ¹⁹F-NMR (376 MHz; DMSO-d6) δ −56.70; MS (APCI+) m/z 405.0 (M+1), MS (APCI−) m/z 403.0 (M−1); HPLC UV purity Rt=7.537 min, 99.27%; Chiral HPLC purity Rt=18.63 min, 99.5%; melting point=185-186° C.; optical rotation [α]²⁵_{D}=+76.8 (c=0.5, 1.0 mL IPA).

Diastereomer 2 (peak 2, 15b) was concentrated under reduced pressure to afford a white solid (0.045 g, 72.5% yield). R_{f} 0.27 with 1:40:60 v/v acetic acid-ethyl acetate-hexane (UV 254 nM); ¹H-NMR (400 MHz; DMSO-d6) δ 12.33 (s, 1H), 8.01 (br s, 1H), 7.83 (d, 1H, J=8.7 Hz), 7.40-7.45 (m, 2H), 7.28 (s, 1H), 7.25 (d, 1H, J=8.0 Hz), 7.1-7.2 (m, 3H), 6.94 (s, 1H), 2.4-2.5 (m, 1H), 1.8-1.9 (m, 1H), 1.46 (td, 1H, J=4.7, 9.2 Hz), 1.38 (ddd, 1H, J=4.4, 6.4, 8.3 Hz); ¹⁹F-NMR (376 MHz; DMSO-d6) δ −56.70; MS (APCI+) m/z 405.0 (M+1), MS (APCI−) m/z 403.0 (M−1); HPLC UV purity Rt=7.537 min, 97.2%; Chiral HPLC purity Rt=17.08 min, 96.4%; melting point=181-182° C.; optical rotation [α]²⁵_{D}=−60.8 (c=0.5 g, 100 mL in MeOH).

### Example 26: Synthesis of 5-(2-carbamoyl-6-(trifluoromethoxy)-1H-indol-1-yl)benzofuran-2-carboxylic acid (Compound 16)

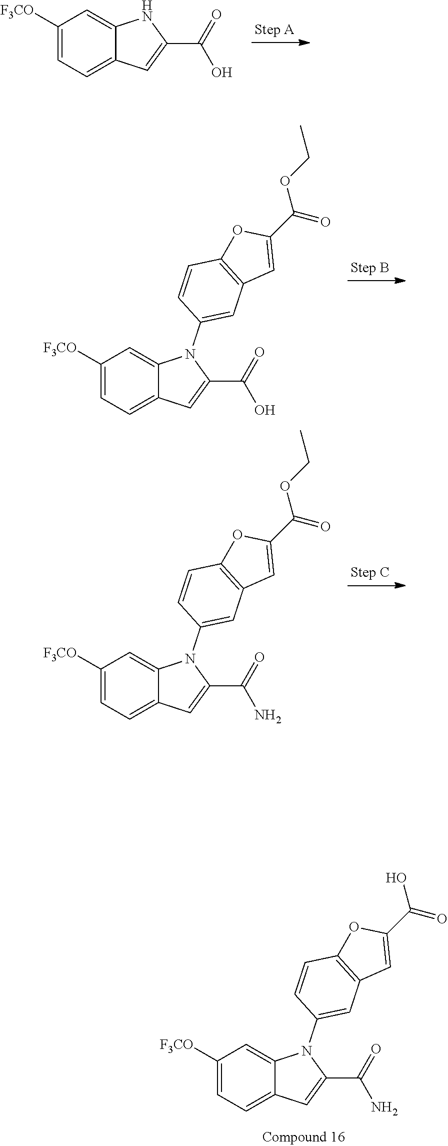

### Step A: Preparation of 1-(2-(ethoxycarbonyl)benzofuran-5-yl)-6-(trifluoromethoxy)-1H-indole-2-carboxylic acid

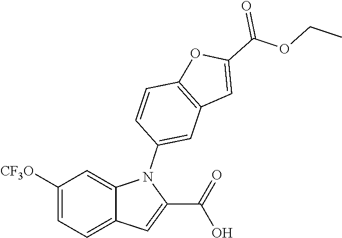

To a mixture consisting of 6-(trifluoromethoxy)-1H-indole-2-carboxylic acid (ChemShuttle, 0.245 g, 1.00 mmol), ethyl 5-bromobenzofuran-2-carboxylate (CombiBlocks, 0.269 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 115° C. under N₂ atmosphere for 7 hours and then stirred overnight at room temperature. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.571 g. The crude oil was purified by flash silica column chromatography on a CombiFlash NextGen 300+ purification system. Elution through a 24 g RediSep Gold Rf flash silica cartridge with 5-30% ethyl acetate in hexanes containing 1% acetic acid afforded the title compound as an orange solid (0.048 g, 11% yield). R_{f} 0.33 with 1:30:70 v/v acetic acid-ethyl acetate-hexane (UV 254 nM); MS (APCI−) m/z 432 (M−1); The product was used in the next step without further characterization.

### Step B: Preparation of ethyl 5-(2-carbamoyl-6-(trifluoromethoxy)-1H-indol-1-yl)benzofuran-2-carboxylate

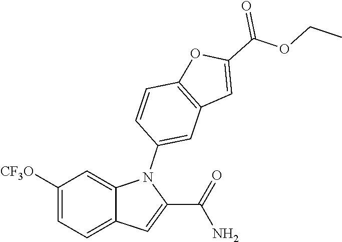

To a mixture consisting of 1-(2-(ethoxycarbonyl)benzofuran-5-yl)-6-(trifluoromethoxy)-1H-indole-2-carboxylic acid (0.048 g, 0.110 mmol) in DMF (3 mL) was added ammonium chloride (Chem-Impex, 0.018 g, 0.33 mmol). Next add TBTU (Oakwood, 0.053 g, 0.16 mmol), followed by the addition of DIPEA (0.17 mL, 0.99 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (15 mL), followed by brine (15 mL). The organic layer was concentrated under reduced pressure to afford the crude product as a white solid (0.043 g, 91% yield). The crude solid was used in the next reaction w/o further purification. R_{f} 0.09 with 1:30:70 v/v acetic acid-ethyl acetate-hexane (UV 254 nM); ¹H-NMR (400 MHz; DMSO-d6) δ 8.07 (br s, 1H), 7.9-8.0 (m, 1H), 7.87 (dd, 2H, J=4.2, 8.6 Hz), 7.83 (s, 1H), 7.79 (d, 1H, J=2.1 Hz), 7.46 (dd, 1H, J=2.3, 8.7 Hz), 7.39 (br s, 1H), 7.35 (s, 1H), 7.17 (d, 1H, J=8.7 Hz), 6.95 (s, 1H), 4.40 (q, 2H, J=7.1 Hz), 1.36 (t, 3H, J=7.1 Hz); ¹⁹F-NMR (376 MHz; DMSO-d6) δ −56.71; MS (APCI+) m/z 433.2 (M+1), (APCI−) m/z 432.0 (M−1); HPLC UV purity, Rt=9.232 min, 91.3%.

### Step C: Preparation of 5-(2-carbamoyl-6-(trifluoromethoxy)-1H-indol-1-yl)benzofuran-2-carboxylic acid (Compound 16)

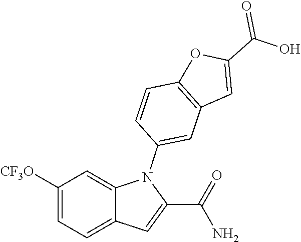

To a mixture consisting of ethyl 5-(2-carbamoyl-6-(trifluoromethoxy)-1H-indol-1-yl)benzofuran-2-carboxylate (0.043 g, 0.09 mmol) in methanol (2 mL) was added 1M lithium hydroxide (0.40 mL, 0.40 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (10 mL), followed by brine (10 mL). The organic layer was concentrated under reduced pressure to afford the crude product as a white solid (0.038 g, 95% yield). R_{f} 0.12 with 1:50:50 v/v acetic acid-ethyl acetate-hexane (UV 254 nM); ¹H-NMR (400 MHz; DMSO-d6) δ 12.09 (s, 1H), 8.06 (br s, 1H), 7.85 (t, 1H, J=8.5 Hz), 7.77 (d, 1H, J=2.1 Hz), 7.73 (s, 1H), 7.4-7.5 (m, 2H), 7.34 (s, 1H), 7.17 (dd, 1H, J=2.2, 8.7 Hz), 6.96 (s, 1H); ¹⁹F-NMR (376 MHz; DMSO-d₆) δ −56.71; MS (APCI⁺) m/z 405.0 (M+1), 403.0 (M-1); HPLC UV purity, Rt=7.39 min, 99.6%; melting point=293-294° C.

### Example 27: Synthesis of 3-(3-(2-carbamoyl-5-(trifluoromethoxy)benzofuran-3-yl)phenyl)propanoic acid (Compound 17)

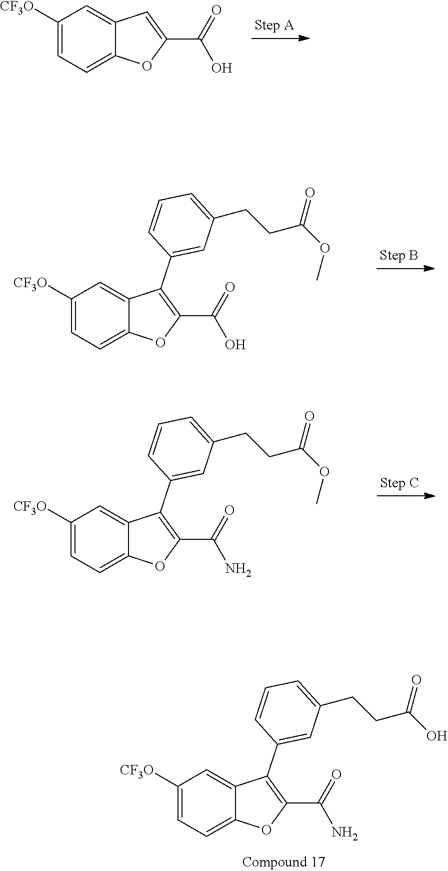

### Step A: Preparation of 3-(3-(3-methoxy-3-oxopropyl)phenyl)-5-(trifluoromethoxy)benzofuran-2-carboxylic acid

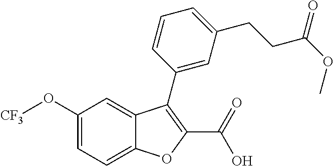 [SMILES: COC(=O)CCc1cccc(-c2c(C(=O)O)oc3ccc(OC(F)(F)F)cc23)c1]

To a 24 mL septa-cap vial was added methyl 3-bronobenzenepropanoate (Combi-Blocks, 0.246 g, 1.0 mmol) and 5-(trifluoromethoxy)benzofuran-2-carboxylic acid (0.243 g, 1.0 mmol); prepared by the method of Gensini, M. et. al., ChemMedChem 2010, 5(1), 65-78. Next was added Bis(dichloro(η⁶-p-cymene)ruthenium) (Strem, 0.025 g, 4 mol %), trimethylphosphonium tetrafluoroborate (Strem, 0.014 g, 8 mol %), K₂CO₃ (VWR, 0.152 g, 1.1 mmol) and NMP (5 mL). The mixture was degassed by bubbling in nitrogen gas for 3 min with stirring then heated to 110° C. overnight. The reaction mixture was subsequently cooled to room temperature and diluted with water (140 mL). The mixture was extracted with ethyl acetate (3×100 mL) and the combined organics dried over anhydrous sodium sulfate, filtered, and concentrated under reduced pressure to give a crude oil which was purified by flash silica column chromatography on a CombiFlash NextGen 300+ purification system. Elution through a 24 g RediSep Gold Rf flash silica cartridge with 10-40% ethyl acetate in heptane containing 1% acetic acid afforded the title compound as an amorphous solid (0.167 g, 41% yield). R_{f} 0.22 with 1:30:70 v/v acetic acid-ethyl acetate-hexane (UV 254 nM); ¹H-NMR (400 MHz; DMSO-d₆) δ 7.88 (d, 1H, J=8.9 Hz), 7.5-7.6 (m, 1H), 7.46 (s, 1H), 7.3-7.4 (m, 3H), 7.3-7.3 (m, 1H), 3.55 (s, 3H), 2.8-2.9 (m, 2H), 2.6-2.7 (m, 2H); MS (APCI+) m/z 409.1 (M+1), m/z 407.1, (M−1).

### Step B: Preparation of methyl 3-(3-(2-carbamoyl-5-(trifluoromethoxy)benzofuran-3-yl)phenyl)propanoate

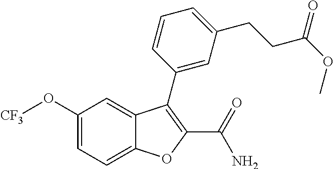 [SMILES: COC(=O)CCc1cccc(-c2c(C(N)=O)oc3ccc(OC(F)(F)F)cc23)c1]

To a mixture consisting of 3-(3-(3-methoxy-3-oxopropyl)phenyl)-5-(trifluoromethoxy)benzofuran-2-carboxylic acid (0.160 g, 0.39 mmol) in DMF (2.0 mL) was added ammonium chloride (Chem-Impex, 0.063 g, 1.17 mmol). Next was added TBTU (Oakwood, 0.189 g, 0.59 mmol) followed by the addition of DIPEA (0.62 mL, 3.5 mmol). The reaction mixture was stirred overnight at room temperature under N₂ atmosphere. The reaction mixture was subsequently diluted with water (60 mL) then extracted with 2×50 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 10-30% ethyl acetate in heptane containing 1% acetic acid afforded the title compound as an off-white solid (0.122 g, 76% yield). R_{f} 0.29 with 1:40:60 v/v acetic acid-ethyl acetate-hexane (UV 254 nM); ¹H-NMR (400 MHz; CDCl₃) δ 7.59 (d, 1H, J=8.9 Hz), 7.46 (d, 3H, J=4.6 Hz), 7.42 (s, 1H), 7.3-7.4 (m, 2H), 6.34 (br s, 1H), 5.74 (br s, 1H), 3.68 (s, 3H), 3.04 (t, 2H, J=7.7 Hz), 2.71 (t, 2H, J=7.7 Hz); MS (APCI⁺) m/z 408.1.0 (M+1), m/z 406.0, (M−1).

### Step C: Preparation of 3-(3-(2-carbamoyl-5-(trifluoromethoxy)benzofuran-3-yl)phenyl)propanoic acid (Compound 17)

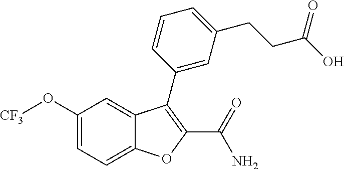 [SMILES: NC(=O)c1oc2ccc(OC(F)(F)F)cc2c1-c1cccc(CCC(=O)O)c1]

To a mixture consisting of methyl 3-(3-(2-carbamoyl-5-(trifluoromethoxy)benzofuran-3-yl)phenyl)propanoate (0.113 g, 0.28 mmol) in MeOH (3 mL) was added dropwise, a 1M aqueous solution of LiOH (0.83 mL, 3 eq). The reaction mixture was stirred overnight at room temperature. The clear solution was subsequently diluted with water (10 mL) and subjected to reduced pressure on a rotary evaporator to remove MeOH. Additional water was added (10 mL) and the solution was acidified with 1N HCl to precipitate product. The suspension was filtered, and solids washed with excess water then dried under high vac overnight to afford the title compound as a white solid (0.096 g, 87% yield). R_{f} 0.22 with 1:40:60 v/v acetic acid-ethyl acetate-hexane (UV 254 nM); ¹H-NMR (400 MHz; DMSO-d₆) δ 12.16 (br s, 1H), 8.06 (br s, 1H), 7.7-7.9 (m, 2H), 7.5-7.6 (m, 2H), 7.4-7.5 (m, 3H), 7.31 (br d, 1H, J=7.1 Hz), 2.90 (t, 2H, J=7.6 Hz), 2.59 (t, 2H, J=7.7 Hz); ¹⁹F-NMR (376 MHz; DMSO-d6) δ −57.02; MS (APCI⁺) m/z 394.0 (M+1), 392.0 (M−1); HPLC UV purity, Rt=7.55 min, 99.5%; melting point=234.0-235.5° C.

### Example 28: Synthesis of 3-(6-(2-carbamoyl-6-(trifluoromethoxy)-1H-indol-1-yl)pyridin-2-yl)propanoic acid (Compound 18)

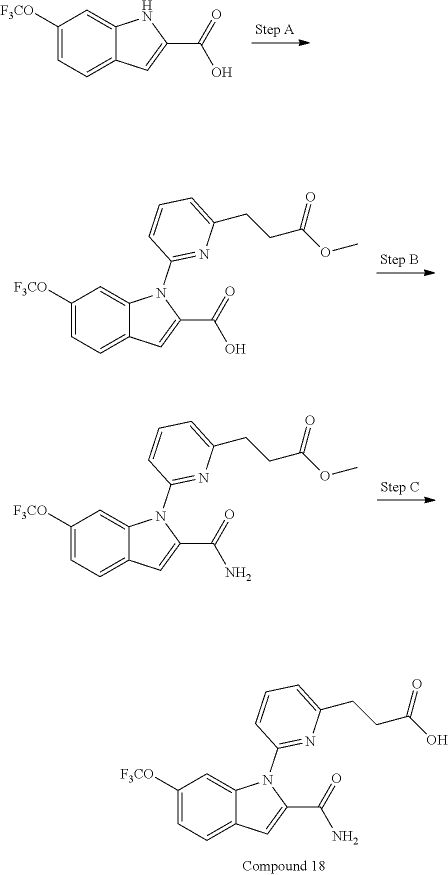 [SMILES: O=C(O)c1cc2ccc(OC(F)(F)F)cc2[nH]1 ; COC(=O)CCc1cccc(-n2c(C(=O)O)cc3ccc(OC(F)(F)F)cc32)n1 ; COC(=O)CCc1cccc(-n2c(C(N)=O)cc3ccc(OC(F)(F)F)cc32)n1 ; NC(=O)c1cc2ccc(OC(F)(F)F)cc2n1-c1cccc(CCC(=O)O)n1]

### Step A: Preparation of 1-(6-(3-methoxy-3-oxopropyl)pyridin-2-yl)-6-(trifluoromethoxy)-1H-indole-2-carboxylic acid

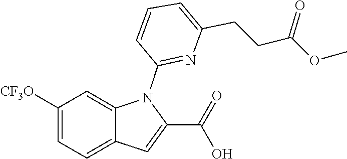

To a mixture consisting of 6-(trifluoromethoxy)-1H-indole-2-carboxylic acid (ChemShuttle, 0.368 g, 1.50 mmol), methyl 3-(6-bromopyridin-2-yl)propanoate (AstaTech, 0.366 g, 1.50 mmol), copper (II) acetate (CombiBlocks, 0.272 g, 1.50 mmol), methyl-α-D-glucopyranoside (CombiBlocks, 0.291 g, 1.50 mmol), potassium iodide (VWR, 0.498 g, 3.00 mmol) in DMSO (15 mL) was added DBU (Oakwood Chemicals, 0.67 mL, 4.50 mmol). The reaction mixture was heated to 115° C. under N₂ atmosphere for 3 hours. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.656 g. The crude oil was purified by flash silica column chromatography on a CombiFlash NextGen 300+ purification system. Elution through a 12 g RediSep Gold Rf flash silica cartridge with 5-30% ethyl acetate in hexanes containing 1% acetic acid afforded the title compound as an orange solid (0.059 g, 10% yield). R_{f} 0.15 with 1:30:70 v/v acetic acid-ethyl acetate-hexane (UV 254 nM); MS (APCI+) m/z 409.0 (M+1), (APCI−) m/z 407 (M−1), HPLC UV purity, Rt=8.54 min, 93.6%. The product was used in the next step without further characterization.

### Step B: Preparation of methyl 3-(6-(2-carbamoyl-6-(trifluoromethoxy)-1H-indol-1-yl)pyridin-2-yl)propanoate

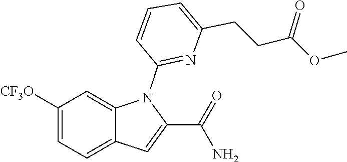

To a mixture consisting of methyl 3-(6-(2-carbamoyl-6-(trifluoromethoxy)-1H-indol-1-yl)pyridin-2-yl)propanoate (0.059 g, 0.144 mmol) in DMF (3 mL) was added ammonium chloride (Chem-Impex, 0.023 g, 0.43 mmol). Next add TBTU (Oakwood, 0.070 g, 0.22 mmol), followed by the addition of DIPEA (0.23 mL, 1.29 mmol). The reaction mixture was stirred overnight at room temperature under N₂ atmosphere. The reaction mixture was subsequently partitioned between ethyl acetate (25 mL) and H₂O (25 mL). The phases were separated, and the organic phase was partitioned with 1M KHSO₄ (25 mL), followed by brine (25 mL). The organic layer was concentrated under reduced pressure to afford the crude product as an orange solid (0.052 g, 90% yield). The crude solid was used in the next reaction w/o further purification. R_{f} 0.06 with 1:30:70 v/v acetic acid-ethyl acetate-hexane (UV 254 nM); MS (APCI+) m/z 408.0 (M+1), (APCI−) m/z 406.0 (M−1); HPLC UV purity, Rt=7.698 min, 86.1%.

### Step C: Preparation of 3-(6-(2-carbamoyl-6-(trifluoromethoxy)-1H-indol-1-yl)pyridin-2-yl)propanoic acid (Compound 18)

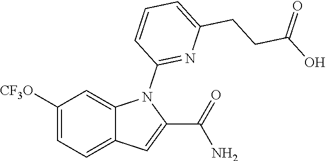

To a mixture consisting of methyl 3-(6-(2-carbamoyl-6-(trifluoromethoxy)-1H-indol-1-yl)pyridin-2-yl)propanoate (0.047 g, 0.12 mmol) in methanol (2 mL) was added 1M lithium hydroxide (0.50 mL, 0.50 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with water (10 mL), followed by brine (10 mL). The organic layer was concentrated under reduced pressure to afford the crude product as a white solid (0.033 g, 74% yield). The crude product was triturated with EA/Heptane (1:1, 10 mL) and filtered over a fritted funnel. The filtered white solid was isolated as the title product (0.013 g, 28% yield). R_{f} 0.12 with 1:50:50 v/v acetic acid-ethyl acetate-hexane (UV 254 nM); ¹H-NMR (400 MHz; DMSO-d6) δ 12.14 (s, 1H), 8.15 (br s, 1H), 7.88 (t, 1H, J=7.8 Hz), 7.84 (d, 1H, J=8.7 Hz), 7.49 (br s, 1H), 7.46 (s, 1H), 7.33 (d, 1H, J=7.6 Hz), 7.25 (s, 1H), 7.18 (d, 2H, J=7.8 Hz), 3.03 (t, 2H, J=7.5 Hz), 2.70 (t, 2H, J=7.5 Hz); ¹⁹F-NMR (376 MHz; DMSO-d6) δ −56.72; MS (APCI⁺) m/z 394.0 (M+1), 392.0 (M−1); HPLC UV purity, Rt=6.605 min, 96.6%; melting point=210-211° C.

### Example 29: Synthesis of 1-(3-(2-carbamoyl-6-(trifluoromethoxy)-1H-indol-1-yl)benzyl)cyclopropane-1-carboxylic acid (Compound 19)

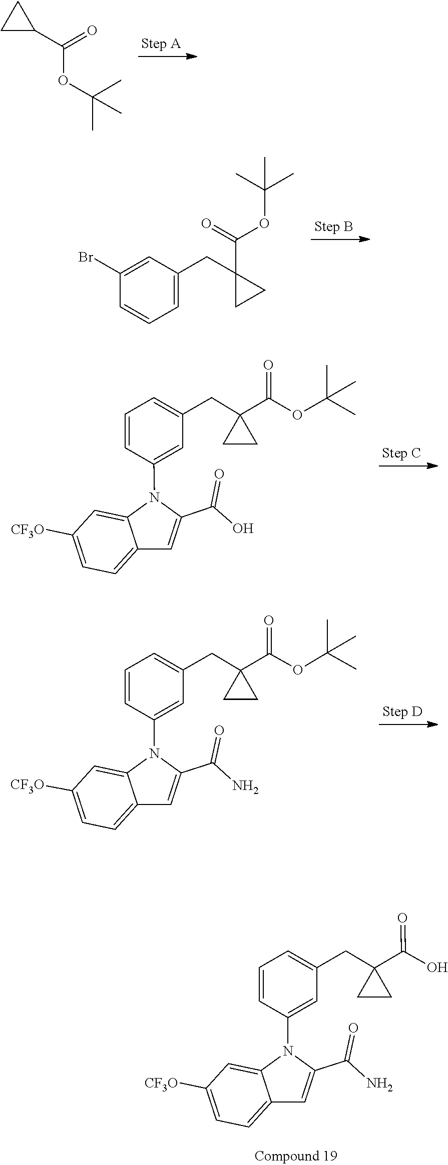

### Step A: Preparation of tert-butyl 1-(3-bromobenzyl)cyclopropane-1-carboxylate

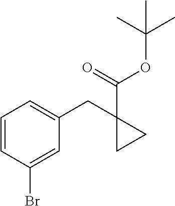 [SMILES: CC(C)(C)OC(=O)C1(Cc2cccc(Br)c2)CC1]

To a −78° C. solution of tert-butyl cyclopropanecarboxylate (Oakwood, 1.00 g, 7.03 mmol) in THF (10 mL) was added freshly prepared LDA (0.5M solution in THF, 21 mL, 10.50 mmol) portion-wise over 15 minutes. The reaction mixture was stirred for 3 hours at −78° C. before the addition of 1-bromo-3-(bromomethyl)benzene (Combi-Blocks, 1.90 g, 7.66 mmol). The reaction mixture was allowed to warm to room temperature after stirring overnight. The reaction mixture was subsequently partitioned between ethyl acetate (50 mL) and saturated ammonium chloride (75 mL). The phases were separated, and the ammonium chloride layer was back extracted an additional (2×) with ethyl acetate (50 mL). The combined organic layer was dried over MgSO₄, filtered over a fritted funnel, and the filtrate concentrated under reduced pressure to afford the crude product as a yellow oil (2.05 g). The crude oil was purified by flash silica column chromatography on a CombiFlash NextGen 300+ purification system. Elution through a 24 g RediSep Gold Rf flash silica cartridge with 5-30% ethyl acetate in hexanes afforded the title compound as a colorless oil (1.42 g, 67% yield). R_{f} 0.75 with 1:30:70 v/v acetic acid-ethyl acetate-hexane (UV 254 nM); ¹H-NMR (400 MHz; CDCl₃) δ 7.43 (br s, 1H), 7.33 (d, 1H, J=7.8 Hz), 7.1-7.2 (m, 2H), 2.88 (s, 2H), 1.36 (s, 9H), 1.2-1.3 (m, 2H), 0.7-0.8 (m, 2H); LC/MS, Rt=8.434 min, MS (APCI−) m/z 254,256 (M-t-butyl, Br present).

### Step B: Preparation of 1-(3-((1-(tert-butoxycarbonyl)cyclopropyl)methyl)phenyl)-6-(trifluoromethoxy)-1H-indole-2-carboxylic acid

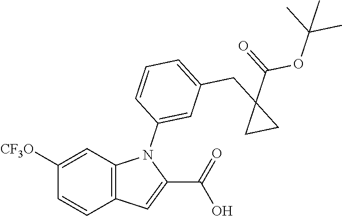

To a mixture consisting of 6-(trifluoromethoxy)-1H-indole-2-carboxylic acid (ChemShuttle, 0.245 g, 1.00 mmol), tert-butyl 1-(3-bromobenzyl)cyclopropane-1-carboxylate (0.311 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.0 mmol). The reaction mixture was heated to 115° C. under N₂ atmosphere for 8 hours and then allowed to stir overnight at room temperature. The reaction mixture was subsequently partitioned between ethyl acetate (75 mL) and 1M KHSO₄ (75 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.588 g. The crude oil was purified by flash silica column chromatography on a CombiFlash NextGen 300+ purification system. Elution through a 12 g RediSep Gold Rf flash silica cartridge with 5-30% ethyl acetate in hexanes containing 1% acetic acid afforded the title compound as a white solid (0.279 g, product is a mixture). R_{f} 0.31 with 1:30:70 v/v acetic acid-ethyl acetate-hexane (UV 254 nM); LC/MS Rt=6.3 min, MS (APCI−) m/z 474.0 (M−1). The product was used in the next step without further characterization or purification.

### Step C: Preparation of tert-butyl 1-(3-(2-carbamoyl-6-(trifluoromethoxy)-1H-indol-1-yl)benzyl)cyclopropane-1-carboxylate

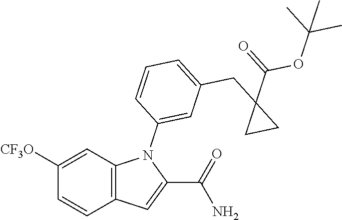

To a mixture consisting of 1-(3-((1-(tert-butoxycarbonyl)cyclopropyl)methyl)phenyl)-6-(trifluoromethoxy)-1H-indole-2-carboxylic acid (0.279 g, 0.586 mmol) in DMF (6 mL) was added ammonium chloride (Chem-Impex, 0.094 g, 1.76 mmol). Next add TBTU (Oakwood, 0.283 g, 0.88 mmol), followed by the addition of DIPEA (0.91 mL, 5.27 mmol). The reaction mixture was stirred overnight at room temperature under N₂ atmosphere. The reaction mixture was subsequently partitioned between ethyl acetate (30 mL) and H₂O (25 mL). The phases were separated, and the organic phase was partitioned with 1M KHSO₄ (25 mL), followed by brine (25 mL). The organic layer was concentrated under reduced pressure to afford the crude product as a white solid (0.253 g, 96% yield). The crude solid was used in the next reaction w/o further purification. R_{f} 0.55 with 1:50:50 v/v acetic acid-ethyl acetate-hexane (UV 254 nM); LC/MS Rt=7.842 min, MS (APCI−) m/z 473.0 (M−1). The product was used in the next step without further characterization or purification.

### Step D: Preparation of 1-(3-(2-carbamoyl-6-(trifluoromethoxy)-1H-indol-1-yl)benzyl)cyclopropane-1-carboxylic acid (Compound 19)

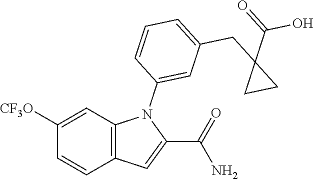

To a mixture consisting of 1-(3-((1-(tert-butoxycarbonyl)cyclopropyl)methyl)phenyl)-6-(trifluoromethoxy)-1H-indole-2-carboxylic acid (0.253 g, 0.53 mmol) in methanol (10 mL) was added 1M lithium hydroxide (2.20 mL, 2.20 mmol). The reaction mixture was stirred at room temperature overnight under N₂ atmosphere. The reaction mixture was subsequently partitioned between ethyl acetate (25 mL) and 1M HCl (15 mL). The phases were separated, and the organic phase was partitioned with water (15 mL), followed by brine (15 mL). The organic layer was concentrated under reduced pressure to afford the crude product as a colorless oil (0.244 g). The crude oil was purified by flash silica column chromatography on a CombiFlash NextGen 300+ purification system. Elution through a 24 g RediSep Gold Rf flash silica cartridge with 5-30% ethyl acetate in hexanes containing 1% acetic acid afforded the desired compound as a mixture (0.053 g). The crude product was repurified by flash silica column chromatography on a CombiFlash NextGen 300+ purification system. Elution through a 4 g RediSep Gold Rf flash silica cartridge with 5% ethyl acetate in hexanes containing 1% acetic acid afforded the desired compound as a white solid (0.027 g, 12% yield). R_{f} 0.49 with 1:50:50 v/v acetic acid-ethyl acetate-hexane (UV 254 nM); ¹H-NMR (400 MHz; DMSO-d6) δ 12.14 (s, 1H), 8.15 (br s, 1H), 7.88 (t, 1H, J=7.8 Hz), 7.84 (d, 1H, J=8.7 Hz), 7.49 (br s, 1H), 7.46 (s, 1H), 7.33 (d, 1H, J=7.6 Hz), 7.25 (s, 1H), 7.18 (d, 2H, J=7.8 Hz), 3.03 (t, 2H, J=7.5 Hz), 2.70 (t, 2H, J=7.5 Hz); ¹⁹F-NMR (376 MHz; DMSO-d6) δ −56.72; MS (APCI⁺) m/z 394.0 (M+1), 392.0 (M−1); HPLC UV purity, Rt=6.605 min, 96.6%; melting point=141-142° C.

### Example 30: Synthesis of 5-(2-carbamoyl-6-(trifluoromethoxy)-1H-indol-1-yl)benzofuran-3-carboxylic acid (Compound 20)

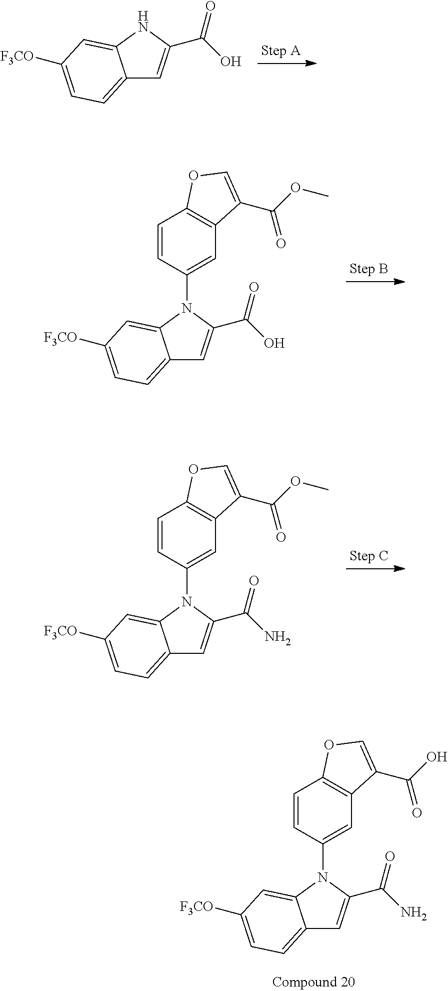 [SMILES: O=C(O)c1cc2ccc(OC(F)(F)F)cc2[nH]1 ; COC(=O)c1coc2ccc(-n3c(C(=O)O)cc4ccc(OC(F)(F)F)cc43)cc12 ; COC(=O)c1coc2ccc(-n3c(C(N)=O)cc4ccc(OC(F)(F)F)cc43)cc12 ; NC(=O)c1cc2ccc(OC(F)(F)F)cc2n1-c1ccc2occ(C(=O)O)c2c1]

### Step A: Preparation of 1-(3-(methoxycarbonyl)benzofuran-5-yl)-6-(trifluoromethoxy)-1H-indole-2-carboxylic acid

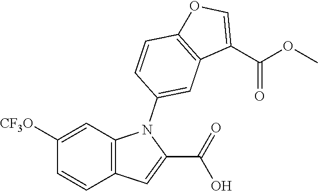

To a mixture consisting of 6-(trifluoromethoxy)-1H-indole-2-carboxylic acid (ChemShuttle, 0.245 g, 1.00 mmol), methyl 5-bromobenzofuran-3-carboxylate (0.255 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.0 mmol). The reaction mixture was heated to 115° C. under N₂ atmosphere for 8 hours and then allowed to stir overnight at room temperature. The reaction mixture was subsequently partitioned between ethyl acetate (75 mL) and 1M KHSO₄ (75 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.633 g. The crude oil was purified by flash silica column chromatography on a CombiFlash NextGen 300+ purification system. Elution through a 12 g RediSep Gold Rf flash silica cartridge with 5-30% ethyl acetate in hexanes containing 1% acetic acid afforded the title compound as a tan solid (0.041 g, 9.7% yield). R_{f} 0.56 with 1:50:50 v/v acetic acid-ethyl acetate-hexane (UV 254 nM); MS (APCI+) m/z 420.0 (M+1), (APCI−) m/z 419.0 (M−1); HPLC UV purity, Rt=9.598 min, 96.8%. The product was used in the next step without further characterization or purification.

### Step B: Preparation of methyl 5-(2-carbamoyl-6-(trifluoromethoxy)-1H-indol-1-yl)benzofuran-3-carboxylate

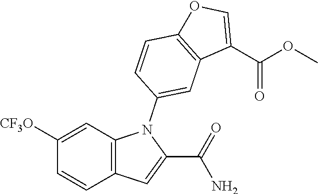

To a mixture consisting of 1-(3-(methoxycarbonyl)benzofuran-5-yl)-6-(trifluoromethoxy)-1H-indole-2-carboxylic acid (0.040 g, 0.095 mmol) in DMF (2 mL) was added ammonium chloride (Chem-Impex, 0.015 g, 0.143 mmol). Next add TBTU (Oakwood, 0.046 g, 0.143 mmol), followed by the addition of DIPEA (0.15 mL, 0.855 mmol). The reaction mixture was stirred for 4 hours at room temperature under N₂ atmosphere. The reaction mixture was subsequently partitioned between ethyl acetate (15 mL) and H₂O (15 mL). The phases were separated, and the organic phase was partitioned with 1M KHSO₄ (10 mL), followed by brine (10 mL). The organic layer was concentrated under reduced pressure to afford the crude product as a yellow film (0.36 g, 92% yield). The crude solid was used in the next reaction w/o further purification. R_{f} 0.34 with 1:50:50 v/v acetic acid-ethyl acetate-hexane (UV 254 nM): MS (APCI+) m/z 419.0 (M+1); HPLC UV purity, Rt=8.816 min, 93.3%.

### Step C: Preparation of 5-(2-carbamoyl-6-(trifluoromethoxy)-1H-indol-1-yl)benzofuran-3-carboxylic acid (Compound 20)

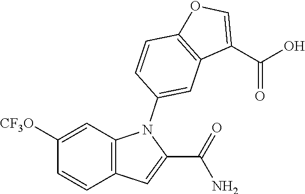

To a mixture consisting of methyl 5-(2-carbamoyl-6-(trifluoromethoxy)-1H-indol-1-yl)benzofuran-3-carboxylate (0.036 g, 0.087 mmol) in methanol (2 mL) was added 1M lithium hydroxide (0.35 mL, 0.35 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (10 mL), followed by brine (10 mL). The organic layer was concentrated under reduced pressure to afford the crude product as a tan solid (0.030 g). TLC and LC/MS showed the presence of starting material, so the crude material was redissolved in Methanol (2 mL) and 1M LiOH (1 mL) was added. The reaction mixture was heated to 45° C. for 5 hours until the reaction showed complete conversion. The reaction mixture was subsequently partitioned between ethyl acetate (25 mL) and 1M HCl (15 mL). The phases were separated, and the organic phase was partitioned with water (15 mL), followed by brine (15 mL). The organic layer was concentrated under reduced pressure to afford the crude product as an orange solid (0.026 g). The product was triturated with (EA/Hep 40:60, 6 mL) and filtered over a fritted funnel to afford the desired compound as tan solid (0.016 g, 46% yield). R_{f} 0.22 with 1:50:50 v/v acetic acid-ethyl acetate-hexane (UV 254 nM); ¹H-NMR (400 MHz; DMSO-d6) δ 13.14 (s, 1H), 8.82 (s, 1H), 8.07 (br s, 1H), 7.86 (dd, 1H, J=2.1 Hz), 7.40-7.45 (m, 2H), 7.34 (s, 1H), 7.18 (d, 1H, J=7.8 Hz), 6.93 (s, 1H); ¹⁹F-NMR (376 MHz; DMSO-d6) δ −56.74; MS (APCI⁺) m/z 405.0 (M+1), 403.0 (M−1); HPLC UV purity, Rt=7.569 min, 96.5%; melting point=277-278° C.

### Example 31: Synthesis of 5-(2-carbamoyl-6-(trifluoromethoxy)-1H-indol-1-yl)benzofuran-3-carboxylic acid (Compound 21)

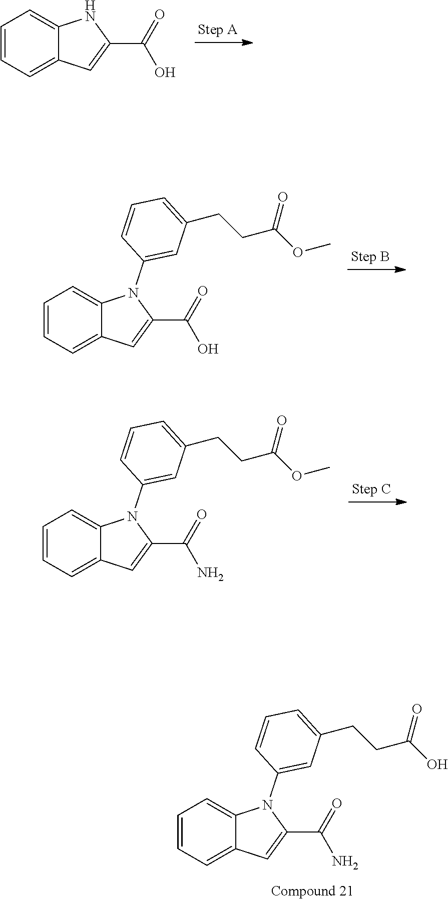 [SMILES: O=C(O)c1cc2ccccc2[nH]1 ; COC(=O)CCc1cccc(-n2c(C(=O)O)cc3ccccc32)c1 ; COC(=O)CCc1cccc(-n2c(C(N)=O)cc3ccccc32)c1 ; NC(=O)c1cc2ccccc2n1-c1cccc(CCC(=O)O)c1]

### Step A: Preparation of 1-(3-(3-methoxy-3-oxopropyl)phenyl)-1H-indole-2-carboxylic acid

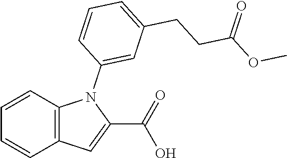 [SMILES: COC(=O)CCc1cccc(-n2c(C(=O)O)cc3ccccc32)c1]

To a mixture consisting of 1H-indole-2-carboxylic acid (Ambeed, 0.483 g, 3.00 mmol), methyl 3-(3-bromophenyl)propanoate (CombiBlocks, 0.729 g, 3.00 mmol), copper (II) acetate (CombiBlocks, 0.543 g, 3.00 mmol), methyl-α-D-glucopyranoside (CombiBlocks, 0.582 g, 3.00 mmol), potassium iodide (VWR, 0.996 g, 6.00 mmol) in DMSO (20 mL) was added DBU (Oakwood Chemicals, 1.34 mL, 9.0 mmol). The reaction mixture was heated to 115° C. under N₂ atmosphere for 8 hours and then allowed to stir overnight at room temperature. The reaction mixture was subsequently partitioned between ethyl acetate (100 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 1.15 g. The crude oil was purified by flash silica column chromatography on a CombiFlash NextGen 300+ purification system. Elution through a 24 g RediSep Gold Rf flash silica cartridge with 5-30% ethyl acetate in hexanes containing 1% acetic acid afforded the title compound as a yellow oil (0.283 g, 29% yield). R_{f} 0.30 with 1:30:70 v/v acetic acid-ethyl acetate-hexane (UV 254 nM); MS (APCI+) m/z 324.0 (M+1), (APCI−) m/z 322.0 (M−1); LC/MS, Rt=5.401 min, (APCI+) m/z 324.0 (M+1). The product was used in the next step without further characterization or purification.

### Step B: Preparation of methyl 3-(3-(2-carbamoyl-1H-indol-1-yl)phenyl)propanoate

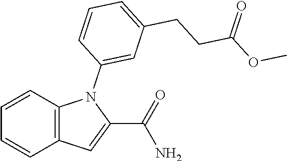 [SMILES: COC(=O)CCc1cccc(-n2c(C(N)=O)cc3ccccc32)c1]

To a mixture consisting of 1-(3-(3-methoxy-3-oxopropyl)phenyl)-1H-indole-2-carboxylic acid (0.265 g, 0.819 mmol) in DMF (6 mL) was added ammonium chloride (Chem-Impex, 0.131 g, 2.46 mmol). Next add TBTU (Oakwood, 0.395 g, 1.23 mmol), followed by the addition of DIPEA (1.28 mL, 7.37 mmol). The reaction mixture was stirred overnight at room temperature under N₂ atmosphere. The reaction mixture was subsequently partitioned between ethyl acetate (50 mL) and H₂O (50 mL). The phases were separated, and the organic phase was partitioned with 1M KHSO₄ (25 mL), followed by brine (25 mL). The organic layer was concentrated under reduced pressure to afford the crude product as a yellow oil (0.49 g). The crude oil was purified by flash silica column chromatography on a CombiFlash NextGen 300+ purification system. Elution through a 24 g RediSep Gold Rf flash silica cartridge with 5-30% ethyl acetate in hexanes containing 1% acetic acid afforded the title compound as a yellow solid (0.069 g, 26% yield). R_{f} 0.12 with 1:30:70 v/v acetic acid-ethyl acetate-hexane (UV 254 nM): MS (APCI+) m/z 323.2 (M+1); HPLC UV purity, Rt=7.428 min, 98.9%. The product was used in the next step without further characterization or purification.

### Step C: Preparation of 5-(2-carbamoyl-6-(trifluoromethoxy)-1H-indol-1-yl)benzofuran-3-carboxylic acid (Compound 21)

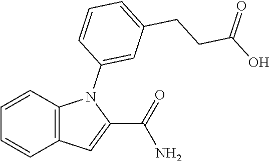 [SMILES: NC(=O)c1cc2ccccc2n1-c1cccc(CCC(=O)O)c1]

To a mixture consisting of methyl 3-(3-(2-carbamoyl-1H-indol-1-yl)phenyl)propanoate (0.069 g, 0.217 mmol) in methanol (4 mL) was added 1M lithium hydroxide (0.87 mL, 0.87 mmol). The reaction mixture was stirred at room temperature overnight under N₂ atmosphere. The reaction mixture was subsequently partitioned between ethyl acetate (25 mL) and 1M HCl (10 mL). The phases were separated, and the organic phase was partitioned with water (25 mL), followed by brine (25 mL). The organic layer was concentrated under reduced pressure to afford the title compound as a white solid (0.054 g, 82% yield). R_{f} 0.71 with 1:10:90 v/v acetic acid-methanol-dichloromethane (UV 254 nM); ¹H-NMR (400 MHz; DMSO-d6) δ 12.15 (s, 1H), 7.89 (br s, 1H), 7.66 (d, 1H, J=7.8 Hz), 7.37 (t, 1H, J=7.7 Hz), 7.25 (br d, 2H, J=7.8 Hz), 7.15-7.19 (m, 3H), 7.05-7.13 (m, 3H), 2.86 (t, 2H, J=7.7 Hz), 2.55 (t, 2H, J=7.7 Hz); MS (APCI⁺) m/z 309.0 (M+1), 307.0 (M−1); HPLC UV purity, Rt=6.251 min, 99.7%; melting point=221-222° C.

### Example 32: Synthesis of 3-(3-(2-carbamoyl-6-(trifluoromethoxy)-1H-indol-1-yl)phenyl)-3-methylbutanoic acid (Compound 22)

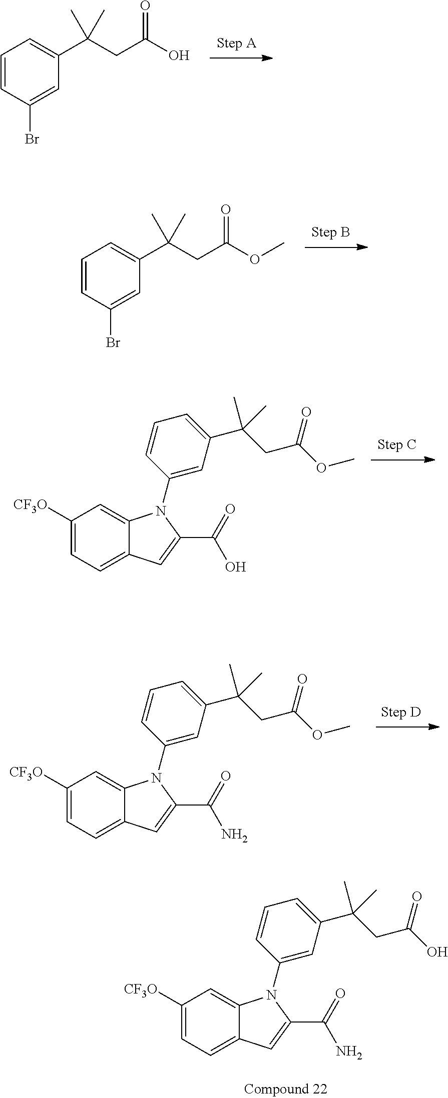

### Step A: Preparation of methyl 3-(3-bromophenyl)-3-methylbutanoate

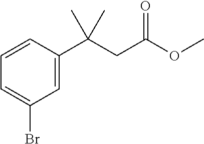

To an ice bath (0° C.) reaction mixture consisting of 3-(3-bromophenyl)-3-methylbutanoic acid (0.50 g, 1.94 mmol) in toluene (6 mL) and methanol (4 mL) was added TMS-Diazomethane (2M solution in Ether, 1.45 mL, 2.91 mmol) dropwise. The reaction was stirred at 0° C. for 30 minutes then allowed to warm to room temperature for 2 hours. The reaction mixture was concentrated under reduced pressure to afford the crude product as a yellow oil (0.550 g). The crude oil was purified by flash silica column chromatography on a CombiFlash NextGen 300+ purification system. Elution through a 12 g RediSep Gold Rf flash silica cartridge with 5-100% ethyl acetate in heptane afforded the title compound as a colorless oil (0.380 g, 72% yield). R_{f} 0.69 with 25:75 v/v ethyl acetate-heptane (UV 254 nM); ¹H-NMR (400 MHz; CDCl₃) δ 7.50 (t, 1H, J=1.8 Hz), 7.30-7.40 (m, 2H), 7.1-7.2 (m, 1H), 3.55 (s, 3H), 2.62 (s, 2H), 1.44 (s, 6H); MS (APCI+) m/z 271.0 (M+1); HPLC UV purity, Rt=9.620 min, 100%.

### Step B: Preparation of 1-(3-(4-methoxy-2-methyl-4-oxobutan-2-yl)phenyl)-6-(trifluoromethoxy)-1H-indole-2-carboxylic acid

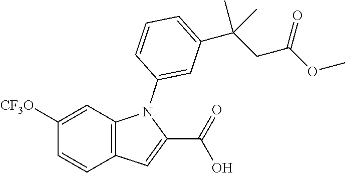

To a mixture consisting of 6-(trifluoromethoxy)-1H-indole-2-carboxylic acid (ChemShuttle, 0.339 g, 1.38 mmol), methyl 3-(3-bromophenyl)-3-methylbutanoate (0.375 g, 1.38 mmol), copper (II) acetate (CombiBlocks, 0.250 g, 1.38 mmol), methyl-α-D-glucopyranoside (CombiBlocks, 0.268 g, 1.38 mmol), potassium iodide (VWR, 0.458 g, 2.76 mmol) in DMSO (20 mL) was added DBU (Oakwood Chemicals, 0.61 mL, 4.14 mmol). The reaction mixture was heated to 115° C. under N₂ atmosphere for 8 hours and then allowed to stir overnight at room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.724 g. The crude oil was purified by flash silica column chromatography on a CombiFlash NextGen 300+ purification system. Elution through a 24 g RediSep Gold Rf flash silica cartridge with 5-75% ethyl acetate in heptane containing 1% acetic acid afforded the title compound as a white solid (0.283 g). The product obtained contained two impurities and was used in the next reaction without further purification; R_{f} 0.30 with 1:30:70 v/v acetic acid-ethyl acetate-heptane (UV 254 nM); MS (APCI+) m/z 436.2 (M+1), (APCI−) m/z 434.0 (M−1); HPLC UV purity, Rt=10.071 min, 51.2%.

### Step C: Preparation of methyl 3-(3-(2-carbamoyl-6-(trifluoromethoxy)-1H-indol-1-yl)phenyl)-3-methylbutanoate

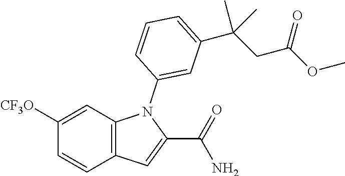

To a mixture consisting of 1-(3-(4-methoxy-2-methyl-4-oxobutan-2-yl)phenyl)-6-(trifluoromethoxy)-1H-indole-2-carboxylic acid (0.320 g, 0.735 mmol) in DMF (6 mL) was added ammonium chloride (Chem-Impex, 0.118 g, 2.21 mmol). Next add TBTU (Oakwood, 0.354 g, 1.10 mmol), followed by the addition of DIPEA (1.15 mL, 6.62 mmol). The reaction mixture was stirred for 3 hours at room temperature under N₂ atmosphere. The reaction mixture was subsequently partitioned between ethyl acetate (50 mL) and H₂O (50 mL). The phases were separated, and the organic phase was partitioned with 1M KHSO₄ (25 mL), followed by brine (25 mL). The organic layer was concentrated under reduced pressure to afford the crude product as a yellow oil (0.421 g). The crude oil was purified by flash silica column chromatography on a CombiFlash NextGen 300+ purification system. Elution through a 24 g RediSep Gold Rf flash silica cartridge with 5-75% ethyl acetate in heptane containing 1% acetic acid afforded the title compound as a white solid (0.108 g, 34% yield). R_{f} 0.17 with 1:30:70 v/v acetic acid-ethyl acetate-heptane (UV 254 nM): MS (APCI+) m/z 435.2 (M+1); HPLC UV purity, Rt=9.323 min, 96.5%.

### Step D: Preparation of 3-(3-(2-carbamoyl-6-(trifluoromethoxy)-1H-indol-1-yl)phenyl)-3-methylbutanoic acid (Compound 22)

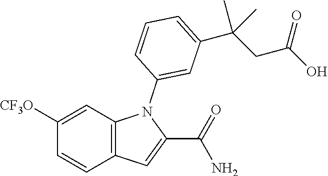

To a mixture consisting of methyl 3-(3-(2-carbamoyl-6-(trifluoromethoxy)-1H-indol-1-yl)phenyl)-3-methylbutanoate (0.108 g, 0.248 mmol) in methanol (4 mL) was added 1M lithium hydroxide (1.00 mL, 1.00 mmol). The reaction mixture was stirred at room temperature overnight under N₂ atmosphere. The reaction mixture was subsequently partitioned between ethyl acetate (25 mL) and 1M HCl (10 mL). The phases were separated, and the organic phase was partitioned with water (25 mL), followed by brine (25 mL). The organic layer was concentrated under reduced pressure to afford the title compound as a white solid (0.054 g, 82% yield). R_{f} 0.71 with 1:10:90 v/v acetic acid-methanol-dichloromethane (UV 254 nM); ¹H-NMR (400 MHz; DMSO-d6) δ 11.92 (br s, 1H), 7.99 (br s, 1H), 7.83 (d, 1H, J=8.7 Hz), 7.40-7.46 (m, 4H), 7.27 (s, 1H), 7.11-7.16 (m, 2H), 7.08 (br s, 1H), 2.62 (s, 2H), 1.41 (s, 6H); ¹⁹F-NMR (376 MHz; DMSO-d6) δ −56.77; MS (APCI⁺) m/z 421.2 (M+1), 419.2 (M−1); HPLC UV purity, Rt=8.166 min, 98.8%; melting point=163-164° C.

### Example 33: Synthesis of (R)-2-amino-3-(3-(2-carbamoyl-6-(trifluoromethoxy)-1H-indol-1-yl)phenyl)propanoic acid TFA salt (Compound 23)

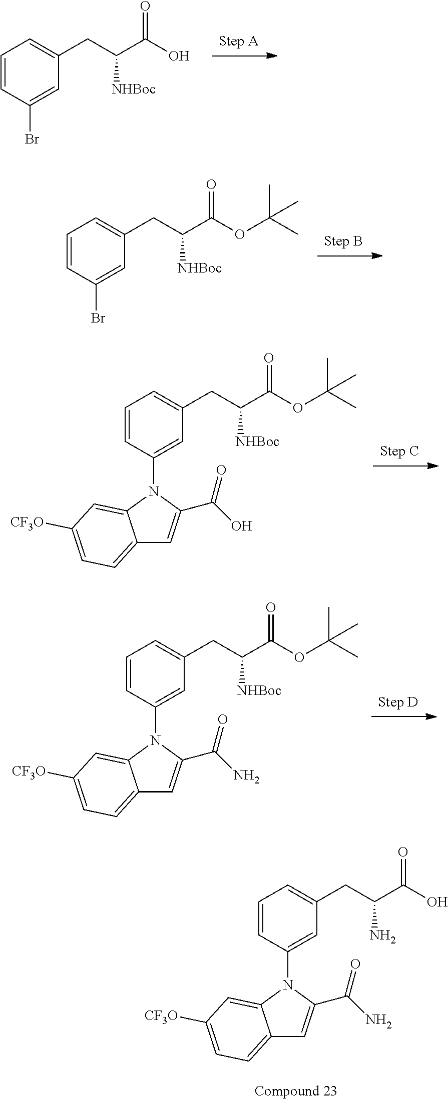

### Step A: Preparation of tert-butyl (R)-3-(3-bromophenyl)-2-((tert-butoxycarbonyl)amino)propanoate

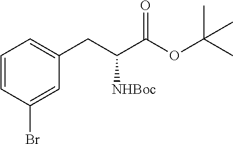 [SMILES: CC(C)(C)OC(=O)N[C@H](Cc1cccc(Br)c1)C(=O)OC(C)(C)C]

To a 20 mL tBuOH solution of (R)-3-(3-bromophenyl)-2-((tert-butoxycarbonyl)amino)propanoic acid (2.00 g, 5.80 mmol) at room temperature was added DMAP (0.07 g, 0.58 mmol) and di-tert-butyl dicarbonate (1.65 g, 7.50 mmol) under N₂ atmosphere and allowed the reaction to stir at room temperature for 14 hours. The reaction mixture was evaporated to dryness and partitioned between 100 mL ethyl acetate and 150 mL water. The organic layer was sequentially washed with water (100 mL) and brine (100 mL), then dried over anhydrous sodium sulfate. The crude reaction mixture was purified by flash silica column chromatography on a CombiFlash NextGen 300+ purification system. Elution through an 80 g RediSep Gold Rf flash silica cartridge with 0-5% methanol in DCM afforded the title compound as a clear and colorless oil (1.1 g, 47% yield). R_{f} 0.70 with 5:95 v/v methanol-dichloromethane (UV 254 nM); ¹H NMR (400 MHz, CHLOROFORM-d) δ 7.35 (br d, J=7.57 Hz, 1H), 7.25 (d, J=1.38 Hz, 1H), 7.05-7.19 (m, 2H), 4.70-5.12 (m, 1H), 4.31-4.59 (m, 1H), 2.88-3.12 (m, 2H), 1.41 (br d, J=8.71 Hz, 18H); MS (FIA MS+) m/z 400 (M+1).

### Step B: Preparation of (R)-1-(3-(3-(tert-butoxy)-2-((tert-butoxycarbonyl)amino)-3-oxopropyl)phenyl)-6-(trifluoromethoxy)-1H-indole-2-carboxylic acid

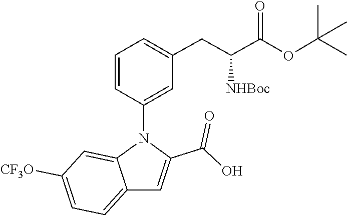

To a mixture consisting of 6-(trifluoromethoxy)-1H-indole-2-carboxylic acid (ChemShuttle, 0.24 g, 0.98 mmol), tert-butyl (R)-3-(3-bromophenyl)-2-((tert-butoxycarbonyl)amino)propanoate (0.43 g, 1.00 mmol), copper (II) acetate (CombiBlocks, 0.18 g, 0.98 mmol), methyl-α-D-glucopyranoside (CombiBlocks, 0.19 g, 0.98 mmol), potassium iodide (VWR, 0.16 g, 0.98 mmol) in DMSO (10 mL) was added DBU (Oakwood Chemicals, 0.45 mL, 2.90 mmol). The reaction mixture was heated to 115° C. under N₂ atmosphere for 14 hours and then stirred at room temperature for 1 hour. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude gummy mass of 0.500 g. The crude was purified by flash silica column chromatography on a CombiFlash NextGen 300+ purification system. Elution through a 24 g RediSep Gold Rf flash silica cartridge with 5-39% ethyl acetate in hexanes containing 1% acetic acid afforded the title compound as an off-white solid (0.18 g, 33% yield). R_{f} 0.32 with 1:39:60 v/v acetic acid-ethyl acetate-hexane (UV 254 nM); ¹H NMR (400 MHz, CHLOROFORM-d) δ 8.03-8.86 (m, 1H), 7.69 (br d, 1H, J=8.48 Hz), 7.48 (s, 1H), 7.32-7.44 (m, 1H), 7.20-7.31 (m, 2H), 7.17 (br d, 1H, J=7.57 Hz), 7.07 (br d, 1H, J=8.48 Hz), 6.95 (br s, 1H), 5.18 (br s, 1H), 4.67 (br s, 1H), 2.79-3.41 (m, 2H), 1.36 (br s, 19H); MS (FIA-) m/z 565.0 (M+1).

### Step C: Preparation of tert-butyl (R)-2-((tert-butoxycarbonyl)amino)-3-(3-(2-carbamoyl-6-(trifluoromethoxy)-1H-indol-1-yl)phenyl)propanoate

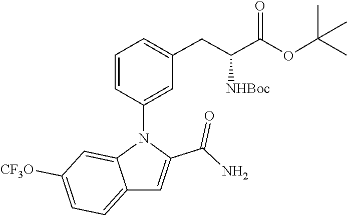

To a mixture consisting of (R)-1-(3-(3-(tert-butoxy)-2-((tert-butoxycarbonyl)amino)-3-oxopropyl)phenyl)-6-(trifluoromethoxy)-1H-indole-2-carboxylic acid (0.165 g, 0.29 mmol) in DMF (8 mL) was added ammonium chloride (Chem-Impex, 0.047 g, 0.88 mmol). Next add TBTU (Oakwood, 0.141 g, 0.43 mmol), followed by the addition of DIPEA (0.34 mL, 2.61 mmol). The reaction mixture was stirred overnight at room temperature under N₂ atmosphere. The reaction mixture was subsequently partitioned between ethyl acetate (50 mL) and H₂O (50×3 mL). The phases were separated, and the organic phase was partitioned with 1M KHSO₄ (50 mL), followed by brine (50 mL). The organic layer was concentrated under reduced pressure to afford the crude product as an orange solid. The crude was purified by flash silica column chromatography on a CombiFlash NextGen 300+ purification system. Elution through a 12 g RediSep Gold Rf flash silica cartridge with 5-39% ethyl acetate in hexanes containing 1% acetic acid afforded the title compound as an off-white solid (0.14 g, 87% yield). R_{f} 0.25 with 1:39:60 v/v acetic acid-ethyl acetate-hexane (UV 254 nM); ¹H NMR (400 MHz, CHLOROFORM-d) δ 7.71 (d, 1H, J=8.71 Hz), 7.43-7.55 (m, 1H), 7.28-7.37 (m, 1H), 7.30 (br d, 3H, J=11.00 Hz), 7.06-7.16 (m, 1H), 6.93-7.04 (m, 1H), 5.47-5.93 (m, 2H), 5.01-5.31 (m, 1H), 4.21-4.66 (m, 1H), 2.90-3.35 (m, 2H), 1.40-1.49 (m, 9H), 1.32-1.39 (m, 9H); MS (FIA−) m/z 564.0 (M+1).

### Step D: Preparation of (R)-2-amino-3-(3-(2-carbamoyl-6-(trifluoromethoxy)-1H-indol-1-yl)phenyl)propanoic acid TFA salt

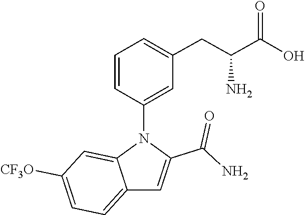

To a mixture consisting of tert-butyl (R)-2-((tert-butoxycarbonyl)amino)-3-(3-(2-carbamoyl-6-(trifluoromethoxy)-1H-indol-1-yl)phenyl)propanoate (0.14 g, 0.248 mmol) in DCM (3 mL) was added trifluoroacetic acid (3.00 mL, 39.7 mmol). The reaction mixture was stirred at room temperature overnight under N₂ atmosphere. The reaction mixture was concentrated under reduced pressure to dryness then added 10 mL of DCM and evaporated the process repeated for four times to afford crystalline material. Which was washed with diethyl ether and dried to afford the title compound as a white solid (0.080 g, 79% yield). Rf 0.2 with 1:40:60 v/v acetic acid-ethyl acetate-heptane (UV 254 nM); ¹H-NMR (400 MHz; DMSO-d6) δ 7.87-8.10 (m, 1H), 7.78 (d, 1H, J=8.48 Hz), 7.29-7.47 (m, 3H), 7.17-7.28 (m, 3H), 7.05-7.16 (m, 2H), 3.48-3.62 (m, 2H), 3.05-3.27 (m, 4H), 2.92 (br dd, 1H, J=14.10, 8.14 Hz); ¹⁹F-NMR (376 MHz; DMSO-d6) δ −59.43, −76.88; MS (FIA MS+) m/z 408 (M+1); HPLC UV purity, Rt=5.88 min, 94.2%; melting point=173-174° C.

### Example 34: Synthesis of (S)-2-amino-3-(3-(2-carbamoyl-6-(trifluoromethoxy)-1H-indol-1-yl)phenyl)propanoic acid TFA salt (Compound 24)

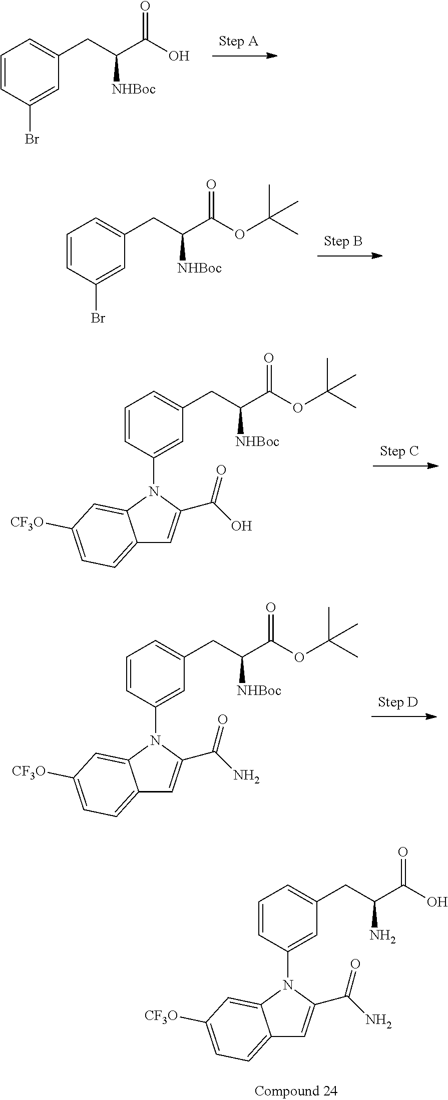

### Step A: Preparation of tert-butyl (S)-3-(3-bromophenyl)-2-((tert-butoxycarbonyl)amino)propanoate

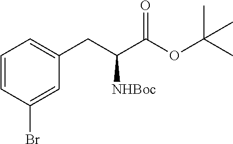 [SMILES: CC(C)(C)OC(=O)N[C@@H](Cc1cccc(Br)c1)C(=O)OC(C)(C)C]

To a 20 mL tBuOH solution of (S)-3-(3-bromophenyl)-2-((tert-butoxycarbonyl)amino)propanoic acid (2.00 g, 5.80 mmol) at room temperature was added DMAP (0.07 g, 0.58 mmol) and di-tert-butyl decarbonate (1.65 g, 7.50 mmol) under N₂ atmosphere and allowed the reaction to stir at room temperature for 14 hours. The reaction mixture was evaporated to dryness and partitioned between 100 mL ethyl acetate and 150 mL water. The organic layer was sequentially washed with water (100 mL) and brine (100 mL), then dried over anhydrous sodium sulfate. The crude reaction mixture was purified by flash silica column chromatography on a CombiFlash NextGen 300+ purification system. Elution through a 80 g RediSep Gold Rf flash silica cartridge with 0-5% methanol in DCM afforded the title compound as a clear and colorless oil (1.1 g, 47% yield); R_{f} 0.70 with 5:95 v/v methanol-dichloromethane (UV 254 nM); ¹H NMR (400 MHz, CHLOROFORM-d) δ 7.27-7.40 (m, 2H), 7.05-7.19 (m, 2H), 4.70-5.14 (m, 1H), 4.28-4.63 (m, 1H), 2.80-3.25 (m, 2H), 1.41 (d, 18H, J=8.94 Hz); MS (FIA MS+) m/z 400 (M+1).

### Step B: Preparation of (S)-1-(3-(3-(tert-butoxy)-2-((tert-butoxycarbonyl)amino)-3-oxopropyl)phenyl)-6-(trifluoromethoxy)-1H-indole-2-carboxylic acid

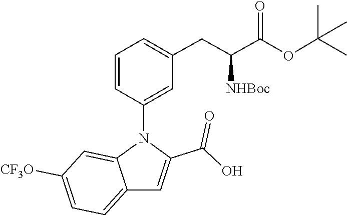

To a mixture consisting of 6-(trifluoromethoxy)-1H-indole-2-carboxylic acid (ChemShuttle, 0.24 g, 0.98 mmol), tert-butyl (S)-3-(3-bromophenyl)-2-((tert-butoxycarbonyl)amino)propanoate (0.43 g, 1.00 mmol), copper (II) acetate (CombiBlocks, 0.18 g, 0.98 mmol), methyl-α-D-glucopyranoside (CombiBlocks, 0.19 g, 0.98 mmol), potassium iodide (VWR, 0.16 g, 0.98 mmol) in DMSO (10 mL) was added DBU (Oakwood Chemicals, 0.45 mL, 2.90 mmol). The reaction mixture was heated to 115° C. under N₂ atmosphere for 14 hours and then stirred at room temperature for 1 hour. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to afford a crude mass (0.500 g). The crude product was purified by flash silica column chromatography on a CombiFlash NextGen 300+ purification system. Elution through a 24 g RediSep Gold Rf flash silica cartridge with 5-39% ethyl acetate in hexanes containing 1% acetic acid afforded the title compound as an off-white solid (0.135 g, 24% yield). R_{f} 0.32 with 1:39:60 v/v acetic acid-ethyl acetate-hexane (UV 254 nM); ¹H NMR (400 MHz, CHLOROFORM-d) δ 7.62-7.78 (m, 1H), 7.37-7.55 (m, 2H), 7.26-7.31 (m, 1H), 7.15-7.23 (m, 1H), 7.04-7.15 (m, 2H), 6.91-7.04 (m, 1H), 4.92-5.39 (m, 1H), 4.34-4.70 (m, 2H), 2.82-3.33 (m, 2H), 1.27-1.45 (m, 18H); MS (FIA−) m/z 565.0 (M+1).

### Step C: Preparation of tert-butyl (S)-2-((tert-butoxycarbonyl)amino)-3-(3-(2-carbamoyl-6-(trifluoromethoxy)-1H-indol-1-yl)phenyl)propanoate

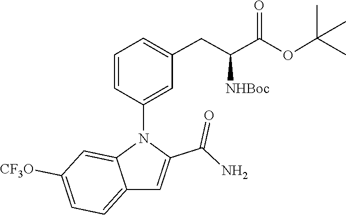

To a mixture consisting of (S)-1-(3-(3-(tert-butoxy)-2-((tert-butoxycarbonyl)amino)-3-oxopropyl)phenyl)-6-(trifluoromethoxy)-1H-indole-2-carboxylic acid (0.125 g, 0.22 mmol) in DMF (8 mL) was added ammonium chloride (Chem-Impex, 0.036 g, 0.66 mmol). Next add TBTU (Oakwood, 0.107 g, 0.33 mmol), followed by the addition of DIPEA (0.26 mL, 1.99 mmol). The reaction mixture was stirred overnight at room temperature under N₂ atmosphere. The reaction mixture was subsequently partitioned between ethyl acetate (50 mL) and H₂O (50×3 mL). The phases were separated, and the organic phase was partitioned with 1M KHSO₄ (50 mL), followed by brine (50 mL). The organic layer was concentrated under reduced pressure to afford the crude product as an orange solid. The crude was purified by flash silica column chromatography on a CombiFlash NextGen 300+ purification system. Elution through a 12 g RediSep Gold Rf flash silica cartridge with 5-39% ethyl acetate in hexanes containing 1% acetic acid afforded the title compound as an off-white solid (0.12 g, 96% yield). R_{f} 0.25 with 1:39:60 v/v acetic acid-ethyl acetate-hexane (UV 254 nM); ¹H NMR (400 MHz, CHLOROFORM-d) δ 7.69 (d, 1H, J=8.71 Hz), 7.40-7.54 (m, 1H), 7.25-7.35 (m, 4H), 7.08 (br d, 1H, J=8.71 Hz), 6.96 (br s, 1H), 5.64 (br s, 2H), 5.16 (br d, 1H, J=7.34 Hz), 4.26-4.61 (m, 1H), 3.21 (br dd, 1H, J=13.64, 5.39 Hz), 2.98 (br dd, 1H, J=13.53, 7.34 Hz), 1.40 (s, 9H), 1.33 (s, 9H); MS (FIA MS+) m/z 564 (M+1).

### Step D: Preparation of (S)-2-amino-3-(3-(2-carbamoyl-6-(trifluoromethoxy)-1H-indol-1-yl)phenyl)propanoic acid TFA salt (Compound 24)

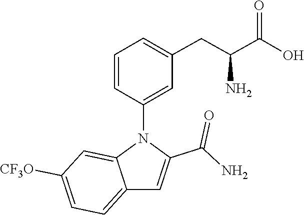

To a mixture consisting of tert-butyl (S)-2-((tert-butoxycarbonyl)amino)-3-(3-(2-carbamoyl-6-(trifluoromethoxy)-1H-indol-1-yl)phenyl)propanoate (0.11 g, 0.195 mmol) in DCM (3 mL) was added trifluoroacetic acid (3.00 mL, 39.7 mmol). The reaction mixture was stirred at room temperature overnight under N₂ atmosphere. The reaction mixture was concentrated under reduced pressure to dryness then added 10 mL of DCM and evaporated the process repeated for four times to afford crystalline material. Which was washed with diethyl ether and dried to afford the title compound as a white solid (0.050 g, 62% yield). Rf 0.2 with 1:40:60 v/v acetic acid-ethyl acetate-heptane (UV 254 nM); ¹H-NMR (400 MHz; DMSO-d6) δ 7.90-8.05 (m, 1H), 7.73-7.85 (m, 1H), 7.39-7.47 (m, 1H), 7.27-7.39 (m, 2H), 7.25 (s, 1H), 7.23 (s, 1H), 7.06-7.20 (m, 3H), 3.61-3.86 (m, 2H), 3.18 (br dd, 3H, J=14.21, 4.81 Hz), 2.97 (br dd, 2H, J=14.21, 8.02 Hz); ¹⁹F-NMR (376 MHz; DMSO-d6) δ −59.43, -76.88; MS (FIA MS+) m/z 408 (M+1); HPLC UV purity, Rt=5.88 min, 91.3%; melting point=173-174° C.

### Example 35: Synthesis of 3-(3-(2-carbamoylbenzofuran-3-yl)phenyl)propanoic acid (Compound 25)

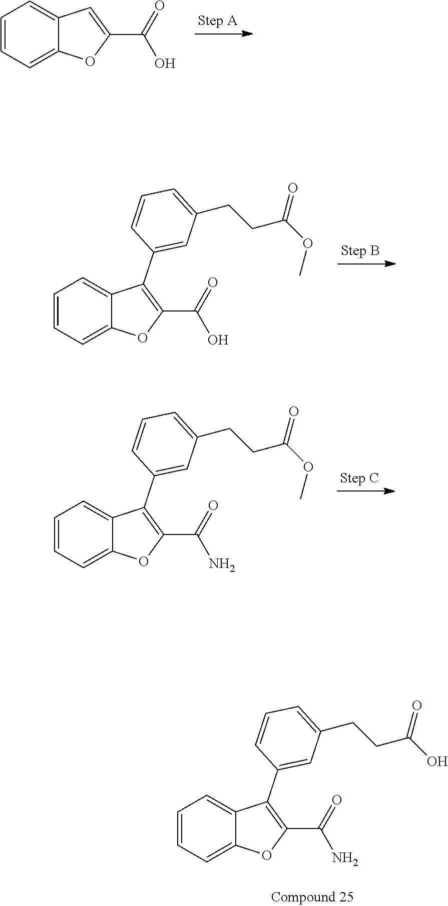 [SMILES: O=C(O)c1cc2ccccc2o1 ; COC(=O)CCc1cccc(-c2c(C(=O)O)oc3ccccc23)c1 ; COC(=O)CCc1cccc(-c2c(C(N)=O)oc3ccccc23)c1 ; NC(=O)c1oc2ccccc2c1-c1cccc(CCC(=O)O)c1]

### Step A Preparation of 3-(3-(3-methoxy-3-oxopropyl)phenyl)benzofuran-2-carboxylic acid

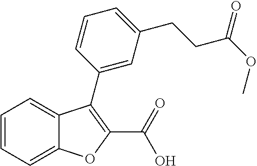 [SMILES: COC(=O)CCc1cccc(-c2c(C(=O)O)oc3ccccc23)c1]

To a 24 mL septa-cap vial was added methyl 3-bromobenzenepropanoate (Combi-Blocks, 0.547 g, 2.25 mmol) and benzofuran-2-carboxylic acid (Combi-Blocks, 0.243 g, 1.5 mmol). Next was added Rhodium diacetate (ArkPharm, 0.027 g, 4 mol %), K₂CO₃ (VWR, 0.311 g, 2.25 mmol) and DMF (6 mL). The mixture was degassed by bubbling in nitrogen gas for 3 min with stirring then heated to 140° C. overnight. The reaction mixture was subsequently cooled to room temperature and diluted with water (140 mL). The mixture was extracted with ethyl acetate (3×100 mL) and the combined organics dried with Na₂SO₄, filtered and solvent evaporated under reduced pressure to give a crude oil which was purified by flash silica column chromatography on a CombiFlash NextGen 300+ purification system. Elution through a 24 g RediSep Gold Rf flash silica cartridge with 5-55% ethyl acetate: heptane containing 1% acetic acid afforded the title compound as an amorphous solid (0.051 g, 10% yield). R_{f} 0.37 with 1:40:60 v/v acetic acid-ethyl acetate-hexane (UV 254 nM); ¹H-NMR (400 MHz; CD₃OD) δ 7.63 (d, 1H, J=8.5 Hz), 7.57 (d, 1H, J=8.0 Hz), 7.53 (dt, 1H, J=0.9, 7.8 Hz), 7.3-7.5 (m, 5H), 3.65 (s, 3H), 3.01 (t, 2H, J=7.6 Hz), 2.70 (t, 2H, J=7.6 Hz); MS (APCI⁺) m/z 325.0 (M+1), m/z 323.2 (M−1).

### Step B: Preparation of methyl 3-(3-(2-carbamoylbenzofuran-3-yl)phenyl)propanoate

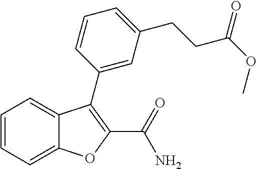 [SMILES: COC(=O)CCc1cccc(-c2c(C(N)=O)oc3ccccc23)c1]

To a mixture consisting of 3-(3-(3-methoxy-3-oxopropyl)phenyl)benzofuran-2-carboxylic acid (0.048 g, 0.15 mmol) in DMF (1.0 mL) was added ammonium chloride (Chem-Impex, 0.024 g, 0.44 mmol). Next was added TBTU (Oakwood, 0.071 g, 0.22 mmol) followed by the addition of DIPEA (0.23 mL, 1.3 mmol). The reaction mixture was stirred overnight at room temperature under N₂ atmosphere. The reaction mixture was subsequently evaporated to a residue which was diluted with water (40 mL) and extracted with 3×20 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 24 g RediSep Gold Rf flash silica cartridge with 10-100% ethyl acetate in heptane containing 1% acetic acid afforded the title compound as a white semi-solid (0.032 g, 68% yield). R_{f} 0.25 with 1:30:70 v/v acetic acid-ethyl acetate-hexane (UV 254 nM); ¹H-NMR (400 MHz; CD₃OD) δ 7.60 (dd, 2H, J=8.1, 18.5 Hz), 7.4-7.5 (m, 4H), 7.3-7.4 (m, 2H), 3.65 (s, 3H), 3.00 (t, 2H, J=7.6 Hz), 2.70 (t, 2H, J=7.6 Hz); MS (APCI⁺) m/z 324.1 (M+1).

### Step C: Preparation of 3-(3-(2-carbamoylbenzofuran-3-yl)phenyl)propanoic acid (Compound 25)

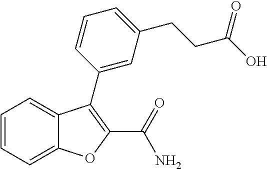 [SMILES: NC(=O)c1oc2ccccc2c1-c1cccc(CCC(=O)O)c1]

To a mixture consisting of methyl 3-(3-(2-carbamoylbenzofuran-3-yl)phenyl)propanoate (0.032 g, 0.10 mmol) in MeOH (2 mL) was added dropwise, a 1M aqueous solution of LiOH (0.3 mL, 3 eq). The reaction mixture was stirred overnight at room temperature. The clear solution was subsequently diluted with water (10 mL) and subjected to reduced pressure on a rotary evaporator to remove MeOH. Additional water was added (10 mL) and the solution was acidified with 1N HCl to precipitate product. The suspension was filtered, and solids washed with excess water, then dried under high vac overnight to afford the title compound as a white solid (0.020 g, 65% yield). R_{f} 0.13 with 1:40:60 v/v acetic acid-ethyl acetate-heptane (UV 254 nM); ¹H-NMR (400 MHz; CD₃OD) δ 7.61 (dd, 2H, J=8.1, 11.3 Hz), 7.5-7.5 (m, 2H), 7.4-7.5 (m, 2H), 7.3-7.4 (m, 2H), 3.00 (t, 2H, J=7.6 Hz), 2.67 (t, 2H, J=7.7 Hz); MS (APCI⁺) m/z 310.1 (M+1), 308.1 (M−1). HPLC UV purity, Rt=6.36 min, 98.3%. Melting point 186.0-187.5° C. (dec.).

### Example 36: Synthesis of tert-butyl ((6-(2-carbamoyl-6-(trifluoromethoxy)-1H-indol-1-yl)pyridin-2-yl)methyl)carbamate (Compound 26) and 1-(6-(aminomethyl)pyridin-2-yl)-6-(trifluoromethoxy)-1H-indole-2-carboxamide TFA salt (Compound 27)

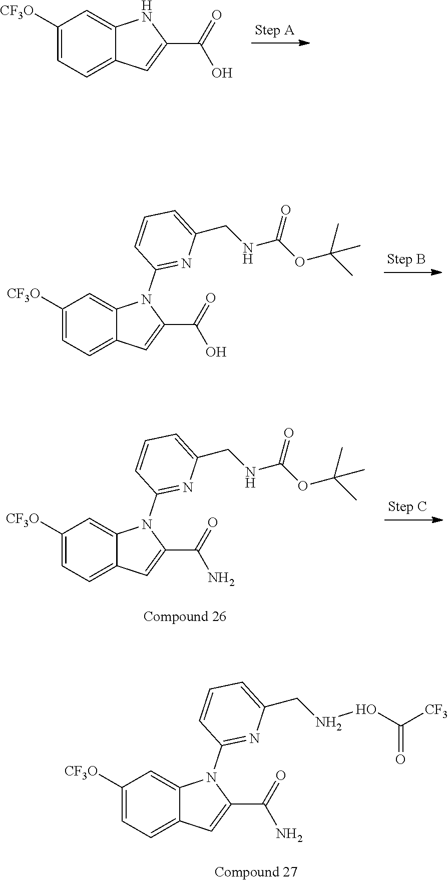

### Step A: Preparation of 1-(6-(((tert-butoxycarbonyl)amino)methyl)pyridin-2-yl)-6-(trifluoromethoxy)-1H-indole-2-carboxylic acid

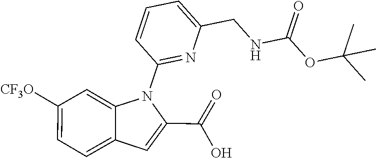

To a mixture consisting of 6-(trifluoromethoxy)-1H-indole-2-carboxylic acid (ChemShuttle, 0.368 g, 1.50 mmol), tert-butyl ((6-bromopyridin-2-yl)methyl) carbamate (ChemScene, 0.386 g, 1.50 mmol), copper (II) acetate (CombiBlocks, 0.272 g, 1.50 mmol), methyl-α-D-glucopyranoside (CombiBlocks, 0.291 g, 1.50 mmol), potassium iodide (VWR, 0.498 g, 3.00 mmol) in DMSO (20 mL) was added DBU (Oakwood Chemicals, 0.67 mL, 4.50 mmol). The reaction mixture was heated to 115° C. under N₂ atmosphere for 4 hours and then allowed to cool to room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 1.02 g. The crude oil was purified by flash silica column chromatography on a CombiFlash NextGen 300+ purification system. Elution through a 24 g RediSep Gold Rf flash silica cartridge with 5-75% ethyl acetate in hexanes containing 1% acetic acid afforded the title compound as a tan solid (0.087 g, 12% yield); R_{f} 0.14 with 1:30:70 v/v acetic acid-ethyl acetate-hexane (UV 254 nM); MS (APCI+) m/z 452.0 (M+1), (APCI−) m/z 450.0 (M−1); HPLC UV purity, Rt=9.02 min, 83.8%. The product was used in the next step without further characterization.

### Step B: Preparation of tert-butyl ((6-(2-carbamoyl-6-(trifluoromethoxy)-1H-indol-1-yl)pyridin-2-yl)methyl)carbamate (Compound 26)

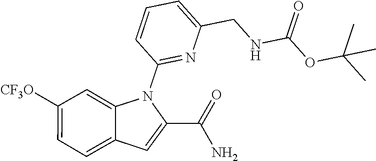

To a mixture consisting of 1-(6-(((tert-butoxycarbonyl)amino)methyl)pyridin-2-yl)-6-(trifluoromethoxy)-1H-indole-2-carboxylic acid (0.083 g, 0.183 mmol) in DMF (2 mL) was added ammonium chloride (Chem-Impex, 0.029 g, 0.55 mmol). Next add TBTU (Oakwood, 0.088 g, 0.275 mmol), followed by the addition of DIPEA (0.29 mL, 1.65 mmol). The reaction mixture was stirred for 5 hours at room temperature under N₂ atmosphere. The reaction mixture was subsequently partitioned between ethyl acetate (25 mL) and H₂O (20 mL). The phases were separated, and the organic phase was partitioned with 1M KHSO₄ (15 mL) followed by brine (15 mL). The organic layer was concentrated under reduced pressure to afford the crude product as a yellow oil (0.081 g). The crude oil was purified by flash silica column chromatography on a CombiFlash NextGen 300+ purification system. Elution through a 12 g RediSep Gold Rf flash silica cartridge with 10-70% ethyl acetate in heptane containing 1% acetic acid afforded the title compound as a white solid (0.058 g, 71% yield). Rf 0.07 with 1:30:70 v/v acetic acid-ethyl acetate-heptane (UV 254 nM); ¹H-NMR (400 MHz; DMSO-d6) δ 8.16 (br s, 1H), 8.07 (br s, 1H), 7.94 (t, 1H, J=7.8 Hz), 7.84 (d, 1H, J=8.7 Hz), 7.45-7.51 (m, 2H), 7.44 (s, 1H), 7.25-7.35 (m, 2H), 7.17-7.21 (m, 2H), 4.26 (d, 2H, J=6.0 Hz), 1.40 (s, 9H); ¹⁹F-NMR (376 MHz; DMSO-d6) δ −56.62; MS (APCI+) m/z 451.0 (M+1), (APCI−) m/z 449.0 (M−1); HPLC UV purity, Rt=8.271 min, 99.0%.

### Step C: Preparation of 1-(6-(aminomethyl)pyridin-2-yl)-6-(trifluoromethoxy)-1H-indole-2-carboxamide TFA salt (Compound 27)

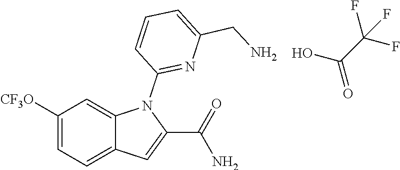

To a mixture consisting of tert-butyl((6-(2-carbamoyl-6-(trifluoromethoxy)-1H-indol-1-yl)pyridin-2-yl)methyl)carbamate (0.058 g, 0.129 mmol) in DCM (2 mL) was added trifluoroacetic acid (Chem-Impex, 0.5 mL, 6.74 mmol). The reaction mixture was stirred for 1 hour at room temperature under N₂ atmosphere. The reaction mixture was subsequently concentrated under reduced pressure with co-evaporation with Toluene (25 mL×4) to afford the crude product as a white solid (0.052 g, 88% yield). R_{f} 0.05 with 1:60:40 v/v acetic acid-ethyl acetate-heptane (UV 254 nM); ¹H-NMR (400 MHz; DMSO-d6) δ 8.32 (br s, 3H), 8.20 (br s, 1H), 8.04 (t, 1H, J=7.8 Hz), 7.86 (d, 1H, J=8.7 Hz), 7.55 (d, 1H, J=7.8 Hz), 7.45-7.50 (m, 2H), 7.35 (s, 1H), 7.32 (d, 1H, J=7.8 Hz), 7.21 (d, 1H, J=8.7 Hz), 4.31 (d, 2H, J=5.3 Hz); ¹⁹F-NMR (376 MHz; DMSO-d6) δ −56.58, −73.45; MS (APCI+) m/z 351.0 (M+1); HPLC UV purity, Rt=5.437 min, 99.5%; melting point=187-188° C.

### Example 37: Synthesis of trans-(rac)-3-(3-(2-(methylcarbamoyl)-6-(trifluoromethoxy)-1H-indol-1-yl)phenyl)propanoic acid (Compound 28)

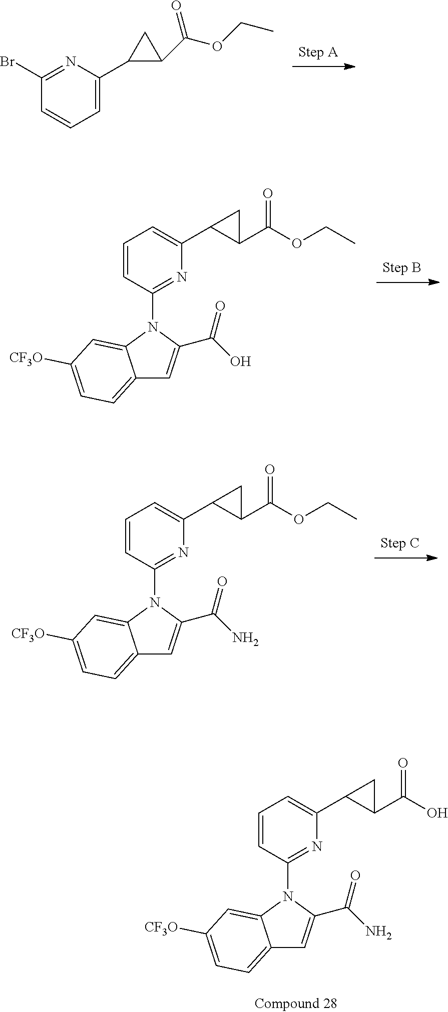

### Step A: Preparation of (rac)-1-(3-(trans-2-(methoxycarbonyl)cyclopropyl)phenyl)-6-(trifluoromethoxy)-1H-indole-2-carboxylic acid

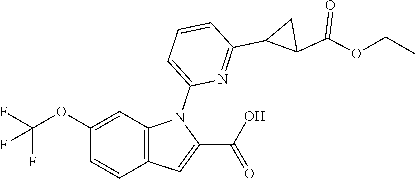 [SMILES: CCOC(=O)C1CC1c1cccc(-n2c(C(=O)O)cc3ccc(OC(F)(F)F)cc32)n1]

To a mixture consisting of 6-(trifluoromethoxy)-1H-indole-2-carboxylic acid (ChemShuttle, 0.726 g, 2.96 mmol), ethyl trans-2-(6-bromopyridin-2-yl)cyclopropane-1-carboxylate (prepared according to WO2020022470 A1, 0.800 g, 2.96 mmol), copper (II) acetate (CombiBlocks, 0.536 g, 2.96 mmol), methyl-α-D-glucopyranoside (CombiBlocks, 0.575 g, 2.96 mmol), potassium iodide (VWR, 0.983 g, 5.92 mmol) in DMSO (40 mL) was added DBU (Oakwood Chemicals, 1.32 mL, 8.88 mmol). The reaction mixture was heated to 105° C. under N₂ atmosphere for 4 hours and then allowed to cool to room temperature. The reaction mixture was subsequently partitioned between ethyl acetate (125 mL) and 1M KHSO₄ (60 mL). An emulsion formed which was broken up by the addition of 25 mL of brine. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 1.82 g. The crude oil was purified by flash silica column chromatography on a CombiFlash NextGen 300+ purification system. Elution through a 40 g RediSep Gold Rf flash silica cartridge with 5-40% ethyl acetate in hexanes containing 1% acetic acid afforded the title compound as a yellow solid (0.272 g, 21% yield). R_{f} 0.20 with 1:30:70 v/v acetic acid-ethyl acetate-hexane (UV 254 nM); ¹H-NMR (400 MHz; DMSO-d6) δ 13.16 (br s, 1H), 7.9-8.0 (m, 2H), 7.58 (d, 1H J=7.3 Hz), 7.44 (s, 1H), 7.39 (d, 1H, J=7.8 Hz), 7.28 (s, 1H), 7.17-7.23 (m, 1H), 4.09 (q, 1H, J=7.1 Hz), 2.7-2.8 (m, 1H), 2.0-2.1 (m, 1H), 1.4-1.5 (m, 2H), 1.2-1.3 (m, 3H); ¹⁹F-NMR (376 MHz; DMSO-d6) δ −56.74; MS (APCI+) m/z 435.0 (M+1), (APCI−) m/z 433.0 (M−1); HPLC UV purity Rt=9.719 min, 85.3%.

### Step B: Preparation of (rac)-ethyl trans-2-(6-(2-carbamoyl-6-(trifluoromethoxy)-1H-indol-1-yl)pyridin-2-yl)cyclopropane-1-carboxylate

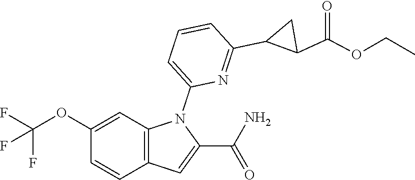 [SMILES: CCOC(=O)C1CC1c1cccc(-n2c(C(N)=O)cc3ccc(OC(F)(F)F)cc32)n1]

To a mixture consisting of 1-(3-(trans-2-(methoxycarbonyl)cyclopropyl)phenyl)-6-(trifluoromethoxy)-1H-indole-2-carboxylic acid (0.261 g, 0.601 mmol) in DMF (5 mL) was added ammonium chloride (Chem-Impex, 0.096 g, 1.80 mmol). Next add TBTU (Oakwood, 0.289 g, 0.901 mmol), followed by the addition of DIPEA (1.0 mL, 5.41 mmol). The reaction mixture was stirred overnight at room temperature under N₂ atmosphere. The reaction mixture was subsequently partitioned between ethyl acetate (50 mL) and H₂O (50 mL). The phases were separated, and the organic phase was partitioned with 1M KHSO₄ (25 mL), followed by brine (25 mL). The organic layer was concentrated under reduced pressure to afford the crude product as a colorless oil which solidified to a white solid (0.272 g). The crude solid was triturated with ethyl acetate and heptane (5 mL, 1:1 mixture) and filtered over a fritted funnel to provide the titled compound as a white solid (0.170 g, 65% yield). R_{f} 0.13 with 1:30:70 v/v acetic acid-ethyl acetate-hexane (UV 254 nM); ¹H-NMR (400 MHz; DMSO-d6) δ 8.15 (br s, 1H), 7.90 (t, 1H, J=7.7 Hz), 7.84 (d, 1H, J=8.7 Hz), 7.5-7.6 (m, 2H), 7.38 (s, 1H), 7.27 (d, 1H J=7.8 Hz), 7.23 (s, 1H), 7.19 (dd, 1H, J=1.1, 8.7 Hz), 4.06-4.13 (m, 2H), 2.7-2.8 (m, 1H), 2.09-2.11 (m, 1H), 1.50 (t, 2H, J=7.2 Hz), 1.19 (t, 3H, J=7.1 Hz); ¹⁹F-NMR (376 MHz; DMSO-d6) δ −56.76; MS (APCI+) m/z 434.0 (M+1), (APCI−) m/z 432.0 (M−1); HPLC UV purity Rt=8.81 min, 92.1%.

### Step C: Preparation of trans-(rac)-2-(6-(2-carbamoyl-6-(trifluoromethoxy)-1H-indol-1-yl)pyridin-2-yl)cyclopropane-1-carboxylic acid (Compound 28)

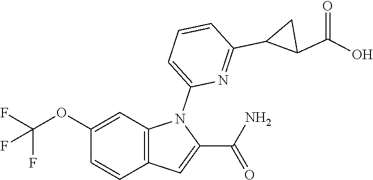 [SMILES: NC(=O)c1cc2ccc(OC(F)(F)F)cc2n1-c1cccc(C2CC2C(=O)O)n1]

To a mixture consisting of ethyl trans-2-(6-(2-carbamoyl-6-(trifluoromethoxy)-1H-indol-1-yl)pyridin-2-yl)cyclopropane-1-carboxylate (0.055 g, 0.127 mmol) in methanol (2 mL) was added 1M lithium hydroxide (0.51 mL, 0.51 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with water (10 mL), followed by brine (10 mL). The organic layer was concentrated under reduced pressure to afford the title product as a white solid (0.045 g, 89% yield). R_{f} 0.28 with 1:60:40 v/v acetic acid-ethyl acetate-hexane (UV 254 nM); ¹H-NMR (400 MHz; DMSO-d6) δ 12.38 (s, 1H), 8.14 (br s, 1H), 7.89 (t, 1H, J=7.8 Hz), 7.84 (d, 1H, J=8.7 Hz), 7.52 (br d, 2H, J=7.3 Hz), 7.38 (s, 1H), 7.2-7.3 (m, 1H), 7.18-7.22 (m, 2H), 2.6-2.8 (m, 1H), 1.98-2.00 (m, 1H), 1.46 (t, 2H, J=7.1 Hz); ¹⁹F-NMR (376 MHz; DMSO-d6) δ −56.73; MS (APCI⁺) m/z 406.0 (M+1), (APCI−) m/z 404.0 (M−1); HPLC UV purity, Rt=7.18 min, 96.7%; Chiral HPLC purity, Rt=10.4 min, 45.0%, Rt=15.1 min, 49.8%; melting point=241-242° C.

### Example 38: Synthesis of trans-(−)- and trans-(+)-2-(6-(2-carbamoyl-6-(trifluoromethoxy)-1H-indol-1-yl)pyridin-2-yl)cyclopropane-1-carboxylic acid (Compound 28a and Compound 28b)

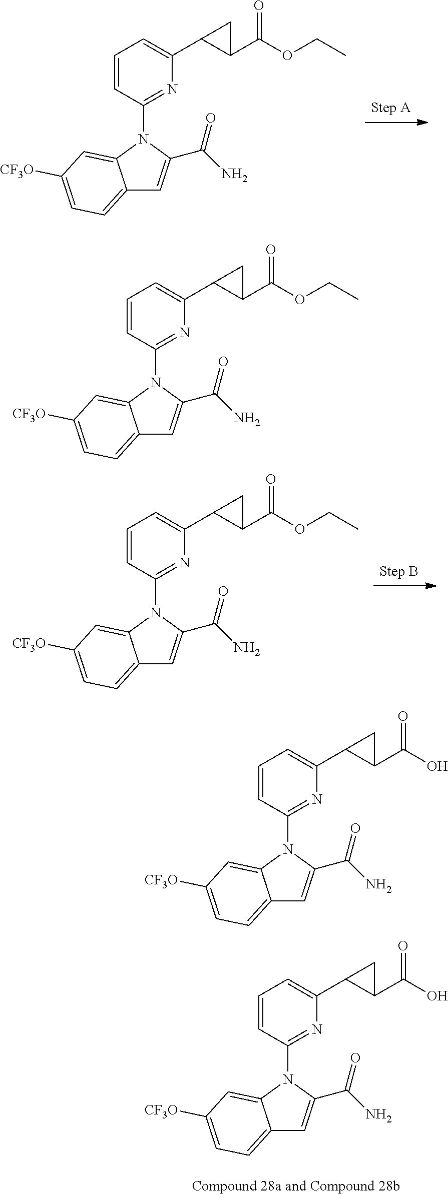

### Step A: Chiral separation of ethyl trans-2-(6-(2-carbamoyl-6-(trifluoromethoxy)-1H-indol-1-yl)pyridin-2-yl)cyclopropane-1-carboxylate

The diastereomers of ethyl trans-2-(6-(2-carbamoyl-6-(trifluoromethoxy)-1H-indol-1-yl)pyridin-2-yl)cyclopropane-1-carboxylate (0.173 g) was separated by Chiral HPLC on a (R, R) Whelk-01 column (250 mm×4.6 mm) using 75:25:0.1 Heptane:IPA:TEA at a flow rate of 1 mL/min with UV detection at 290 nm. Peak 1 eluted at 16.2 minutes and Peak 2 eluted at 23.1 minutes.

Diastereomer 1 (peak 1) was concentrated under reduced pressure to afford a white solid (0.60 mg, 34% recovery); R_{f} 0.40 with 1:60:40 v/v acetic acid-ethyl acetate-hexane (UV 254 nM); MS (APCI+) m/z 434.0 (M+1), (APCI−) m/z 432.0 (M−1); HPLC UV purity Rt=8.794 min, 99.8%; Chiral HPLC purity Rt=14.11 min, 100%.

Diastereomer 2 (peak 2) was concentrated under reduced pressure to afford a white solid (44.6 mg, 26% recovery); R_{f} 0.40 with 1:60:40 v/v acetic acid-ethyl acetate-hexane (UV 254 nM); MS (APCI+) m/z 434.0 (M+1), (APCI−) m/z 432.0 (M−1); HPLC UV purity Rt=8.796 min, 99.4%; Chiral HPLC purity Rt=20.87 min, 99.3%.

### Step B: Preparation of trans-(−)- and trans-(+)-2-(3-(2-carbamoyl-6-(trifluoromethoxy)-1H-indol-1-yl)phenyl)cyclopropane-1-carboxylic acid (Compound 28a and Compound 28b)

To the individually separated diastereomers of methyl trans-2-(3-(2-carbamoyl-6-(trifluoromethoxy)-1H-indol-1-yl)phenyl)cyclopropane-1-carboxylate (Peak 1, 0. 051 g, 0.119 mmol), (Peak 2, 0.043 g, 0.100 mmol) in methanol (5.0 mL) was added 1M lithium hydroxide (0.60 mL, 0.60 mmol). The reaction mixture was stirred at room temperature overnight under N₂ atmosphere. The reaction mixture was subsequently partitioned between ethyl acetate (15 mL) and 1M HCl (10 mL). The phases were separated, and the organic phase was partitioned with H₂O (10 mL), followed by brine (10 mL). The organic layer was concentrated under reduced pressure to afford the crude products as a white solid.

Diastereomer 1 (peak 1, 28a) was concentrated under reduced pressure to afford a white solid (0.014 g, 30% yield). R_{f} 0.22 with 1:60:40 v/v acetic acid-ethyl acetate-hexane (UV 254 nM); ¹H-NMR (400 MHz; DMSO-d6) δ 12.36 (s, 1H), 8.14 (br s, 1H), 7.89 (t, 1H, J=7.8 Hz), 7.84 (d, 1H, J=8.5 Hz), 7.52 (d, 2H, J=7.3 Hz), 7.52 (br s, 2H), 7.38 (s, 1H), 7.26 (d, 1H, J=7.8 Hz), 7.22 (s, 1H), 7.19 (dd, 1H, J=0.9, 8.7 Hz), 7.8 Hz), 2.6-2.7 (m, 1H), 1.9-2.0 (m, 1H), 1.45 (t, 2H, J=7.2 Hz); ¹⁹F-NMR (376 MHz; DMSO-d6) δ −56.74; MS (APCI+) m/z 406.0 (M+1), MS (APCI−) m/z 404.0 (M−1); HPLC UV purity Rt=7.17 min, 98.0%; Chiral HPLC purity Rt=10.39 min, 97.1%; optical rotation [α]²⁵_{D}=−207.6 (c=0.5, 100 mL MeOH); melting point=161.5-162.0° C.

Diastereomer 2 (peak 2, 28b) was concentrated under reduced pressure to afford a white solid (0.014 g, 35% yield). R_{f} 0.21 with 1:60:40 v/v acetic acid-ethyl acetate-hexane (UV 254 nM); ¹H-NMR (400 MHz; DMSO-d6) δ 12.36 (s, 1H), 8.14 (br s, 1H), 7.89 (t, 1H, J=7.8 Hz), 7.84 (d, 1H, J=8.5 Hz), 7.52 (d, 2H, J=7.3 Hz), 7.52 (br s, 2H), 7.38 (s, 1H), 7.26 (dd, 1H, J=0.7, 8.0 Hz), 7.22 (d, 1H, J=0.7 Hz), 7.19 (td, 1H, J=1.0, 9.7 Hz), 2.6-2.7 (m, 1H), 1.9-2.0 (m, 1H), 1.45 (t, 2H, J=7.2 Hz); ¹⁹F-NMR (376 MHz; DMSO-d6) δ −56.74; MS (APCI+) m/z 406.0 (M+1), MS (APCI−) m/z 404.0 (M−1); HPLC UV purity Rt=7.17 min, 99.8%; Chiral HPLC purity Rt=15.24 min, 99.1%; optical rotation [α]²⁵_{D}=+217.6 (c=0.5, 100 mL MeOH); melting point=192-193° C.

### Example 39: Alternate synthesis of trans (−)-2-(6-(2-carbamoyl-6-(trifluoromethoxy)-1H-indol-1-yl)pyridin-2-yl)cyclopropane-1-carboxylic acid (Compound 28a)

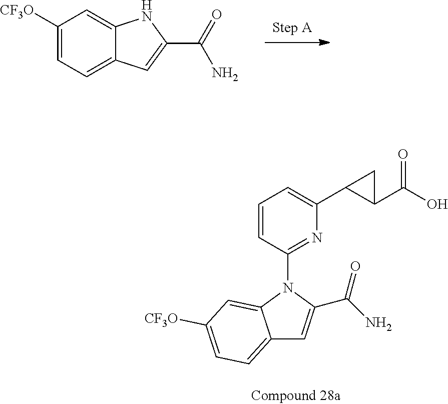

### Step A: Preparation of trans-(−)-2-(6-(2-carbamoyl-6-(trifluoromethoxy)-1H-indol-1-yl)pyridin-2-yl)cyclopropane-1-carboxylic acid (Compound 28a)

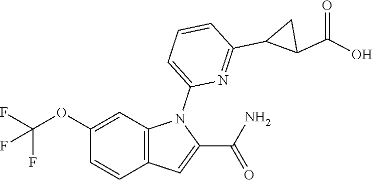 [SMILES: NC(=O)c1cc2ccc(OC(F)(F)F)cc2n1-c1cccc(C2CC2C(=O)O)n1]

To a mixture consisting of 6-(trifluoromethoxy)-1H-indole-2-carboxamide (0.244 g, 1.00 mmol), trans-(−)-2-(6-bromopyridin-2-yl)cyclopropane-1-carboxylic acid (0.266 g, 1.1 mmol), copper (I) iodide (Strem, 0.190 g, 1.00 mmol) in DMSO (6 mL) was added DBU (Oakwood Chemicals, 0.3 mL, 2.00 mmol). The reaction mixture was heated to 110° C. under N₂ atmosphere for 2 hours and then allowed to cool to room temperature. The reaction mixture was subsequently partitioned between ethyl acetate (50 mL) and 1M KHSO₄ (30 mL). A precipitate formed which was collected by filtration over a fritted funnel and set aside. The filtrate was poured in a separatory funnel. The phases were separated, and the organic phase was partitioned with H₂O (75 mL), followed by brine (75 mL). The organic layer was separated and dried over anhydrous magnesium sulfate, filtered, and concentrated under reduced pressure to give a crude brown oil, 0.465 g. The crude oil was purified by flash silica column chromatography on a CombiFlash NextGen 300+ purification system. Elution through a 12 g RediSep Gold Rf flash silica cartridge with 10-70% ethyl acetate in hexanes containing 1% acetic acid afforded the title compound as an off-white solid (0.091 g, 22% yield). R_{f} 0.21 with 1:60:40 v/v acetic acid-ethyl acetate-hexane (UV 254 nM); ¹H-NMR (400 MHz; DMSO-d6) δ 12.40 (s, 1H), 8.14 (br s, 1H), 7.89 (t, 1H, J=7.7 Hz), 7.84 (d, 1H, J=8.7 Hz), 7.52 (br d, 2H, J=7.6 Hz), 7.38 (s, 1H), 7.2-7.3 (m, 1H), 7.18-7.22 (m, 2H), 2.6-2.8 (m, 1H), 1.98-2.00 (m, 1H), 1.46 (t, 2H, J=7.1 Hz); ¹⁹F-NMR (376 MHz; DMSO-d6) δ −56.73; MS (APCI⁺) m/z 406.0 (M+1), (APCI−) m/z 404.0 (M−1); HPLC UV purity, Rt=7.13 min, 99.5%; Chiral HPLC purity Rt=15.26 min, 91.5%; optical rotation [α]²⁵_{D}=−260.0 (c=0.25, 2.24 mL IPA); melting point=161.5-162.0° C.

### Example 40: Synthesis of (1S,2S)-2-(6-bromopyridin-2-yl)cyclopropane-1-carboxylic acid (Compound 28a)

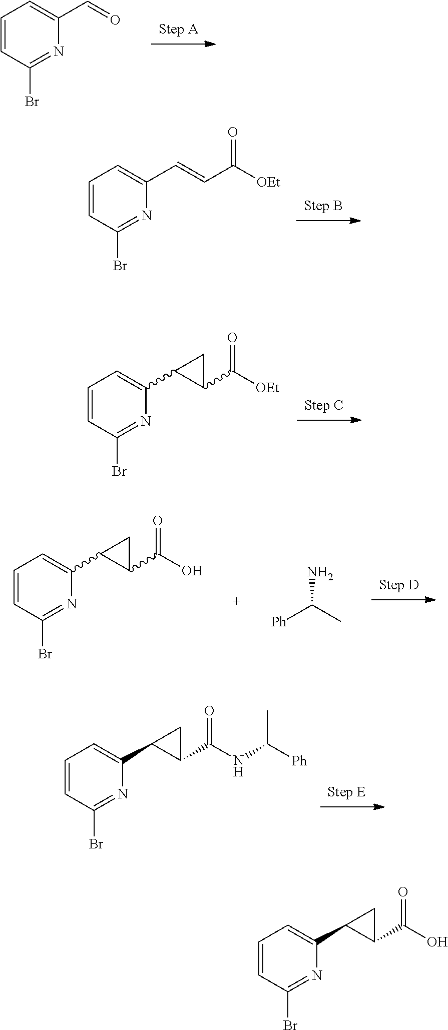

### Step A: Preparation of ethyl (E)-3-(6-bromopyridin-2-yl)acrylate

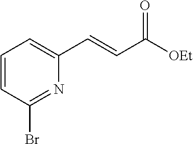 [SMILES: CCOC(=O)/C=C/c1cccc(Br)n1]

A cleaned and nitrogen-purged round bottom flask fitted with a mechanical stirrer, a temperature probe, nitrogen inlet/outlet, addition funnel and connected to a circulator was charged with LiCl (6.24 g, 14.7 mmoles, 1.1 equiv.) and THE (300 mL) and stirred until most of the solid dissolved. 6-Bromopyridine-2-carboaldehyde (25.0 g, 13.4 mmoles 1.0 equiv) was added to the mixture followed by triethyl phosphonium acetate (33.17 g, 14.8 mmoles, 1.1 equiv.). The homogenous mixture was cooled to −20° C. Triethylamine (14.98 g, 14.8 mmole, 1.1 equiv) was added dropwise via addition funnel at a rate where the reaction internal temperature was below −10° C. After complete addition, the reaction mixture was warmed up to 0° C. and stirred under nitrogen overnight. After 18 h, the reaction was quenched with 4% aqueous citric acid (0.4 L) to pH 3-4 then warmed up to 20° C. Ethyl acetate (0.2 L) was added, and the mixture stirred for 10 minutes before separating the layers. The upper organic phase was collected and the lower aqueous phase was re-extracted with ethyl acetate (0.2 L). The combined organic fractions were washed with 14% aqueous sodium chloride solution (0.3 L). The organic solution was concentrated under reduced pressure to give a tan solid (41.5 g). The tan solid was suspended in EtOAc (40 mL). The suspension was heated to 35° C. to dissolve. The heat was turned off and heptane (200 mL) was added dropwise. A white solid precipitated and the mixture was cooled to below 5° C. using an ice/water bath then stirred overnight. The solid was collected by filtration, washed with heptane (2×25 mL). The solid was dried under vacuum at 40° C. to give the product as a white solid (18.07 g, 54%). Purity by HPLC >99%. ¹H NMR (400 MHz, CDCl₃) δ 7.61-7.53 (m, 2H), 7.44 (d, J=7.9 Hz, 1H), 7.35 (d, J=7.5 Hz, 1H), 6.95 (d, J=15.6 Hz, 1H), 4.26 (q, J=7.1 Hz, 2H), 1.33 (t, J=7.1 Hz, 3H).

### Step B: Preparation of trans-ethyl 2-(6-bromopyridin-2-yl)cyclopropane-1-carboxylate

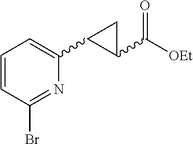 [SMILES: CCOC(=O)C1CC1c1cccc(Br)n1]

A cleaned and nitrogen-purged 250-mL three-neck round bottom flask was equipped with a magnetic stir bar, a temperature probe, and nitrogen inlet/outlet, was charged with Trimethylsulfoxonium iodide (5.1 g, 23.4 mmol, 1.2 equiv.) followed by KOtBu (2.4 g, 21.5 mmol, 1.1 equiv.) and DMSO (50 mL) under nitrogen. The mixture was stirred at room temperature for 1 h. to give a clear solution. A solution of ethyl (E)-3-(6-bromopyridin-2-yl)acrylate (5.0 g, 19.5 mmol, 1.0 equiv.) in DMSO (50 mL) was added quickly while maintaining the internal temperature below 30° C. The reaction turned red and was allowed to stir at room temperature overnight. After the reaction was deemed complete by TLC, the mixture was quenched with 1N HCl (5 mL) to pH 4-5. The mixture was diluted with water (200 mL). The mixture was transferred to a separatory funnel using ethyl acetate (100 mL) for washing and transferring. The upper organic layer was collected, and then the aqueous layer was re-extracted with ethyl acetate (3×100 mL). The combined organic layers were dried with sodium sulfate, filtered, and the filtrate was concentrated under reduced pressure to give a yellow oil. The oil was purified on a prepacked 40-g RediSep silica column on a Combiflash automatic system using a gradient eluent (heptane/EtOAc (90:10) for 5 min then heptane/EtOAc (80:20) for 5 min). The product containing fractions (R_{f}=0.33) were combined and concentrated under reduced pressure to give the product as a colorless oil (3.06 g, 11.4 mmol, 58%). ¹H NMR (400 MHz, CDCl₃) δ 7.39 (t, J=7.7 Hz, 1H), 7.25 9d, J=7.8 Hz, 1H), 7.16 (d, J=7.5 Hz, 1H), 4.14 (q, J=7.2 Hz, 2H), 2.54-2.49 (m, 1H), 2.30-2.21 (m, 1H), 1.62-1.55 (m, 2H), 1.26 (t, J=7.2 Hz, 3H).

### Step C: Preparation of trans-2-(6-bromopyridin-2-yl)cyclopropane-1-carboxylic acid

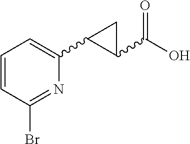 [SMILES: O=C(O)C1CC1c1cccc(Br)n1]

A cleaned and nitrogen-purged 200-mL three-neck round bottom flask equipped with a temperature probe, heating mantle, and magnetic stir bar was charged with trans-ethyl 2-(6-bromopyridin-2-yl)cyclopropane-1-carboxylate (17.12 g, 63.3 mmol, 1.0 equiv.) and DMSO (20 mL). An aqueous solution of NaOH (1.ON, 95 mL, 95 mmol, 1.5 equiv.) was then added and the mixture was stirred at 40° C. for 2 h. After cooling to 23° C., the mixture was acidified to pH 2 with 1N HCl. A white solid precipitated and was collected by filtration. The filter cake was washed with water (100 mL) and dried under vacuum to give the product as a white solid (14.48 g, 59.8 mmol, 95%). The product was used in the next step without further purification. ¹H NMR (400 MHz, DMSO-d₆) δ 12.41 (br s, 1H), 7.61 (t, J=7.7 Hz, 1H), 7.46 (d, J=7.6 Hz, 1H), 7.41 (d, J=7.8 Hz, 1H), 2.58-2.50 (m, 1H), 1.96-1.92 (m, 1H), 1.45-1.34 (m, 2H). ¹³C{¹H} NMR (101 MHz, DMSO-d₆) δ 173.3, 160.7, 141.1, 139.7, 125.7, 121.8, 25.7, 24.3, 17.0.

### Step D: Preparation of (1S,2S)-2-(6-bromopyridin-2-yl)-N-((R)-1-phenylethyl)cyclopropane-1-carboxamide

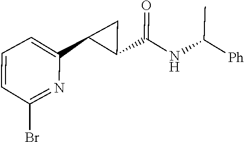 [SMILES: C[C@@H](NC(=O)[C@H]1C[C@@H]1c1cccc(Br)n1)c1ccccc1]

A cleaned and nitrogen-purged 200-mL three-neck round bottom flask equipped with a magnetic stir bar was charged with trans-2-(6-bromopyridin-2-yl)cyclopropane-1-carboxylic acid (12.7 g, 52.4 mmol, 1.0 equiv.), (R)-1-phenylethylamine (8.48 mL, 65.8 mmol, 1.2 equiv.), DMAP (0.803 g, 6.58 mmol, 12 mol %), and dichloromethane (100 mL). The mixture was stirred at room temperature until all components were dissolved to give a clear solution. EDC HCl (12.6 g, 65.8 mmol, 1.2 equiv.) was then added and the mixture was stirred at room temperature overnight. The reaction mixture was concentrated under reduced pressure and the residue was filtered through a plug of silica gel (30 g). The silica plug was washed with (70:30) heptane/EtOAc (500 mL). The filtrate was heated to reflux to give a clear solution and was then cooled to room temperature overnight. The solids that precipitated were collected by filtration and washed with (80:20) heptane/EtOAc (100 mL). The filter cake was dried under vacuum to give the product as a white solid (4.70 g, 13.6 mmol, 26%). ¹H NMR (400 MHz, CDCl₃) δ 7.41-7.29 (m. 6H), 7.23 (d, J=7.5 Hz, 1H), 7.18 (d, J=7.5 Hz, 1H), 5.93 (br d, J=7.5 Hz, 1H), 5.14 (p, J=7.3 Hz, 1H), 2.55-2.46 (m, 1H), 2.09-2.05 (m, 1H), 1.63-1.59 (m, 1H), 1.52-1.44 (d overlaps with m, 4H). ¹³C{¹H}(101 MHz, CDCl₃) δ 170.7, 161.6, 143.3, 142.0, 138.3, 128.8, 127.5, 126.3, 125.3, 121.7, 49.3, 26.8, 25.6, 22.1, 17.6.

### Step E: Preparation of (1S,2S)-2-(6-bromopyridin-2-yl)cyclopropane-1-carboxylic acid

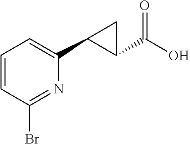 [SMILES: O=C(O)[C@H]1C[C@@H]1c1cccc(Br)n1]

A cleaned and nitrogen-purge 100-mL round bottom flask equipped with a temperature probe, heating mantle, and magnetic stir bar was charged with (1S,2S)-2-(6-bromopyridin-2-yl)-N-((R)-1-phenylethyl)cyclopropane-1-carboxamide (4.70 g, 13.6 mmol) and conc. HCl (15 mL). The mixture was heated to 80° C. and stirred for 5 h. The mixture was cooled to 0° C. in an ice/water bath and diluted with water (100 mL). The mixture was transferred to a separatory funnel with the aid of EtOAc (50 mL). The upper organic layer was collected, and the aqueous layer was re-extracted with EtOAc (4×20 mL). The combined organic layers were dried with sodium sulfate and concentrated under vacuum to give the product (3.7 g, 13.6 mmol, >99%) as a white solid. ¹H NMR (400 MHz, DMSO-d₆) δ 12.42 (br s, 1H), 7.64 (t, J=7.7 Hz, 1H), 7.49 (d, J=7.6 Hz, 1H), 7.43 (d, J=7.8 Hz, 1H), 2.61-2.52 (m, 1H), 2.01-1.94 (m, 1H), 1.48-1.37 (m, 2H). HPLC analysis gave e.r.=98:2 (Regis Whelk 01, Hexane/ⁱPrOH (70:30), 1.2 mL/min, t₍₋₎=15.95 min, t(+)=17.47 min).

### Example 41: Alternate synthesis of trans-(+)-2-(6-(2-carbamoyl-6-(trifluoromethoxy)-1H-indol-1-yl)pyridin-2-yl)cyclopropane-1-carboxylic acid (Compound 28b)

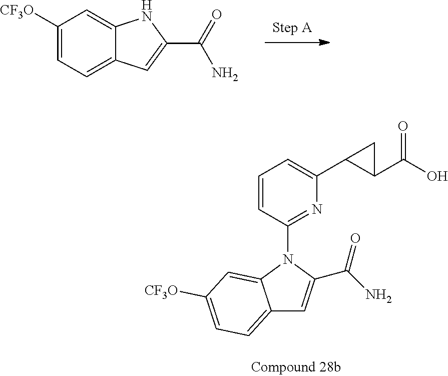

### Step A: Preparation of trans-(+)-2-(6-(2-carbamoyl-6-(trifluoromethoxy)-1H-indol-1-yl)pyridin-2-yl)cyclopropane-1-carboxylic acid (Compound 28b)

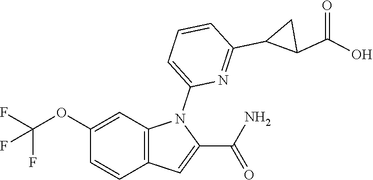 [SMILES: NC(=O)c1cc2ccc(OC(F)(F)F)cc2n1-c1cccc(C2CC2C(=O)O)n1]

To a mixture consisting of 6-(trifluoromethoxy)-1H-indole-2-carboxamide (0.244 g, 1.00 mmol), trans-(+)-2-(6-bromopyridin-2-yl)cyclopropane-1-carboxylic acid (0.266 g, 1.1 mmol), copper (I) iodide (Strem, 0.190 g, 1.00 mmol) in DMSO (6 mL) was added DBU (Oakwood Chemicals, 0.3 mL, 2.00 mmol). The reaction mixture was heated to 110° C. under N₂ atmosphere for 6 hours and then allowed to cool to room temperature. The reaction mixture was subsequently partitioned between ethyl acetate (75 mL) and 1M KHSO₄ (50 mL). A precipitate formed which was collected by filtration over a fritted funnel and set aside. The filtrate was poured in a separatory funnel. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.565 g. The crude oil was purified by flash silica column chromatography on a CombiFlash NextGen 300+ purification system. Elution through a 12 g RediSep Gold Rf flash silica cartridge with 10-70% ethyl acetate in hexanes containing 1% acetic acid afforded the title compound as a tan solid (0.165 g, 40% yield); R_{f} 0.21 with 1:60:40 v/v acetic acid-ethyl acetate-hexane (UV 254 nM); ¹H-NMR (400 MHz; DMSO-d6) δ 12.35 (s, 1H), 8.10 (br s, 1H), 7.85 (t, 1H, J=7.8 Hz), 7.80 (d, 1H, J=8.7 Hz), 7.48 (br d, 2H, J=7.6 Hz), 7.34 (s, 1H), 7.2-7.3 (m, 1H), 7.18-7.22 (m, 2H), 2.61-2.70 (m, 1H), 1.93-2.00 (m, 1H), 1.41 (t, 2H, J=7.1 Hz); ¹⁹F-NMR (376 MHz; DMSO-d6) δ −56.73; MS (APCI⁺) m/z 406.0 (M+1), (APCI−) m/z 404.0 (M−1); HPLC UV purity, Rt=7.13 min, 98.4%; Chiral HPLC purity Rt=15.27 min, 91.5%; optical rotation [α]²⁵_{D}=+254.4 (c=0.25, 2.24 mL IPA); melting point=192-193° C.

### Example 42: Synthesis of cis-(rac)-2-(6-(2-carbamoyl-6-(trifluoromethoxy)-1H-indol-1-yl)pyridin-2-yl)cyclopropane-1-carboxylic acid (Compound 28c)

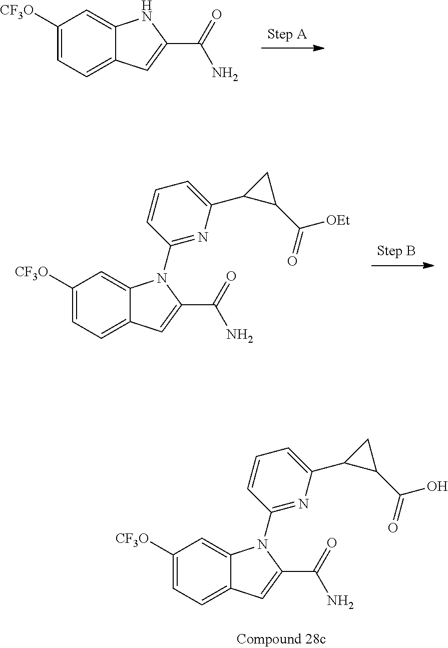

### Step A: Preparation of cis-(rac)-ethyl 2-(6-(2-carbamoyl-6-(trifluoromethoxy)-1H-indol-1-yl)pyridin-2-yl)cyclopropane-1-carboxylate

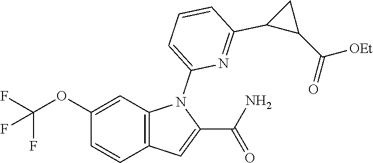 [SMILES: CCOC(=O)C1CC1c1cccc(-n2c(C(N)=O)cc3ccc(OC(F)(F)F)cc32)n1]

To a mixture consisting of 6-(trifluoromethoxy)-1H-indole-2-carboxamide (0.244 g, 1.00 mmol), cis-(rac)-2-(6-bromopyridin-2-yl)cyclopropane-1-carboxylic acid (0.297 g, 1.1 mmol), copper (I) iodide (Strem, 0.190 g, 1.00 mmol) in DMSO (6 mL) was added DBU (Oakwood Chemicals, 0.3 mL, 2.00 mmol). The reaction mixture was heated to 110° C. under N₂ atmosphere for 5 hours and then allowed to cool to room temperature. The reaction mixture was subsequently partitioned between ethyl acetate (100 mL) and 1M KHSO₄ (50 mL). A precipitate formed which was collected by filtration over a fritted funnel and set aside. The filtrate was poured in a separatory funnel. The phases were separated, and the organic phase was partitioned with H₂O (2×50 mL), followed by brine (50 mL). The organic layer was separated and dried over anhydrous magnesium sulfate, filtered, and concentrated under reduced pressure to give a crude red oil, 0.539 g. The crude oil was purified by flash silica column chromatography on a CombiFlash NextGen 300+ purification system. Elution through a 12 g RediSep Gold Rf flash silica cartridge with 10-50% ethyl acetate in hexanes containing 1% acetic acid afforded the title compound as a tan solid (0.191 g, 44% yield). R_{f} 0.22 with 1:60:40 v/v acetic acid-ethyl acetate-hexane (UV 254 nM); ¹H-NMR (400 MHz; DMSO-d6) δ 8.15 (br s, 1H), 7.80-7.85 (m, 2H), 7.38-7.51 (m, 2H), 7.37 (d, 1H, J=7.6 Hz), 7.28 (s, 1H), 7.17 (dd, 1H, J=1.0, 8.6 Hz), 7.12 (d, 1H, J=7.8 Hz), 3.72-3.85 (m, 2H), 2.79-2.82 (m, 1H), 1.79-1.82 (m, 1H), 1.42 (dt, 1H, J=4.6, 8.1 Hz), 0.87 (t, 3H, J=7.1 Hz); ¹⁹F-NMR (376 MHz; DMSO-d6) δ −56.73; MS (APCI⁺) m/z 434.0 (M+1), (APCI−) m/z 432.0 (M−1); HPLC UV purity, Rt=8.075 min, 97.6%; melting point=110.5-111.0° C.

### Step B: Preparation of cis-(rac)-2-(6-(2-carbamoyl-6-(trifluoromethoxy)-1H-indol-1-yl)pyridin-2-yl)cyclo propane-1-carboxylic acid (Compound 28C)

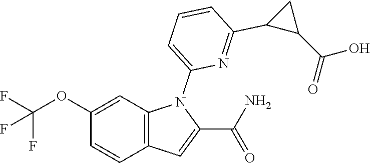 [SMILES: NC(=O)c1cc2ccc(OC(F)(F)F)cc2n1-c1cccc(C2CC2C(=O)O)n1]

To a mixture consisting of cis-racemic-ethyl 2-(6-(2-carbamoyl-6-(trifluoromethoxy)-1H-indol-1-yl)pyridin-2-yl)cyclopropane-1-carboxylate (0.156 g, 0.360 mmol) in methanol (5 mL) was added 1M lithium hydroxide (1.80 mL, 1.80 mmol). The reaction mixture was stirred at room temperature for 2 hours and then H₂O (1 mL) and THE (2 mL) was added. The reaction mixture was subsequently stirred overnight under N₂ atmosphere. The reaction mixture was subsequently partitioned between ethyl acetate (30 mL) and 1M HCl (10 mL). The phases were separated, and the organic phase was partitioned with water (15 mL), followed by brine (15 mL). The organic layer was concentrated under reduced pressure to afford the desired compound as a white solid (0.135 g, 92.4%). R_{f} 0.11 with 1:70:30 v/v acetic acid-ethyl acetate-heptane (UV 254 nM); ¹H-NMR (400 MHz; DMSO-d6) δ 12.01 (s, 1H), 8.16 (br s, 1H), 7.80-7.90 (m, 2H), 7.72 (s, 1H), 7.50 (br s, 1H), 7.35 (d, 1H, J=7.8 Hz), 7.26 (s, 1H), 7.17 (dd, 1H, J=1.1, 8.7 Hz), 7.08 (d, 1H, J=8.0 Hz), 2.80 (q, 1H, J=8.4 Hz), 2.11-2.15 (m, 1H), 1.79-1.82 (m, 1H), 1.37 (dt, 1H, J=4.6, 8.1 Hz); ¹⁹F-NMR (376 MHz; DMSO-d6) δ −56.72; MS (APCI⁺) m/z 406.0 (M+1), (APCI⁻) m/z 404.0 (M−1); HPLC UV purity, Rt=6.585 min, 95.8%; Chiral HPLC purity Rt=12.1 min, 48.2%, 15.1 min, 50.3%; melting point=219-220° C.

### Example 43: Synthesis of 3-(3-(2-carbamoyl-5-(trifluoromethoxy)benzo[b]selenophen-3-yl)phenyl)propanoic acid (Compound 29)

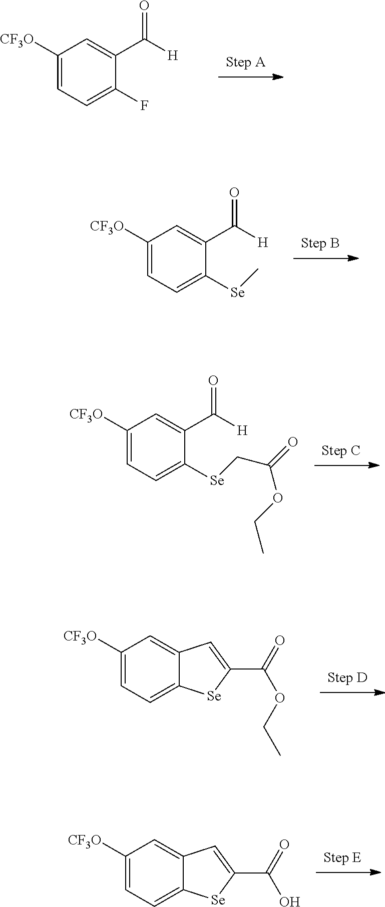

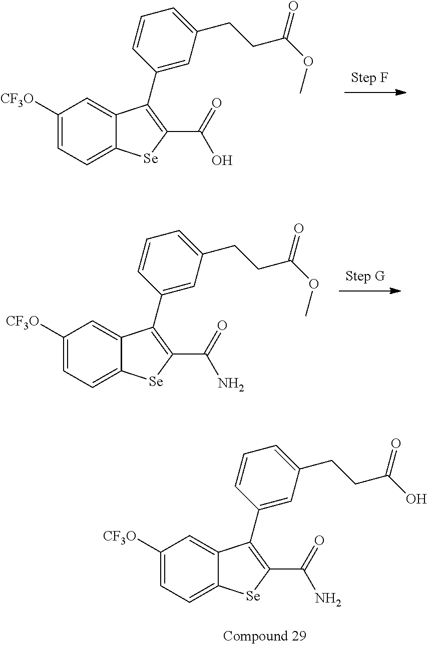

### Step A: Preparation of 2-(methylselanyl)-5-(trifluoromethoxy)benzaldehyde

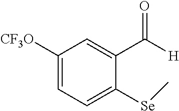

To a 200 mL round bottom flask was added DL-Dithiothreitol (Chem-Impex, 3.31 g, 15.9 mmol) and anhydrous DMF (35 mL). To this solution was added dimethyl diselenide (AK Scientific, 2.0 g, 10.6 mmol) and the solution stirred for 1 hour at room temperature. Next was added 2-fluoro-5-(trifluoromethoxy)benzaldehyde (Combi-Blocks, 3.31 g, 15.9 mmol) followed by DBU (Oakwood Products, 6.05 g, 39.7 mmol) and the mixture stirred at room temperature overnight. The reaction mixture was poured into 2 L of cold water to precipitate a yellow solid which was filtered and washed with water. The crude solid was purified by flash silica column chromatography on a CombiFlash NextGen 300+ purification system. Elution through a 80 g RediSep Gold Rf flash silica cartridge with 0-20% ethyl acetate in heptane afforded the title compound as a pale yellow solid (2.69 g, 60% yield). R_{f} 0.53 with 1:9 v/v ethyl acetate-heptane (UV 254 nM); ¹H-NMR (400 MHz; CDCl₃) δ 10.14 (s, 1H), 7.67 (d, 1H, J=1.8 Hz), 7.50 (d, 1H, J=8.7 Hz), 7.37 (dd, 1H, J=1.9, 8.6 Hz), 2.33 (s, 3H). Target mass not observed.

### Step B: Preparation of ethyl 2-((2-formyl-4-(trifluoromethoxy)phenyl)selanyl)acetate

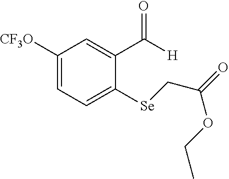

To a 100 mL round bottom flask was added 2-(methylselanyl)-5-(trifluoromethoxy)benzaldehyde (2.69 g, 9.50 mmol) followed by ethyl bromoacetate (Oakwood Products, 5.9 g, 35.3 mmol) and the mixture heated with stirring at 150° C. for 2.5 hours at which time TLC (1:9, v/v ethyl acetate-heptane) indicates consumption of starting material and formation of a new lower Rf spot. Cooled to room temperature and used crude for the next step. ¹H-NMR (400 MHz; CDCl₃) δ 10.1-10.2 (s, 1H), 7.84 (d, 1H, J=8.7 Hz), 7.71 (d, 1H, J=1.8 Hz), 7.41 (dd, 1H, J=1.9, 8.6 Hz), 4.1-4.2 (q, 2H, J=7.1 Hz), 3.58 (s, 2H), 1.24 (t, 3H, J=7.1 Hz). Note: also contains ethyl bromoacetate impurities.

### Step C: Preparation of ethyl 5-(trifluoromethoxy)benzo[b]selenophene-2-carboxylate

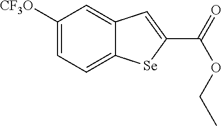

To a 100 mL round bottom flask containing crude ethyl 2-((2-formyl-4-(trifluoromethoxy)phenyl)selanyl)acetate (3.37 g, 9.50 mmol; theoretical) was added acetonitrile (20 mL) and potassium carbonate (3.8vg, 27.5 mmol). The mixture was stirred at reflux for 6 hours at which time TLC (1:9, ethyl acetate: heptane) indicated consumption of starting material and formation of a new higher Rf spot. Cooled to room temperature and filtered through a medium frit sintered glass funnel. Evaporated solvent. The crude oil material was purified by flash silica column chromatography on a CombiFlash NextGen 300+ purification system. Elution through a 80 g RediSep Gold Rf flash silica cartridge with 1-30% ethyl acetate: heptane afforded the title compound as pale yellow crystals (2.74 g, 85% yield over 2 steps); R_{f} 0.55 with 1:9 v/v ethyl acetate-heptane (UV 254 nM); ¹H-NMR (400 MHz; CDCl₃) δ 8.27 (s, 1H), 7.91 (d, 1H, J=8.7 Hz), 7.7-7.8 (d, 1H, J=1.4 Hz), 7.25-7.29 (dd, 1H, J=1.4 Hz, 8.7 Hz), 4.40 (q, 2H, J=7.1 Hz), 1.41 (t, 3H, J=7.2 Hz) MS (APCI⁺) m/z 324.1 (M+1). Target mass not observed. melting point=96.5-98.0° C.

### Step D: Preparation of 5-(trifluoromethoxy)benzo[b]selenophene-2-carboxylic acid (Intermediate X)

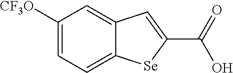

To a 100 mL round bottom flask was added ethyl 5-(trifluoromethoxy)benzo[b]selenophene-2-carboxylate (1.35 g, 4.0 mmol) and methanol (35 mL). A 1M aqueous solution of LiOH (12.0 mL, 3 eq) was next added dropwise with stirring. The reaction mixture was stirred for four hours at room temperature after which time TLC (1:30:70 v/v acetic acid-ethyl acetate-heptane) indicated consumption of starting material and formation of a new lower Rf spot. Solvent was removed by evaporation and the residue was subsequently diluted with water (300 mL) and the solution was acidified with 1N HCl to precipitate product. The suspension was filtered, and solids washed with excess water then dried under high vac overnight to afford the title compound as a white solid (1.20 g, 99% yield). Rf 0.43 with 1:30:70 v/v acetic acid-ethyl acetate-heptane (UV 254 nM); ¹H-NMR (400 MHz; CDCl₃) δ 8.40 (s, 1H), 7.94 (br d, 1H, J=8.5 Hz), 7.79 (br s, 1H), 7.3-7.3 (br d, 1H, J=8.5 Hz); ¹⁹F-NMR (376 MHz; CDCl₃) δ −57.90; MS (APCI⁺) m/z 309.0 (M−1, with Se isotope distribution); melting point=179.5-180.2° C.

### Step E: Preparation of 3-(3-(3-methoxy-3-oxopropyl)phenyl)-5-(trifluoromethoxy)benzo[b]selenophene-2-carboxylic acid

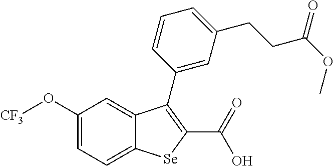 [SMILES: COC(=O)CCc1cccc(-c2c(C(=O)O)[se]c3ccc(OC(F)(F)F)cc23)c1]

To a 24 mL septa-cap vial was added methyl 3-bromobenzenepropanoate (Combi-Blocks, 0.365 g, 1.5 mmol) and 5-(trifluoromethoxy)benzo[b]selenophene-2-carboxylic acid (0.463 g, 1.5 mmol). Next was added Bis(dichloro(η⁶-p-cymene)ruthenium) (Strem, 0.037 g, 4 mol %), trimethylphosphonium tetrafluoroborate (Strem, 0.020 g, 8 mol %), K₂CO₃ (VWR, 0.228 g, 1.65 mmol) and NMP (6 mL). The mixture was degassed by bubbling in nitrogen gas for 3 min with stirring then heated to 110° C. overnight. The reaction mixture was subsequently cooled to room temperature and diluted with water (140 mL). The mixture was extracted with ethyl acetate (3×100 mL) and the combined organics dried with Na₂SO₄, filtered and solvent evaporated under reduced pressure to give a crude oil which was purified by flash silica column chromatography on a CombiFlash NextGen 300+ purification system. Elution through a 40 g RediSep Gold Rf flash silica cartridge with 10-30% ethyl acetate in heptane containing 1% acetic acid afforded the title compound as a tacky oil (0.127 g, 18% yield). R_{f} 0.24 with 1:30:70 v/v acetic acid-ethyl acetate-hexane (UV 254 nM); ¹H-NMR (400 MHz; CDCl₃) δ 7.93 (d, 1H, J=8.7 Hz), 7.4-7.5 (m, 1H), 7.32 (br d, 2H, J=7.8 Hz), 7.2-7.2 (m, 2H), 3.65 (s, 3H), 3.02 (t, 2H, J=7.7 Hz), 2.68 (t, 2H, J=7.7 Hz); ¹⁹F-NMR (376 MHz; CDCl₃) δ −57.89; MS (APCI⁺) m/z 471.0 (M−1).

### Step F: Preparation of methyl 3-(3-(2-carbamoyl-5-(trifluoromethoxy)benzo[b]selenophen-3-yl)phenyl)propanoate

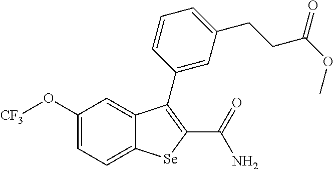 [SMILES: COC(=O)CCc1cccc(-c2c(C(N)=O)[se]c3ccc(OC(F)(F)F)cc23)c1]

To a mixture consisting of 3-(3-(3-methoxy-3-oxopropyl)phenyl)-5-(trifluoromethoxy)benzo[b]selenophene-2-carboxylic acid (0.120 g, 0.25 mmol) in DMF (1.5 mL) was added ammonium chloride (Chem-Impex, 0.041 g, 0.76 mmol). Next was added TBTU (Oakwood, 0.123 g, 0.38 mmol) followed by the addition of DIPEA (0.400 mL, 2.3 mmol). The reaction mixture was stirred overnight at room temperature under N₂ atmosphere. The reaction mixture was subsequently diluted with water (40 mL) then extracted with 3×20 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 10-40% ethyl acetate in hexanes containing 1% acetic acid afforded the title compound as a clear oil (0.101 g, 86% yield). R_{f} 0.22 with 1:30:70 v/v acetic acid-ethyl acetate-hexane (UV 254 nM); ¹H-NMR (400 MHz; CDCl₃) δ 7.94 (d, 1H, J=8.7 Hz), 7.5-7.6 (m, 1H), 7.41 (d, 1H, J=7.8 Hz), 7.3-7.3 (m, 3H), 7.1-7.1 (m, 1H), 5.91 (br s, 1H), 5.44 (br s, 1H), 3.66 (s, 3H), 3.05 (t, 2H, J=7.5 Hz), 2.70 (t, 2H, J=7.5 Hz); ¹⁹F-NMR (376 MHz; CDCl₃) δ −57.89; MS (APCI⁺) m/z 472.0 (M+1), m/z 470.0 (M−1).

### Step G: Preparation of 3-(3-(2-carbamoyl-5-(trifluoromethoxy)benzo[b]selenophen-3-yl)phenyl)propanoic acid (Compound 29)

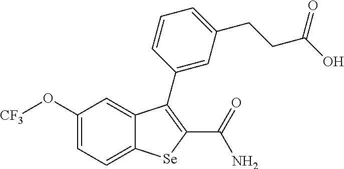 [SMILES: NC(=O)c1[se]c2ccc(OC(F)(F)F)cc2c1-c1cccc(CCC(=O)O)c1]

To a mixture consisting of methyl 3-(3-(2-carbamoyl-5-(trifluoromethoxy)benzo[b]selenophen-3-yl)phenyl)propanoate (0.100 g, 0.21 mmol) in MeOH (3 mL) was added dropwise, a 1M aqueous solution of LiOH (0.64 mL, 3 eq). The reaction mixture was stirred overnight at room temperature. Solvent was removed by evaporation and the residue was subsequently diluted with water (30 mL) and the solution was acidified with 1N HCl to precipitate product. The suspension was filtered, and solids washed with excess water then dried under high vac overnight to afford the title compound as a white solid (0.081 g, 84% yield). Rf 0.15 with 1:30:70 v/v acetic acid-ethyl acetate-heptane (UV 254 nM); ¹H-NMR (400 MHz; CDCl₃) δ 7.93 (d, 1H, J=8.7 Hz), 7.51 (t, 2H, J=7.6 Hz), 7.40 (br d, 1H, J=7.8 Hz), 7.3-7.3 (m, 2H), 7.18 (d, 1H, J=7.6 Hz), 7.10 (s, 1H), 5.63 (br s, 1H), 3.04 (br t, 2H, J=6.4 Hz), 2.74 (br t, 2H, J=6.4 Hz); ¹⁹F-NMR (376 MHz; CDCl₃) δ −57.87; MS (APCI⁺) m/z 458.0 (M+1), 456.0 (M−1). HPLC UV purity, Rt=8.14 min, 98.4%; melting point=179.5-181.5° C.

### Example 44: Synthesis of 2-(1-(3-(2-carbamoyl-6-(trifluoromethoxy)-1H-indol-1-yl)phenyl)cyclobutyl)acetic acid (Compound 30)

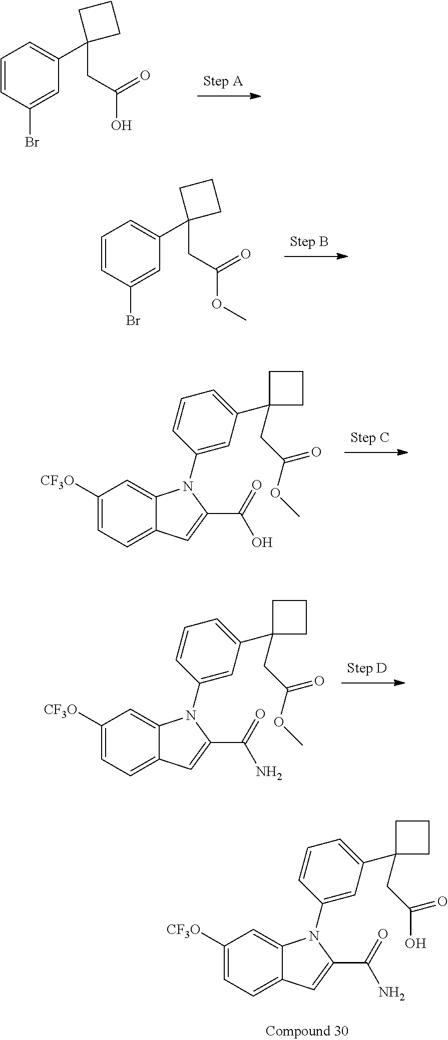

### Step A: Preparation of meth yl 2-(1-(3-bromophenyl)cyclobutyl)acetate

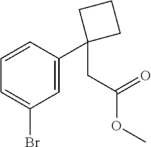 [SMILES: COC(=O)CC1(c2cccc(Br)c2)CCC1]

To an ice bath (0° C.) reaction mixture consisting of 2-(1-(3-bromophenyl)cyclobutyl)acetic acid (Key Organics, 0.50 g, 1.96 mmol) in toluene (6 mL) and methanol (4 mL) was added TMS-Diazomethane (2M solution in Ether, 1.5 mL, 2.94 mmol) dropwise. The reaction was stirred at 0° C. for 45 minutes then allowed to warm to room temperature for 2 hours. The reaction mixture was concentrated under reduced pressure to afford the crude product as a yellow oil (0.563 g). The crude oil was purified by flash silica column chromatography on a CombiFlash NextGen 300+ purification system. Elution through a 24 g RediSep Gold Rf flash silica cartridge with 2-30% ethyl acetate in heptane afforded the title compound as a colorless oil (0.349 g, 67% yield). R_{f} 0.62 with 25:75 v/v ethyl acetate-heptane (UV 254 nM); ¹H-NMR (400 MHz; CDCl₃) δ 7.32 (s, 1H), 7.31 (d, 1H, J=7.6 Hz), 7.17 (t, 1H, J=7.4 Hz), 7.10 (d, 1H, J=7.3 Hz), 3.51 (s, 3H), 2.79 (s, 2H), 2.35-2.47 (m, 4H), 2.08-2.13 (m, 1H), 1.80-1.90 (m, 1H); MS (APCI+) m/z 284.0; HPLC UV purity, Rt=10.027 min, 98.4%.

### Step B: Preparation of 1-(3-(1-(2-methoxy-2-oxoethyl)cyclobutyl)phenyl)-6-(trifluoromethoxy)-1H-indole-2-carboxylic acid

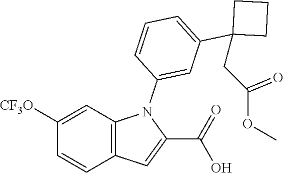

To a mixture consisting of 6-(trifluoromethoxy)-1H-indole-2-carboxylic acid (ChemShuttle, 0.286 g, 1.16 mmol), methyl 2-(1-(3-bromophenyl)cyclobutyl)acetate (0.330 g, 1.16 mmol), copper (II) acetate (CombiBlocks, 0.210 g, 1.16 mmol), methyl-α-D-glucopyranoside (CombiBlocks, 0.225 g, 1.16 mmol), potassium iodide (VWR, 0.385 g, 2.32 mmol) in DMSO (20 mL) was added DBU (Oakwood Chemicals, 0.52 mL, 3.48 mmol). The reaction mixture was heated to 115° C. under N₂ atmosphere for 6 hours and then allowed to cool to room temperature. The reaction mixture was subsequently partitioned between ethyl acetate (50 mL) and 1M KHSO₄ (30 mL). An emulsion formed which was broken up by the addition of 25 mL of brine. The phases were separated, and the organic phase was partitioned with H₂O (30 mL), followed by brine (30 mL). The organic layer was separated and dried over anhydrous magnesium sulfate, filtered, and concentrated under reduced pressure to give a crude brown oil, 0.926 g. The crude oil was purified by flash silica column chromatography on a CombiFlash NextGen 300+ purification system. Elution through a 24 g RediSep Gold Rf flash silica cartridge with 5-25% ethyl acetate in heptane containing 1% acetic acid afforded the title compound as a white solid (0.108 g). The product obtained contained two impurities and was used in the next reaction without further purification. R_{f} 0.30 with 1:30:70 v/v acetic acid-ethyl acetate-heptane (UV 254 nM); MS (APCI+) m/z 448.0 (M+1), (APCI−) m/z 446.0 (M−1); LC/MS UV purity, Rt=8.905 min, 49.7%.

### Step C: Preparation of methyl 2-(1-(3-(2-carbamoyl-6-(trifluoromethoxy)-1H-indol-1-yl)phenyl)cyclobutyl)acetate

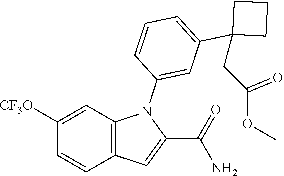

To a mixture consisting of 1-(3-(1-(2-methoxy-2-oxoethyl)cyclobutyl)phenyl)-6-(trifluoromethoxy)-1H-indole-2-carboxylic acid (0.108 g, 0.241 mmol) in DMF (2 mL) was added ammonium chloride (Chem-Impex, 0.039 g, 0.72 mmol). Next add TBTU (Oakwood, 0.116 g, 0.36 mmol), followed by the addition of DIPEA (0.38 mL, 2.17 mmol). The reaction mixture was stirred for 72 hours at room temperature under N₂ atmosphere. The reaction mixture was subsequently partitioned between ethyl acetate (50 mL) and H₂O (50 mL). The phases were separated, and the organic phase was partitioned with 1M KHSO₄ (25 mL), followed by brine (25 mL). The organic layer was concentrated under reduced pressure to afford the crude product as a yellow oil (0.110 g). The crude oil was purified by flash silica column chromatography on a CombiFlash NextGen 300+ purification system. Elution through a 12 g RediSep Gold Rf flash silica cartridge with 5-70% ethyl acetate in heptane containing 1% acetic acid afforded the title compound as a white solid (0.027 g, 43% yield). R_{f} 0.14 with 1:30:70 v/v acetic acid-ethyl acetate-heptane (UV 254 nM); MS (APCI+) m/z 447.0 (M+1), (APCI−) m/z 445.0 (M−1); HPLC UV purity, Rt=9.65 min, 96.0%.

### Step D: Preparation of 2-(1-(3-(2-carbamoyl-6-(trifluoromethoxy)-1H-indol-1-yl)phenyl)cyclobutyl)acetic acid (Compound 30)

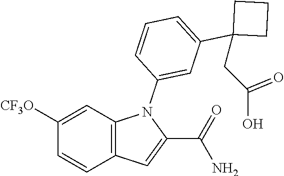

To a mixture consisting of methyl 2-(1-(3-(2-carbamoyl-6-(trifluoromethoxy)-1H-indol-1-yl)phenyl)cyclobutyl)acetate (0.027 g, 0.060 mmol) in methanol (2 mL) was added 1M lithium hydroxide (0.25 mL, 0.25 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with water (10 mL), followed by brine (10 mL). The organic layer was concentrated under reduced pressure to afford the title compound as a white solid (0.024 g, 95% yield). R_{f} 0.13 with 1:40:60 v/v acetic acid-ethyl acetate-heptane (UV 254 nM); ¹H-NMR (400 MHz; DMSO-d6) δ 11.88 (br s, 1H), 8.00 (br s, 1H), 7.84 (d, 1H, J=8.7 Hz), 7.42-7.48 (m, 2H), 7.28 (d, 1H, J=7.3 Hz), 7.27 (s, 1H), 7.1-7.2 (m, 3H), 7.04 (s, 1H), 2.79 (s, 2H), 2.3-2.4 (m, 4H), 2.08-2.12 (m, 1H), 1.8-1.9 (m, 1H); ¹⁹F-NMR (376 MHz; DMSO-d6) δ −56.74; MS (APCI⁺) m/z 433.0 (M+1), 431.0 (M−1); HPLC UV purity, Rt=8.46 min, 96.5%; melting point=109-110° C.

### Example 45: Synthesis of 2-(1-(3-(2-carbamoyl-6-(trifluoromethoxy)-1H-indol-1-yl)phenyl)cyclopropyl)acetic acid (Compound 31)

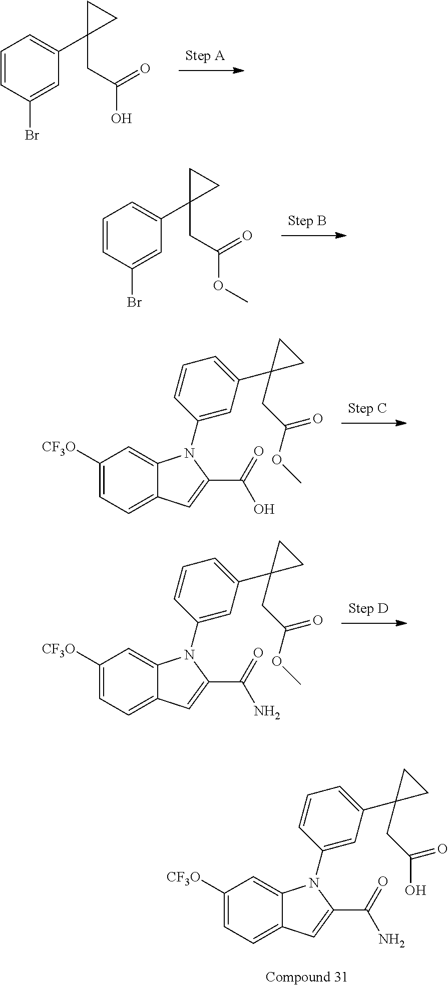

### Step A: Preparation of methyl 2-(1-(3-bromophenyl)cyclopropyl)acetate

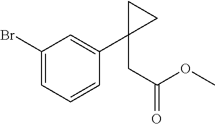

To an ice bath (0° C.) reaction mixture consisting of 2-(1-(3-bromophenyl)cyclopropyl)acetic acid (Key Organics, 0.50 g, 1.96 mmol) in toluene (6 mL) and methanol (4 mL) was added TMS-Diazomethane (2M solution in Ether, 1.5 mL, 2.94 mmol) dropwise. The reaction was stirred at 0° C. for 45 minutes then allowed to warm to room temperature for 2 hours. The reaction mixture was concentrated under reduced pressure to afford the crude product as a yellow oil (0.576 g). The crude oil was purified by flash silica column chromatography on a CombiFlash NextGen 300+ purification system. Elution through a 24 g RediSep Gold Rf flash silica cartridge with 2-50% ethyl acetate in heptane afforded the title compound as a colorless oil (0.353 g, 67% yield). R_{f} 0.56 with 25:75 v/v ethyl acetate-heptane (UV 254 nM); ¹H-NMR (400 MHz; CDCl₃) δ 7.46 (t, 1H, J=1.7 Hz), 7.32 (d, 1H, J=8.2 Hz), 7.25 (d, 1H, J=1.4 Hz), 7.10-7.16 (m, 1H), 3.61 (s, 3H), 2.60 (s, 2H), 0.87-0.98 (m, 4H); LC/MS (APCI+) m/z 269.0; HPLC UV purity, Rt=9.251 min, 99.7%.

### Step B: Preparation of 1-(3-(1-(2-methoxy-2-oxoethyl)cyclopropyl)phenyl)-6-(trifluoromethoxy)-1H-indole-2-carboxylic acid

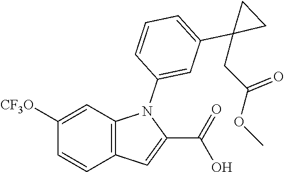

To a mixture consisting of 6-(trifluoromethoxy)-1H-indole-2-carboxylic acid (ChemShuttle, 0.301 g, 1.23 mmol), methyl 2-(1-(3-bromophenyl)cyclopropyl)acetate (0.330 g, 1.23 mmol), copper (II) acetate (CombiBlocks, 0.222 g, 1.23 mmol), methyl-α-D-glucopyranoside (CombiBlocks, 0.239 g, 1.23 mmol), potassium iodide (VWR, 0.408 g, 2.46 mmol) in DMSO (20 mL) was added DBU (Oakwood Chemicals, 0.55 mL, 3.69 mmol). The reaction mixture was heated to 115° C. under N₂ atmosphere for 6 hours and then allowed to cool to room temperature. The reaction mixture was subsequently partitioned between ethyl acetate (50 mL) and 1M KHSO₄ (30 mL). An emulsion formed which was broken up by the addition of 25 mL of brine. The phases were separated, and the organic phase was partitioned with H₂O (30 mL), followed by brine (30 mL). The organic layer was separated and dried over anhydrous magnesium sulfate, filtered, and concentrated under reduced pressure to give a crude brown oil, 0.725 g. The crude oil was purified by flash silica column chromatography on a CombiFlash NextGen 300+ purification system. Elution through a 24 g RediSep Gold Rf flash silica cartridge with 5-30% ethyl acetate in heptane containing 1% acetic acid afforded the title compound as a white solid (0.071 g). The product obtained contained two impurities and was used in the next reaction without further purification. R_{f} 0.32 with 1:30:70 v/v acetic acid-ethyl acetate-heptane (UV 254 nM); MS (APCI+) m/z 434.0 (M+1), (APCI−) m/z 432.0 (M−1); LC/MS UV purity, Rt=9.791 min, 47.5%.

### Step C: Preparation of methyl 2-(1-(3-(2-carbamoyl-6-(trifluoromethoxy)-1H-indol-1-yl)phenyl)cyclopropyl)acetate

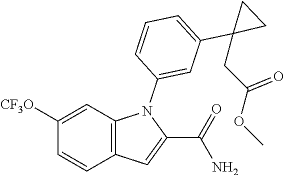

To a mixture consisting of 1-(3-(1-(2-methoxy-2-oxoethyl)cyclopropyl)phenyl)-6-(trifluoromethoxy)-1H-indole-2-carboxylic acid (0.068 g, 0.157 mmol) in DMF (2 mL) was added ammonium chloride (Chem-Impex, 0.025 g, 0.47 mmol). Next add TBTU (Oakwood, 0.076 g, 0.23 mmol), followed by the addition of DIPEA (0.25 mL, 1.41 mmol). The reaction mixture was stirred for 72 hours at room temperature under N₂ atmosphere. The reaction mixture was subsequently partitioned between ethyl acetate (25 mL) and H₂O (50 mL). The phases were separated, and the organic phase was partitioned with 1M KHSO₄ (15 mL), followed by brine (15 mL). The organic layer was concentrated under reduced pressure to afford the crude product as a colorless film (0.066 g). The crude product was purified by flash silica column chromatography on a CombiFlash NextGen 300+ purification system. Elution through a 12 g RediSep Gold Rf flash silica cartridge with 5-75% ethyl acetate in heptane containing 1% acetic acid afforded the title compound as a white solid (0.054 g, 81% yield). R_{f} 0.14 with 1:30:70 v/v acetic acid-ethyl acetate-heptane (UV 254 nM); MS (APCI+) m/z 433.0 (M+1), (APCI−) m/z 431.0 (M−1); HPLC UV purity, Rt=9.04 min, 97.7%.

### Step D: Preparation of 2-(1-(3-(2-carbamoyl-6-(trifluoromethoxy)-1H-indol-1-yl)phenyl)cyclopropyl)acetic acid (Compound 31)

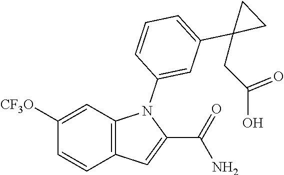

To a mixture consisting of methyl 2-(1-(3-(2-carbamoyl-6-(trifluoromethoxy)-1H-indol-1-yl)phenyl)cyclopropyl)acetate (0.052 g, 0.121 mmol) in methanol (2 mL) was added 1M lithium hydroxide (0.50 mL, 0.50 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with water (10 mL), followed by brine (10 mL). The organic layer was concentrated under reduced pressure to afford the title compound as a white solid (0.048 g, 97% yield). R_{f} 0.11 with 1:40:60 v/v acetic acid-ethyl acetate-heptane (UV 254 nM); ¹H-NMR (400 MHz; DMSO-d6) δ 12.03 (s, 1H), 8.01 (br s, 1H), 7.84 (d, 1H, J=8.7 Hz), 7.40-7.44 (m, 2H), 7.32 (d, 1H, J=8.3 Hz), 7.27 (s, 1H), 7.20 (t, 1H, J=1.7 Hz), 7.11-7.17 (m, 2H), 7.01 (s, 1H), 2.63 (s, 2H), 0.95 (br d, 4H, J=3.9 Hz); ¹⁹F-NMR (376 MHz; DMSO-d6) δ −56.71; MS (APCI⁺) m/z 419.0 (M+1), 417.0 (M−1); HPLC UV purity, Rt=7.86 min, 96.6%; melting point=150-151° C.

### Example 46: Synthesis of 1-(6-(acetamidomethyl)pyridin-2-yl)-6-(trifluoromethoxy)-1H-indole-2-carboxamide (Compound 32)

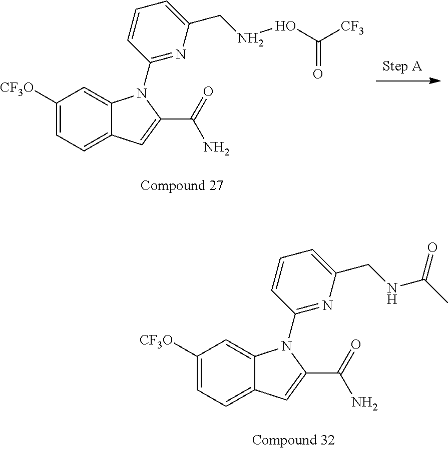

### Step A: Preparation of 1-(6-(acetamidomethyl)pyridin-2-yl)-6-(trifluoromethoxy)-1H-indole-2-carboxamide (Compound 32)

To a mixture consisting of Compound 27 (0.022 g, 0.047 mmol) in dichloromethane (1.5 mL) was added N,N-diisopropylethylamine (0.02 mL, 0.10 mmol). While the reaction mixture was stirred at room temperature, acetyl chloride (Alfa Aesar, 0.01 mL, 0.047 mmol) was added dropwise. The reaction was stirred for 30 minutes at room temperature. The reaction mixture was subsequently partitioned between dichloromethane (10 mL) and water (10 mL). The phases were separated, and the organic phase was washed with brine (10 mL). The organic layer was concentrated under reduced pressure to afford a crude yellow oil (0.029 g). The crude product was purified by flash silica column chromatography on a CombiFlash NextGen 300+ purification system. Elution through a 4 g RediSep Gold Rf flash silica cartridge with 5-75% ethyl acetate in heptane containing 1% acetic acid afforded the title compound as a white solid (0.005 g, 29% yield). R_{f} 0.94 with 1:80:20 v/v acetic acid-ethyl acetate-heptane (UV 254 nM); ¹H-NMR (400 MHz; DMSO-d6) δ 8.52 (t, 1H, J=6.0 Hz), 8.02 (t, 1H, J=7.9 Hz), 7.93-7.99 (m, 2H), 7.59 (br s, 1H), 7.41 (t, 2H, J=8.9 Hz), 7.31 (br d, 1H, J=8.9 Hz), 4.43 (d, 2H, J=6.0 Hz), 2.37 (s, 3H); ¹⁹F-NMR (376 MHz; DMSO-d6) δ −56.77; MS (APCI−) m/z 319.0 (M−1); HPLC UV purity, Rt=7.88 min, 97.3%.

### Example 47: Synthesis of 1-(6-(methylsulfonamidoethyl)pyridin-2-yl)-6-(trifluoromethoxy)-1H-indole-2-carboxamide (Compound 33)

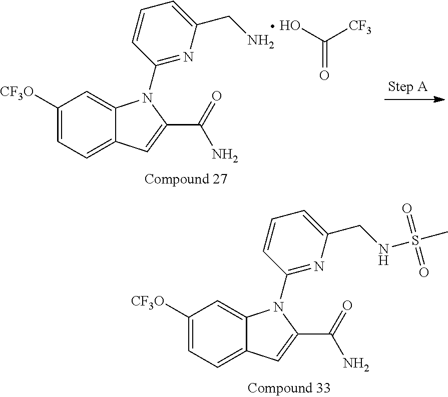

### Step A: Preparation of 1-(6-(methylsulfonamidoethyl)pyridin-2-yl)-6-(trifluoromethoxy)-1H-indole-2-carboxamide (Compound 33)

To a mixture consisting of Compound 27 (0.025 g, 0.053 mmol) in dichloromethane (1.5 mL) was added triethylamine (0.02 mL, 0.10 mmol). Next, methanesulfonyl chloride (0.01 mL, 0.06 mmol) was added dropwise and the reaction was allowed to stir for 2 hours at room temperature. The reaction mixture was subsequently partitioned between dichloromethane (5 mL) and water (5 mL). The phases were separated, and the organic phase was washed with brine (5 mL). The organic layer was concentrated under reduced pressure to afford a crude yellow oil (0.025 g). The crude product was purified by flash silica column chromatography on a CombiFlash NextGen 300+ purification system. Elution through a 4 g RediSep Gold Rf flash silica cartridge with 10-90% ethyl acetate in heptane containing 1% acetic acid afforded the title compound as a white solid (0.013 g, 59% yield). R_{f} 0.89 with 0.5:20:80 v/v acetic acid-methanol-dichloromethane (UV 254 nM); ¹H-NMR (400 MHz; DMSO-d6) δ 8.16 (br s, 1H), 8.00 (t, 1H, J=7.8 Hz), 7.85 (d, 1H, J=8.7 Hz), 7.75 (t, 1H, J=6.3 Hz), 7.5-7.6 (m, 2H), 7.41 (s, 1H), 7.30 (s, 1H), 7.29 (d, 1H, J=8.7 Hz), 7.19 (br d, 1H, J=8.7 Hz), 4.31 (d, 2H, J=6.2 Hz), 2.93 (s, 3H); ¹⁹F-NMR (376 MHz; DMSO-d6) δ −56.64; MS (APCI⁺) m/z 429.0 (M+1), (APCI−) m/z 427.0 (M−1); HPLC UV purity, Rt=6.679 min, 98.8%; melting point=160-161° C.

### Example 48: Synthesis of 2-(1-(3-(2-carbamoyl-5-(trifluoromethoxy)benzo[b]selenophen-3-yl)phenyl)cyclopropyl)acetic acid (Compound 34)

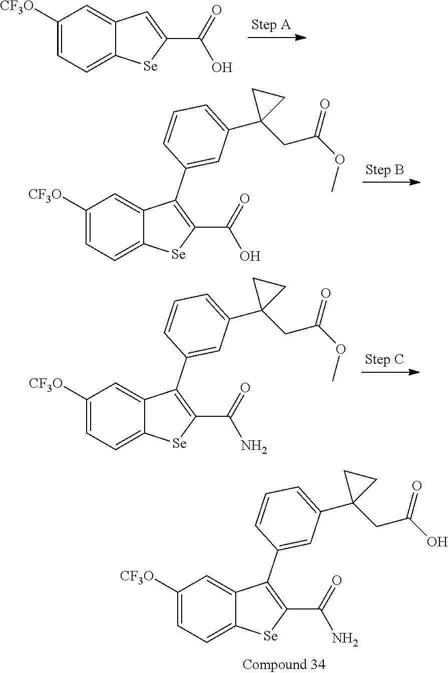

### Step A: Preparation of 3-(3-(1-(2-methoxy-2-oxoethyl)cyclopropyl)phenyl)-5-(trifluoromethoxy)benzo[b]selenophene-2-carboxylic acid

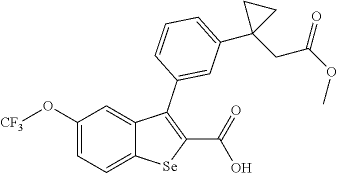 [SMILES: COC(=O)CC1(c2cccc(-c3c(C(=O)O)[se]c4ccc(OC(F)(F)F)cc34)c2)CC1]

To a 24 mL septa-cap vial was added methyl 2-(1-(3-bromophenyl)cyclopropyl)acetate (0.202 g, 0.75 mmol) and 5-(trifluoromethoxy)benzo[b]selenophene-2-carboxylic acid (0.232 g, 0.75 mmol). Next was added Bis(dichloro(η⁶)-cymene)ruthenium) (Strem, 0.019 g, 4 mol %), trimethylphosphonium tetrafluoroborate (Strem, 0.010 g, 8 mol %), K₂CO₃ (VWR, 0.114 g, 0.83 mmol) and NMP (3 mL). The mixture was degassed by bubbling in nitrogen gas for 3 min with stirring then heated to 110° C. overnight. The reaction mixture was subsequently cooled to room temperature and diluted with water (50 mL). The mixture was extracted with ethyl acetate (3×40 mL) and the combined organics dried with Na₂SO₄, filtered and solvent evaporated under reduced pressure to give a crude oil which was purified by flash silica column chromatography on a CombiFlash NextGen 300+ purification system. Elution through a 24 g RediSep Gold Rf flash silica cartridge with 5-35% ethyl acetate in heptane containing 1% acetic acid afforded the title compound as an off-white semi-solid (0.130 g, 38% yield). R_{f} 0.46 with 1:40:60 v/v acetic acid-ethyl acetate-hexane (UV 254 nM); ¹H-NMR (400 MHz; CDCl₃) δ 7.91-7.88 (m, 1H), 7.44-7.36 (m, 5H), 7.22 (d, 1H, J=6.6 Hz), 5.26 (br s, 1H), 3.57 (s, 3H), 2.64 (s, 2H), 0.9-1.0 (m, 4H); MS (APCI⁺) m/z 449.0 (M−1).

### Step B: Preparation of methyl 2-(1-(3-(2-carbamoyl-5-(trifluoromethoxy)benzo[b]selenophen-3-yl)phenyl)cyclopropyl)acetate

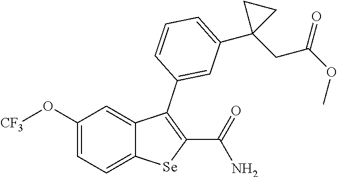 [SMILES: COC(=O)CC1(c2cccc(-c3c(C(N)=O)[se]c4ccc(OC(F)(F)F)cc34)c2)CC1]

To a mixture consisting of 3-(3-(1-(2-methoxy-2-oxoethyl)cyclopropyl)phenyl)-5-(trifluoromethoxy)benzo[b]selenophene-2-carboxylic acid (0.076 g, 0.15 mmol) in DMF (1.5 mL) was added ammonium chloride (Chem-Impex, 0.025 g, 0.46 mmol). Next was added TBTU (Oakwood, 0.074 g, 0.23 mmol) followed by the addition of DIPEA (0.24 mL, 1.4 mmol). The reaction mixture was stirred overnight at room temperature under N₂ atmosphere. The reaction mixture was subsequently diluted with water (25 mL) then extracted with 2×30 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 5-40% ethyl acetate in hexanes containing 1% acetic acid afforded the title compound as a clear oil (0.059 g, 77% yield). R_{f} 0.26 with 1:30:70 v/v acetic acid-ethyl acetate-hexane (UV 254 nM); ¹H-NMR (400 MHz; CDCl₃) δ 7.94 (d, 1H, J=8.7 Hz), 7.5-7.5 (m, 2H), 7.37 (s, 1H), 7.3-7.3 (m, 1H), 7.2-7.3 (m, 1H), 7.11 (s, 1H), 5.61 (d, 2H, J=91.5 Hz), 3.59 (s, 3H), 2.6-2.7 (m, 2H), 1.0-1.1 (m, 2H), 0.97 (s, 2H); MS (APCI⁺) m/z 498.0 (M+1), m/z 496.1 (M−1).

### Step C: Preparation of 2-(1-(3-(2-carbamoyl-5-(trifluoromethoxy)benzo[b]selenophen-3-yl)phenyl)cyclopropyl)acetic acid (Compound 34)

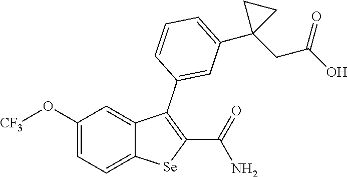 [SMILES: NC(=O)c1[se]c2ccc(OC(F)(F)F)cc2c1-c1cccc(C2(CC(=O)O)CC2)c1]

To a solution of methyl 2-(1-(3-(2-carbamoyl-5-(trifluoromethoxy)benzo[b]selenophen-3-yl)phenyl)cyclopropyl)acetate (0.053 g, 0.11 mmol) in MeOH (2 mL) was added dropwise, a 1M aqueous solution of LiOH (0.32 mL, 3 eq). The reaction mixture was stirred overnight at room temperature. Solvent was removed by evaporation and the residue was subsequently diluted with water (30 mL) and the solution was acidified with 1N HCl to precipitate product. The suspension was filtered, and solids washed with excess water then dried under high vac overnight to afford the title compound as an off-white solid. The solid was further purified by flash silica column chromatography on a CombiFlash NextGen 300+ purification system. Elution through a 4 g RediSep Gold Rf flash silica cartridge with 10-40% ethyl acetate in hexanes containing 1% acetic acid afforded the title compound as a white solid (0.025 g, 48% yield). Rf 0.32 with 1:40:60 v/v acetic acid-ethyl acetate-heptane (UV 254 nM); ¹H-NMR (400 MHz; CDCl₃) δ 7.93 (d, 1H, J=8.7 Hz), 7.88 (br s, 1H), 7.49 (t, 1H, J=7.6 Hz), 7.3-7.4 (m, 2H), 7.3-7.3 (m, 1H), 7.12 (br d, 1H, J=7.6 Hz), 7.05 (s, 1H), 5.74 (br s, 1H), 2.68 (br d, 1H, J=14.9 Hz), 2.53 (br d, 1H, J=15.1 Hz), 1.1-1.3 (m, 1H), 1.0-1.1 (m, 1H), 1.0-1.0 (m, 2H); ¹⁹F-NMR (376 MHz; CDCl₃) δ −57.90; MS (APCI⁺) m/z 484.0 (M+1), 482.0 (M−1). HPLC UV purity, Rt=8.63 min, 99.7%; melting point=170.0-172.0° C.

### Example 49: Synthesis of methyl 2-(1-(3-(2-carbamoyl-5-(trifluoromethoxy)benzo[b]thiophen-3-yl)phenyl)cyclopropyl)acetate (Compound 35) and 2-(1-(3-(2-carbamoyl-5-(trifluoromethoxy)benzo[b]thiophen-3-yl)phenyl)cyclopropyl)acetic acid (Compound 36)

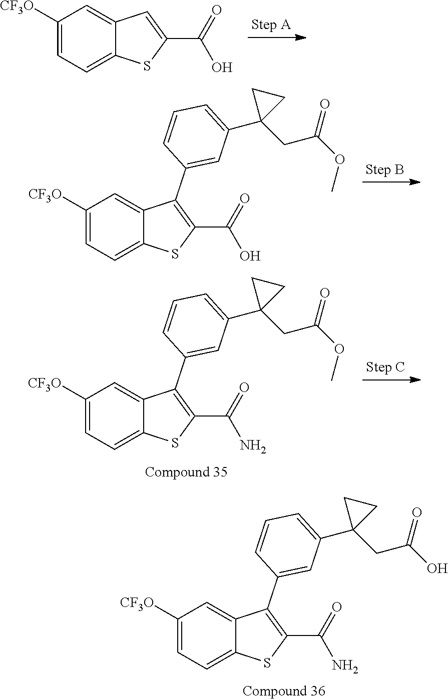

### Step A: Preparation of 3-(3-(1-(2-methoxy-2-oxoethyl)cyclopropyl)phenyl)-5-(trifluoromethoxy)benzo[b]thiophene-2-carboxylic acid

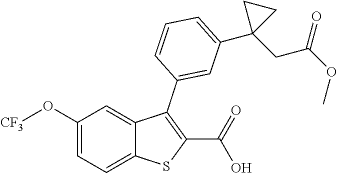 [SMILES: COC(=O)CC1(c2cccc(-c3c(C(=O)O)sc4ccc(OC(F)(F)F)cc34)c2)CC1]

To a 24 mL septa-cap vial was added methyl 2-(1-(3-bromophenyl)cyclopropyl)acetate (0.202 g, 0.75 mmol) and 5-(trifluoromethoxy)benzo[b]thiophene-2-carboxylic acid (0.197 g, 0.75 mmol). Next was added Bis(dichloro(η⁶-p-cymene)ruthenium) (Strem, 0.019 g, 4 mol %), trimethylphosphonium tetrafluoroborate (Strem, 0.010 g, 8 mol %), K₂CO₃ (VWR, 0.114 g, 0.83 mmol) and NMP (3 mL). The mixture was degassed by bubbling in nitrogen gas for 3 min with stirring then heated to 110° C. overnight. The reaction mixture was subsequently cooled to room temperature and diluted with water (50 mL). The mixture was extracted with ethyl acetate (3×40 mL) and the combined organics dried with Na₂SO₄, filtered and solvent evaporated under reduced pressure to give a crude oil which was purified by flash silica column chromatography on a CombiFlash NextGen 300+ purification system. Elution through a 12 g RediSep Gold Rf flash silica cartridge with 5-35% ethyl acetate in heptane containing 1% acetic acid afforded the title compound as an off-white semi-solid (0.130 g, 38% yield). R_{f} 0.46 with 1:40:60 v/v acetic acid-ethyl acetate-hexane (UV 254 nM); ¹H-NMR (400 MHz; CDCl₃) δ 7.91-7.88 (m, 1H), 7.44-7.36 (m, 5H), 7.22 (d, 1H, J=6.6 Hz), 5.26 (br s, 1H), 3.57 (s, 3H), 2.64 (s, 2H), 0.9-1.0 (m, 4H); MS (APCI⁺) m/z 449.0 (M−1).

### Step B: Preparation of methyl 2-(1-(3-(2-carbamoyl-5-(trifluoromethoxy)benzo[b]thiophen-3-yl)phenyl)cyclopropyl)acetate (Compound 35)

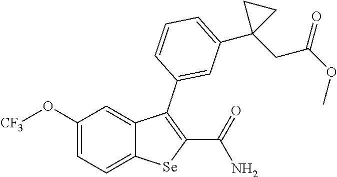 [SMILES: COC(=O)CC1(c2cccc(-c3c(C(N)=O)[se]c4ccc(OC(F)(F)F)cc34)c2)CC1]

To a mixture consisting of 3-(3-(1-(2-methoxy-2-oxoethyl)cyclopropyl)phenyl)-5-(trifluoromethoxy)benzo[b]thiophene-2-carboxylic acid (0.123 g, 0.27 mmol) in DMF (3.0 mL) was added ammonium chloride (Chem-Impex, 0.044 g, 0.82 mmol). Next was added TBTU (Oakwood, 0.132 g, 0.41 mmol) followed by the addition of DIPEA (0.43 mL, 2.5 mmol). The reaction mixture was stirred overnight at room temperature under N₂ atmosphere. The reaction mixture was subsequently diluted with water (40 mL) then extracted with 2×30 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 10-40% ethyl acetate in heptane containing 1% acetic acid afforded the title compound as a white solid (0.086 g, 71% yield). R_{f} 0.46 with 1:40:60 v/v acetic acid-ethyl acetate-hexane (UV 254 nM); ¹H-NMR (400 MHz; CDCl₃) δ 7.91 (d, 1H, J=8.7 Hz), 7.5-7.5 (m, 2H), 7.40 (s, 1H), 7.33 (d, 1H, J=9.0 Hz), 7.28 (t, 1H, J=1.7 Hz), 7.21 (s, 1H), 5.68 (br s, 1H), 5.63 (br s, 1H), 3.60 (s, 3H), 2.63 (s, 2H), 0.9-1.1 (m, 4H); MS (APCI⁺) m/z 450.1 (M+1), m/z 448.1 (M−1). HPLC UV purity, Rt=9.68 min, 98.8%; melting point=136.0-138.0° C.

### Step C: Preparation of 2-(1-(3-(2-carbamoyl-5-(trifluoromethoxy)benzo[b]thiophen-3-yl)phenyl)cyclopropyl)acetic acid (Compound 36)

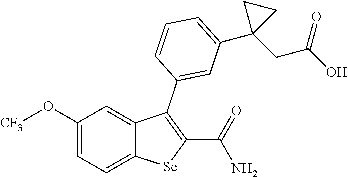 [SMILES: NC(=O)c1[se]c2ccc(OC(F)(F)F)cc2c1-c1cccc(C2(CC(=O)O)CC2)c1]

To a mixture consisting of methyl 2-(1-(3-(2-carbamoyl-5-(trifluoromethoxy)benzo[b]thiophen-3-yl)phenyl)cyclopropyl)acetate (Compound 35, 0.076 g, 0.17 mmol) in MeOH (4 mL) and THE (2 mL) was added dropwise a 1M aqueous solution of LiOH (0.51 mL, 3 eq). The reaction mixture was stirred overnight at 40° C. The solution was subjected to reduced pressure on a rotary evaporator to remove MeOH/THF. Additional water was added (30 mL) and the aqueous mixture acidified with 1N HCl. Extracted the aqueous mixture with ethyl acetate (2×30 mL). Dry the combined organics with Na₂SO₄. Filter and evaporate solvent to obtain crude product which was purified by flash silica column chromatography on a CombiFlash NextGen 300+ purification system. Elution through a 12 g RediSep Gold Rf flash silica cartridge with 10-40% ethyl acetate in heptane containing 1% acetic acid afforded the title compound as a tacky oil which was triturated in hexanes to precipitate title compound as a white solid after high vacuum at 40° C. overnight (0.027 g, 37% yield). Rf 0.22 with 1:40:60 v/v acetic acid-ethyl acetate-heptane (UV 254 nM); ¹H-NMR (400 MHz; CDCl₃) δ 12.53 (br s, 1H), 7.8-7.9 (m, 2H), 7.5-7.5 (m, 1H), 7.3-7.4 (m, 3H), 7.1-7.2 (m, 2H), 5.88 (br s, 1H), 2.62 (br s, 2H), 1.07 (br s, 2H), 0.97 (br s, 2H); ¹⁹F-NMR (376 MHz; CDCl₃) δ −57.89; MS (APCI⁺) m/z 436.0 (M+1), m/z 434.0 (M−1), HPLC UV purity, Rt=8.53 min, 99.9%; melting point=153.0-155.0° C.

### Example 50: Synthesis of 1-(6-((2,2,2-trifluoroacetamido)methyl)pyridin-2-yl)-6-(trifluoromethoxy)-1H-indole-2-carboxamide (Compound 37)

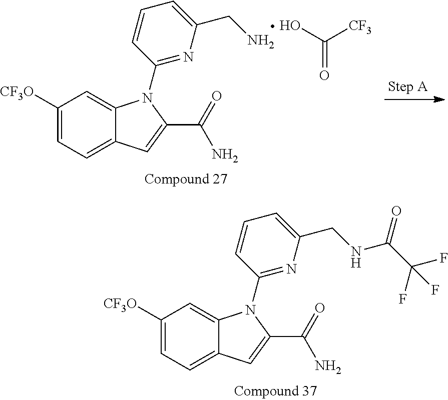

To a mixture consisting of Compound 27 (0.033 g, 0.071 mmol) in dichloromethane (2.0 mL) was added triethylamine (0.02 mL, 0.15 mmol). Next, trifluoroacetic anhydride (0.01 mL, 0.08 mmol) was added dropwise and the reaction was allowed to stir for one hour at room temperature. The reaction mixture was subsequently partitioned between dichloromethane (10 mL) and water (10 mL). The phases were separated, and the organic phase was washed with 1N HCl (10 mL), followed by saturated aqueous sodium bicarbonate (10 mL). The organic layer was concentrated under reduced pressure to afford the title compound as a tan solid (0.030 g, 86% yield). R_{f} 0.94 with 1:80:20 v/v acetic acid-ethyl acetate-heptane (UV 254 nM); ¹H-NMR (400 MHz; DMSO-d6) δ 10.08 (br t, 1H, J=5.6 Hz), 8.18 (t, 1H, J=7.8 Hz), 7.95 (d, 1H, J=8.7 Hz), 7.88 (s, 1H), 7.7-7.8 (m, 2H), 7.53 (d, 1H, J=7.8 Hz), 7.3-7.4 (m, 1H), 4.62 (d, 2H, J=5.7 Hz); ¹⁹F-NMR (376 MHz; DMSO-d6) δ −56.74, −74.29; MS (APCI⁺) m/z 447.0 (M+1), (APCI−) m/z 445.0 (M−1); HPLC UV purity, Rt=9.449 min, 98.8%; melting point=142-143° C.

### Example 51: Synthesis of cis-2-(3-(2-carbamoyl-6-(trifluoromethoxy)-1H-indol-1-yl)phenyl)-2-methylcyclopropane-1-carboxylic acid (Compound 38a)

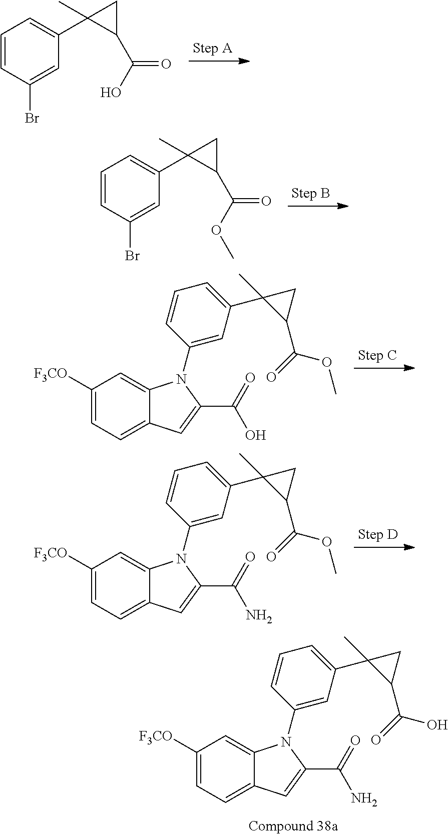

### Step A: Preparation of cis-2-(3-bromophenyl)-2-methylcyclopropane-1-carboxylate

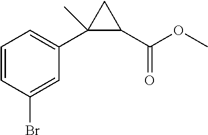

To a mixture consisting of 2-(3-bromophenyl)-2-methyl cyclopropane carboxylic acid (Princeton Bio, 0.50 g, 2.0 mmol) in DMF (5 mL) was added potassium carbonate (0.415 g, 3.00 mmol) dropwise. The reaction was stirred at room temperature for 5 minutes then iodomethane (Oakwood, 0.25 mL, 4.0 mmol) was added dropwise. The reaction mixture was stirred at room temperature for 3 hours. The reaction mixture was cooled to 0° C. before water (15 mL) was added to quench the reaction. The reaction mixture was subsequently partitioned between MTBE (40 mL) and water (30 mL). The phases were separated, and the organic phase was washed with saturated aqueous sodium bicarbonate (25 mL), followed by an additional water (25 mL) wash. The organic layer was concentrated under reduced pressure to afford the crude compound as a yellow oil (0.511 g). The crude oil was purified by flash silica column chromatography on a CombiFlash NextGen 300+ purification system. Elution through a 12 g RediSep Gold Rf flash silica cartridge with 4-50% ethyl acetate in heptane afforded the title compound as a colorless oil (0.213 g, 40% yield). R_{f} 0.65 with 30:70 v/v ethyl acetate-heptane (UV 254 nM); ¹H-NMR (400 MHz; CDCl₃) δ 7.42 (t, 1H, J=1.7 Hz), 7.34 (td, 1H, J=1.6, 7.3 Hz), 7.19 (td, 1H, J=1.5, 7.7 Hz), 7.15 (t, 1H, J=7.6 Hz), 3.46 (s, 3H), 1.94 (dd, 1H, J=5.4, 7.9 Hz), 1.75 (t, 1H, J=5.1 Hz), 1.45 (s, 3H), 1.19 (dd, 1H, J=4.8, 7.8 Hz); MS (APCI+) m/z 269.0 (M+1); HPLC UV purity, Rt=9.160 min, 99.4%.

### Step B: Preparation of cis-1-(3-(2-(methoxycarbonyl)-1-methylcyclopropyl)phenyl)-6-(trifluoromethoxy)-1H-indole-2-carboxylic acid

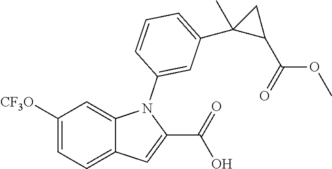

To a mixture consisting of 6-(trifluoromethoxy)-1H-indole-2-carboxylic acid (ChemShuttle, 0.317 g, 1.29 mmol), cis-2-(3-bromophenyl)-2-methylcyclopropane-1-carboxylate (0.348 g, 1.29 mmol), copper (II) acetate (CombiBlocks, 0.234 g, 1.29 mmol), methyl-α-D-glucopyranoside (CombiBlocks, 0.250 g, 1.29 mmol), potassium iodide (VWR, 0.428 g, 2.58 mmol) in DMSO (20 mL) was added DBU (Oakwood Chemicals, 0.58 mL, 3.87 mmol). The reaction mixture was heated to 110° C. under N₂ atmosphere for 12 hours and then allowed to cool to room temperature overnight. The reaction mixture was subsequently partitioned between ethyl acetate (50 mL) and 1M KHSO₄ (40 mL). An emulsion formed which was broken up by the addition of 25 mL of brine. The phases were separated, and the organic phase was partitioned with H₂O (40 mL), followed by brine (40 mL). The organic layer was separated and dried over anhydrous magnesium sulfate, filtered, and concentrated under reduced pressure to give a crude brown oil, 0.789 g. The crude oil was purified by flash silica column chromatography on a CombiFlash NextGen 300+ purification system. Elution through a 24 g RediSep Gold Rf flash silica cartridge with 5-15% ethyl acetate in heptane containing 1% acetic acid afforded the title compound as a white solid (0.130 g, 23% yield). R_{f} 0.38 with 1:30:70 v/v acetic acid-ethyl acetate-heptane (UV 254 nM); ¹H-NMR (400 MHz; DMSO-d6) δ 12.97 (br s, 1H), 7.88 (d, 1H, J=8.7 Hz), 7.39-7.47 (m, 3H), 7.25 (s, 1H), 7.17-7.20 (m, 2H), 6.91 (br s, 1H), 3.35 (s, 3H), 2.01-2.05 (m, 1H), 1.70 (br t, 1H, J=4.6 Hz), 1.48 (s, 3H), 1.22-1.28 (m, 2H); ¹⁹F-NMR (376 MHz; DMSO-d6) δ −56.80; MS (APCI+) m/z 416.0 (M+1-H₂O), (APCI−) m/z 432.0 (M−1); HPLC UV purity, Rt=9.79 min, 98.5%.

### Step C: Preparation of cis-methyl 2-(3-(2-carbamoyl-6-(trifluoromethoxy)-1H-indol-1-yl)phenyl)-2-methylcyclopropane-1-carboxylate

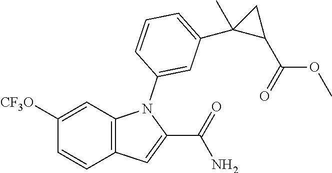

To a mixture consisting of cis-1-(3-(2-(methoxycarbonyl)-1-methylcyclopropyl)phenyl)-6-(trifluoromethoxy)-1H-indole-2-carboxylic acid (0.130 g, 0.300 mmol) in DMF (2 mL) was added ammonium chloride (Chem-Impex, 0.050 g, 0.90 mmol). Next add TBTU (Oakwood, 0.145 g, 0.45 mmol), followed by the addition of DIPEA (0.45 mL, 2.7 mmol). The reaction mixture was stirred for 72 hours at room temperature under N₂ atmosphere. The reaction mixture was subsequently partitioned between ethyl acetate (20 mL) and H₂O (15 mL). The phases were separated, and the organic phase was partitioned with 1M KHSO₄ (15 mL), followed by brine (15 mL). The organic layer was concentrated under reduced pressure to afford the crude product as a colorless oil (0.135 g). The crude product was purified by flash silica column chromatography on a CombiFlash NextGen 300+ purification system. Elution through a 12 g RediSep Gold Rf flash silica cartridge with 10-70% ethyl acetate in heptane containing 1% acetic acid afforded the title compound as a white solid (0.100 g, 78% yield). R_{f} 0.28 with 1:40:60 v/v acetic acid-ethyl acetate-heptane (UV 254 nM); ¹H-NMR (400 MHz; DMSO-d6) δ 7.99 (br s, 1H), 7.83 (d, 1H, J=8.7 Hz), 7.35-7.45 (m, 3H), 7.28 (s, 1H), 7.21 (s, 1H), 7.15 (br d, 1H, J=8.7 Hz), 7.11 (br d, 1H, J=7.8 Hz), 6.97 (br s, 1H), 3.35 (s, 3H), 2.01-2.05 (m, 1H), 1.69 (t, 1H, J=4.9 Hz), 1.48 (s, 3H), 1.22-1.28 (m, 2H); ¹⁹F-NMR (376 MHz; DMSO-d6) δ −56.83; MS (APCI+) m/z 433.0 (M+1), (APCI−) m/z 431.0 (M−1); HPLC UV purity, Rt=9.04 min, 100%.

### Step D: Preparation of cis-2-(3-(2-carbamoyl-6-(trifluoromethoxy)-1H-indol-1-yl)phenyl)-2-methylcyclopropane-1-carboxylic acid (Compound 38a)

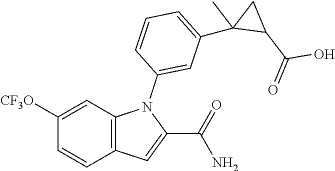

To a mixture consisting of cis-methyl 2-(3-(2-carbamoyl-6-(trifluoromethoxy)-1H-indol-1-yl)phenyl)-2-methylcyclopropane-1-carboxylate (0.100 g, 0.231 mmol) in methanol (3 mL) was added 1M lithium hydroxide (2.00 mL, 2.00 mmol). The reaction mixture was stirred at room temperature overnight under N₂ atmosphere. The reaction was not complete after stirring overnight so the reaction was heated to 55° C. for 6 hours and then allowed to stir overnight at room temperature. The reaction mixture was subsequently partitioned between ethyl acetate (50 mL) and 1M HCl (25 mL). The phases were separated, and the organic phase was partitioned with water (25 mL), followed by brine (25 mL). The organic layer was concentrated under reduced pressure to afford the crude compound as a colorless oil (0.110 g). The crude product was purified by flash silica column chromatography on a CombiFlash NextGen 300+ purification system. Elution through a 12 g RediSep Gold Rf flash silica cartridge with 10-80% ethyl acetate in heptane containing 1% acetic acid afforded the title compound as a white solid (0.063 g, 66% yield). R_{f} 0.14 with 1:40:60 v/v acetic acid-ethyl acetate-heptane (UV 254 nM); ¹H-NMR (400 MHz; DMSO-d6) δ 11.90 (br s, 1H), 8.00 (br s, 1H), 7.83 (d, 1H, J=8.7 Hz), 7.37-7.44 (m, 3H), 7.27 (s, 1H), 7.24 (s, 1H), 7.11-7.17 (m, 3H), 1.90-1.94 (m, 1H), 1.63 (t, 1H, J=4.8 Hz), 1.48 (s, 3H), 1.20-1.30 (m, 1H); ¹⁹F-NMR (376 MHz; DMSO-d6) δ −56.73; MS (APCI⁺) m/z 419.0 (M+1), 417.0 (M−1); HPLC UV purity, Rt=8.012 min, 99.1%; melting point=122-123° C.

### Example 52: Synthesis of trans-2-(3-(2-carbamoyl-6-(trifluoromethoxy)-1H-indol-1-yl)phenyl)-2-methylcyclopropane-1-carboxylic acid (Compound 38b)

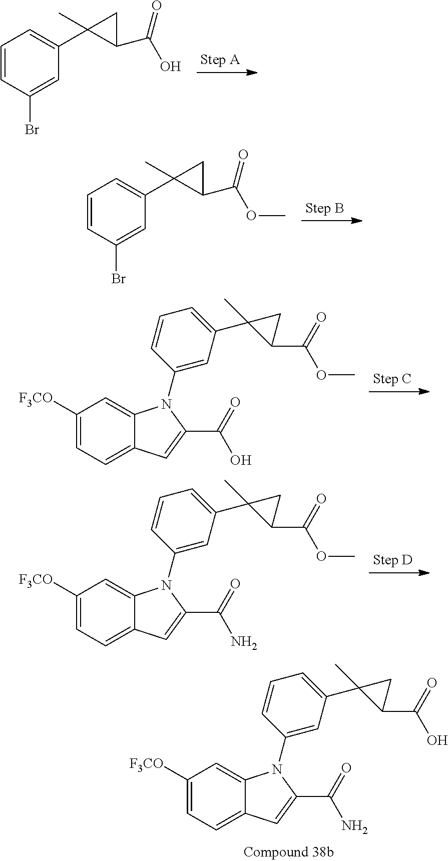

### Step A: Preparation of trans-2-(3-bromophenyl)-2-methylcyclopropane-1-carboxylate

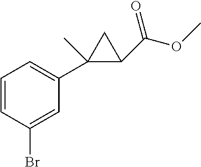

To a mixture consisting of 2-(3-bromophenyl)-2-methyl cyclopropane carboxylic acid (Princeton Bio, 0.50 g, 2.0 mmol) in DMF (5 mL) was added potassium carbonate (0.415 g, 3.00 mmol) dropwise. The reaction was stirred at room temperature for 5 minutes then iodomethane (Oakwood, 0.25 mL, 4.0 mmol) was added dropwise. The reaction mixture was stirred at room temperature for 3 hours. The reaction mixture was cooled to 0° C. before water (15 mL) was added to quench the reaction. The reaction mixture was subsequently partitioned between MTBE (40 mL) and water (30 mL). The phases were separated, and the organic phase was washed with saturated aqueous sodium bicarbonate (25 mL), followed by an additional water (25 mL) wash. The organic layer was concentrated under reduced pressure to afford the crude compound as a yellow oil (0.511 g). The crude oil was purified by flash silica column chromatography on a CombiFlash NextGen 300+ purification system. Elution through a 12 g RediSep Gold Rf flash silica cartridge with 4-50% ethyl acetate in heptane afforded the title compound as a colorless oil (0.174 g, 32% yield). R_{f} 0.68 with 30:70 v/v ethyl acetate-heptane (UV 254 nM); ¹H-NMR (400 MHz; CDCl₃) δ 7.45 (t, 1H, J=1.8 Hz), 7.35 (ddd, 1H, J=1.2, 1.3, 7.5 Hz), 7.23 (td, 1H, J=1.4, 7.8 Hz), 7.17 (t, 1H, J=7.7 Hz), 3.75 (s, 3H), 1.96 (dd, 1H, J=6.0, 8.5 Hz), 1.51 (s, 3H), 1.46 (dd, 1H, J=4.8, 6.0 Hz), 1.42 (dd, 1H, J=4.8, 8.5 Hz); MS (APCI+) m/z 269.0 (M+1); HPLC UV purity, Rt=9.757 min, 99.4%.

### Step B: Preparation of trans-1-(3-(2-(methoxycarbonyl)-1-methylcyclopropyl)phenyl)-6-(trifluoromethoxy)-1H-indole-2-carboxylic acid

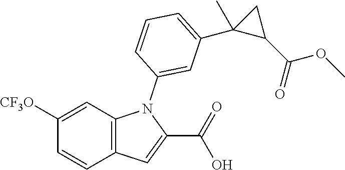

To a mixture consisting of 6-(trifluoromethoxy)-1H-indole-2-carboxylic acid (ChemShuttle, 0.245 g, 1.00 mmol), trans-2-(3-bromophenyl)-2-methylcyclopropane-1-carboxylate (0.269 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (15 mL) was added DBU (Oakwood Chemicals, 0.45 mL, 3.00 mmol). The reaction mixture was heated to 110° C. under N₂ atmosphere for 12 hours and then allowed to cool to room temperature overnight. The reaction mixture was subsequently partitioned between ethyl acetate (50 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude brown oil, 0.579 g. The crude oil was purified by flash silica column chromatography on a CombiFlash NextGen 300+ purification system. Elution through a 24 g RediSep Gold Rf flash silica cartridge with 5-30% ethyl acetate in heptane containing 1% acetic acid afforded the title compound as a white solid (0.055 g, 13% yield). R_{f} 0.38 with 1:30:70 v/v acetic acid-ethyl acetate-heptane (UV 254 nM); MS (APCI+) m/z 416.0 (M+1-H₂O), (APCI−) m/z 432.0 (M−1); HPLC UV purity, Rt=9.955 min, 96.5%.

### Step C: Preparation of trans-methyl 2-(3-(2-carbamoyl-6-(trifluoromethoxy)-1H-indol-1-yl)phenyl)-2-methylcyclopropane-1-carboxylate

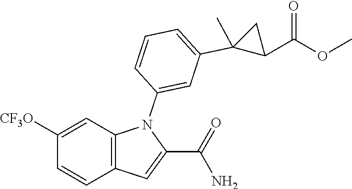

To a mixture consisting of trans-i-(3-(2-(methoxycarbonyl)-1-methylcyclopropyl)phenyl)-6-(trifluoromethoxy)-1H-indole-2-carboxylic acid (0.055 g, 0.126 mmol) in DMF (3 mL) was added ammonium chloride (Chem-Impex, 0.020 g, 0.38 mmol). Next add TBTU (Oakwood, 0.061 g, 0.19 mmol), followed by the addition of DIPEA (0.19 mL, 1.13 mmol). The reaction mixture was stirred for 72 hours at room temperature under N₂ atmosphere. The reaction mixture was subsequently partitioned between ethyl acetate (25 mL) and H₂O (15 mL). The phases were separated, and the organic phase was partitioned with 1M KHSO₄ (15 mL), followed by brine (15 mL). The organic layer was concentrated under reduced pressure to afford the crude product as a colorless oil (0.066 g). The crude product was purified by flash silica column chromatography on a CombiFlash NextGen 300+ purification system. Elution through a 12 g RediSep Gold Rf flash silica cartridge with 5-50% ethyl acetate in heptane containing 1% acetic acid afforded the title compound as a white solid (0.043 g, 80% yield). R_{f} 0.14 with 1:30:70 v/v acetic acid-ethyl acetate-heptane (UV 254 nM); MS (APCI+) m/z 433.0 (M+1), (APCI−) m/z 431.0 (M−1).

### Step D: Preparation of trans-2-(3-(2-carbamoyl-6-(trifluoromethoxy)-1H-indol-1-yl)phenyl)-2-methylcyclopropane-1-carboxylic acid (Compound 38b)

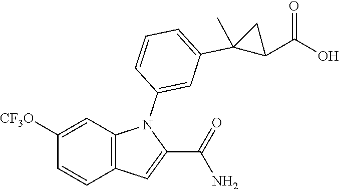

To a mixture consisting of trans-methyl 2-(3-(2-carbamoyl-6-(trifluoromethoxy)-1H-indol-1-yl)phenyl)-2-methylcyclopropane-1-carboxylate (0.043 g, 0.100 mmol) in methanol (2 mL) was added 1M lithium hydroxide (1.40 mL, 1.40 mmol). The reaction mixture was stirred at room temperature overnight under N₂ atmosphere. The reaction mixture was subsequently partitioned between ethyl acetate (15 mL) and 1M HCl (8 mL). The phases were separated, and the organic phase was partitioned with water (10 mL), followed by brine (10 mL). The organic layer was concentrated under reduced pressure to afford the crude compound as a colorless oil (0.033 g). The crude product was purified by flash silica column chromatography on a CombiFlash NextGen 300+ purification system. Elution through a 4 g RediSep Gold Rf flash silica cartridge with 10-80% ethyl acetate in heptane containing 1% acetic acid afforded the title compound as a white solid (0.014 g, 34% yield). R_{f} 0.27 with 1:60:40 v/v acetic acid-ethyl acetate-heptane (UV 254 nM); ¹H-NMR (400 MHz; DMSO-d6) δ 12.31 (br s, 1H), 8.03 (br s, 1H), 7.84 (d, 1H, J=8.7 Hz), 7.40-7.52 (m, 2H), 7.38 (br d, 1H, J=8.0 Hz), 7.29 (s, 1H), 7.21-7.24 (m, 2H), 7.16 (br d, 1H, J=8.7 Hz), 6.97 (s, 1H), 1.91 (t, 1H, J=4.8 Hz), 1.50 (s, 3H), 1.25-1.30 (m, 2H); ¹⁹F-NMR (376 MHz; DMSO-d6) δ −56.73; MS (APCI⁺) m/z 419.0 (M+1), 417.0 (M−1); HPLC UV purity, Rt=7.8 min, 92.1%.

### Example 53: Synthesis of 1-(6-(1-carboxycyclopropyl)pyridin-2-yl)-6-(trifluoromethoxy)-1H-indole-2-carboxylic acid (Compound 39)

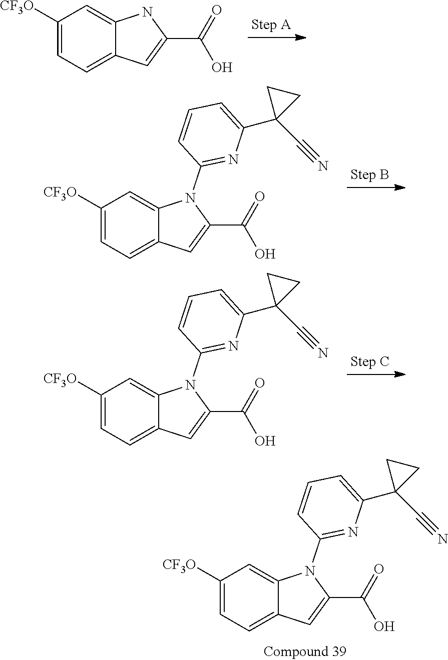

### Step A: Preparation of 1-(6-(1-cyanocyclopropyl)pyridin-2-yl)-6-(trifluoromethoxy)-1H-indole-2-carboxylic acid

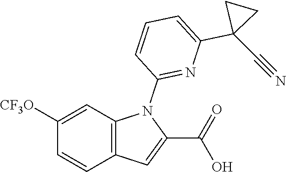

To a mixture consisting of 6-(trifluoromethoxy)-1H-indole-2-carboxylic acid (ChemShuttle, 0.368 g, 1.50 mmol), 1-(6-Bromopyridin-2-y)cyclopropanecarbonitrile (1ClickChemistry, 0.334 g, 1.50 mmol), copper (II) acetate (CombiBlocks, 0.272 g, 1.50 mmol), methyl-α-D-glucopyranoside (CombiBlocks, 0.291 g, 1.50 mmol), potassium iodide (VWR, 0.498 g, 3.00 mmol) in DMSO (15 mL) was added DBU (Oakwood Chemicals, 0.67 mL, 4.50 mmol). The reaction mixture was heated to 105° C. under N₂ atmosphere for 7 hours and then allowed to cool to room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude brown oil, 1.61 g. The crude oil was purified by flash silica column chromatography on a CombiFlash NextGen 300+ purification system. Elution through a 12 g RediSep Gold Rf flash silica cartridge with 5-80% ethyl acetate in heptane containing 1% acetic acid afforded the title compound as a white solid (0.066 g, 12% yield). R_{f} 0.42 with 1:40:60 v/v acetic acid-ethyl acetate-heptane (UV 254 nM); MS (APCI+) m/z 388.0 (M+1), (APCI−) m/z 386.0 (M−1); HPLC UV purity, Rt=8.992 min, 97.3%.

### Step B: Preparation of 1-(6-(1-cyanocyclopropyl)pyridin-2-yl)-6-(trifluoromethoxy)-1H-indole-2-carboxamide

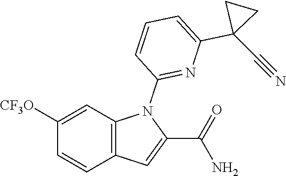

To a mixture consisting of 1-(6-(1-cyanocyclopropyl)pyridin-2-yl)-6-(trifluoromethoxy)-1H-indole-2-carboxylic acid (0.066 g, 0.172 mmol) in DMF (2 mL) was added ammonium chloride (Chem-Impex, 0.028 g, 0.52 mmol). Next add TBTU (Oakwood, 0.083 g, 0.26 mmol), followed by the addition of DIPEA (0.28 mL, 1.55 mmol). The reaction mixture was stirred for 72 hours at room temperature under N₂ atmosphere. The reaction mixture was subsequently partitioned between ethyl acetate (25 mL) and H₂O (25 mL). The phases were separated, and the organic phase was partitioned with 1M KHSO₄ (15 mL), followed by brine (15 mL). The organic layer was concentrated under reduced pressure to afford the crude product as a white solid (0.059 g, 90% yield). R_{f} 0.23 with 1:40:60 v/v acetic acid-ethyl acetate-heptane (UV 254 nM); MS (APCI+) m/z 387.0 (M+1), (APCI−) m/z 386.0 (M−1); HPLC UV purity, Rt=8.184 min, 85.7%. The product was used without further purification or characterization.

### Step C: Preparation of 1-(6-(1-carboxycyclopropyl)pyridin-2-yl)-6-(trifluoromethoxy)-1H-indole-2-carboxylic acid (Compound 39)

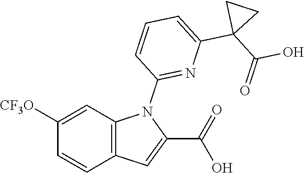

To a mixture consisting of 1-(6-(1-cyanocyclopropyl)pyridin-2-yl)-6-(trifluoromethoxy)-1H-indole-2-carboxamide (0.059 g, 0.153 mmol) in ethanol (1.5 mL) was added potassium hydroxide (3M aqueous solution, 2.00 mL, 6.00 mmol). The reaction mixture was heated to 90° C. for 5 hours and then allowed to cool to room temperature. The reaction mixture was subsequently partitioned between ethyl acetate (10 mL) and 1M HCl (25 mL). The phases were separated, and the organic phase was partitioned with water (10 mL), followed by brine (10 mL). The organic layer was concentrated under reduced pressure to afford the crude compound as a white solid (0.049 g). The crude product was purified by flash silica column chromatography on a CombiFlash NextGen 300+ purification system. Elution through a 4 g RediSep Gold Rf flash silica cartridge with 10-80% ethyl acetate in heptane containing 1% acetic acid afforded the title compound as a white solid (0.042 g, 67% yield). R_{f} 0.20 with 1:40:60 v/v acetic acid-ethyl acetate-heptane (UV 254 nM); ¹H-NMR (400 MHz; DMSO-d6) δ 13.12 (br s, 1H), 12.74 (br s, 1H), 7.96 (t, 1H, J=7.8 Hz), 7.89 (d, 1H, J=8.7 Hz), 7.69 (d, 1H, J=7.6 Hz), 7.46 (s, 1H), 7.40-7.42 (m, 2H), 7.22 (br d, 1H, J=8.7 Hz), 1.52-1.54 (m, 2H), 1.39-1.42 (m, 2H); ¹⁹F-NMR (376 MHz; DMSO-d6) δ −56.69; MS (APCI⁺) m/z 407.0 (M+1), 405.0 (M−1); HPLC UV purity, Rt=7.728 min, 100%; melting point=200-201° C.

### Example 54: Synthesis of (S)-3-amino-3-(3-(2-carbamoyl-6-(trifluoromethoxy)-1H-indol-1-yl)phenyl)propanoic acid (Compound 40) and (S)-1-(3-(1-amino-2-carboxyethyl)phenyl)-6-(trifluoromethoxy)-1H-indole-2-carboxylic acid (Compound 41)

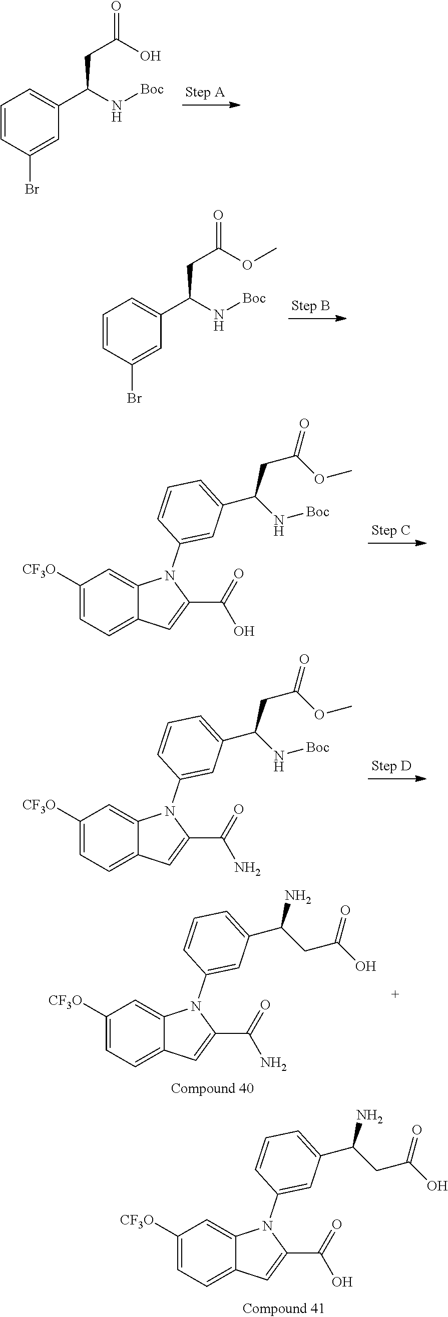

### Step A: Preparation of methyl (R)-3-(3-bromophenyl)-3-((tert-butoxycarbonyl)amino)propanoate

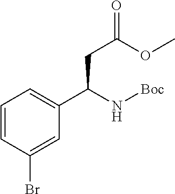 [SMILES: COC(=O)C[C@@H](NC(=O)OC(C)(C)C)c1cccc(Br)c1]

To a mixture consisting of (R)-3-(3-bromophenyl)-3-((tert-butoxycarbonyl)amino)propanoic acid (Ambeed, 0.688 g, 2.0 mmol) in DMF (5 mL) was added potassium carbonate (0.415 g, 3.00 mmol) dropwise. The reaction was stirred at room temperature for 5 minutes then iodomethane (Oakwood, 0.25 mL, 4.0 mmol) was added dropwise. The reaction mixture was stirred at room temperature for 3 hours. The reaction mixture was cooled to 0° C. before water (15 mL) was added to quench the reaction. The reaction mixture was subsequently partitioned between MTBE (40 mL) and water (30 mL). The phases were separated, and the organic phase was washed with saturated aqueous sodium bicarbonate (25 mL), followed by an additional water (25 mL) wash. The organic layer was concentrated under reduced pressure to afford the crude compound as a colorless oil (0.788 g). The crude oil was purified by flash silica column chromatography on a CombiFlash NextGen 300+ purification system. Elution through a 24 g RediSep Gold Rf flash silica cartridge with 2-30% ethyl acetate in heptane afforded the title compound as a white solid (0.716 g, quantitative yield). R_{f} 0.45 with 30:70 v/v ethyl acetate-heptane (UV 254 nM); ¹H-NMR (400 MHz; CDCl₃) δ 7.45 (s, 1H), 7.40 (td, 1H, J=1.7, 7.2 Hz), 7.19-7.25 (m, 2H), 5.55 (br s, 1H), 5.07 (br s, 1H), 3.64 (s, 3H), 2.83 (br s, 2H), 1.44 (br s, 9H); MS (APCI+) m/z 380.0 (M+Na); HPLC UV purity, Rt=8.819 min, 100%.

### Step B: Preparation of (R)-1-(3-(1-((tert-butoxycarbonyl)amino)-3-methoxy-3-oxopropyl)phenyl)-6-(trifluoromethoxy)-1H-indole-2-carboxylic acid

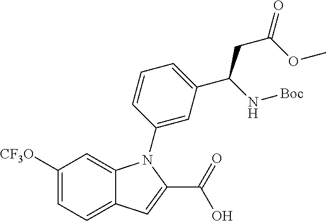

To a mixture consisting of 6-(trifluoromethoxy)-1H-indole-2-carboxylic acid (ChemShuttle, 0.245 g, 1.00 mmol), methyl (R)-3-(3-bromophenyl)-3-((tert-butoxycarbonyl)amino)propanoate (0.358 g, 1.00 mmol), copper (II) acetate (CombiBlocks, 0.182 g, 1.00 mmol), methyl-α-D-glucopyranoside (CombiBlocks, 0.194 g, 1.00 mmol), potassium iodide (VWR, 0.332 g, 2.00 mmol) in DMSO (20 mL) was added DBU (Oakwood Chemicals, 0.45 mL, 3.00 mmol). The reaction mixture was heated to 115° C. under N₂ atmosphere for 30 hours and then allowed to cool to room temperature. The reaction mixture was subsequently partitioned between ethyl acetate (50 mL) and 1M KHSO₄ (40 mL). An emulsion formed which was broken up by the addition of 25 mL of brine. The phases were separated, and the organic phase was partitioned with H₂O (40 mL), followed by brine (40 mL). The organic layer was separated and dried over anhydrous magnesium sulfate, filtered, and concentrated under reduced pressure to give a crude brown oil, 0.737 g. The crude oil was purified by flash silica column chromatography on a CombiFlash NextGen 300+ purification system. Elution through a 24 g RediSep Gold Rf flash silica cartridge with 5-75% ethyl acetate in heptane containing 1% acetic acid afforded the title compound as a yellow solid (0.050 g, 9% yield). R_{f} 0.21 with 1:30:70 v/v acetic acid-ethyl acetate-heptane (UV 254 nM); MS (APCI+) m/z 423.0 (M+1-Boc), (APCI−) m/z 521.0 (M−1). The product obtained was used in the next reaction without further characterization.

### Step C: Preparation of methyl (R)-3-((tert-butoxycarbonyl)amino)-3-(3-(2-carbamoyl-6-(trifluoromethoxy)-1H-indol-1-yl)phenyl)propanoate

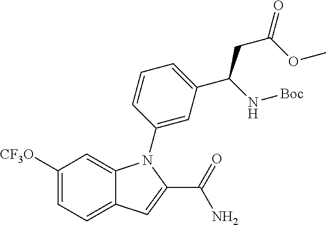

To a mixture consisting of (R)-1-(3-(1-((tert-butoxycarbonyl)amino)-3-methoxy-3-oxopropyl)phenyl)-6-(trifluoromethoxy)-1H-indole-2-carboxylic acid (0.050 g, 0.096 mmol) in DMF (3 mL) was added ammonium chloride (Chem-Impex, 0.015 g, 0.29 mmol). Next add TBTU (Oakwood, 0.046 g, 0.14 mmol), followed by the addition of DIPEA (0.15 mL, 0.86 mmol). The reaction mixture was stirred overnight at room temperature under N₂ atmosphere. The reaction mixture was subsequently partitioned between ethyl acetate (30 mL) and H₂O (20 mL). The phases were separated, and the organic phase was partitioned with 1M KHSO₄ (15 mL), followed by brine (15 mL). The organic layer was concentrated under reduced pressure to afford the crude product as an orange oil (0.055 g, quantitative yield). R_{f} 0.17 with 1:40:60 v/v acetic acid-ethyl acetate-heptane (UV 254 nM); MS (APCI+) m/z 544 (M+Na+), (APCI−) m/z 566.0 (M+Cl); HPLC UV purity, Rt=8.816 min, 76.6%. The product was used without further purification or characterization.

### Step D: Preparation of (S)-3-amino-3-(3-(2-carbamoyl-6-(trifluoromethoxy)-1H-indol-1-yl)phenyl)propanoic acid (Compound 40) and (S)-1-(3-(1-amino-2-carboxyethyl)phenyl)-6-(trifluoromethoxy)-1H-indole-2-carboxylic acid (Compound 41)

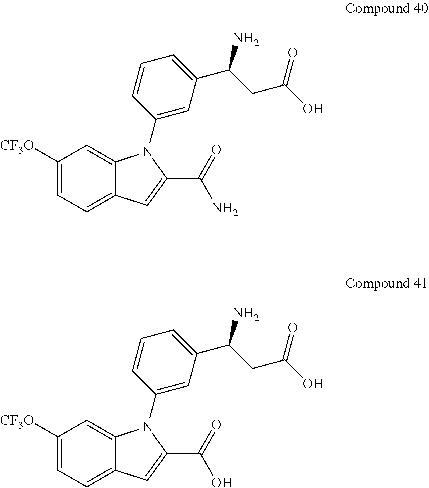

To a mixture consisting of methyl (R)-3-((tert-butoxycarbonyl)amino)-3-(3-(2-carbamoyl-6-(trifluoromethoxy)-1H-indol-1-yl)phenyl)propanoate (0.055 g, 0.105 mmol) in 1,4-Dioxane (1 mL) was added 6N HCl (1.00 mL, 6.00 mmol). The reaction mixture was stirred at 85° C. under N₂ atmosphere for 2 hours and then allowed to cool to room temperature. Water (15 mL) was added, and then the aqueous layer was separated. The aqueous layer was adjusted to pH=8-9 with saturated aqueous sodium carbonate, and then extracted with ethyl acetate (2×15 mL). The phases were separated, and the combined organic phase was washed with brine (25 mL). The organic layer was concentrated under reduced pressure to afford the crude compound as a colorless oil (0.045 g). The crude oil was purified by flash silica column chromatography on a CombiFlash NextGen 300+ purification system. Elution through a 4 g RediSep Gold Rf flash silica cartridge with 1-10% methanol in dichloromethane containing 1% acetic acid afforded the two title compounds as a white solid.

Compound 40 (0.005 g) Rf 0.05 with 1:20:80 v/v acetic acid-methanol-dichloromethane (UV 254 nM); MS (APCI+) m/z 408.0 (M+1), (APCI−) m/z 406.0 (M−1); HPLC UV purity, Rt=5.67 min, 45.4%.

Compound 41 (0.007 g) R_{f} 0.06 with 1:20:80 v/v acetic acid-methanol-dichloromethane (UV 254 nM); ¹H-NMR (400 MHz; CDCl₃) δ 7.66 (d, 1H, J=8.7 Hz), 7.33-7.41 (m, 3H), 7.16 (br d, 1H, J=8.5 Hz), 7.02 (br d, 1H, J=8.5 Hz), 6.94 (s, 1H), 6.81 (s, 1H), 4.24 (br s, 1H), 2.31 (br s, 2H); ¹⁹F-NMR (376 MHz; DMSO-d6) δ −56.67; MS (APCI+) m/z 409.0 (M+1), (APCI−) m/z 407.0 (M−1); HPLC UV purity, Rt=6.17 min, 96.5%.

### Example 55: Synthesis of (R)-3-amino-3-(3-(2-carbamoyl-6-(trifluoromethoxy)-1H-indol-1-yl)phenyl)propanoic acid (Compound 42) and (R)-1-(3-(1-amino-2-carboxyethyl)phenyl)-6-(trifluoromethoxy)-1H-indole-2-carboxylic acid (Compound 43)

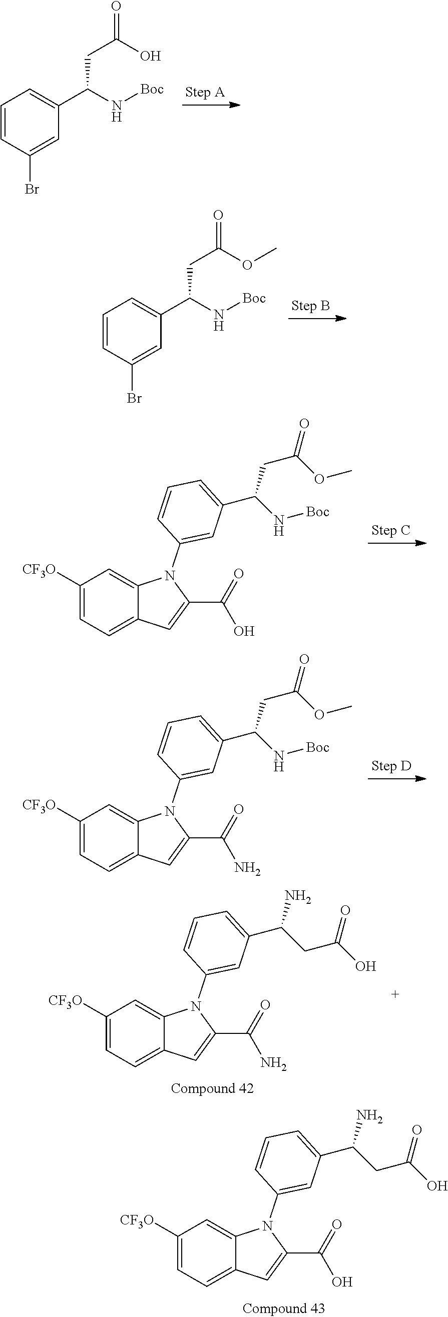

### Step A: Preparation of methyl (S)-3-(3-bromophenyl)-3-((tert-butoxycarbonyl)amino)propanoate

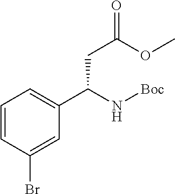 [SMILES: COC(=O)C[C@H](NC(=O)OC(C)(C)C)c1cccc(Br)c1]

To a mixture consisting of (S)-3-(3-bromophenyl)-3-((tert-butoxycarbonyl)amino)propanoic acid (Ambeed, 0.688 g, 2.0 mmol) in DMF (5 mL) was added potassium carbonate (0.415 g, 3.00 mmol) dropwise. The reaction was stirred at room temperature for 5 minutes then iodomethane (Oakwood, 0.25 mL, 4.0 mmol) was added dropwise. The reaction mixture was stirred at room temperature for 3 hours. The reaction mixture was cooled to 0° C. before water (15 mL) was added to quench the reaction. The reaction mixture was subsequently partitioned between MTBE (40 mL) and water (30 mL). The phases were separated, and the organic phase was washed with saturated aqueous sodium bicarbonate (25 mL), followed by an additional water (25 mL) wash. The organic layer was concentrated under reduced pressure to afford the crude compound as a colorless oil (0.788 g). The crude oil was purified by flash silica column chromatography on a CombiFlash NextGen 300+ purification system. Elution through a 24 g RediSep Gold Rf flash silica cartridge with 2-30% ethyl acetate in heptane afforded the title compound as a white solid (0.716 g, quantitative yield). R_{f} 0.48 with 30:70 v/v ethyl acetate-heptane (UV 254 nM); ¹H-NMR (400 MHz; CDCl₃) δ 7.44 (s, 1H), 7.40 (td, 1H, J=1.8, 7.2 Hz), 7.19-7.25 (m, 2H), 5.55 (br s, 1H), 5.07 (br s, 1H), 3.64 (s, 3H), 2.83 (br s, 2H), 1.44 (br s, 9H); MS (APCI+) m/z 258.0 (M+1-Boc); HPLC UV purity, Rt=8.878 min, 99.5%.

### Step B: Preparation of (S)-1-(3-(1-((tert-butoxycarbonyl)amino)-3-methoxy-3-oxopropyl)phenyl)-6-(trifluoromethoxy)-1H-indole-2-carboxylic acid

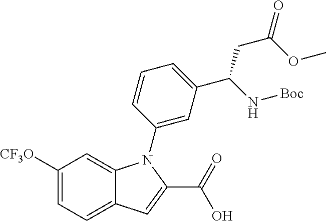

To a mixture consisting of 6-(trifluoromethoxy)-1H-indole-2-carboxylic acid (ChemShuttle, 0.184 g, 0.751 mmol), methyl (S)-3-(3-bromophenyl)-3-((tert-butoxycarbonyl)amino)propanoate (0.269 g, 0.75 mmol), copper (II) acetate (CombiBlocks, 0.136 g, 0.75 mmol), methyl-α-D-glucopyranoside (CombiBlocks, 0.146 g, 0.75 mmol), potassium iodide (VWR, 0.249 g, 1.50 mmol) in DMSO (20 mL) was added DBU (Oakwood Chemicals, 0.33 mL, 2.25 mmol). The reaction mixture was heated to 115° C. under N₂ atmosphere for 30 hours and then allowed to cool to room temperature. The reaction mixture was subsequently partitioned between ethyl acetate (50 mL) and 1M KHSO₄ (40 mL). An emulsion formed which was broken up by the addition of 25 mL of brine. The phases were separated, and the organic phase was partitioned with H₂O (40 mL), followed by brine (40 mL). The organic layer was separated and dried over anhydrous magnesium sulfate, filtered, and concentrated under reduced pressure to give a crude brown oil, 0.656 g. The crude oil was purified by flash silica column chromatography on a CombiFlash NextGen 300+ purification system. Elution through a 24 g RediSep Gold Rf flash silica cartridge with 5-75% ethyl acetate in heptane containing 1% acetic acid afforded the title compound as a yellow solid (0.093 g, 14% yield). R_{f} 0.21 with 1:30:70 v/v acetic acid-ethyl acetate-heptane (UV 254 nM); MS (APCI+) m/z 423.0 (M+1-Boc), (APCI−) m/z 521.0 (M−1). The product obtained was used in the next reaction without further characterization.

### Step C: Preparation of methyl (S)-3-((tert-butoxycarbonyl)amino)-3-(3-(2-carbamoyl-6-(trifluoromethoxy)-1H-indol-1-yl)phenyl)propanoate

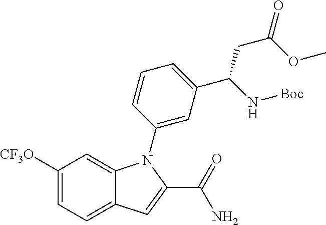

To a mixture consisting of (S)-1-(3-(1-((tert-butoxycarbonyl)amino)-3-methoxy-3-oxopropyl)phenyl)-6-(trifluoromethoxy)-1H-indole-2-carboxylic acid (0.093 g, 0.179 mmol) in DMF (3 mL) was added ammonium chloride (Chem-Impex, 0.028 g, 0.54 mmol). Next add TBTU (Oakwood, 0.086 g, 0.27 mmol), followed by the addition of DIPEA (0.28 mL, 1.61 mmol). The reaction mixture was stirred overnight at room temperature under N₂ atmosphere. The reaction mixture was subsequently partitioned between ethyl acetate (30 mL) and H₂O (20 mL). The phases were separated, and the organic phase was partitioned with 1M KHSO₄ (15 mL), followed by brine (15 mL). The organic layer was concentrated under reduced pressure to afford the crude product as an orange oil (0.106 g, quantitative yield). R_{f} 0.17 with 1:40:60 v/v acetic acid-ethyl acetate-heptane (UV 254 nM); MS (APCI+) m/z 544 (M+Na+), (APCI−) m/z 566.0 (M+Cl); HPLC UV purity, Rt=8.816 min, 72.1%. The product was used without further purification or characterization.

### Step D: Preparation of (R)-3-amino-3-(3-(2-carbamoyl-6-(trifluoromethoxy)-1H-indol-1-yl)phenyl)propanoic acid (Compound 42) and (R)-1-(3-(1-amino-2-carboxyethyl)phenyl)-6-(trifluoromethoxy)-1H-indole-2-carboxylic acid (Compound 43)

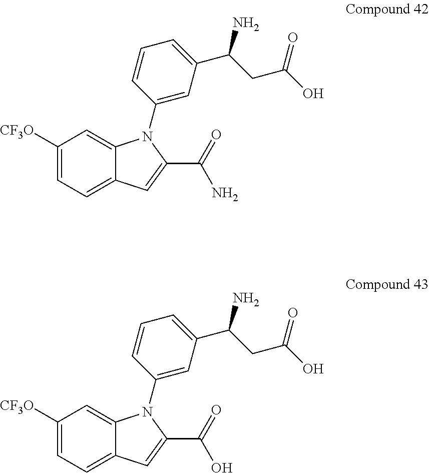

To a mixture consisting of methyl (S)-3-((tert-butoxycarbonyl)amino)-3-(3-(2-carbamoyl-6-(trifluoromethoxy)-1H-indol-1-yl)phenyl)propanoate (0.106 g, 0.203 mmol) in 1,4-Dioxane (1 mL) was added 6N HCl (1.00 mL, 6.00 mmol). The reaction mixture was stirred at 85° C. under N₂ atmosphere for 2 hours and then allowed to cool to room temperature. Water (15 mL) was added, and then the aqueous layer was separated. The aqueous layer was adjusted to pH=8-9 with saturated aqueous sodium carbonate, and then extracted with ethyl acetate (2×15 mL). The phases were separated, and the combined organic phase was washed with brine (25 mL). The organic layer was concentrated under reduced pressure to afford the crude compound as a colorless oil (0.045 g). The crude oil was purified by flash silica column chromatography on a CombiFlash NextGen 300+ purification system. Elution through a 4 g RediSep Gold Rf flash silica cartridge with 1-25% methanol in dichloromethane containing 1% acetic acid afforded the two title compounds as a white solid.

Compound 42 (0.005 g) Rf 0.05 with 1:20:80 v/v acetic acid-methanol-dichloromethane (UV 254 nM); MS (APCI+) m/z 408.0 (M+1), (APCI−) m/z 406.0 (M−1); HPLC UV purity, Rt=5.67 min, 80.0%.

Compound 43 (0.004 g) Rf 0.06 with 1:20:80 v/v acetic acid-methanol-dichloromethane (UV 254 nM); ¹H-NMR (400 MHz; CDCl₃) δ 7.79 (d, 1H, J=8.7 Hz), 7.46-7.51 (m, 3H), 7.30 (br d, 1H, J=6.9 Hz), 7.26 (s, 1H), 7.11 (br d, 1H, J=8.7 Hz), 6.96 (s, 1H), 4.47 (br t, 1H, J=7.0 Hz), 3.53 (br s, 1H)), 2.73 (br dd, 1H, J=8.1, 16.4 Hz), 2.62 (br dd, 1H, J=5.8, 16.4 Hz); ¹⁹F-NMR (376 MHz; DMSO-d6) δ −56.62; MS (APCI+) m/z 409.0 (M+1), (APCI−) m/z 407.0 (M−1); HPLC UV purity, Rt=6.18 min, 98.4%.

### Example 56: Synthesis of 2-((6-(2-carbamoyl-6-(trifluoromethoxy)-1H-indol-1-yl)pyridin-2-yl)thio)acetic acid (Compound 44) and 2-((6-(2-carbamoyl-6-(trifluoromethoxy)-1H-indol-1-yl)pyridin-2-yl)sulfonyl)acetic acid (Compound 45)

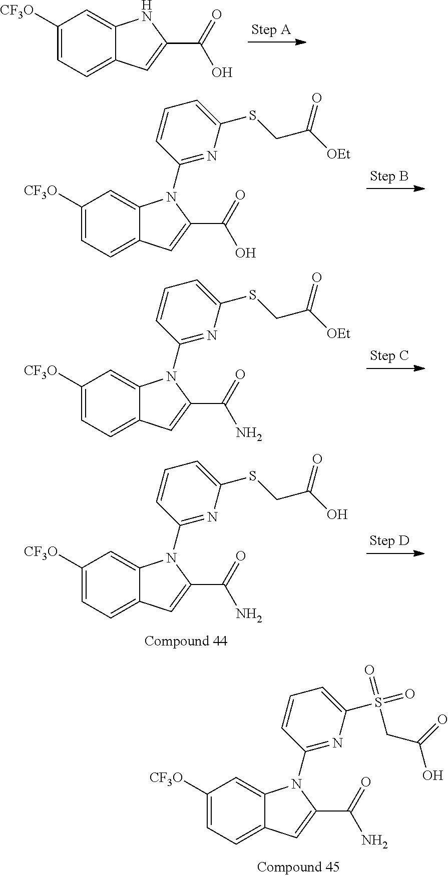

### Step A: Preparation of 1-(6-((2-ethoxy-2-oxoethyl)thio)pyridin-2-yl)-6-(trifluoromethoxy)-1H-indole-2-carboxylic acid

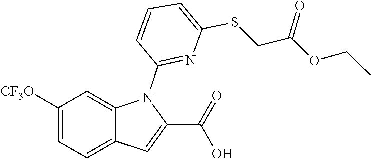

To a mixture consisting of 6-(trifluoromethoxy)-1H-indole-2-carboxylic acid (ChemShuttle, 0.368 g, 1.50 mmol), ethyl 2-((6-bromopyridin-2-yl)thio)acetate (Enamine, 0.414 g, 1.50 mmol), copper (II) acetate (CombiBlocks, 0.272 g, 1.50 mmol), methyl-α-D-glucopyranoside (CombiBlocks, 0.291 g, 1.50 mmol), potassium iodide (VWR, 0.498 g, 3.00 mmol) in DMSO (20 mL) was added DBU (Oakwood Chemicals, 0.67 mL, 4.50 mmol). The reaction mixture was heated to 115° C. under N₂ atmosphere for 6 hours and then allowed to cool to room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.652 g. The crude oil was purified by flash silica column chromatography on a CombiFlash NextGen 300+ purification system. Elution through a 24 g RediSep Gold Rf flash silica cartridge with 5-75% ethyl acetate in hexanes containing 1% acetic acid afforded the title compound as a white solid (0.106 g, 16% yield). R_{f} 0.26 with 1:30:70 v/v acetic acid-ethyl acetate-hexane (UV 254 nM); ¹H-NMR (400 MHz; DMSO-d6) δ 13.15 (br s, 1H), 7.88-7.91 (m, 2H), 7.45-7.50 (m, 2H), 7.22-7.28 (m, 3H), 3.95 (s, 2H), 3.89 (q, 2H, J=7.1 Hz), 0.88 (t, 3H, J=7.1 Hz); ¹⁹F-NMR (376 MHz; DMSO-d6) δ −56.70; MS (APCI+) m/z 441.0 (M+1), (APCI−) m/z 439.0 (M−1); HPLC UV purity, Rt=9.22 min, 91.7%.

### Step B: Preparation of ethyl 2-((6-(2-carbamoyl-6-(trifluoromethoxy)-1H-indol-1-yl)pyridin-2-yl)thio)acetate

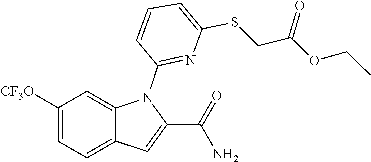

To a mixture consisting of 1-(6-((2-ethoxy-2-oxoethyl)thio)pyridin-2-yl)-6-(trifluoromethoxy)-1H-indole-2-carboxylic acid (0.100 g, 0.227 mmol) in DMF (5 mL) was added ammonium chloride (Chem-Impex, 0.036 g, 0.68 mmol). Next add TBTU (Oakwood, 0.109 g, 0.34 mmol), followed by the addition of DIPEA (0.36 mL, 2.04 mmol). The reaction mixture was stirred overnight at room temperature under N₂ atmosphere. The reaction mixture was subsequently partitioned between ethyl acetate (25 mL) and H₂O (15 mL). The phases were separated, and the organic phase was partitioned with 1M KHSO₄ (15 mL), followed by brine (15 mL). The organic layer was concentrated under reduced pressure to afford the crude product as a white solid (0.081 g, 82% yield). R_{f} 0.08 with 1:30:70 v/v acetic acid-ethyl acetate-heptane (UV 254 nM); ¹H-NMR (400 MHz; DMSO-d6) δ 8.15 (br s, 1H), 7.83-7.87 (m, 2H), 7.51 (br s, 1H), 7.42 (d, 1H, J=7.8 Hz), 7.33 (s, 1H), 7.30 (s, 1H), 7.21 (d, 1H, J=8.7 Hz), 7.11 (d, 1H, J=7.6 Hz), 4.00 (s, 2H), 3.95 (q, 2H, J=7.1 Hz), 0.91 (t, 3H, J=7.1 Hz); ¹⁹F-NMR (376 MHz; DMSO-d6) δ −56.74; MS (APCI+) m/z 440.0 (M+1), (APCI−) m/z 438.0 (M−1); HPLC UV purity, Rt=8.454 min, 67.9%. The product was used without further purification or characterization.

### Step C: Preparation of 2-((6-(2-carbamoyl-6-(trifluoromethoxy)-1H-indol-1-yl)pyridin-2-yl)thio)acetic acid (Compound 44)

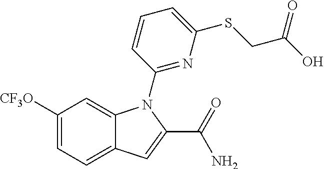

To a mixture consisting of ethyl 2-((6-(2-carbamoyl-6-(trifluoromethoxy)-1H-indol-1-yl)pyridin-2-yl)thio)acetate (0.080 g, 0.182 mmol) in methanol (5 mL) was added 1M lithium hydroxide (1.00 mL, 1.00 mmol). The reaction mixture was stirred at room temperature overnight under N₂ atmosphere. The reaction mixture was subsequently partitioned between ethyl acetate (10 mL) and 1M HCl (10 mL). The phases were separated, and the organic phase was partitioned with water (10 mL), followed by brine (10 mL). The organic layer was concentrated under reduced pressure to afford the crude compound as a white solid (0.063 g). The crude product was triturated with EA/Hep (5 mL, 30/70 mixture) and the solution was filtered over a fritted funnel to provide the title compound as a white solid (0.058 g, 79% yield). R_{f} 0.11 with 1:60:40 v/v acetic acid-ethyl acetate-heptane (UV 254 nM); ¹H-NMR (400 MHz; DMSO-d6) δ 12.73 (br s, 1H), 8.19 (br s, 1H), 7.83-7.87 (m, 2H), 7.52 (br s, 1H), 7.46 (s, 1H), 7.38 (d, 1H, J=7.8 Hz), 7.28 (s, 1H), 7.20 (d, 1H, J=8.8 Hz), 7.12 (d, 1H, J=7.8 Hz), 3.98 (s, 2H); ¹⁹F-NMR (376 MHz; DMSO-d6) δ −56.68; MS (APCI+) m/z 412.0 (M+1), 410.0 (M−1); HPLC UV purity, Rt=7.079 min, 98.0%; melting point=234-235° C.

### Step D: Preparation of 2-((6-(2-carbamoyl-6-(trifluoromethoxy)-1H-indol-1-yl)pyridin-2-yl)sulfonyl)acetic acid (Compound 45)

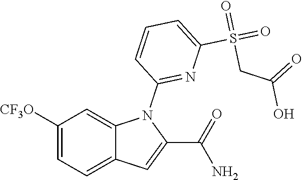

To a mixture consisting of 2-((6-(2-carbamoyl-6-(trifluoromethoxy)-1H-indol-1-yl)pyridin-2-yl)thio)acetic acid (0.025 g, 0.061 mmol) in acetic acid (0.75 mL) was added hydrogen peroxide (30 wt. % in H₂O, 0.5 mL). The reaction mixture was stirred at room temperature for 20 minutes then warmed to 60° C. for one hour. After cooling to room temperature, the reaction was partitioned between ethyl acetate (5 mL) and H₂O (5 mL). The organic layer was separated and washed a second time with H₂O (5 mL) and then brine (5 mL). The organic layer was concentrated under reduced pressure to afford a tan solid (17 mg). The crude solid was purified by flash silica column chromatography on a CombiFlash NextGen 300+ purification system. Elution through (2) 4 g RediSep Gold Rf flash silica cartridge with 10-30% methanol in dichloromethane containing 1% acetic acid afforded the title compound as a white solid (0.006 g, 23% yield). R_{f} 0.08 with 1:10:90 v/v acetic acid-methanol-dichloromethane (UV 254 nM); ¹H-NMR (400 MHz; DMSO-d6) δ 8.23-8.34 (m, 2H), 8.19 (t, 1H, J=7.8 Hz), 7.80-7.90 (m, 3H), 7.62 (br s, 1H), 7.56 (d, 1H, J=8.0 Hz), 7.38 (s, 1H), 7.20-7.24 (m, 1H)), 4.00 (s, 2H); ¹⁹F-NMR (376 MHz; DMSO-d6) δ −56.78; MS (APCI+) m/z 444.0 (M+1); HPLC UV purity, Rt=6.52 min, 85.2%.

### Example 57: Synthesis of 1-(3-(1,1-difluoro-2-hydroxyethyl)phenyl)-6-(trifluoromethoxy)-1H-indole-2-carboxamide (Compound 46) and 2-(3-(2-carbamoyl-6-(trifluoromethoxy)-1H-indol-1-yl)phenyl)-2,2-difluoroethyl methanesulfonate (Compound 47)

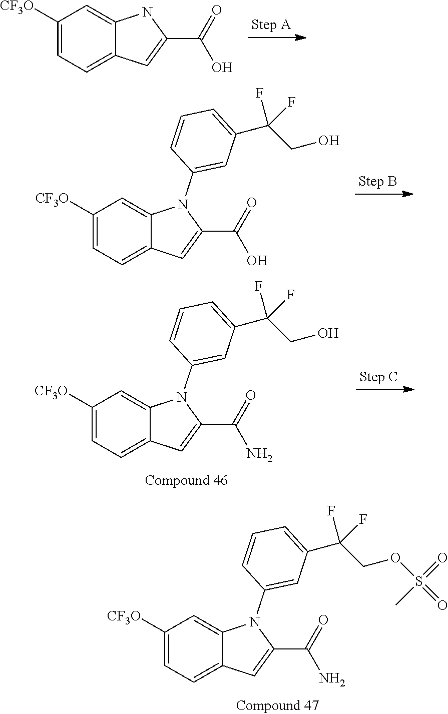

### Step A: Preparation of 1-(3-(1,1-difluoro-2-hydroxyethyl)phenyl)-6-(trifluoromethoxy)-1H-indole-2-carboxylic acid

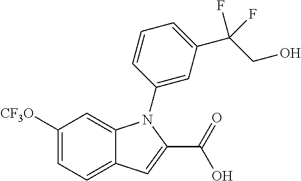

To a mixture consisting of 6-(trifluoromethoxy)-1H-indole-2-carboxylic acid (ChemShuttle, 0.490 g, 2.00 mmol), 2-(3-bromophenyl)-2,2-difluoroethanol (AABlocks, 0.474 g, 2.00 mmol), copper (II) acetate (CombiBlocks, 0.363 g, 2.00 mmol), methyl-α-D-glucopyranoside (CombiBlocks, 0.388 g, 2.00 mmol), potassium iodide (VWR, 0.664 g, 4.00 mmol) in DMSO (20 mL) was added DBU (Oakwood Chemicals, 0.90 mL, 6.00 mmol). The reaction mixture was heated to 115° C. under N₂ atmosphere for 16 hours and then allowed to cool to room temperature. The reaction mixture was subsequently partitioned between ethyl acetate (75 mL) and 1M KHSO₄ (50 mL). An emulsion formed which was broken up by the addition of 25 mL of brine. The phases were separated, and the organic phase was partitioned with H₂O (50 mL), followed by brine (50 mL). The organic layer was separated and dried over anhydrous magnesium sulfate, filtered, and concentrated under reduced pressure to give a crude brown oil, 1.34 g. The crude oil was purified by flash silica column chromatography on a CombiFlash NextGen 300+ purification system. Elution through a 24 g RediSep Gold Rf flash silica cartridge with 5-40% ethyl acetate in hexanes containing 1% acetic acid afforded the title compound as a tan solid (0.38 g, 48% yield). R_{f} 0.14 with 1:30:70 v/v acetic acid-ethyl acetate-hexane (UV 254 nM); ¹H-NMR (400 MHz; DMSO-d6) δ 13.06 (br s, 1H), 7.91 (d, 1H, J=8.7 Hz), 7.60-7.70 (m, 2H), 7.55-7.60 (m, 1H), 7.54 (s, 1H), 7.51 (s, 1H), 7.21 (d, 1H, J=7.9 Hz), 6.96 (s, 1H), 5.69 (t, 1H, J=6.4 Hz), 3.94 (dt, 2H, J=6.2, 13.9 Hz); ¹⁹F-NMR (376 MHz; DMSO-d6) δ −56.70; MS (APCI−) m/z 400.0 (M−1); HPLC UV purity, Rt=8.39 min, 98.6%.

### Step B: Preparation of 1-(3-(1,1-difluoro-2-hydroxyethyl)phenyl)-6-(trifluoromethoxy)-1H-indole-2-carboxamide (Compound 45)

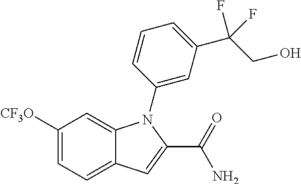

To a mixture consisting of 1-(3-(1,1-difluoro-2-hydroxyethyl)phenyl)-6-(trifluoromethoxy)-1H-indole-2-carboxylic acid (0.248 g, 0.617 mmol) in DMF (5 mL) was added ammonium chloride (Chem-Impex, 0.099 g, 1.85 mmol). Next add TBTU (Oakwood, 0.297 g, 0.93 mmol), followed by the addition of DIPEA (0.94 mL, 5.55 mmol). The reaction mixture was stirred overnight at room temperature under N₂ atmosphere. The reaction mixture was subsequently partitioned between ethyl acetate (25 mL) and H₂O (15 mL). The phases were separated, and the organic phase was partitioned with 1M KHSO₄ (10 mL), followed by brine (25 mL). The organic layer was concentrated under reduced pressure to afford the crude product as an orange solid (0.175 g). The crude product was triturated with a 2.0/0.5/0.5 mixture of Heptane/DCM/Ethyl Acetate (5 mL) and then filtered over a fritted funnel to provide the title compound as a tan solid (0.120 g, 49% yield). R_{f} 0.05 with 1:30:70 v/v acetic acid-ethyl acetate-heptane (UV 254 nM); ¹H-NMR (400 MHz; DMSO-d6) δ 8.09 (br s, 1H), 7.86 (d, 1H, J=8.7 Hz), 7.60-7.70 (m, 2H), 7.40-7.50 (m, 3H), 7.35 (s, 1H), 7.18 (br d, 1H, J=8.3 Hz), 7.01 (s, 1H), 5.70 (t, 1H, J=6.4 Hz), 3.90 (dt, 2H, J=6.3, 13.9 Hz); ¹⁹F-NMR (376 MHz; DMSO-d6) δ −56.72, −103.23, −103.26; MS (APCI+) m/z 401.0 (M+1), (APCI−) m/z 399.0 (M−1); HPLC UV purity, Rt=7.70 min, 92.9%.

### Step C: Preparation of 2-(3-(2-carbamoyl-6-(trifluoromethoxy)-1H-indol-1-yl)phenyl)-2,2-difluoroethyl methanesulfonate (Compound 46)

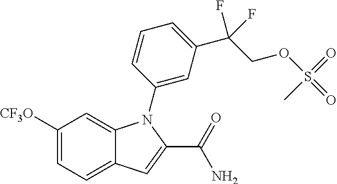

To a mixture consisting of 1-(3-(1,1-difluoro-2-hydroxyethyl)phenyl)-6-(trifluoromethoxy)-1H-indole-2-carboxamide (0.110 g, 0.275 mmol) in DCM (2 mL) and THE (2 mL) was added triethylamine (Oakwood, 0.04 mL, 0.275 mmol). The reaction mixture was cooled in ice/salt bath (3:1 ratio, −20° C.). Next while stirring add methanesulfonyl chloride (Aldrich, 0.032 mL, 0.41 mmol). The reaction mixture was stirred at −20° C. for 1 hour and then allowed to warm to room temperature for 2 hours. The reaction was quenched with the addition of saturated aqueous ammonium chloride (20 mL) and subsequently partitioned between DCM (25 mL) and H₂O (15 mL). The organic layer was separated and subsequently washed with brine (25 mL). The organic layer was concentrated under reduced pressure to afford the crude product as an orange oil (0.166 g). The crude oil was purified by flash silica column chromatography on a CombiFlash NextGen 300+ purification system. Elution through a 12 g RediSep Gold Rf flash silica cartridge with 0-15% methanol in dichloromethane afforded the title compound as a tan solid (0.030 g, 23% yield). R_{f} 0.36 with 5:95 v/v methanol-dichloromethane (UV 254 nM); ¹H-NMR (400 MHz; DMSO-d6) δ 8.10 (br s, 1H), 7.87 (d, 1H, J=8.5 Hz), 7.66-7.72 (m, 2H), 7.62 (s, 1H), 7.53-7.56 (m, 1H), 7.43 (br s, 1H), 7.38 (s, 1H), 7.18 (br d, 1H, J=8.7 Hz), 6.97 (s, 1H), 4.86 (t, 2H, J=13.5 Hz), 3.23 (s, 3H); ¹⁹F-NMR (376 MHz; DMSO-d6) δ −56.72, −102.22; MS (APCI+) m/z 479.0 (M+1), (APCI−) m/z 477.0 (M−1); HPLC UV purity, Rt=8.51 min, 93.3%; melting point=110-111° C.

### Example 58: Synthesis of tert-butyl (1-(6-(2-carbamoyl-6-(trifluoromethoxy)-1H-indol-1-yl)pyridin-2-yl)cyclobutyl)carbamate (Compound 48), 1-(6-(1-aminocyclobutyl)pyridin-2-yl)-6-(trifluoromethoxy)-1H-indole-2-carboxamide TFA salt (Compound 49) and (1-(6-(2-carbamoyl-6-(trifluoromethoxy)-1H-indol-1-yl)pyridin-2-yl)cyclobutyl)glycine (Compound 50)

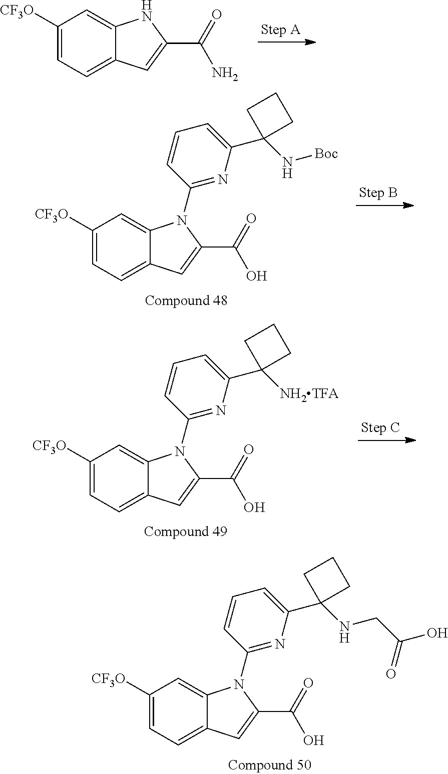

### Step A: Preparation of tert-butyl (1-(6-(2-carbamoyl-6-(trifluoromethoxy)-1H-indol-1-yl)pyridin-2-yl)cyclobutyl)carbamate (Compound 48)

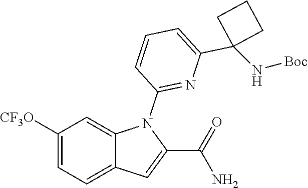

To a mixture consisting of 6-(trifluoromethoxy)-1H-indole-2-carboxamide (0.300 g, 1.23 mmol), tert-butyl (1-(6-bromopyridin-2-yl)cyclobutyl)carbamate (1Click Chemistry, 0.250 g, 0.764 mmol), copper (I) iodide (Strem, 0.132 g, 0.694 mmol) in DMSO (10 mL) was added DBU (Oakwood Chemicals, 0.2 mL, 1.39 mmol). The reaction mixture was heated to 120° C. under N₂ atmosphere for 8 hours and then allowed to cool to room temperature. The reaction mixture was subsequently partitioned between ethyl acetate (25 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (30 mL), followed by brine (30 mL). The organic layer was separated and dried over anhydrous magnesium sulfate, filtered, and concentrated under reduced pressure to give a crude brown oil, 0.740 g. The crude oil was purified by flash silica column chromatography on a CombiFlash NextGen 300+ purification system. Elution through a 12 g RediSep Gold Rf flash silica cartridge with 10-60% ethyl acetate in hexanes containing 1% acetic acid afforded the title compound as a tan solid (0.243 g, 64% yield). R_{f} 0.09 with 1:30:70 v/v acetic acid-ethyl acetate-hexane (UV 254 nM); ¹H-NMR (400 MHz; DMSO-d6) δ 8.19 (br s, 1H), 7.91-7.93 (m, 2H), 7.86 (d, 1H, J=8.7 Hz), 7.79 (br s, 1H), 7.60-7.63 (m, 1H), 7.54 (br s, 1H), 7.27-7.30 (m 2H), 7.15-7.22 (m, 2H), 2.56-2.63 (m, 2H), 2.30-2.40 (m, 2H), 1.90-2.00 (m, 2H), 1.39 (s, 9H); ¹⁹F-NMR (376 MHz; DMSO-d6) δ −56.78; MS (APCI⁺) m/z 491.0 (M+1), (APCI⁻) m/z 489.0 (M−1); HPLC UV purity, Rt=9.142 min, 98.5%; melting point=95-97° C.

### Step B: Preparation of 1-(6-(1-aminocyclobutyl)pyridin-2-yl)-6-(trifluoromethoxy)-1H-indole-2-carboxamide (TFA salt) (Compound 49)

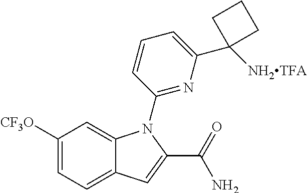

To a mixture consisting of tert-butyl (1-(6-(2-carbamoyl-6-(trifluoromethoxy)-1H-indol-1-yl)pyridin-2-yl)cyclobutyl)carbamate (0.141 g, 0.287 mmol) in dichloromethane (2 mL) was added trifluoroacetic acid (Chem-Impex, 0.5 mL, 6.74 mmol). The reaction mixture was stirred at room temperature for 3.5 hours then briefly heated to 80° C. for 5 minutes. After cooling to room temperature, the reaction was concentrated under reduced pressure in the presence of toluene (10 mL). The crude product was concentrated with toluene (10 mL) an additional two times which afforded the title compound as tan solid (0.142, 98% yield). Rf 0.14 with 10:90 v/v methanol-dichloromethane (UV 254 nM); ¹H-NMR (400 MHz; DMSO-d6) δ 8.75 (br s, 2H), 8.22 (br s, 1H), 8.13 (t, 1H, J=7.6 Hz), 7.87 (d, 1H, J=8.7 Hz), 7.75 (d, 1H, J=7.6 Hz), 7.54 (s, 2H), 7.42 (d, 1H, J=8.0 Hz), 7.34 (s, 1H), 7.22 (d, 1H, J=8.0 Hz), 2.60-2.68 (m, 2H), 2.50-2.60 (m, 2H), 2.02-2.17 (m, 2H); ¹⁹F-NMR (376 MHz; DMSO-d6) δ −56.71, −73.47; MS (APCI⁺) m/z 391.0 (M+1), (APCI−) m/z 389.0 (M−1); HPLC UV purity, Rt=6.080 min, 97.6%; melting point=96-98° C.

### Step C: Preparation of (1-(6-(2-carbamoyl-6-(trifluoromethoxy)-1H-indol-1-yl)pyridin-2-yl)cyclobutyl)glycine (Compound 50)

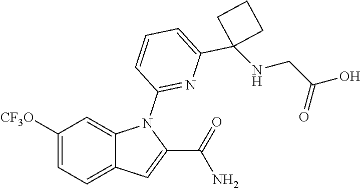

To a reaction mixture consisting of 1-(6-(1-aminocyclobutyl)pyridin-2-yl)-6-(trifluoromethoxy)-1H-indole-2-carboxamide (TFA salt) (0.100 g, 0.198 mmol) in acetonitrile (3 mL) and water (0.5 mL) was added potassium carbonate (0.082 g, 0.594 mmol). Next while stirring at room temperature, ethyl bromoacetate was added (0.024 mL, 0.218 mmol). The reaction mixture was stirred at room temperature for 1 hour and then an additional 0.01 mL of ethyl bromoacetate was added. The reaction was heated to 45° C. for 3 hours. After cooling to room temperature, water (25 mL) and ethyl acetate (25 mL) was added, and the reaction mixture was placed in a separatory funnel. The organic layer is separated, and the aqueous layer is extracted a second time with ethyl acetate (25 mL). The combined organic layers were washed with brine (25 mL) and then concentrated under reduced pressure to afford a crude orange oil, 0.189 g. The crude oil was purified by flash silica column chromatography on a CombiFlash NextGen 300+ purification system. Elution through a 12 g RediSep Gold Rf flash silica cartridge with 0-10% methanol in dichloromethane afforded ethyl (1-(6-(2-carbamoyl-6-(trifluoromethoxy)-1H-indol-1-yl)pyridin-2-yl)cyclobutyl)glycinate as a white solid (0.061 g, 64% yield). R_{f} 0.75 with 10:90 v/v methanol-dichloromethane (UV 254 nM); MS (APCI⁺) m/z 477.0 (M+1), (APCI−) m/z 475.0.0 (M−1); HPLC UV purity, Rt=6.633 min, 99.3%. The ethyl ester, ethyl (1-(6-(2-carbamoyl-6-(trifluoromethoxy)-1H-indol-1-yl)pyridin-2-yl)cyclobutyl)glycinate (0.061 g, 0.128 mmol) was subsequently dissolved in THF (1 mL) and methanol (0.5 mL). Lithium hydroxide (1M in H₂O, 0.64 mL, 0.64 mmol) was added and the reaction mixture was heated to 35° C. for 30 minutes. After cooling to room temperature, the reaction mixture was partitioned between ethyl acetate (15 mL) and 1N HCl (15 mL). The organic layer was separated and next washed with water (15 mL) and then brine (15 mL). The organic layer is concentrated under reduced pressure to afford the title compound as a white solid (0.010 g, 18% yield). R_{f} 0.04 with 10:90 v/v methanol-dichloromethane (UV 254 nM); ¹H-NMR (400 MHz; DMSO-d6) δ 12.60 (br s, 1H), 8.14 (br s, 1H), 8.04 (t, 1H, J=7.6 Hz), 7.82 (d, 1H, J=8.7 Hz), 7.70 (m, 1H), 7.35-7.44 (m, 3H), 7.26 (s, 1H), 7.17 (br d, 1H, J=8.3 Hz), 3.42 (s, 2H), 2.50-2.60 (m, 2H), 1.93-1.98 (m, 2H), 1.74-1.93 (m, 2H); ¹⁹F-NMR (376 MHz; DMSO-d6) δ −56.72; MS (APCI⁺) m/z 449.0 (M+1), (APCI−) m/z 447.0 (M−1); HPLC UV purity, Rt=6.056 min, 92.8%.

### Example 59: Synthesis of trans-2-(6-(2-carbamoyl-6-methoxy-1H-pyrrolo[3,2-b]pyridin-1-yl)pyridin-2-yl)cyclopropane-1-carboxylic acid (Compound 51)

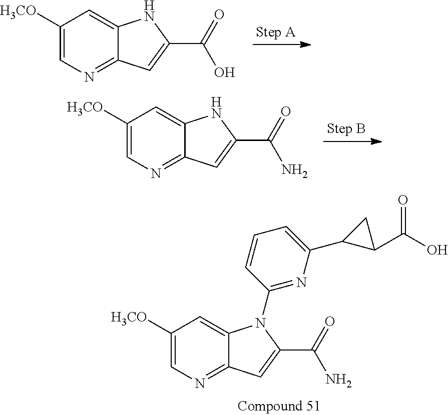

### Step A: Preparation of 6-methoxy-1H-pyrrolo[3,2-b]pyridine-2-carboxamide

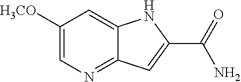 [SMILES: COc1cnc2cc(C(N)=O)[nH]c2c1]

To a mixture consisting of 6-methoxy-1H-pyrrolo[3,2-b]pyridine-2-carboxylic acid (Enamine, 0.384 g, 2.0 mmol) in thionyl chloride (7 mL) was heated to reflux for 4 hours. After cooling the reaction to room temperature, THE (40 mL) was added, and the reaction mixture was concentrated under reduced pressure. Next a reaction flask containing ammonium hydroxide (28%, 20 mL) was cooled to 0° C. in an ice water bath. The crude acid chloride (mixture in 25 mL of tetrahydrofuran) was added, and the reaction mixture was stirred for 20 minutes. The contents of the Erlenmeyer flask was transferred to a separatory funnel. The organic layer was separated and then washed with brine (25 mL). The organic layer was concentrated under reduced pressure to afford a crude tan solid, 1.138 g. The crude material was triturated with ethyl acetate/methanol (10 mL, 1:1) and filtered over a fritted funnel to collect the desired product (0.096 g). The filtrate was subsequently purified by flash silica column chromatography on a CombiFlash NextGen 300+ purification system. Elution through a 12 g RediSep Gold Rf flash silica cartridge with 0-15% methanol in dichloromethane afforded the title compound as a tan solid (0.207 g, 54% yield). R_{f} 0.12 with 5:95 v/v methanol-dichloromethane (UV 254 nM); ¹H-NMR (400 MHz; DMSO-d6) δ 11.60 (br s, 1H), 8.14 (d, 1H, J=2.5 Hz), 7.99 (br s, 1H), 7.41 (br s, 1H), 7.21 (t, 2H, J=2.5 Hz), 3.82 (s, 3H); MS (APCI⁺) m/z 192.0 (M+1), (APCI−) m/z 190.0 (M−1); HPLC UV purity, Rt=1.938 min, 99.4%.

### Step B: Preparation of trans-(−)-2-(6-(2-carbamoyl-6-methoxy-1H-pyrrolo[3,2-b]pyridin-1-yl)pyridin-2-yl)cyclopropane-1-carboxylic acid (Compound 51)

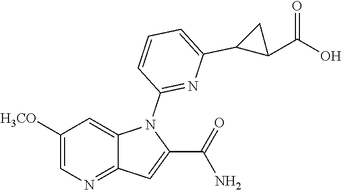 [SMILES: COc1cnc2cc(C(N)=O)n(-c3cccc(C4CC4C(=O)O)n3)c2c1]

To a mixture consisting of 6-methoxy-1H-pyrrolo[3,2-b]pyridine-2-carboxamide (0.090 g, 0.47 mmol), trans-(−)-2-(6-bromopyridin-2-yl)cyclopropane-1-carboxylic acid (0.125 g, 0.518 mmol), copper (I) iodide (Strem, 0.089 g, 0.47 mmol) in DMSO (6 mL) was added DBU (Oakwood Chemicals, 0.14 mL, 0.94 mmol). The reaction mixture was heated to 120° C. under N₂ atmosphere for 2 hours and then allowed to cool to room temperature. The reaction mixture was subsequently partitioned between ethyl acetate (25 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (30 mL), followed by brine (30 mL). The organic layer was separated and dried over anhydrous magnesium sulfate, filtered, and concentrated under reduced pressure to give a crude yellow residue, 0.045 g. The aqueous layer was acidified with 1N HCl to pH=5 and then extracted back with dichloromethane. The organic extracts were combined with the previous ethyl acetate extract to provide a crude yellow oil (0.120 g). The crude was purified by flash silica column chromatography on a CombiFlash NextGen 300+ purification system. Elution through a 12 g RediSep Gold Rf flash silica cartridge with 10-30% methanol in dichloromethane containing 1% acetic acid afforded the title compound as a tan solid (0.004 g, 3% yield). R_{f} 0.28 with 1:10:90 v/v acetic acid-methanol-dichloromethane (UV 254 nM); ¹H-NMR (400 MHz; DMSO-d6) δ 12.35 (br s, 1H), 8.26 (br s, 1H), 8.08 (br s, 1H), 7.83 (t, 1H, J=6.5 Hz), 7.46 (br s, 1H), 7.30-7.40 (m, 2H), 7.20 (s, 2H), 3.82 (s, 3H), 2.54 (s, 1H), 1.88-2.00 (m, 2H), 1.50-1.60 (m, 1H); MS (APCI⁺) m/z 353.0 (M+1), (APCI−) m/z 351.0 (M−1); HPLC UV purity, Rt=3.679 min, 94.0%; melting point=143.5-144.5° C.

### Example 60: Synthesis of 1-(6-(2-carbamoyl-6-(trifluoromethoxy)-1H-indol-1-yl)pyridin-2-yl)azetidine-3-carboxylic acid (Compound 52)

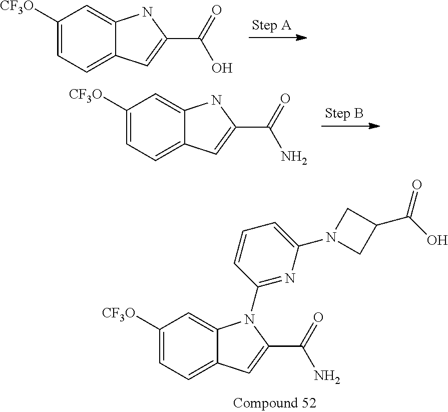

### Step A: Preparation of 6-(trifluoromethoxy)-1H-indole-2-carboxamide

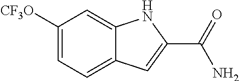

To a mixture consisting of 6-(trifluoromethoxy)-1H-indole-2-carboxylic acid (ChemShuttle, 3.00 g, 12.23 mmol) in DMF (75 mL) was added ammonium chloride (Chem-Impex, 3.28 g, 61.32 mmol). Next add TBTU (Oakwood, 5.90 g, 18.37 mmol), followed by the addition of DIPEA (14 mL, 80.37 mmol). The reaction mixture was stirred overnight at room temperature under N₂ atmosphere. The reaction mixture was subsequently partitioned between ethyl acetate (100 mL) and H₂O (2×100 mL). The phases were separated, and the organic phase was partitioned with 1M KHSO₄ (50 mL), followed by brine (50 mL). The organic layer was concentrated under reduced pressure to afford the crude product as a white solid (7.90 g, DMF present). The crude solid was purified by flash silica column chromatography on a CombiFlash NextGen 300+ purification system. Elution through an 80 g RediSep Gold Rf flash silica cartridge with 20-50% ethyl acetate in hexanes afforded the title compound as a white solid (1.68 g, 56% yield). R_{f} 0.53 with 1:60:40 v/v acetic acid-ethyl acetate-hexane (UV 254 nM); ¹H-NMR (400 MHz; DMSO-d6) δ 11.76 (br s, 1H), 8.01 (br s, 1H), 7.68 (d, 1H, J=8.5 Hz), 7.42 (br s, 1H), 7.29 (s, 1H), 7.10-7.14 (m, 1H), 6.97 (br d, 1H, J=8.7 Hz), 2.65 (s, 2H); ¹⁹F-NMR (376 MHz; DMSO-d6) δ −56.73; MS (APCI−) m/z 243.0 (M−1); melting point=143-145° C.

### Step B: Preparation of 1-(6-(2-carbamoyl-6-(trifluoromethoxy)-1H-indol-1-yl)pyridin-2-yl)azetidine-3-carboxylic acid (Compound 52)

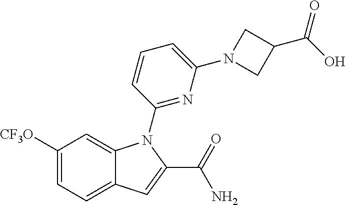

To a mixture consisting of 6-(trifluoromethoxy)-1H-indole-2-carboxamide (0.244 g, 1.00 mmol), 1-(6-bromopyridin-2-yl)azetidine-3-carboxylic acid (BLD Pharm, 0.282 g, 1.1 mmol), copper (I) iodide (Strem, 0.190 g, 1.00 mmol) in DMSO (6 mL) was added DBU (Oakwood Chemicals, 0.3 mL, 2.00 mmol). The reaction mixture was heated to 120° C. under N₂ atmosphere for 5 hours and then allowed to cool to room temperature. The reaction mixture was subsequently partitioned between ethyl acetate (30 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (30 mL), followed by brine (30 mL). The organic layer was separated and dried over anhydrous magnesium sulfate, filtered, and concentrated under reduced pressure to give a crude brown oil, 0.405 g. The crude oil was purified by flash silica column chromatography on a CombiFlash NextGen 300+ purification system. Elution through a 12 g RediSep Gold Rf flash silica cartridge with 10-70% ethyl acetate in hexanes containing 1% acetic acid afforded the title compound as a tan solid (0.125 g, 30% yield). R_{f} 0.12 with 1:60:40 v/v acetic acid-ethyl acetate-hexane (UV 254 nM); ¹H-NMR (400 MHz; DMSO-d6) δ 12.66 (br s, 1H), 8.09 (br s, 1H), 7.81 (d, 1H, J=8.7 Hz), 7.69 (t, 1H, J=7.8 Hz), 7.52 (s, 1H), 7.47 (br s, 1H), 7.17 (d, 2H, J=9.1 Hz), 7.15 (s, 1H), 6.61 (d, 1H, J=7.6 Hz), 6.43 (d, 1H, J=8.0 Hz), 4.10-4.20 (m, 2H), 4.00-4.10 (m, 2H), 3.55-3.60 (m, 1H); ¹⁹F-NMR (376 MHz; DMSO-d6) δ −56.73; MS (APCI⁺) m/z 421.0 (M+1), (APCI⁻) m/z 419.0 (M−1); HPLC UV purity, Rt=6.74 min, 98.5%; melting point=214.5-215.0° C.

### Example 61: Synthesis of 1-(6-(2-carbamoyl-6-(trifluoromethoxy)-1H-indol-1-yl)pyridin-2-yl)azetidine-3-carboxylic acid (Compound 53)

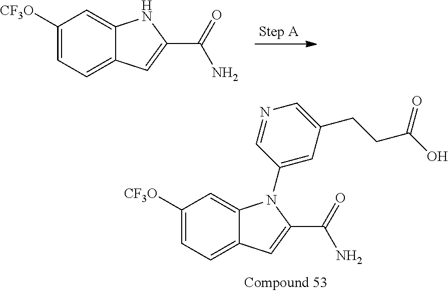

To a mixture consisting of 6-(trifluoromethoxy)-1H-indole-2-carboxamide (0.120 g, 0.49 mmol), 3-(5-bromopyridin-3-yl)propanoic acid (BioNet, 0.124 g, 0.540 mmol), copper (I) iodide (Strem, 0.093 g, 0.49 mmol) in DMSO (3 mL) was added DBU (Oakwood Chemicals, 0.15 mL, 1.00 mmol). The reaction mixture was heated to 120° C. under N₂ atmosphere for 5 hours and then allowed to cool to room temperature. The reaction mixture was subsequently partitioned between ethyl acetate (75 mL) and 1M KHSO₄ (40 mL). A precipitate formed which was collected by filtration over a fritted funnel and set aside. The filtrate was poured in a separatory funnel. The phases were separated, and the organic phase was partitioned with H₂O (50 mL), followed by brine (50 mL). The organic layer was separated and dried over anhydrous magnesium sulfate, filtered, and concentrated under reduced pressure to give a crude red oil, 0.155 g. The crude oil was purified by flash silica column chromatography on a CombiFlash NextGen 300+ purification system. Elution through a 4 g RediSep Gold Rf flash silica cartridge with 10-90% ethyl acetate in hexanes containing 1% acetic acid afforded the title compound as a brown solid (0.011 g, 5.7% yield). Rf 0.29 with 1:10:90 v/v acetic acid-methanol-dichloromethane (UV 254 nM); ¹H-NMR (400 MHz; DMSO-d6) δ 12.24 (br s, 1H), 8.53 (s, 1H), 8.36 (d, 1H, J=2.1), Hz 8.11 (br s, 1H), 7.87 (d, 1H, J=8.7 Hz), 7.77 (s, 1H), 7.40-7.47 (m, 2H), 7.19 (br d, 1H, J=8.9 Hz), 7.00 (s, 1H), 2.90-2.95 (m, 2H), 2.62-2.66 (m, 2H); ¹⁹F-NMR (376 MHz; DMSO-d6) δ −56.69; MS (APCI⁺) m/z 394.0 (M+1), (APCI−) m/z 392.0 (M−1); LCMS, Rt=2.98 min. m/z 394.0 (M+1), (APCI⁻) m/z 392.0 (M−1); melting point=186-187° C.

### Example 62: Synthesis of trans-(rac)-2-(6-(2-carbamoyl-5-(trifluoromethoxy)benzo[b]thiophen-3-yl)pyridin-2-yl)cyclopropane-1-carboxylic acid (Compound 54)

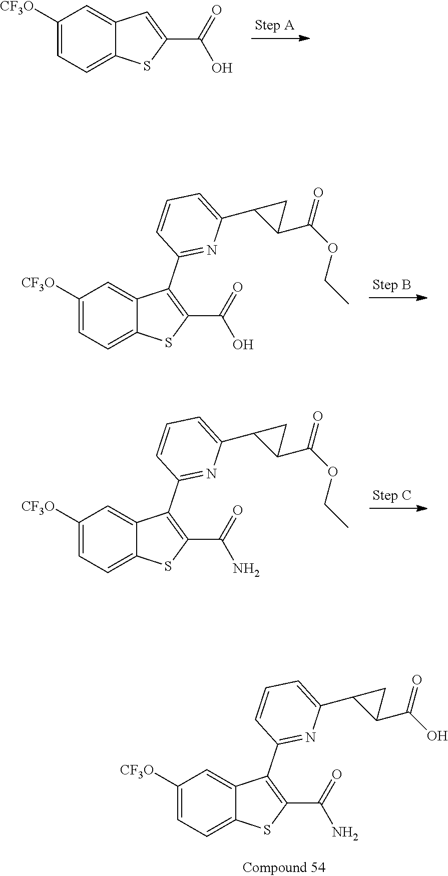

### Step A: Preparation of trans-(rac)-3-(6-(2-(ethoxycarbonyl)cyclopropyl)pyridin-2-yl)-5-(trifluoromethoxy)benzo[b]thiophene-2-carboxylic acid

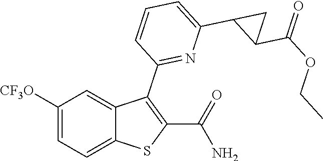

To a 40 mL septa-cap vial was added racemic trans-ethyl-2-(6-bromopyridin-2-yl)cyclopropane-1-carboxylate (prepared according to WO2020022470 A1), (0.541 g, 2.00 mmol) and 5-(trifluoromethoxy)benzo[b]thiophene-2-carboxylic acid (0.525 g, 2.00 mmol). Next was added Bis(dichloro(1′-p-cymene)ruthenium) (Strem, 0.049 g, 4 mol %), trimethylphosphonium tetrafluoroborate (Strem, 0.026 g, 8 mol %), K₂CO₃ (VWR, 0.304 g, 2.20 mmol) and NMP (14 mL). The mixture was degassed by bubbling in nitrogen gas for 3 min with stirring then heated to 110° C. overnight. The reaction mixture was subsequently cooled to room temperature and diluted with water (300 mL). The mixture was extracted with ethyl acetate (3×200 mL) and the combined organics dried with Na₂SO₄, filtered and solvent evaporated under reduced pressure to give a crude oil which was purified by flash silica column chromatography on a CombiFlash NextGen 300+ purification system. Elution through a 80 g RediSep Gold Rf flash silica cartridge with 5-95% ethyl acetate in heptane containing 1% acetic acid afforded the title compound as an off-white semi-solid (0.055 g, 6% yield). R_{f} 0.40 with 1:40:60 v/v acetic acid-ethyl acetate-hexane (UV 254 nM); ¹H-NMR (400 MHz; CDCl₃) δ 7.9-8.0 (m, 2H), 7.8-7.8 (m, 1H), 7.69 (d, 1H, J=7.8 Hz), 7.47 (d, 1H, J=7.8 Hz), 7.41 (br d, 1H, J=8.7 Hz), 4.19 (q, 2H, J=7.0 Hz), 2.7-2.8 (m, 1H), 2.3-2.4 (m, 1H), 1.7-1.8 (m, 2H), 1.3-1.3 (m, 3H); MS (APCI⁺) m/z 452.1 (M+1).

### Step B: Preparation of trans-(rac)-ethyl 2-(6-(2-carbamoyl-5-(trifluoromethoxy)benzo[b]thiophen-3-yl)pyridin-2-yl)cyclopropane-1-carboxylate

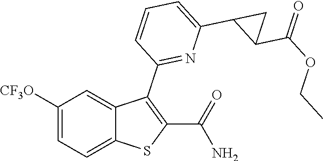

To a mixture consisting of trans-(rac)-3-(6-(2-(ethoxycarbonyl)cyclopropyl)pyridin-2-yl)-5-(trifluoromethoxy)benzo[b]thiophene-2-carboxylic acid (0.055 g, 0.12 mmol) in DMF (3.0 mL) was added ammonium chloride (Chem-Impex, 0.020 g, 0.37 mmol). Next was added TBTU (Oakwood, 0.059 g, 0.18 mmol) followed by the addition of DIPEA (0.20 mL, 1.1 mmol). The reaction mixture was stirred overnight at room temperature under N₂ atmosphere. The reaction mixture was subsequently diluted with water (60 mL) then extracted with 2×50 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 12 g RediSep Gold Rf flash silica cartridge with 5-65% ethyl acetate in heptane containing 1% acetic acid afforded the title compound as a brown oil (0.043 g, 80% yield). Rf 0.39 with 1:40:60 v/v acetic acid-ethyl acetate-hexane (UV 254 nM); ¹H-NMR (400 MHz; CDCl₃) δ 7.9-8.0 (m, 2H), 7.86 (t, 1H, J=7.8 Hz), 7.3-7.5 (m, 4H), 6.04 (br s, 1H), 4.1-4.2 (m, 2H), 2.74 (ddd, 1H, J=4.0, 6.0, 9.0 Hz), 2.2-2.3 (m, 1H), 1.6-1.7 (m, 2H), 1.29 (t, 3H, J=7.1 Hz); MS (APCI⁺) m/z 451.0 (M+1), m/z 449.0 (M−1).

### Step C: Preparation of trans-(rac)-2-(6-(2-carbamoyl-5-(trifluoromethoxy)benzo[b]thiophen-3-yl)pyridin-2-yl)cyclopropane-1-carboxylic acid (Compound 54)

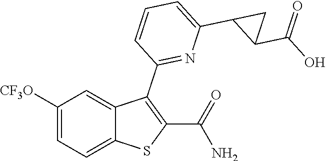

To a mixture consisting of trans-(rac)-ethyl 2-(6-(2-carbamoyl-5-(trifluoromethoxy)benzo[b]thiophen-3-yl)pyridin-2-yl)cyclopropane-1-carboxylate (0.038 g, 0.08 mmol) in MeOH (3 mL) was added dropwise a 1M aqueous solution of LiOH (0.26 mL, 3 eq). The reaction mixture was stirred overnight at room temperature. The solution was subjected to reduced pressure on a rotary evaporator to remove MeOH. Additional water was added (30 mL) and the aqueous mixture acidified with 1N HCl. Extracted the aqueous mixture with ethyl acetate (2×30 mL). Dry the combined organics with Na₂SO₄. Filter and evaporate solvent to obtain crude product which was purified by flash silica column chromatography on a CombiFlash NextGen 300+ purification system. Elution through a 4 g RediSep Gold Rf flash silica cartridge with 10-100% ethyl acetate in heptane containing 1% acetic acid afforded the title compound as a white solid after high vacuum at 40° C. overnight (0.021 g, 60% yield). Rf 0.33 with 1:60:40 acetic acid-ethyl acetate-heptane (UV 254 nM); H NMR (METHANOL-d4, 400 MHz) δ 8.07 (d, 1H, J=8.9 Hz), 7.88 (t, 1H, J=7.7 Hz), 7.64 (s, 1H), 7.47 (dd, 2H, J=4.2, 7.7 Hz), 7.4-7.4 (m, 1H), 2.7-2.8 (m, 1H, J=3.9, 5.7, 9.2 Hz), 2.2-2.3 (m, 1H), 1.6-1.7 (m, 1H), 1.5-1.6 (m, 1H, J=3.9, 5.3, 8.9 Hz); ¹⁹F-NMR (376 MHz; METHANOL-d4) δ −59.52; MS (APCI+) m/z 423.0 (M+1), m/z 421.0 (M−1), HPLC UV purity, Rt=7.10 min, 96.0%; melting point=205.0-207.0° C. (dec).

### Example 63: Synthesis of trans-(rac)-2-(6-(2-carbamoyl-5-(trifluoromethoxy)benzo[b]selenophen-3-yl)pyridin-2-yl)cyclopropane-1-carboxylic acid (Compound 55)

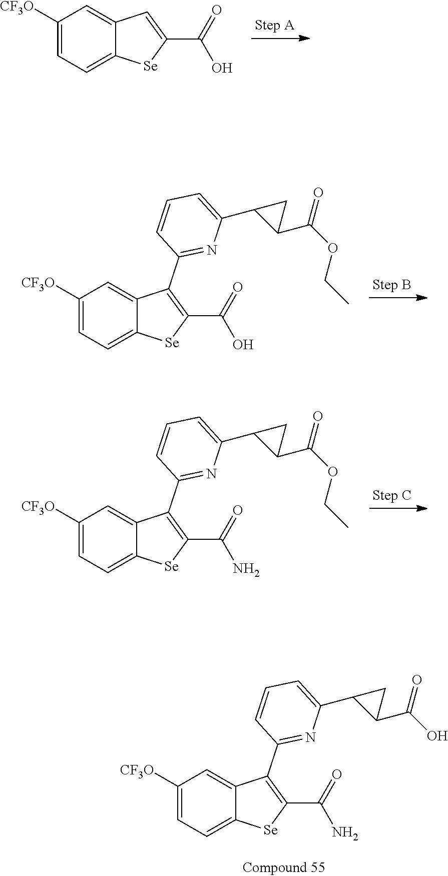

### Step A: Preparation of trans-(rac)-3-(6-(2-(ethoxycarbonyl)cyclopropyl)pyridin-2-yl)-5-(trifluoromethoxy)benzo[b]selenophene-2-carboxylic acid

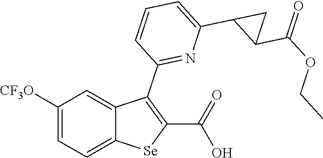

To a 20 mL septa-cap vial was added trans-(rac)-ethyl-2-(6-bromopyridin-2-yl)cyclopropane-1-carboxylate (prepared according to WO2020022470 A1), (0.486 g, 1.80 mmol) and 5-(trifluoromethoxy)benzo[b]selenophene-2-carboxylic acid (0.463 g, 1.50 mmol). Next was added Bis(dichloro(η⁶-p-cymene)ruthenium) (Strem, 0.046 g, 4 mol %), trimethylphosphonium tetrafluoroborate (Strem, 0.025 g, 8 mol %), K₂CO₃ (VWR, 0.311 g, 2.25 mmol) and NMP (8 mL). The mixture was degassed by bubbling in nitrogen gas for 3 min with stirring then heated to 110° C. overnight. The reaction mixture was subsequently cooled to room temperature and diluted with water (300 mL). The mixture was extracted with ethyl acetate (3×200 mL) and the combined organics dried with Na₂SO₄, filtered and solvent evaporated under reduced pressure to give a crude oil which was purified by flash silica column chromatography on a CombiFlash NextGen 300+ purification system. Elution through a 40 g RediSep Gold Rf flash silica cartridge with 5-40% ethyl acetate in heptane containing 1% acetic acid afforded the title compound as a dark oil (0.072 g, 10% yield). Rf 0.36 with 1:40:60 v/v acetic acid-ethyl acetate-hexane (UV 254 nM); ¹H-NMR (400 MHz; CDCl₃) δ 8.0-8.0 (m, 2H), 7.7-7.8 (m, 2H), 7.60 (d, 1H, J=7.8 Hz), 7.47 (d, 1H, J=7.8 Hz), 7.35 (br d, 1H, J=7.6 Hz), 4.19 (dq, 2H, J=1.4, 7.1 Hz), 2.77 (ddd, 1H, J=4.1, 6.4, 9.0 Hz), 2.38 (ddd, 1H, J=4.2, 5.9, 8.4 Hz), 1.7-1.8 (m, 2H), 1.29 (br t, 3H, J=7.1 Hz); LC-MS 2 peaks (ret. time 3.66, 3.81 min) both m/z 500.0 (M+1) and Se isotope distribution.

### Step B: Preparation of trans-(rac)-ethyl 2-(6-(2-carbamoyl-5-(trifluoromethoxy)benzo[b]selenophen-3-yl)pyridin-2-yl)cyclopropane-1-carboxylate

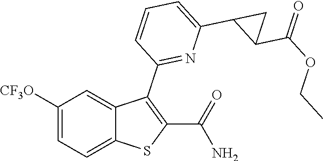

To a mixture consisting of trans-(rac)-3-(6-(2-(ethoxycarbonyl)cyclopropyl)pyridin-2-yl)-5-(trifluoromethoxy)benzo[b]selenophene-2-carboxylic acid (0.069 g, 0.14 mmol) in DMF (4.0 mL) was added ammonium chloride (Chem-Impex, 0.022 g, 0.41 mmol). Next was added TBTU (Oakwood, 0.067 g, 0.21 mmol) followed by the addition of DIPEA (0.22 mL, 1.2 mmol). The reaction mixture was stirred overnight at room temperature under N₂ atmosphere. The reaction mixture was subsequently diluted with water (100 mL) then extracted with 2×80 mL of ethyl acetate. The combined organic phases were dried with Na₂SO₄, filtered and solvent evaporated under reduced pressure to afford the crude product as a colorless oil. The crude oil was purified by flash silica column chromatography on a CombiFlash NextGen 300+ purification system. Elution through a 24 g RediSep Gold Rf flash silica cartridge with 10-50% ethyl acetate in heptane containing 1% acetic acid afforded the title compound as a brown oil (0.047 g, 69% yield). Rf 0.32 with 1:40:60 v/v acetic acid-ethyl acetate-hexane (UV 254 nM); ¹H-NMR (400 MHz; CDCl₃) δ 8.31 (d, 1H, J=8.7 Hz), 7.86 (t, 1H, J=7.8 Hz), 7.69 (br s, 1H), 7.5-7.6 (m, 3H), 7.44 (br d, 1H, J=8.9 Hz), 7.39 (d, 1H, J=7.8 Hz), 4.1-4.1 (m, 2H), 2.7-2.8 (m, 1H), 2.1-2.2 (m, 1H), 1.5-1.6 (m, 2H), 1.20 (t, 3H, J=7.1 Hz); LC-MS 2 peaks (ret. time 4.40, 4.60 min) both m/z 499.0 (M+1) and Se isotope distribution.

### Step C: Preparation of trans-(rac)-2-(6-(2-carbamoyl-5-(trifluoromethoxy)benzo[b]selenophen-3-yl)pyridin-2-yl)cyclopropane-1-carboxylic acid (Compound 55)

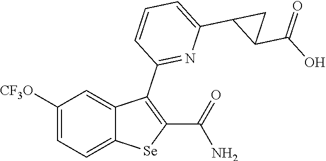

To a mixture consisting of trans-(rac)-ethyl 2-(6-(2-carbamoyl-5-(trifluoromethoxy)benzo[b]selenophen-3-yl)pyridin-2-yl)cyclopropane-1-carboxylate (0.045 g, 0.09 mmol) in MeOH (3 mL) was added dropwise a 1M aqueous solution of LiOH (0.27 mL, 3 eq). The reaction mixture was stirred overnight at room temperature. The solution was subjected to reduced pressure on a rotary evaporator to remove MeOH. Additional water was added (30 mL) and the aqueous mixture acidified with 1N HCl to precipitate product. Filter and wash solids with water. The crude product was purified by flash silica column chromatography on a CombiFlash NextGen 300+ purification system. Elution through a 4 g RediSep Gold Rf flash silica cartridge with 10-80% ethyl acetate in heptane containing 1% acetic acid afforded the title compound as an off-white solid after high vacuum at 40° C. overnight (0.014 g, 33% yield). Rf 0.18 with 1:40:60 acetic acid-ethyl acetate-heptane (UV 254 nM); ¹H NMR (DMSO-d6, 400 MHz) δ 12.35 (br s, 1H), 8.31 (br d, 1H, J=7.8 Hz), 7.86 (br s, 1H), 7.70 (br s, 1H), 7.57 (br s, 3H), 7.45 (br s, 1H), 7.4-7.4 (m, 1H), 2.6-2.8 (m, 1H), 2.0-2.1 (m, 1H), 1.48 (br s, 2H); LC-MS 2 peaks (ret. time 3.25, 3.34 min) both m/z 471.0 (M+1) and 469.0 (M−1) with Se isotope distribution; HPLC UV combined purity, Rt=7.14, 7.67 min, 96.2%; melting point=135.0-137.0° C. (dec).

### Example 64: Synthesis of trans-2-(6-(2-carbamoyl-5-(trifluoromethoxy)benzo[b]thiophen-3-yl)pyridin-2-yl)cyclopropane-1-carboxylic acid (Compound 56)

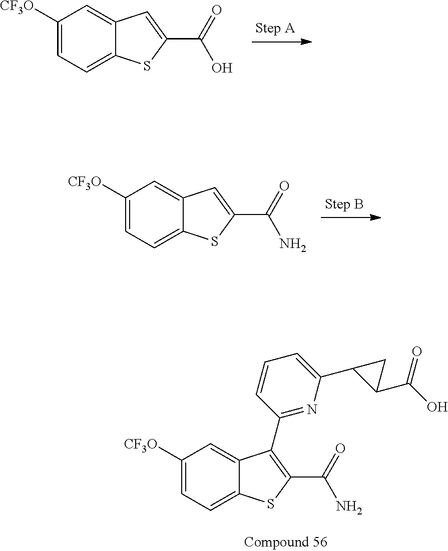

### Step A: Preparation of 5-(trifluoromethoxy)benzo[b]thiophene-2-carboxamide

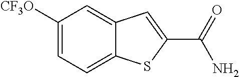

To a mixture consisting of 5-(trifluoromethoxy)benzo[b]thiophene-2-carboxylic acid (0.524 g, 2.0 mmol) in DMF (12.0 mL) was added ammonium chloride (Chem-Impex, 0.321 g, 6.0 mmol). Next was added TBTU (Oakwood, 0.963 g, 3.0 mmol) followed by the addition of DIPEA (3.1 mL, 18 mmol). The reaction mixture was stirred overnight at room temperature under N₂ atmosphere. The reaction mixture was subsequently diluted with water (400 mL) then extracted with 2×300 mL of ethyl acetate. The combined organic phases were dried with Na₂SO₄, filtered and solvent evaporated under reduced pressure to afford the crude product as an oil. The crude oil was purified by flash silica column chromatography on a CombiFlash NextGen 300+ purification system. Elution through a 40 g RediSep Gold Rf flash silica cartridge with 10-50% ethyl acetate in heptane containing 1% acetic acid afforded the title compound as a white crystalline solid (0.485 g, 92% yield). R_{f} 0.26 with 1:30:70 v/v acetic acid-ethyl acetate-hexane (UV 254 nM); ¹H-NMR (400 MHz; CDCl₃) δ 7.89 (d, 1H, J=8.9 Hz), 7.79 (s, 1H), 7.71 (s, 1H), 7.3-7.4 (m, 1H), 6.16 (br s, 2H); LC-MS ret. time 3.55 min m/z 259.9 (M−1); melting point=146.0-148.0° C.

### Step B: Preparation trans-2-(6-(2-carbamoyl-5-(trifluoromethoxy)benzo[b]thiophen-3-yl)pyridin-2-yl)cyclopropane-1-carboxylic acid (Compound 56)

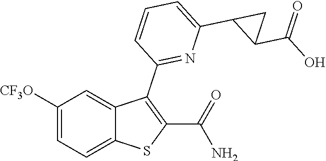

To a 20 mL septa-cap vial was added trans-2-(6-bromopyridin-2-yl)cyclopropane-1-carboxylic acid (0.095 g, 0.39 mmol) and 5-(trifluoromethoxy)benzo[b]thiophene-2-carboxamide (0.086 g, 0.32 mmol). Next was added Bis(dichloro(η⁶-p-cymene)ruthenium) (Strem, 0.010 g, 5 mol %), trimethylphosphonium tetrafluoroborate (Strem, 0.006 g, 10 mol %), K₂CO₃ (VWR, 0.102 g, 0.67 mmol) and NMP (3 mL). The mixture was degassed by bubbling in nitrogen gas for 3 min with stirring then heated to 110° C. overnight. Solvent was evaporated by a stream of nitrogen under mild heat to give a crude oil which was purified by flash silica column chromatography on a CombiFlash NextGen 300+ purification system. Elution through a 24 g RediSep Gold Rf flash silica cartridge with 10-100% ethyl acetate in heptane containing 1% acetic acid afforded the title compound as a dark oil. The oil was taken up into small amount of ethyl acetate (1 mL) then further diluted with hexane to precipitate additional solids. Solids were filtered and washed with hexane then air dried followed by high vacuum overnight at 40° C. to give the title compound as an off-white powder (0.011 g, 8% yield). R_{f} 0.33 with 1:60:40 v/v acetic acid-ethyl acetate-hexane (UV 254 nM); ¹H NMR (METHANOL-d4, 400 MHz) δ 8.07 (d, 1H, J=8.9 Hz), 7.88 (t, 1H, J=7.7 Hz), 7.64 (s, 1H), 7.47 (dd, 2H, J=4.0, 7.7 Hz), 7.42 (d, 1H, J=9.0 Hz), 2.7-2.8 (m, 1H), 2.21 (ddd, 1H, J=3.9, 5.1, 8.7 Hz), 1.6-1.7 (m, 2H); ¹⁹F-NMR (376 MHz; METHANOL-d4) δ −59.51; MS (APCI+) m/z 423.0 (M+1), m/z 421.0 (M−1), HPLC UV purity, Rt=7.11 min, 96.6%; melting point=219.0-221.0° C. (dec).

### Example 65: Synthesis of trans-2-(6-(2-carbamoyl-5-(trifluoromethoxy)benzo[b]selenophen-3-yl)pyridin-2-yl)cyclopropane-1-carboxylic acid (Compound 57)

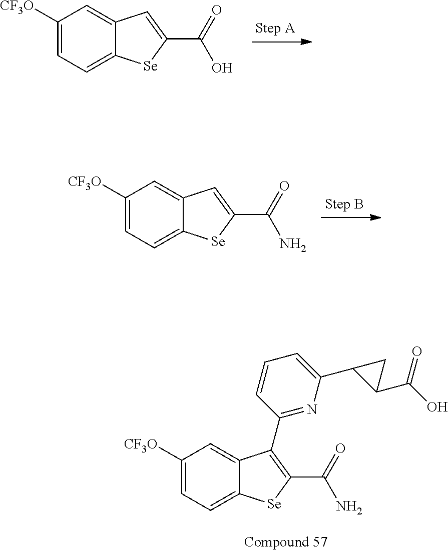

### Step A: Preparation of 5-(trifluoromethoxy)benzo[b]selenophene-2-carboxamide

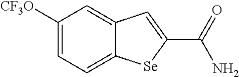

To a mixture consisting of 5-(trifluoromethoxy)benzo[b]selenophene-2-carboxylic acid (0.516 g, 1.7 mmol) in DMF (6.0 mL) was added ammonium chloride (Chem-Impex, 0.268 g, 5.0 mmol). Next was added TBTU (Oakwood, 0.804 g, 2.5 mmol) followed by the addition of DIPEA (2.6 mL, 15 mmol). The reaction mixture was stirred overnight at room temperature under N₂ atmosphere. The reaction mixture was subsequently diluted with water (250 mL) then extracted with 2×130 mL of ethyl acetate. The combined organic phases were washed with water (200 mL) then brine (100 mL) and dried with Na₂SO₄. The organics were then filtered, and solvent evaporated under reduced pressure to afford crude product as an oil. The crude oil was purified by flash silica column chromatography on a CombiFlash NextGen 300+ purification system. Elution through a 24 g RediSep Gold Rf flash silica cartridge with 10-50% ethyl acetate in heptane containing 1% acetic acid afforded the title compound as an off-white crystalline solid (0.480 g, 93% yield). R_{f} 0.32 with 1:40:60 v/v acetic acid-ethyl acetate-hexane (UV 254 nM); ¹H-NMR (400 MHz; CDCl₃) δ 7.93 (s, 1H), 7.92 (d, 1H, J=7.6 Hz), 7.71 (d, 1H, J=1.4 Hz), 7.27 (dd, 1H, J=1.4, 7.6 Hz), 6.25 (br s, 2H); LC-MS ret. time 3.58 min m/z 307.9 (M−1) with Se isotope distribution; melting point=170.0-172.0° C.

### Step B: Preparation trans-2-(6-(2-carbamoyl-5-(trifluoromethoxy)benzo[b]selenophen-3-yl)pyridin-2-yl)cyclopropane-1-carboxylic acid (Compound 57)

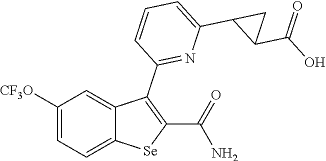

To a 20 mL septa-cap vial was added trans-2-(6-bromopyridin-2-yl)cyclopropane-1-carboxylic acid (0.095 g, 0.39 mmol) and 5-(trifluoromethoxy)benzo[b]selenophene-2-carboxamide (0.100 g, 0.32 mmol). Next was added Bis(dichloro(η⁶-p-cymene)ruthenium) (Strem, 0.010 g, 5 mol %), trimethylphosphonium tetrafluoroborate (Strem, 0.006 g, 10 mol %), K₂CO₃ (VWR, 0.93 g, 0.67 mmol) and NMP (3 mL). The mixture was degassed by bubbling in nitrogen gas for 3 min with stirring then heated to 130° C. overnight. The reaction mixture was subsequently cooled to room temperature and diluted with water (150 mL). The mixture was extracted with ethyl acetate (2×90 mL) and the combined organics dried with Na₂SO₄. Organics were filtered and solvent evaporated to give a crude oil which was purified by flash silica column chromatography on a CombiFlash NextGen 300+ purification system. Elution through a 24 g RediSep Gold Rf flash silica cartridge with 10-100% ethyl acetate in heptane containing 1% acetic acid gave product as residue. The residue was taken up into a small amount of ethyl acetate (1 mL) then further diluted with hexane to further precipitate solids. Solids were filtered and washed with hexane then air dried followed by high vacuum overnight at 40° C. to give the title compound (0.012 g, 8% yield) as an off-white powder. R_{f} 0.18 with 1:40:60 v/v acetic acid-ethyl acetate-hexane (UV 254 nM); ¹H NMR (DMSO-d6, 400 MHz) δ 12.37 (br s, 1H), 8.31 (d, 1H, J=8.9 Hz), 7.86 (t, 1H, J=7.8 Hz), 7.70 (br s, 1H), 7.5-7.6 (m, 3H), 7.45 (dd, 1H, J=1.6, 8.7 Hz), 7.39 (d, 1H, J=7.3 Hz), 2.69 (ddd, 1H, J=3.9, 5.7, 9.0 Hz), 2.0-2.1 (m, 1H), 1.4-1.5 (m, 2H); ¹⁹F-NMR (376 MHz; DMSO-d6) δ −56.84; MS (APCI+) m/z 471.0 (M+1), m/z 469.0 (M−1) with Se isotope distribution, HPLC UV purity, Rt=3.35 min, 98.5%; melting point 213.0-215.0° C. (dec).

### Example 66: Synthesis of trans-2-(6-(2-carbamoyl-6-(trifluoromethoxy)-1H-benzo[d]imidazol-1-yl)pyridin-2-yl)cyclopropane-1-carboxylic acid (Compound 58)

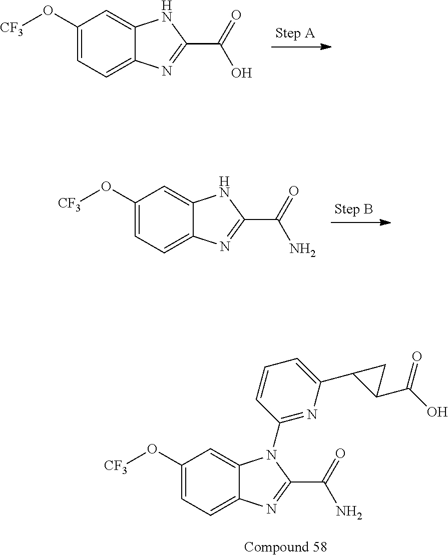 [SMILES: O=C(O)c1nc2ccc(OC(F)(F)F)cc2[nH]1 ; NC(=O)c1nc2ccc(OC(F)(F)F)cc2[nH]1 ; NC(=O)c1nc2ccc(OC(F)(F)F)cc2n1-c1cccc(C2CC2C(=O)O)n1]

### Step A: Preparation of 6-(trifluoromethoxy)-1H-benzo[d]imidazole-2-carboxamide

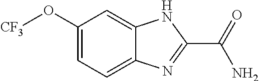 [SMILES: NC(=O)c1nc2ccc(OC(F)(F)F)cc2[nH]1]

To a mixture consisting of 6-(trifluoromethoxy)-1H-benzo[d]imidazole-2-carboxylic acid (0.700 g, 2.840 mmol) in DMF (8.0 mL) was added ammonium chloride (Chem-Impex, 0.450 g, 8.530 mmol). Next was added TBTU (Oakwood, 1.370 g, 4.260 mmol) followed by the addition of DIPEA (2.980 mL, 17.0 mmol). The reaction mixture was stirred overnight at room temperature under N₂ atmosphere. The reaction mixture was poured onto ice cold water (100 mL) and stirred for 30 minutes, solid formation was observed and filtered washed with excess water (100 mL), heptane (50 mL), diethyl ether (50 mL) and dried to afford product as white solid (0.460 g, 66% yield); Rf 0.4 with 1:10:90 v/v acetic acid-methanol-DCM (UV 254 nM); ¹H-NMR (400 MHz; DMSO-d₆) δ 12.89-13.76 (m, 1H), 8.29 (br s, 1H), 7.03-8.06 (m, 4H); MS (APCI⁺) m/z 244.0 (M−H); melting point=198-201° C.

### Step B: Preparation of trans-2-(6-(2-carbamoyl-6-(trifluoromethoxy)-1H-benzo[d]imidazol-1-yl)pyridin-2-yl)cyclopropane-1-carboxylic acid (Compound 58)

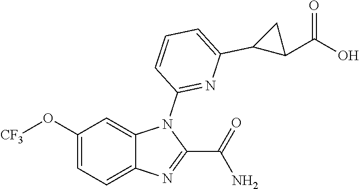 [SMILES: NC(=O)c1nc2ccc(OC(F)(F)F)cc2n1-c1cccc(C2CC2C(=O)O)n1]

To a mixture consisting of 6-(trifluoromethoxy)-1H-benzo[d]imidazole-2-carboxamide (0.245 g, 1.00 mmol), trans-(−)-2-(6-bromopyridin-2-yl)cyclopropane-1-carboxylic acid (0.265 g, 1.10 mmol), copper (I) iodide (Strem, 0.190 g, 1.00 mmol) in DMSO (5 mL) was added DBU (Oakwood Chemicals, 0.30 mL, 2.00 mmol). The reaction mixture was heated to 120° C. under N₂ atmosphere for 6 hours and then allowed to cool to room temperature. The reaction mixture was subsequently partitioned between ethyl acetate (100 mL) and 1M KHSO₄ (50 mL). A precipitate formed which was collected by filtration over a fritted funnel and set aside. The filtrate was poured in a separatory funnel. The phases were separated, and the organic phase was partitioned with H₂O (50 mL), followed by brine (50 mL). The organic layer was separated and dried over anhydrous sodium sulfate, filtered, and concentrated under reduced pressure to give a crude gummy mass, 0.300 g. The crude was purified by flash silica column chromatography on a CombiFlash NextGen 300+purification system. Elution through a 12 g RediSep Gold Rf flash silica cartridge with 10-90% ethyl acetate in hexanes containing 1% acetic acid afforded the title compound as a brown solid (0.088 g, 22% yield). R_{f} 0.3 with 1:60:40 v/v acetic acid-ethyl acetate-hexane (UV 254 nM); ¹H-NMR (400 MHz; DMSO-d6) δ 12.32 (br s, 1H), 10.02 (s, 1H), 7.96 (d, J=8.02 Hz, 1H), 7.37-7.94 (m, 4H), 7.33 (br d, J=8.48 Hz, 1H), 7.26 (d, J=7.34 Hz, 1H), 2.54 (ddd, J=8.88, 5.56, 3.90 Hz, 1H), 2.04-2.09 (m, 1H), 3.21 Hz, 1H), 1.50 (td, J=5.62, 1.39-1.43 (m, 1H); ¹⁹F-NMR (376 MHz; DMSO-d6) δ −56.9; MS (APCI⁺) m/z 407.0 (M+H), (APCI⁻) m/z 405.0 (M−H); HPLC UV purity, Rt=7.959 min, 99.0%; melting point=194-195° C.

### Example 67: Synthesis of 2-(1-(6-(2-carbamoyl-6-(trifluoromethoxy)-1H-indol-1-yl)pyridin-2-yl)cyclopropyl)acetic acid (Compound 59)

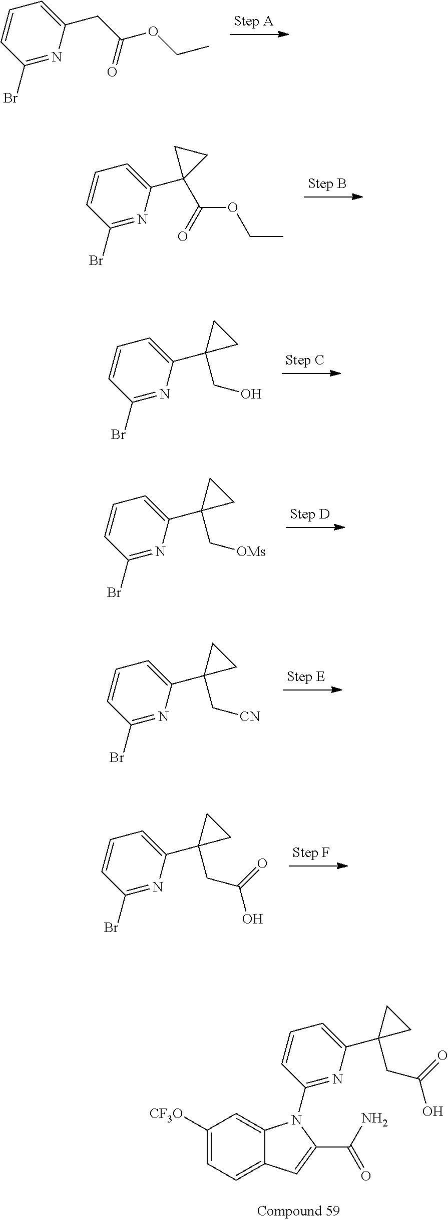

### Step A: Preparation of Ethyl 1-(6-bromopyridin-2-yl)cyclopropane-1-carboxylate

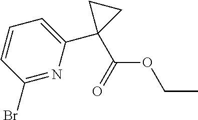

To a mixture consisting of ethyl 2-(6-bromopyridin-2-yl)acetate (Enamine, 1.50 g, 6.15 mmol) in dry DMSO (50 mL) was stirred under N₂. Sodium hydride (Sigma Aldrich, 60% dispersion, 0.54 g, 13.52 mmol) was added slowly in small portions over 15 minutes. The cloudy reaction mixture was stirred at room temperature for 10 minutes and then 1,2-dibromoethane (AK Scientific, 1.21 g, 6.45 mmol) was added dropwise via syringe over 5 minutes. The reaction was left to stir at room temperature overnight under a N₂ atmosphere, and then quenched with H₂O (60 mL). The mixture was transferred to a separatory funnel and partitioned with ethyl acetate (3×75 mL). The organics were combined, partitioned with brine (30 mL), dried over MgSO₄, filtered, and concentrated under reduced pressure to afford a crude clear yellow/brown oil (1.64 g). The crude oil was purified by flash silica column chromatography on a CombiFlash NextGen 300+ purification system. Elution through a 40 g RediSep Gold Rf flash silica cartridge with 0-5% ethyl acetate in heptane afforded the title compound as a clear pale-yellow oil (1.08 g, 65% yield). R_{f} 0.67 with 40:60 v/v ethyl acetate-hexane (UV 254 nM); ¹H NMR (CHLOROFORM-d, 400 MHz) δ 7.4-7.5 (m, 2H), 7.32 (dd, 1H, J=0.9, 7.6 Hz), 4.13 (q, 2H, J=7.1 Hz), 1.6-1.7 (m, 2H), 1.5-1.5 (m, 2H), 1.19 (t, 3H, J=7.1 Hz) Rf 0.69 with 25:75 v/v ethyl acetate-heptane (UV 254 nM); MS (APCI+) m/z 270.0 (M+1); HPLC UV purity, 210 nm, Rt=8.045 min, 99.05%.

### Step B: Preparation of (1-(6-bromopyridin-2-yl)cyclopropyl)methanol

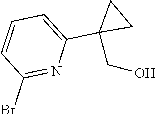 [SMILES: OCC1(c2cccc(Br)n2)CC1]

To a mixture consisting of ethyl 1-(6-bromopyridin-2-yl)cyclopropane-1-carboxylate (0.80 g, 2.96 mmol) in dry THE (50 mL) was stirred under N₂. A solution of 1M BH₃·THE in THE (Sigma Aldrich, 9 mL, 6 mmol) was added slowly dropwise via syringe over 5 minutes. The reaction mixture was heated to 60° C. and stirred overnight under N₂ atmosphere. Additional 1M BH₃ THE in THE (Sigma Aldrich, 6 mL, 6 mmol) was added to the reaction mixture and it was heated to 80° C. overnight under a N₂ atmosphere. The reaction mixture was concentrated under reduced pressure to afford a clear pale oil. This material was partitioned between methyl tert-butyl ether (75 mL) and H₂O (50 mL). The aqueous portion was partitioned with additional methyl tert-butyl ether (2×30 mL). The organics were combined and partitioned with brine (30 mL), dried over Na₂SO₄, filtered, and concentrated to afford 0.92 g clear colorless oil. The crude oil was purified by flash silica column chromatography on a CombiFlash NextGen 300+ purification system. Elution through a 40 g RediSep Gold Rf flash silica cartridge with 0-25% ethyl acetate in heptane afforded the title compound as a white solid (0.500 g, 74% yield). R_{f} 0.22 with 25:75 v/v ethyl acetate-hexane (UV 254 nM); ¹H NMR (CHLOROFORM-d, 400 MHz) δ 7.45 (t, 1H, J=7.7 Hz), 7.3-7.3 (m, 1H), 6.97 (d, 1H, J=7.8 Hz), 3.84 (br s, 2H), 3.34 (br s, 1H), 1.1-1.2 (m, 2H), 1.1-1.1 (m, 2H). MS (APCI+) m/z 228.0 (M+1); HPLC UV purity, 210 nm, Rt=5.863 min, 99.76%; melting point=85.5-86.5° C.

### Step C: Preparation of (1-(6-bromopyridin-2-yl)cyclopropyl)methyl methanesulfonate

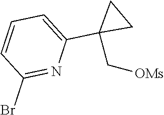 [SMILES: CS(=O)(=O)OCC1(c2cccc(Br)n2)CC1]

To a mixture consisting of 1-(6-bromopyridin-2-yl)cyclopropyl)methanol (0.20 g, 0.877 mmol) in dry DCM (8 mL) was stirred under N₂.The reaction was cooled 0° C., and triethylamine (TCI, 0.243 mL, 1.75 mmol) was added in one portion. Methanesulfonyl chloride (Sigma Aldrich, 0.12 g, 1.05 mmol) was added slowly dropwise over 5 minutes. The reaction was left to warm slowly to room temperature overnight under a N₂ atmosphere. The reaction mixture was concentrated under reduced pressure to afford a yellow solid. This material was triturated in ethyl acetate at room temperature for 30 minutes and the solids were removed by filtration. The clear yellow filtrate solution was then concentrated under reduced pressure; the yellow solids (0.370 g) were carried forward to the next step without further purification or characterization.

### Step D: Preparation of 2-(1-(6-bromopyridin-2-yl)cyclopropyl)acetonitrile

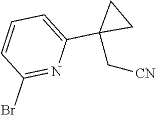 [SMILES: N#CCC1(c2cccc(Br)n2)CC1]

To a mixture consisting of (1-(6-bromopyridin-2-yl)cyclopropyl)methyl methanesulfonate (0.37 g, 0.877 mmol) in dry DMF (10 mL) was stirred under N₂. Sodium cyanide (0.257 g, 5.25 mmol) was added in one portion and the reaction was heated to 60° C. and stirred overnight under N₂. The reaction mixture was partitioned with 1:1 H₂O: saturated sodium bicarbonate solution (100 mL) and methyl tert-butyl ether (100 mL). The aqueous was partitioned with additional methyl tert-butyl ether (2×50 mL). The organic layers were combined, partitioned with brine (30 mL), and dried over Na₂SO₄, filtered, and concentrated under reduced pressure to afford a clear brown oil (0.189 g). The crude oil was purified by flash silica column chromatography on a CombiFlash NextGen 300+ purification system. Elution through a 4 g RediSep Gold Rf flash silica cartridge with 0-15% ethyl acetate in heptane afforded the title compound as a white solid (0.174 g, 82% yield). R_{f} 0.43 with 35:65 v/v ethyl acetate-heptane (UV 254 nM); ¹H NMR (CHLOROFORM-d, 400 MHz) δ 7.49 (t, 1H, J=7.8 Hz), 7.33 (d, 1H, J=8.0 Hz), 7.06 (d, 1H, J=7.8 Hz), 2.89 (s, 2H), 1.3-1.3 (m, 2H), 1.1-1.2 (m, 2H). ¹³C NMR (CHLOROFORM-d, 101 MHz) δ 162.4, 141.6, 139.0, 125.7, 118.2, 117.6, 24.5, 22.1, 16.7. MS (APCI+) m/z 237.0 (M+1); HPLC UV purity, 210 nm, Rt=3.579 min, 98.70%; melting point=43° C.-44.5° C.

### Step E: Preparation of 2-(1-(6-bromopyridin-2-yl)cyclopropyl)acetic acid

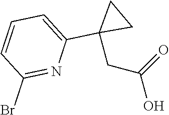 [SMILES: O=C(O)CC1(c2cccc(Br)n2)CC1]

To a mixture consisting of 2-(1-(6-bromopyridin-2-yl)cyclopropyl)acetonitrile (0.17 g, 0.717 mmol) in methanol (5.8 mL) stirred under a N₂ atmosphere, was added 1M NaOH aqueous solution (5.8 mL) and the reaction mixture was heated to 80° C. for 72 hours. The reaction mixture was diluted with H₂O (10 mL) and partitioned with ethyl acetate (20 mL). The aqueous layer was then acidified with 2M HCl to pH ˜2. The aqueous layer was partitioned with ethyl acetate (2×25 mL) and the organics were combined, partitioned with brine (30 mL), dried over Na₂SO₄, filtered, and concentrated under reduced pressure to afford a white solid (0.130 g, 67% yield). R_{f} 0.69 with 1:60:40 v/v acetic acid-ethyl acetate-hexane (UV 254 nM); ¹H NMR (CHLOROFORM-d, 400 MHz) δ 11.8-12.6 (m, 1H), 7.53 (t, 1H, J=7.8 Hz), 7.36 (d, 1H, J=7.8 Hz), 6.97 (d, 1H, J=8.0 Hz), 2.83 (s, 2H), 1.2-1.3 (m, 2H), 1.1-1.2 (m, 2H). MS (APCI+) m/z 255.9 (M+1); HPLC UV purity, 210 nm, Rt=6.339 min, 95.31%; melting point=138.5° C.-140.5° C.

### Step F: Preparation of 2-(1-(6-(2-carbamoyl-6-(trifluoromethoxy)-1H-indol-1-yl)pyridin-2-yl)cyclopropyl)acetic acid (Compound 59)

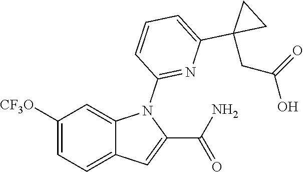

To a mixture consisting of 2-(1-(6-bromopyridin-2-yl)cyclopropyl)acetic acid (0.125 g, 0.488 mmol), 6-(trifluoromethoxy)-1H-indole-2-carboxamide (0.108 g, 0.444 mmol), and copper(I) iodide (Strem, 0.085 g, 0.444 mmol) in dry DMSO (3 mL) was stirred under N₂. DBU (Sigma Aldrich, 0.14 g, 0.888 mmol) was added last and the reaction was heated to 120° C. under a N₂ atmosphere for 2 hours and then allowed to cool to room temperature. The reaction mixture was subsequently partitioned between ethyl acetate (30 mL) and 1M KHSO4 (10 mL). The organics layer was partitioned with H₂O (30 mL), followed by brine (30 mL), and concentrated under reduced pressure to afford 0.187 g brown solid. The crude solid was purified by flash silica column chromatography on a CombiFlash NextGen 300+ purification system. Elution through a 12 g RediSep Gold Rf flash silica cartridge with a gradient of 10-100% 99:1 ethyl acetate: acetic acid in heptane afforded the title compound as a brown solid (0.039 g, 19% yield). R_{f} 0.08 with 1:60:40 v/v acetic acid-ethyl acetate-hexane (UV 254 nM); ¹H NMR (DMSO-d₆, 400 MHz) δ 12.10 (s, 1H), 8.14 (br s, 1H), 7.8-7.9 (m, 2H), 7.51 (br s, 1H), 7.47 (s, 1H), 7.28 (d, 1H, J=7.8 Hz), 7.21 (s, 1H), 7.19 (dd, 1H, J=0.9, 8.7 Hz), 7.13 (d, 1H, J=7.8 Hz), 2.81 (s, 2H), 1.2-1.3 (m, 2H), 0.9-1.0 (m, 2H). ¹⁹F NMR (DMSO-d₆, 376 MHz) δ −56.70 (s, 1F); MS (APCI+) m/z 420.1 (M+1); HPLC UV purity, 210 nm, Rt=7.321 min, 94.78%; melting point=195-198° C. (dec).

### Example 68: Synthesis of trans-(rac)-2-(1-(6-(2-carbamoyl-6-(trifluoromethoxy)-1H-indol-1-yl)pyridin-2-yl)cyclopropyl)acetic acid (Compound 60)

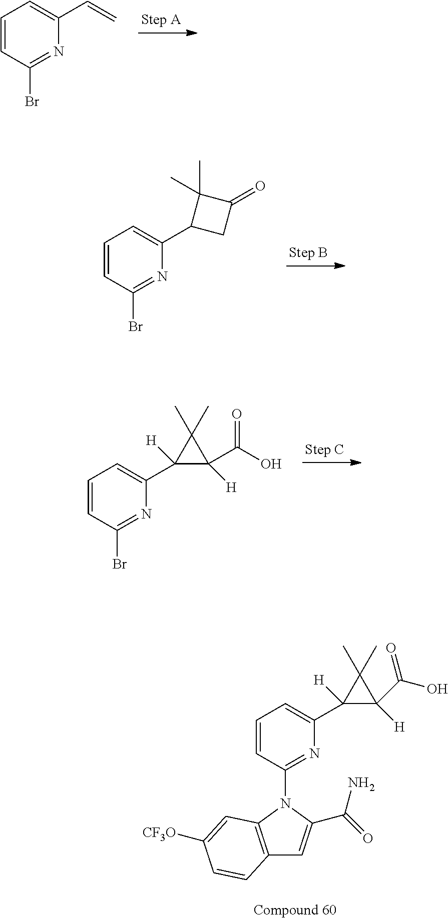

### Step A: Preparation of 3-(6-bromopyridin-2-yl)-2,2-dimethylcyclobutan-1-one

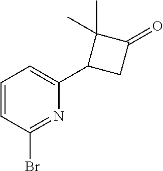

To a mixture consisting of N,N-Dimethylisobutyramide (Oakwood, 0.360 g, 3.12 mmol) in anhydrous DCM (5.7 mL) cooled to −15° C. was added trifluoromethanesulfonic anhydride (Oakwood, 0.585 mL, 1.28 mmol). The reaction mixture was stirred for 10 minutes after which, a solution of 2-bromo-6-vinylpyridine (95% mix, TBC as stabilizer) (Ambeed, 0.339 mL, 2.72 mmol) and 2,4,6-Trimethylpyridine (Aldrich, 0.46 mL, 3.50 mmol) in DCM (0.86 mL) was added. The reaction mixture was heated to reflux for 20 hours stirring under N₂ atmosphere. The reaction mixture was allowed to cool to room temperature, then concentrated under reduced pressure to afford a crude orange/brown residue (2.5 g). The crude residue was triturated with diethyl ether (3×10 mL). The crude material was dissolved in DCM (4.3 mL) and DI water (4.3 mL), and the reaction heated to reflux for 6 hours after which it was allowed to cool to room temperature. The reaction mixture was transferred to a separatory funnel, where the phases were separated, and the aqueous phase was partitioned with DCM (3×15 mL). The organic phase partitioned with brine (20 mL), dried over magnesium sulfate, filtered through a fritted funnel, and concentrated under reduced pressure to afford the crude orange oil (1.28 g). The crude oil was purified by flash chromatography on a CombiFlash NextGen 300+ purification system. Elution through a 40 g RediSep Gold Rf flash silica cartridge with a gradient of 0-15% ethyl acetate in heptane to afford a pale yellow crystalline solid. (0.409 g, 59% yield). R_{f} 0.29 with v/v 15:85 ethyl acetate-heptane (UV 254 nM); ¹H NMR (CHLOROFORM-d, 400 MHz) δ 7.53-7.51 (m, 1H), 7.37 (d, 1H J=7.8 Hz), 7.13 (d, 1H, J=7.6 Hz), 3.94 (dd, 1H, J=7.8, 17.7 Hz), 3.43 (t, 1H, J=8.4 Hz), 3.25 (dd, 1H, J=8.9, 17.7 Hz), 1.36 (s, 3H), 0.82 (s, 3H); ¹³C NMR (CHLOROFORM-d, 101 MHz) δ 213.3, 142.1, 138.7, 126.9, 126.3, 64.9, 46.1, 43.0, 23.8, 18.6; FIA-MS m/z, 254 (M+H); HPLC UV purity, 210 nm, Rt=8.02 min, 97.7%; melting point=58-61° C.

### Step B: Preparation of 3-(6-bromopyridin-2-yl)-2,2-dimethylcyclopropane-1-carboxylic acid

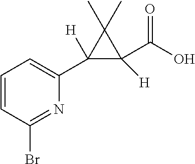

To a mixture consisting of 3-(6-bromopyridin-2-yl)-2,2-dimethylcyclobutan-1-one dissolved in anhydrous THE (5 mL) and cooled to −78° C. under N₂ atmosphere, 1.3 M lithium bis(trimethylsilyl)amide (TCI, 1.36 mL, 1.77 mmol) was added dropwise with internal temperature maintained below −55° C. After addition, the reaction mixture was stirred at −78° C. for 30 minutes then stirred at 0° C. for 15 minutes. The reaction mixture was cooled back to −78° C. and a solution of N-Bromosuccinimide (CHEM-IMPEX, 0.286 g, 1.61 mmol) in THF (4 mL) was added dropwise. After addition, the reaction mixture was stirred for 20 minutes and then was treated with a 2.5 M sodium hydroxide solution (4.02 mL). The cooling bath was removed, and the reaction mixture was stirred at room temperature for 30 minutes. The reaction mixture was concentrated under reduced pressure to remove THF. The remaining aqueous phase was transferred to a separatory funnel and partitioned with methyl tert-butyl ether (2×10 mL). The reaction mixture was acidified with concentrated HCl (2.07 mL) and stirred for 30 minutes. The reaction mixture was then transferred to a separatory funnel, and the aqueous phase was partitioned with methyl tert-butyl ether (2×15 mL). The organic phase was dried over magnesium sulfate, filtered through a fritted funnel, and concentrated under reduced pressure to afford a crude yellow oil (0.412 g). The crude oil was purified using a CombiFlash NextGen 300+ purification system. Elution through a 40 g Redi-Sep Gold column with a gradient of 0-25% ethyl acetate in heptane containing 1% acetic acid. Two crops were isolated containing product and concentrated under reduced pressure to afford a white solid (0.045 g, 7.5% yield), and an oily/solid residue (0.1594 g). R_{f} 0.38 with 1:30:70 v/v acetic acid-ethyl acetate-heptane (UV 254 nM); ¹H NMR (CHLOROFORM-d, 400 MHz) δ 12.31 (br s, 1H), 7.6-7.7 (m, 1H), 7.47 (t, 2H, J=7.1 Hz.), 2.59 (d, 1H, J=5.7 Hz), 2.31 (d, 1H, J=5.7 Hz.), 1.31 (s, 3H), 1.02 (s, 3H). FIA-MS m/z, 270 (M+H); HPLC UV purity, 210 nm, Rt=6.97 min, 73.9%;

### Step C: Preparation of trans-(rac)-3-(6-(2-carbamoyl-6-(trifluoromethoxy)-1H-indol-1-yl)pyridin-2-yl)-2,2-dimethylcyclopropane-1-carboxylic acid (Compound 60)

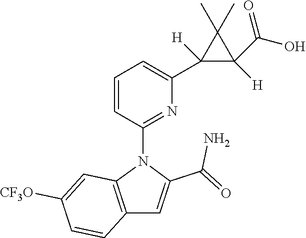

### Example 69: Synthesis of trans-(rac)-3-(3-(2-carbamoyl-6-(trifluoromethoxy)-1H-indol-1-yl)phenyl)-2,2-dimethylcyclopropane-1-carboxylic acid (Compound 61)

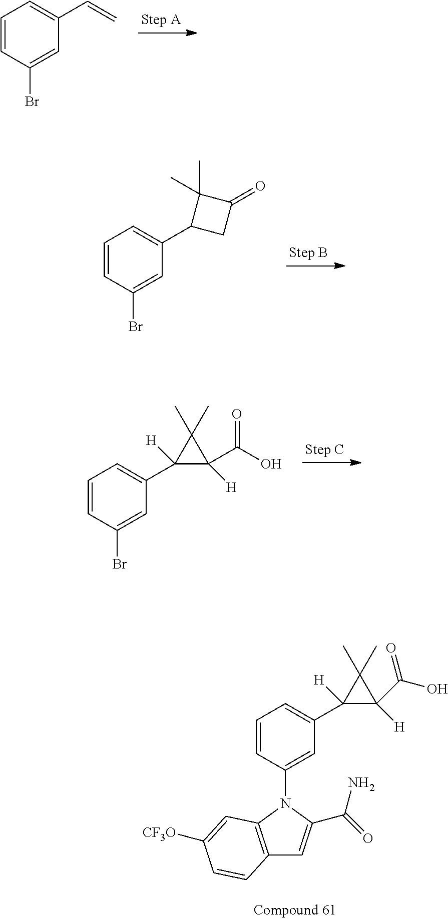

### Step A: Preparation of 3-(3-bromophenyl)-2,2-dimethylcyclobutan-1-one

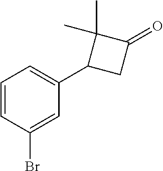

To a mixture consisting of N,N-Dimethylisobutyramide (Oakwood, 0.362 g, 3.14 mmol) in anhydrous DCM (5.7 mL) was cooled to −15° C. Once cooled, trifluoromethanesulfonic anhydride (Oakwood, 0.59 mL, 1.28 mmol) was added. The reaction mixture was stirred for 10 minutes after which, a solution of 3-bromostyrene (stabilized with 100 ppm 4-tert-Butylcatechol) (Oakwood, 0.36 mL, 2.73 mmol) and 2,4,6-trimethylpyridine (Aldrich, 0.47 mL, 3.52 mmol) in DCM (0.86 mL) was added. The reaction mixture was heated to reflux for 20 hours stirring under N₂ atmosphere. The reaction mixture was allowed to cool to room temperature, then concentrated under reduced pressure to afford a crude orange residue (2.71 g). The crude residue was taken up in diethyl ether (10 mL) and swirled to wash. The diethyl ether was decanted off and the wash was repeated (2×10 mL). Excess diethyl ether was concentrated under reduced pressure. The crude material was dissolved in DCM (4.3 mL) and DI water (4.3 mL) and the reaction heated to reflux for 6 hours after which it was allowed to cool to room temperature. The reaction mixture was transferred to a separatory funnel, where the phases were separated, and the aqueous phase was extracted with DCM (3×15 mL). The organic phase was washed with brine (20 mL), dried over magnesium sulfate, filtered through a fritted funnel, and concentrated under reduced pressure to afford a crude orange oil (1.70 g). The crude oil was purified by flash chromatography on a Biotage Isolera purification system. Elution through a 40 g RediSep Gold column with a 0-10% ethyl acetate in heptane to afford a colorless oil (0.593 g, 86% yield). R_{f} 0.33 with 1:1, methyl tert-butyl ether in heptane (UV 254 nM); ¹H NMR (CHLOROFORM-d, 400 MHz) δ 7.39 (d, 1H, J=8.0 Hz), 7.2-7.3 (m, 2H), 7.11 (d, 1H, J=7.6 Hz), 3.3-3.5 (m, 2H), 3.2-3.3 (m, 1H), 1.34 (s, 3H), 0.80 (s, 3H); 13C NMR (CHLOROFORM-d, 101 MHz) δ 212.8, 141.5, 130.8, 130.1, 129.9, 126.3, 122.8, 64.1, 46.0, 41.3, 23.5, 19.1; FIA-MS m/z, 253 (M+H); HPLC UV purity, 210 nm, Rt=9.2 min, 93.5%.

### Step B: Preparation of 3-(3-bromophenyl)-2,2-dimethylcyclopropane-1-carboxylic acid

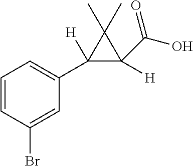

To a mixture consisting of 3-(3-bromophenyl)-2,2-dimethylcyclobutan-1-one (g, mmol) dissolved in anhydrous THE (5 mL) and cooled to −78° C. under N2 atmosphere. Once cooled, 1.3 M lithium bis(trimethylsilyl)amide (TCI, 1.10 mL, 1.54 mmol) was added dropwise with internal temperature maintained below −55° C. After addition, the reaction mixture was stirred at −78° C. for 30 minutes then stirred at 0° C. for 15 minutes. The reaction mixture was cooled back to −78° C. and a solution of N-Bromosuccinimide (CHEM-IMPEX, 0.249 g, 1.40 mmol) in THF (3 mL) was added dropwise. After addition, the reaction mixture was stirred for 20-40 minutes and then was treated with a 2.5 M sodium hydroxide solution (3.5 mL). The cooling bath was removed, and the reaction mixture stirred at room temperature for 30 minutes. The reaction mixture was concentrated under reduced pressure to remove THF. The remaining aqueous layer was transferred to a separatory funnel and washed with MTBE (2×10 mL). The aqueous layer was concentrated under reduced pressure to remove excess MTBE then cooled to 0° C. The reaction mixture was acidified with concentrated HCl (1.8 mL) and stirred for 30 minutes. The reaction mixture was then transferred to a separatory funnel, where the phases were separated, and the aqueous phase was partitioned with methyl tert-butyl ether (2×15 mL). The organic phase was dried over magnesium sulfate, filtered through a fritted funnel, and concentrated under reduced pressure to afford a crude yellow oil, which solidified overnight (0.358 g, 95% yield). R_{f} 0.33 with 1:1, methyl tert-butyl ether in heptane (UV 254 nM); ¹H NMR (DMSO-d6, 400 MHz) δ 12.22 (br s, 1H), 7.4-7.5 (m, 2H), 7.2-7.3 (m, 2H), 2.48 (br s, 1H), 2.07 (d, 1H, J=6.0 Hz), 1.30 (s, 3H), 0.8-0.9 (m, 3H); FIA-MS m/z, 266.9 (M−H); HPLC UV purity, 210 nm, Rt=8.3 min, 96.3%; melting point=95° C.

### Step C: Preparation of 3-(3-(2-carbamoyl-6-(trifluoromethoxy)-1H-indol-1-yl)phenyl)-2,2-dimethylcyclopropane-1-carboxylic acid (Compound 61)

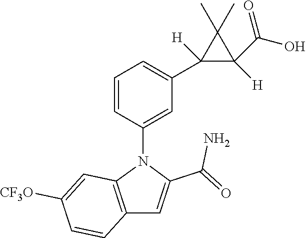

To a mixture of 3-(3-bromophenyl)-2,2-dimethylcyclopropane-1-carboxylic acid (0.077 g, 0.286 mmol), 6-(trifluoromethoxy)-1H-indole-2-carboxamide (0.064 g, 0.262 mmol), and copper(I) iodide (Strem, 0.050 g, 0.263 mmol) in dry DMSO (3 mL) was stirred under a N₂ atmosphere. DBU (Sigma Aldrich, 0.14 g, 0.888 mmol) was added last and the reaction was heated to 120° C. The reaction was cooled to room temperature and partitioned between ethyl acetate (30 mL) and 1M KHSO₄ aqueous solution (8 mL). The mixture filtered, and the filtrate phases were separated. The organic phase was partitioned with H₂O (20 mL), followed by brine (20 mL), and then concentrated under reduced pressure to afford 0.215 g brown residue. The crude solid was purified by flash silica column chromatography on a Biotage Isolera purification system. Elution through a 12 g RediSep Gold Rf flash silica cartridge with a gradient of 10-100% 99:1 ethyl acetate: acetic acid in heptane afforded the title compound as

### Example 70: Synthesis of 3-(6-(2-carbamoyl-6-(trifluoromethoxy)-1H-indol-1-yl)pyridin-2-yl)-3-methylbutanoic acid (Compound 62)

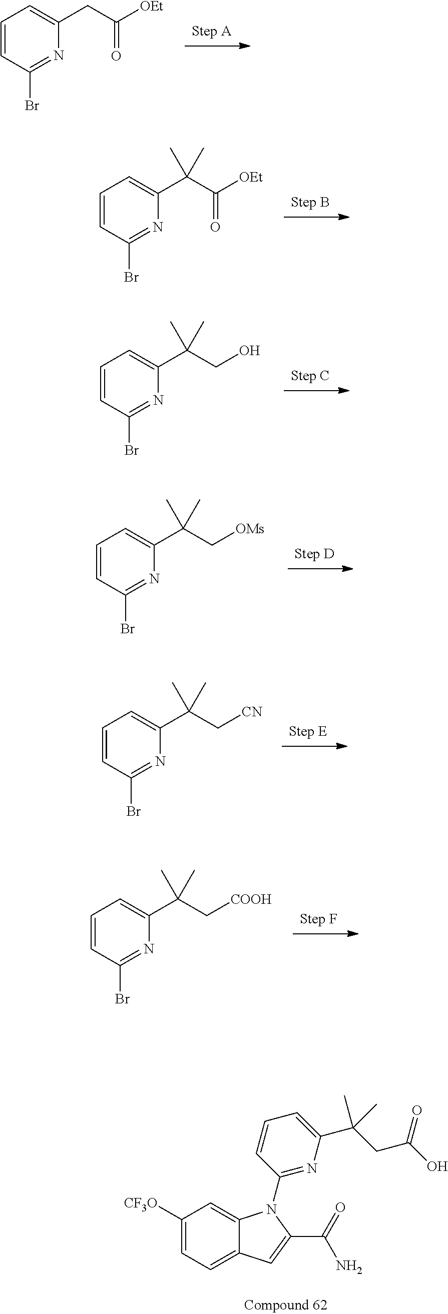

### Step A: Preparation of ethyl 2-(6-bromopyridin-2-yl)-2-methylpropanoate

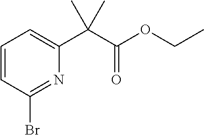

To a mixture consisting of KHMDS (Sigma Aldrich, 1M solution in THF, 18 mL, 18 mmol) in 165 mL THE at −78° C. under a N₂ atmosphere was added ethyl 2-(6-bromopyridin-2-yl)acetate (Combi-Blocks, 4.0 g, 16.4 mmol). After stirring for 20 minutes, iodomethane (Aldrich, 1.3 mL, 20.88 mmol) was added in one portion and the reaction mixture warmed to room temperature for 1 hour and 45 minutes, becoming cloudy and very faint brown. The reaction was again cooled to −78° C. and a second equivalent of 1M KHMDS solution in THE (Aldrich, 18 mL, 18 mmol) was added. The reaction mixture turned yellowish and less cloudy, stirred for 20 minutes, and then added second equivalent of iodomethane (Aldrich, 1.3 mL, 20.88 mmol) in one portion. The reaction mixture was warmed to room temperature and stirred overnight under N₂ atmosphere. To the reaction mixture was added H₂O (40 mL) and the mixture was transferred to a separatory funnel and partitioned with ethyl acetate (2×250 mL), and dichloromethane (150 mL). Organic layers were combined, dried over anhydrous sodium sulfate, filtered, and concentrated under reduced pressure to give a crude orange red oil, 5.01 g. The crude oil was purified by flash silica column chromatography on a CombiFlash NextGen 300+ purification system. Elution through a 120 g RediSep Gold Rf flash silica cartridge with 0-11% dichloromethane in heptane afforded the title compound as a light yellowish oil (4.59 g, quantitative yield); R_{f} 0.5 with 1:1 v/v methyl tert-butyl ether-heptane (UV 254 nM); ¹H-NMR (400 MHz; CHLOROFORM-d) 6 7.49 (t, 1H, J=7.8 Hz), 7.33 (d, 1H, J=7.8 Hz), 7.22 (d, 1H, J=7.4 Hz), 4.16 (q, 2H, J=7.1 Hz), 1.59 (s, 6H), 1.20 (t, 3H, J=7.1 Hz); ¹³C-NMR (400 MHz; CHLOROFORM-d) δ 175.6, 165.2, 141.1, 138.6, 126.0, 119.0, 61.0, 49.5, 25.5, 14.0 MS (APCI+) m/z 272 (M+1); HPLC UV purity Rt=4.15 min, 100%.

### Step B: Preparation of 2-(6-bromopyridin-2-yl)-2-methylpropan-1-ol

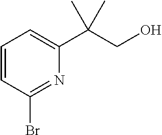

To a mixture consisting of ethyl 2-(6-bromopyridin-2-yl)-2-methylpropanoate (3.47 g, 12.803 mmol) in ethyl ether (105 mL) at 0° C. under a N₂ atmosphere, was slowly added lithium aluminum hydride (Sigma Aldrich, 1M solution in diethyl ether, 14.1 mL, 14.1 mmol). After 2 hours at 0° C. additional lithium aluminum hydride (Sigma Aldrich, 1.0 M solution in diethyl ether, 7 mL, 7 mmol) was slowly added to the reaction mixture, which was stirred at 0° C. for 1 hour. The reaction mixture was quenched at 0° C. with the slow addition of 0.5 mL water, then 0.5 mL 15% NaOH, and finally 15 mL water, stirred for ˜15 minutes, dried over anhydrous sodium sulfate, filtered, and concentrated under reduced pressure to give a crude yellow oil, 2.91 g. The crude oil was purified by flash silica column chromatography on a CombiFlash NextGen 300+ purification system. Elution through a 80 g RediSep Gold Rf flash silica cartridge with 0-47% methyl tert-butyl ether in heptane afforded the title compound as a colorless oil (1.83 g, 62% yield). R_{f} 0.33 with 1:1 v/v methyl tert-butyl ether-heptane (UV 254 nM); ¹H-NMR (400 MHz; CHLOROFORM-d) 6 7.51 (t, 1H, J=7.7 Hz), 7.33 (d, 1H, J=7.8 Hz), 7.27 (d, 1H, J=7.6 Hz), 3.74 (s, 2H), 1.31 (s, 6H); ¹³C-NMR (400 MHz; CHLOROFORM-d) δ 169.6, 140.5, 139.1, 125.6, 119.4, 71.5, 41.8, 25.4. MS (APCI+) m/z 231.9 (M+H); HPLC UV purity Rt=3.1 min, 91.9%.

### Step C: Preparation of 2-(6-bromopyridin-2-yl)-2-methylpropyl methanesulfonate

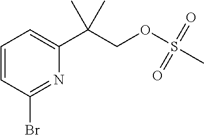 [SMILES: CC(C)(COS(C)(=O)=O)c1cccc(Br)n1]

A mixture of 2-(6-bromopyridin-2-yl)-2-methylpropan-1-ol (1.64 g, 7.16 mmol) in dry DCM (75 mL) was stirred under N₂. To the reaction at 0° C. was added triethylamine (TCI, 1.29 mL, 9.30 mmol) in one portion. Next, methanesulfonyl chloride (Sigma Aldrich, 0.98 g, 8.59 mmol) was added dropwise for five minutes. The reaction was slowly warmed to room temperature overnight under a N₂ atmosphere. The reaction mixture was concentrated under reduced pressure to afford a yellow solid. This material was triturated in ethyl acetate at room temperature for 30 minutes and the solids were removed by filtration. The clear yellow filtrate solution was then concentrated under reduced pressure; the yellow solids (2.07 g) were carried forward to the next step without further purification or characterization.

### Step D: Preparation of 3-(6-bromopyridin-2-yl)-3-methylbutanenitrile

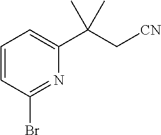

To a mixture consisting of 2-(6-bromopyridin-2-yl)-2-methylpropyl methanesulfonate (1.51 g, 4.92 mmol) in dry DMSO (100 mL) under a N₂ atmosphere was added sodium cyanide (1.43 g, 29.28 mmol) in one portion and the reaction was heated to 140° C. and stirred for 72 hours. The reaction mixture was cooled to room temperature and partitioned with methyl tert-butyl ether (300 mL). The organic phase was separated and partitioned with H₂O (50 mL), followed by brine (50 mL), dried over Na₂SO₄, filtered, and concentrated under reduced pressure to afford a clear red oil (0.803 g). The crude oil was purified by flash silica column chromatography on a CombiFlash NextGen 300+ purification system. Elution through a 40 g RediSep Gold Rf flash silica cartridge with 0-15% ethyl acetate in heptane afforded the title compound as a colorless oil (0.704 g, 60% yield). R_{f} 0.57 with 1:1 v/v methyl tert-butyl ether-heptane (UV 254 nM); ¹H NMR (CHLOROFORM-d, 400 MHz) δ 7.53 (t, 1H, J=7.7 Hz), 7.36 (d, 1H, J=7.6 Hz), 7.31 (d, 1H, J=7.8 Hz), 2.85 (s, 2H), 1.50 (s, 6H). ¹³C-NMR (400 MHz; CHLOROFORM-d) δ 166.1, 141.6, 139.2, 126.5, 118.5, 118.4, 39.9, 30.5, 27.5. MS (APCI+) m/z 240.9 (M+1); HPLC UV purity, 210 nm, Rt=3.8 min, 86.9%.

### Step E: Preparation of 3-(6-bromopyridin-2-yl)-3-methylbutanoic acid

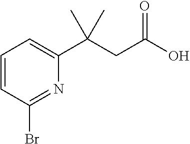

To a mixture consisting of 3-(6-bromopyridin-2-yl)-3-methylbutanenitrile (0.18 g, 0.77 mmol) in methanol (6 mL) stirred under a N₂ atmosphere, was added 1M NaOH aqueous solution (6 mL) and the reaction mixture was heated to 85° C. for 72 hours and then heated up to 110° C. overnight. The reaction mixture was diluted with H₂O (10 mL) and partitioned with ethyl acetate (20 mL). The aqueous layer was then acidified with 2M HCl to pH˜2. The aqueous layer was partitioned with ethyl acetate (2×25 mL) and the organics were combined, partitioned with brine (30 mL), dried over anhydrous sodium sulfate, filtered, and concentrated under reduced pressure to afford an off-white solid (0.090 g, 45% yield). R_{f} 0.08 with 4:6 v/v ethyl acetate-heptane (UV 254 nM); ¹H NMR (CHLOROFORM-d, 400 MHz) δ 7.5-7.6 (m, 1H), 7.35 (d, 1H, J=8 Hz), 7.32 (d, 1H, J=8 Hz), 2.85 (s, 2H), 1.44 (s, 6H); MS (APCI+) m/z 257.9 (M+1); HPLC UV purity, 210 nm, Rt=2.9 min, 90.0%; melting point=109-112° C.

### Step F: Preparation of 3-(6-(2-carbamoyl-6-(trifluoromethoxy)-1H-indol-1-yl)pyridin-2-yl)-3-methylbutanoic acid (Compound 62)

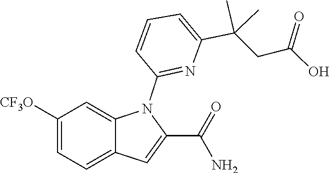

To a mixture consisting of 3-(6-bromopyridin-2-yl)-3-methylbutanoic acid (0.050 g, 0.195 mmol), 6-(trifluoromethoxy)-1H-indole-2-carboxamide (0.044 g, 0.180 mmol), and copper(I) iodide (Strem, 0.034 g, 0.179 mmol) in dry DMSO (3 mL) stirred under a N₂ atmosphere was added DBU (Sigma Aldrich, 0.055 g, 0.36 mmol) and the reaction was heated to 120° C. The reaction was cooled to room temperature and partitioned between ethyl acetate (30 mL) and 1M KHSO₄ aqueous solution (8 mL). The mixture filtered, and the filtrate phases were separated. The organic phase was partitioned with H₂O (30 mL), followed by brine (30 mL), and then concentrated under reduced pressure to afford 0.105 g of a dark red residue. The crude solid was purified by flash silica column chromatography on a CombiFlash NextGen 300+ purification system. Elution through a 10 g Biotage Sfar Silica HC D with a gradient of 0-100% 99:1 ethyl acetate in acetic acid in heptane to afford the title compound as a brown solid (0.016 g, 21% yield). R_{f} 0.09 with 1:60:40 acetic acid-ethyl acetate-heptane (UV 254 nM); ¹H NMR (DMSO-d₆, 400 MHz) δ 8.12 (br s, 1H), 7.85 (t, 1H, J=7.8 Hz), 7.80 (d, 1H, J=8.7 Hz), 7.73 (s, 1H), 7.58 (s, 1H), 7.50 (br dd, 1H, J=10.2, 18.2 Hz), 7.40 (d, 1H, J=7.8 Hz), 7.20 (s, 1H), 7.1-7.2 (m, 2H), 2.69 (s, 2H), 1.38 (s, 6H); ¹⁹F NMR (DMSO-d₆, 376 MHz) δ −56.70; MS (APCI+) m/z 422.1 (M+1); HPLC UV purity, 210 nm, Rt=3.6 min, 82.8%. MP=139-142° C. (decomp).

### Example 71: Synthesis of 3-(6-(2-carbamoyl-6-(trifluoromethoxy)-1H-indol-1-yl)pyridin-2-yl)-3,3-difluoropropanoic acid (Compound 63)

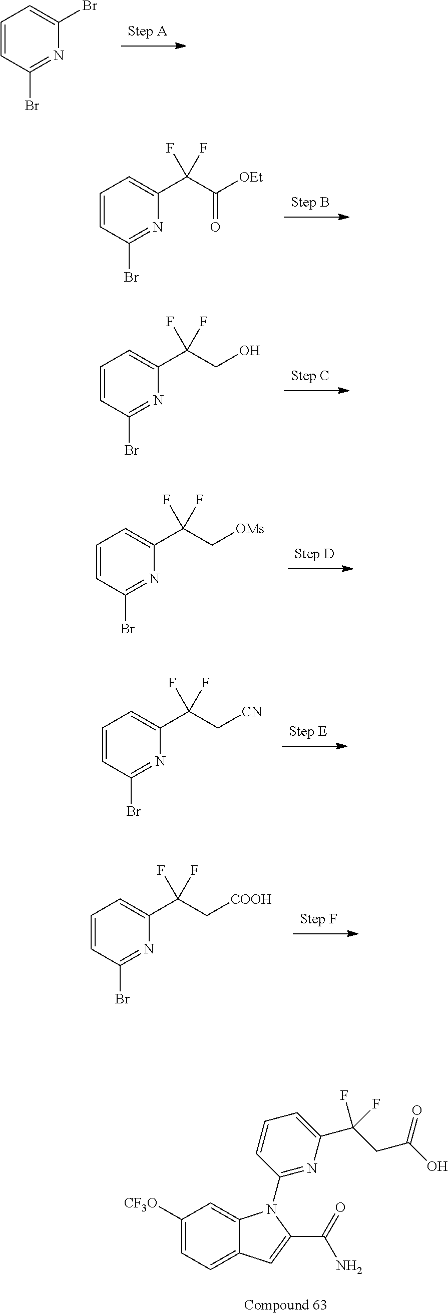

### Step A: Preparation of ethyl 2-(6-bromopyridin-2-yl)-2,2-difluoroacetate

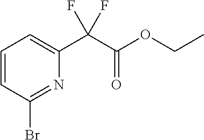

To a mixture consisting of 2,6-dibromopyridine (Oakwood, 6.75 g, 28.49 mmol) in DMSO (40 mL) was added ethyl bromodifluoroacetate (Oakwood, 7.51 g, 37.0 mmol). Next while stirring under N₂ atmosphere add Copper powder (4.1 g, 64.57 mmol). The reaction mixture was heated to 50° C. for 4 hours and then allowed to stir overnight at 60° C. After cooling the reaction mixture to room temperature, the reaction mixture was portioned between ethyl acetate (200 mL) and 1.3 M KH₂PO₄ (200 mL). The reaction mixture was stirred at room temperature for 30 minutes. Next filter the reaction mixture over a Buchner funnel. The filtrate was poured into a separatory funnel and the organic layer was separated. The organic layer was subsequently washed with 1:1 brine/water (2×200 mL). The organic layers was concentrated under reduced pressure to afford a crude orange oil, 6.95 g. The crude oil was purified by flash silica column chromatography on a CombiFlash NextGen 300+ purification system. Elution through a 120 g RediSep Gold Rf flash silica cartridge with 5-60% ethyl acetate in heptane afforded the title compound as a colorless oil (3.53 g, 44%). Rf 0.46 with 25:75 v/v ethyl acetate-heptane (UV 254 nM); ¹H-NMR (400 MHz; DMSO-d6) δ 8.00-8.04 (m, 1H), 7.90-7.93 (m, 2H), 4.36 (q, 2H, J=7.1 Hz), 1.21 (t, 3H, J=7.1 Hz); ¹⁹F-NMR (376 MHz; DMSO-d6) δ −103.82; MS (APCI+) m/z 280, 282 (M+1) Br present; HPLC UV purity Rt=8.230 min, 96.7%.

### Step B: Preparation of 2-(6-bromopyridin-2-yl)-2,2-difluoroethan-1-ol

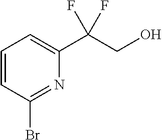 [SMILES: OCC(F)(F)c1cccc(Br)n1]

To a mixture consisting of ethyl 2-(6-bromopyridin-2-yl)-2,2-difluoroacetate (3.38 g, 12.07 mmol) in ethanol (50 mL) was added sodium borohydride (Aldrich, 0.342 g, 9.05 mmol) while in an ice water bath (0° C.). The reaction mixture was stirred overnight while warming to room temperature. The reaction mixture was cooled to 0° C. and then the reaction was quenched with the addition of 1N HCl (10 mL). The reaction mixture was stirred for 10 minutes then 1N NaOH (˜6 mL) was added to raise the pH=10. The reaction was poured into a separatory funnel and partitioned between dichloromethane (100 mL) and 1:1 brine/water (100 mL). The organic layer was separated and then dried over anhydrous sodium sulfate. The organic layer was concentrated under reduced pressure to afford the title product as a colorless oil, (2.62 g, 92.9%). R_{f} 0.27 with 25:75 v/v ethyl acetate-heptane (UV 254 nM); ¹H-NMR (400 MHz; DMSO-d6) δ 7.93 (t, 1H, J=7.8 Hz), 7.82 (d, 1H, J=8.0 Hz), 7.73 (d, 1H, J=7.6 Hz), 5.63 (t, 1H, J=6.3 Hz), 4.36 (t, 1H, J=5.2 Hz), 3.97 (br t, 3H, J=13.8 Hz), 3.95 (br t, 3H, J=13.9 Hz), 3.44 (dq, 1H, J=5.0, 7.0 Hz), 1.05 (t, 1H, J=7.0 Hz); ¹⁹F-NMR (376 MHz; DMSO-d6) δ −107.69, −107.72; MS (APCI+) m/z 237, 239 (M+1) Br present; HPLC UV purity Rt=5.195 min, 98.7%.

### Step C: Preparation of 2-(6-bromopyridin-2-yl)-2,2-difluoroethyl methanesulfonate

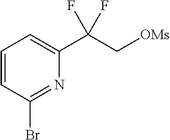 [SMILES: CS(=O)(=O)OCC(F)(F)c1cccc(Br)n1]

To a mixture consisting of 2-(6-bromopyridin-2-yl)-2,2-difluoroethan-1-ol (0.476 g, 2.00 mmol) in dry DCM (4 mL) was stirred under N₂.The reaction was cooled 0° C., and triethylamine (TCI, 0.28 mL, 2.00 mmol) was added in one portion. Methanesulfonyl chloride (Sigma Aldrich, 0.34 g, 3.00 mmol) was added slowly dropwise over 5 minutes. The reaction was left to warm slowly to room temperature overnight under a N₂ atmosphere. The reaction mixture was concentrated under reduced pressure to afford a yellow solid. This material was triturated in ethyl acetate at room temperature for 30 minutes and the solids were removed by filtration. The clear yellow filtrate solution was then concentrated under reduced pressure; the yellow solids (1.01 g) were carried forward to the next step without further purification or characterization.

### Step D: Preparation of 3-(6-bromopyridin-2-yl)-3,3-difluoropropanenitrile

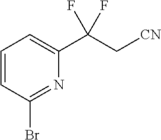 [SMILES: N#CCC(F)(F)c1cccc(Br)n1]

### Example 72: Synthesis of cis-(−)-2-(6-(2-carbamoyl-6-(trifluoromethoxy)-1H-indol-1-yl)pyridin-2-yl)cyclopropane-1-carboxylic acid (Compound 28d)

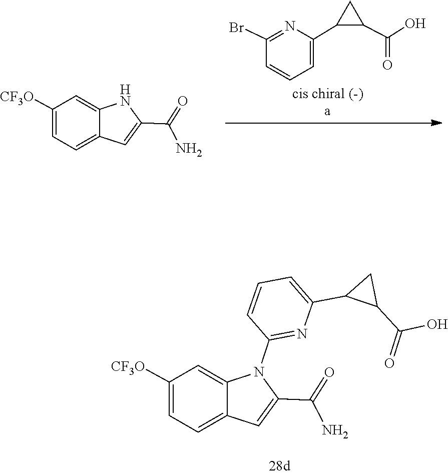

### Step A: Preparation of cis-(−)-2-(6-(2-carbamoyl-6-(trifluoromethoxy)-1H-indol-1-yl)pyridin-2-yl)cyclopropane-1-carboxylic acid (Compound 28d)

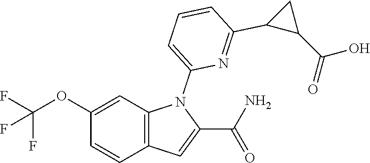 [SMILES: NC(=O)c1cc2ccc(OC(F)(F)F)cc2n1-c1cccc(C2CC2C(=O)O)n1]

To a mixture consisting of 6-(trifluoromethoxy)-1H-indole-2-carboxamide (0.293 g, 1.20 mmol), cis-(−)-2-(6-bromopyridin-2-yl)cyclopropane-1-carboxylic acid (0.242 g, 1.00 mmol), copper (I) iodide (Strem, 0.190 g, 1.00 mmol) in DMSO (20 mL) was added DBU (Oakwood Chemicals, 0.3 mL, 2.00 mmol). The reaction mixture was heated to 120° C. under N₂ atmosphere for 16 hours and then allowed to cool to room temperature. The reaction mixture was subsequently partitioned between ethyl acetate (50 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.308 g. The crude oil was purified by flash silica column chromatography on a CombiFlash NextGen 300+ purification system. Elution through a 12 g RediSep Gold Rf flash silica cartridge with 20-80% ethyl acetate in hexanes containing 1% acetic acid afforded the title compound as a tan solid (0.040 g, 10% yield); R_{f}0.14 with 1:70:30 v/v acetic acid-ethyl acetate-hexane (UV 254 nM); ¹H-NMR (400 MHz; DMSO-d6) δ 12.01 (s, 1H), 8.16 (br s, 1H), 7.81-7.85 (m, 2H), 7.72 (s, 1H), 7.49 (br s, 1H), 7.35 (d, 1H, J=7.8 Hz), 7.26 (s, 1H), 7.17 (br d, 1H, J=8.7 Hz), 7.08 (d, 1H, J=7.8 Hz), 2.80 (q, 1H, J=7.8 Hz), 2.00-2.20 (m, 1H), 1.78-1.82 (m, 1H), 1.37 (dt, 1H, J=4.6, 8.1 Hz); ¹⁹F-NMR (376 MHz; DMSO-d6) δ −56.72; MS (APCI⁺) m/z 406.0 (M+1), (APCI−) m/z 404.0 (M−1); HPLC UV purity, Rt=6.70 min, 96.4%; Chiral HPLC purity Rt=12.51 min, 93.9%; optical rotation [α]²⁵_{D}=−22.4 (c=0.25, 2.24 mL IPA); melting point=91-93° C.

### Example 73: Synthesis of cis-(+)-2-(6-(2-carbamoyl-6-(trifluoromethoxy)-1H-indol-1-yl)pyridin-2-yl)cyclopropane-1-carboxylic acid (Compound 28e)

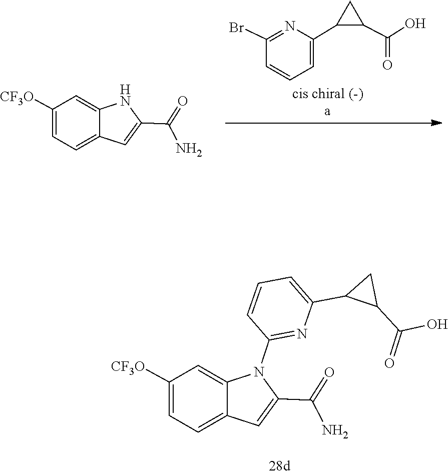

### Step A: Preparation of cis-(+)-2-(6-(2-carbamoyl-6-(trifluoromethoxy)-1H-indol-1-yl)pyridin-2-yl)cyclopropane-1-carboxylic acid (Compound 28e)

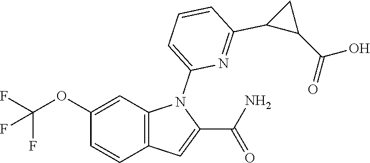 [SMILES: NC(=O)c1cc2ccc(OC(F)(F)F)cc2n1-c1cccc(C2CC2C(=O)O)n1]

To a mixture consisting of 6-(trifluoromethoxy)-1H-indole-2-carboxamide (0.293 g, 1.20 mmol), cis-(−)-2-(6-bromopyridin-2-yl)cyclopropane-1-carboxylic acid (0.242 g, 1.00 mmol), copper (I) iodide (Strem, 0.190 g, 1.00 mmol) in DMSO (20 mL) was added DBU (Oakwood Chemicals, 0.3 mL, 2.00 mmol). The reaction mixture was heated to 120° C. under N₂ atmosphere for 16 hours and then allowed to cool to room temperature. The reaction mixture was subsequently partitioned between ethyl acetate (50 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (75 mL), followed by brine (75 mL). The organic layer was separated and dried over anhydrous magnesium sulfate, filtered, and concentrated under reduced pressure to give a crude red oil, 0.412 g. The crude oil was purified by flash silica column chromatography on a CombiFlash NextGen 300+ purification system. Elution through a 12 g RediSep Gold Rf flash silica cartridge with 20-80% ethyl acetate in hexanes containing 1% acetic acid afforded the title compound as a tan solid (0.036 g, 9% yield); R_{f} 0.14 with 1:70:30 v/v acetic acid-ethyl acetate-hexane (UV 254 nM); ¹H-NMR (400 MHz; DMSO-d6) δ 12.02 (s, 1H), 8.16 (br s, 1H), 7.81-7.85 (m, 2H), 7.72 (s, 1H), 7.49 (br s, 1H), 7.35 (d, 1H, J=7.6 Hz), 7.26 (s, 1H), 7.17 (br d, 1H, J=8.7 Hz), 7.08 (d, 1H, J=7.8 Hz), 2.80 (q, 1H, J=8.4 Hz), 2.00-2.20 (m, 1H), 1.78-1.82 (m, 1H), 1.37 (dt, 1H, J=4.4, 8.0 Hz); ¹⁹F-NMR (376 MHz; DMSO-d6) δ −56.72; MS (APCI⁺) m/z 406.0 (M+1), (APCI−) m/z 404.0 (M−1); HPLC UV purity, Rt=6.70 min, 98.3%; Chiral HPLC purity Rt=15.45 min, 97.9%; optical rotation [α]²⁵_{D}=+17.6 (c=0.25, 2.24 mL IPA); melting point=77-79° C.

### Example 74: Synthesis of 1-(6-(3,3-difluoroazetidin-1-yl)pyridin-2-yl)-6-(trifluoromethoxy)-1H-indole-2-carboxamide (Compound 68)

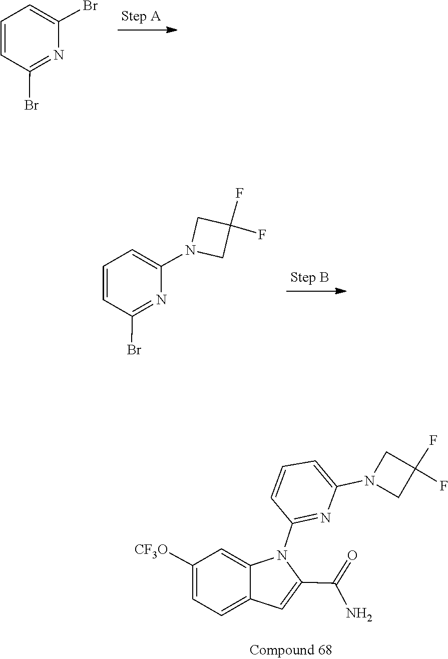

### Step A: Preparation of 2-bromo-6-(3,3-difluoroazetidin-1-yl)pyridine

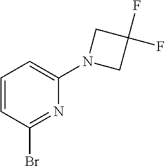 [SMILES: FC1(F)CN(c2cccc(Br)n2)C1]

To a mixture consisting of 2,6-dibromopyridine (Oakwood, 0.474 g, 2.00 mmol) in DMSO (10 mL) was added 3,3-difluoroazetidine hydrochloride (PharmaBlock, 0.259 g, 2.0 mmol) and potassium carbonate (2M aqueous solution, 3.0 mL, 6.00 mmol). The reaction mixture was stirred at 80° C. for 72 hours. The reaction mixture was subsequently cooled to room temperature and then partitioned between ethyl acetate (30 mL) and H₂O (30 mL). The phases were separated, and the organic layer was concentrated under reduced pressure to afford the crude product as a white solid (0.484 g). The crude solid was purified by flash silica column chromatography on a CombiFlash NextGen 300+ purification system. Elution through a 12 g RediSep Gold Rf flash silica cartridge with 2-10% ethyl acetate in hexanes afforded the title compound as a white solid (0.134 g, 26% yield). R_{f} 0.44 with 10:90 v/v ethyl acetate-heptane (UV 254 nM); ¹H-NMR (400 MHz; DMSO-d6) δ 7.53 (t, 1H, J=7.9 Hz), 6.96 (d, 1H, J=7.6 Hz), 6.56 (d, 1H, J=8.3 Hz), 4.40 (t, 4H, J=12.4 Hz); ¹⁹F-NMR (376 MHz; DMSO-d6) δ −98.63; MS (APCI−) m/z 248.0 (M+1); HPLC UV purity, Rt=8.30 min, 92.1%

### Step B: Preparation of 1-(6-(3,3-difluoroazetidin-1-yl)pyridin-2-yl)-6-(trifluoromethoxy)-1H-indole-2-carboxamide (Compound 68)

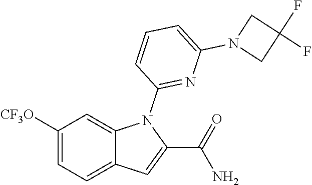

To a mixture consisting of 6-(trifluoromethoxy)-1H-indole-2-carboxamide (0.148 g, 0.607 mmol), 2-bromo-6-(3,3-difluoroazetidin-1-yl)pyridine (0.131 g, 0.526 mmol), copper (I) iodide (Strem, 0.116 g, 0.607 mmol) in DMSO (10 mL) was added DBU (Oakwood Chemicals, 0.18 mL, 1.21 mmol). The reaction mixture was heated to 125° C. under N₂ atmosphere for 4 hours and then allowed to cool to room temperature. The reaction mixture was subsequently partitioned between ethyl acetate (25 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (25 mL), followed by brine (25 mL). The organic layer was separated and dried over anhydrous magnesium sulfate, filtered, and concentrated under reduced pressure to give a crude brown oil, 0.204 g. The crude oil was purified by flash silica column chromatography on a CombiFlash NextGen 300+ purification system. Elution through a 12 g RediSep Gold Rf flash silica cartridge with 10-50% ethyl acetate in hexanes containing 1% acetic acid afforded the title compound as a tan solid (0.061 g, 28% yield). R_{f} 0.56 with 1:70:30 v/v acetic acid-ethyl acetate-hexane (UV 254 nM); ¹H-NMR (400 MHz; DMSO-d6) δ 8.10 (br s, 1H), 7.79-7.85 (m, 2H), 7.69 (t, 1H, J=7.8 Hz), 7.49 (br s, 2H), 7.17-7.20 (m, 2H), 6.79 (d, 1H, J=7.3 Hz), 6.60 (d, 1H, J=8.0 Hz), 4.41 (t, 4H, J=12.4 Hz); ¹⁹F-NMR (376 MHz; DMSO-d6) δ −56.72, −98.38; MS (APCI⁺) m/z 413.0 (M+1), (APCI⁻) m/z 411.0 (M−1); HPLC UV purity, Rt=8.643 min, 99.7%; melting point=206-208.0° C.

### Example 75: Synthesis of (S)-1-(6-(2-carbamoyl-6-(trifluoromethoxy)-1H-indol-1-yl)pyridin-2-yl)azetidine-2-carboxylic acid (Compound 69)

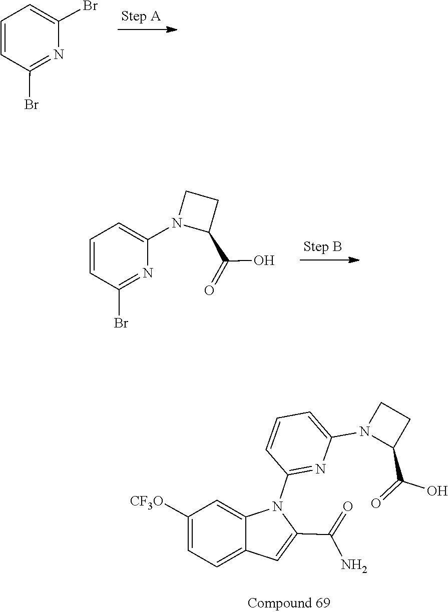

### Step A: Preparation of (S)-1-(6-bromopyridin-2-yl)azetidine-2-carboxylic acid

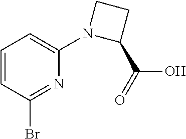 [SMILES: O=C(O)[C@@H]1CCN1c1cccc(Br)n1]

To a mixture consisting of 2,6-dibromopyridine (Oakwood, 0.474 g, 2.00 mmol) in DMSO (10 mL) was added L-azetidine-2-carboxylic acid (Cayman Chemical, 0.202 g, 2.0 mmol) and potassium carbonate (2M aqueous solution, 3.0 mL, 6.00 mmol). The reaction mixture was stirred at 80° C. for 72 hours. The reaction mixture was subsequently cooled to room temperature and then partitioned between ethyl acetate (30 mL) and H₂O (30 mL). The pH of the aqueous phase was adjusted to pH=2-3 with the addition on 1N HCl (6-8 mL). The phases were subsequently separated, and the organic layer was concentrated under reduced pressure to afford the crude product as a white solid (0.472 g, 91.8% yield). The crude solid was used without further purification. R_{f} 0.26 with 1:30:70 v/v ethyl acetate-heptane (UV 254 nM); ¹H-NMR (400 MHz; DMSO-d6) δ 12.81 (br s, 1H), 7.43 (t, 1H, J=7.9 Hz), 6.82 (d, 1H, J=7.6 Hz), 6.38 (d, 1H, J=8.3 Hz), 4.65 (dd, 1H, J=6.1, 9.3 Hz), 3.80-3.95 (m, 2H), 2.54-2.65 (m, 1H), 2.32 (tdd, 1H, J=6.3, 8.8, 11.0 Hz); MS (APCI−) m/z 257.0 (M+1), (APCI−) m/z 255.0 (M−1); HPLC UV purity, Rt.=5.86 min, 98.8%

### Step B: Preparation of 1-(6-(3,3-difluoroazetidin-1-yl)pyridin-2-yl)-6-(trifluoromethoxy)-1H-indole-2-carboxamide (Compound 69)

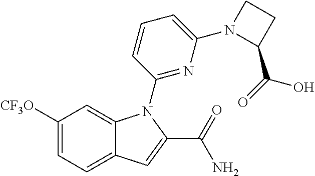

To a mixture consisting of 6-(trifluoromethoxy)-1H-indole-2-carboxamide (0.293 g, 1.2 mmol), (S)-1-(6-bromopyridin-2-yl)azetidine-2-carboxylic acid (0.257 g, 1.00 mmol), copper (I) iodide (Strem, 0.228 g, 1.20 mmol) in DMSO (15 mL) was added DBU (Oakwood Chemicals, 0.30 mL, 2.00 mmol). The reaction mixture was heated to 125° C. under N₂ atmosphere for 4 hours and then allowed to cool to room temperature. The reaction mixture was subsequently partitioned between ethyl acetate (25 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (25 mL), followed by brine (25 mL). The organic layer was separated and dried over anhydrous magnesium sulfate, filtered, and concentrated under reduced pressure to give a crude red oil, 0.544 g. The crude oil was purified by flash silica column chromatography on a CombiFlash NextGen 300+ purification system. Elution through a 12 g RediSep Gold Rf flash silica cartridge with 20-80% ethyl acetate in hexanes containing 1% acetic acid afforded the title compound as a tan solid (0.061 g, 14% yield). R_{f} 0.11 with 1:70:30 v/v acetic acid-ethyl acetate-hexane (UV 254 nM); ¹H-NMR (400 MHz; DMSO-d6) δ 12.75 (br s, 1H), 8.10 (br s, 1H), 7.81 (d, 1H, J=8.5 Hz), 7.68 (t, 1H, J=7.9 Hz), 7.56 (s, 1H), 7.46 (br s, 1H), 7.17 (s, 1H), 7.15 (s, 1H), 6.57 (d, 1H, J=7.6 Hz), 6.44 (d, 1H, J=8.3 Hz), 4.71 (dd, 1H, J=6.4, 9.2 Hz) 3.87-3.97 (m, 2H), 2.61-2.65 (m, 1H), 2.39-2.45 (m, 1H); ¹⁹F-NMR (376 MHz; DMSO-d6) δ −56.67; MS (APCI⁺) m/z 421.0 (M+1), (APCI−) m/z 419.0 (M−1); HPLC UV purity, Rt=7.097 min, 91.3%; Chiral HPLC purity, Rt=26.889 min, 95.9%; melting point=103-105° C.

### Example 76: Synthesis of (R)-1-(6-(2-carbamoyl-6-(trifluoromethoxy)-1H-indol-1-yl)pyridin-2-yl)azetidine-2-carboxylic acid (Compound 70)

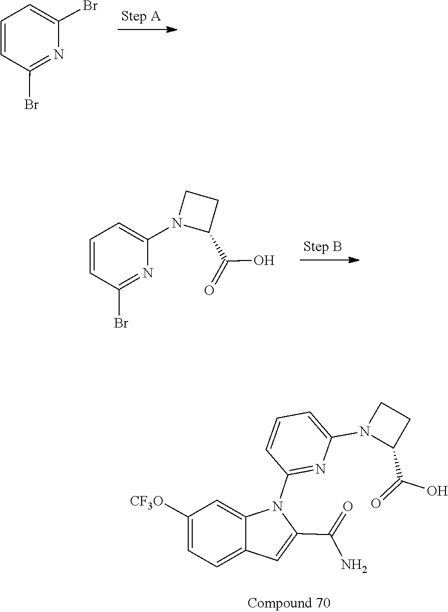

### Step A: Preparation of (R)-1-(6-bromopyridin-2-yl)azetidine-2-carboxylic acid

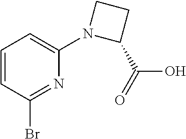 [SMILES: O=C(O)[C@H]1CCN1c1cccc(Br)n1]

To a mixture consisting of 2,6-dibromopyridine (Oakwood, 0.474 g, 2.00 mmol) in DMSO (12 mL) was added D-azetidine-2-carboxylic acid (CombiBlocks, 0.202 g, 2.0 mmol) and potassium carbonate (2M aqueous solution, 3.0 mL, 6.00 mmol). The reaction mixture was stirred at 80° C. for 16 hours. The reaction mixture was subsequently cooled to room temperature and then partitioned between ethyl acetate (30 mL) and H₂O (30 mL). The pH of the aqueous phase was adjusted to pH=2-3 with the addition on 1N HCl (6-8 mL). The phases were subsequently separated, and the organic layer was concentrated under reduced pressure to afford the crude product as a white solid (0.462 g, 89.2% yield). The crude solid was used without further purification. R_{f} 0.57 with 1:70:30 v/v ethyl acetate-heptane (UV 254 nM); ¹H-NMR (400 MHz; DMSO-d6) δ 12.81 (br s, 1H), 7.43 (t, 1H, J=7.8 Hz), 6.83 (d, 1H, J=7.3 Hz), 6.39 (d, 1H, J=8.3 Hz), 4.65 (dd, 1H, J=6.1, 9.3 Hz), 3.80-3.97 (m, 2H), 2.54-2.65 (m, 1H), 2.32 (tdd, 1H, J=6.3, 8.7, 11.0 Hz); MS (APCI−) m/z 257.0 (M+1), (APCI−) m/z 255.0 (M−1); HPLC UV purity, Rt.=5.86 min, 93.2%; Chiral HPLC purity, Rt=17.11 min, 77.5%

### Step B: Preparation of 1-(6-(3,3-difluoroazetidin-1-yl)pyridin-2-yl)-6-(trifluoromethoxy)-1H-indole-2-carboxamide (Compound 70)

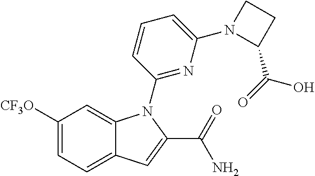

To a mixture consisting of 6-(trifluoromethoxy)-1H-indole-2-carboxamide (0.269 g, 1.1 mmol), (R)-1-(6-bromopyridin-2-yl)azetidine-2-carboxylic acid (0.257 g, 1.00 mmol), copper (I) iodide (Strem, 0.380 g, 2.00 mmol) in DMSO (20 mL) was added DBU (Oakwood Chemicals, 0.30 mL, 2.00 mmol). The reaction mixture was heated to 120° C. under N₂ atmosphere for 3 hours and then allowed to cool to room temperature. The reaction mixture was subsequently partitioned between ethyl acetate (30 mL) and 1M KHSO₄ (10 mL). A precipitate formed which was collected by filtration over a fritted funnel and set aside. The filtrate was poured in a separatory funnel. The phases were separated, and the organic phase was partitioned with H₂O (25 mL), followed by brine (25 mL). The organic layer was separated and dried over anhydrous magnesium sulfate, filtered, and concentrated under reduced pressure to give a crude red oil, 0.447 g. The crude oil was purified by flash silica column chromatography on a CombiFlash NextGen 300+ purification system. Elution through a 12 g RediSep Gold Rf flash silica cartridge with 30-90% ethyl acetate in hexanes containing 1% acetic acid afforded the title compound as a tan solid (0.030 g, 7% yield). R_{f} 0.09 with 1:70:30 v/v acetic acid-ethyl acetate-hexane (UV 254 nM); ¹H-NMR (400 MHz; DMSO-d6) δ 12.77 (br s, 1H), 8.10 (br s, 1H), 7.81 (d, 1H, J=8.7 Hz), 7.68 (t, 1H, J=7.9 Hz), 7.56 (s, 1H), 7.46 (br s, 1H), 7.17 (s, 1H), 7.15 (s, 1H), 6.57 (d, 1H, J=7.6 Hz), 6.44 (d, 1H, J=8.3 Hz), 4.70 (dd, 1H, J=6.6, 9.2 Hz), 3.87-3.97 (m, 2H), 2.61-2.65 (m, 1H), 2.39-2.45 (m, 1H); ¹⁹F-NMR (376 MHz; DMSO-d6) δ −56.67; MS (APCI⁺) m/z 421.0 (M+1), (APCI−) m/z 419.0 (M−1); HPLC UV purity, Rt=7.107 min, 99.0%; Chiral HPLC purity, Rt=26.574 min, 100%; melting point=99-101° C.

### Example 77: SPR and TSA Experiments for sPLA2-X and sPLA₂-IIA

### Step A: SPR Analysis

LY315920 (Varespladib) Against sPLA₂-IIA

SPLA2-IIA protein was captured on flow cell 2-4 with the previously prepared surface (CM5 Series S Sensorchip (Cytiva)) with anti-sPLA2-IIA antibody (Cayman Chemical, item #160500) covalently attached by amine coupling after dilution in 1×HBS-EP+ buffer (Cytiva), 10 mM CaCl2) to 0.015 mg/mL with a flow rate of 10 μL/min for 60 sec. Single cycle kinetic (SCK) binding analysis was performed using a 10 mM stock solution of LY315920 (Cayman Chemical, item #18267) prepared in DMSO diluted 1:5 in running buffer (1×HBS-EP+buffer (Cytiva)+10 mM CaCl2+2% DMSO) from 500 to 1.9 nM. Flow cell 1 was used as a reference surface and 10 mM glycine pH 1.5 (Cytiva) was used to regenerate the surface. DMSO solvent correction was included. A 1:1 fit was used for the kinetics analysis.

Kinetics and affinity of LY315920 were measured as: kₒₙ (M⁻¹ s⁻¹)=6.05×10⁵, k_{off} (s⁻¹)=7.30×10⁻³, K_{D} (M)=12.1 nM.

LY315920 against sPLA2-X

sPLA2-X protein was captured on flow cell 2-4 on activated NTA surface (NTA Series S Sensorchip (Cytiva)) after dilution in 1×HBS-EP+buffer (Cytiva), 10 mM CaCl₂ to 0.01 mg/mL with a flow rate of 5 μL/min for 60 sec. Single cycle kinetic binding analysis was performed using 10 mM stock solution of LY531590 (Cayman Chemical, item #18267) (prepared in DMSO diluted 1:5 in running buffer (lx HBS-EP+buffer (Cytiva)+10 mM CaCl₂+2% DMSO) from 10 to 0.016 μM. Flow cell 1 was used as a reference surface and 350 mM EDTA (Cytiva) was used to regenerate the surface. DMSO solvent correction was included. A 1:1 fit was used for the kinetics analysis.

Kinetics and affinity of LY315920 were measured as: kₒₙ (M⁻¹ s⁻¹)=2.10×10⁴, k_{off} (s⁻¹)=1.40×10⁻², K_{D} (M)=4.0 nM (FIG. 3)

Compound 3 Against sPLA2-X

sPLA2-X protein was captured on flow cell 2, 3, and 4 with the previously prepared surface (CM5 Series S Sensorchip (Cytiva) with anti-His antibody (Cytiva)) covalently attached by amine coupling after dilution in 1×HBS-EP+buffer (Cytiva), 10 mM CaCl2), to 0.02 mg/mL with a flow rate of 10 μL/min for 60 sec. Single cycle kinetic binding analysis was performed using a 50 mM stock solution of Compound 3 prepared in DMSO diluted 1:3 in running buffer (lx HBS-EP+buffer (Cytiva)+10 mM CaCl2)+2% DMSO) from 1000 to 12 nM. Flow cell 1 was used as a reference surface and 10 mM glycine pH 1.5 (Cytiva) was used to regenerate the surface. DMSO solvent correction was included. A 1:1 fit was used for the kinetics analysis.

### Step B: TSA Analysis

LY315920 (Varespladib) has the chemical structure:

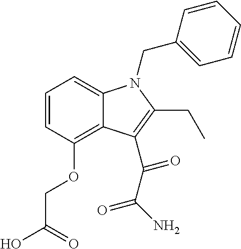 [SMILES: CCc1c(C(=O)C(N)=O)c2c(OCC(=O)O)cccc2n1Cc1ccccc1]

LY315920, Compound 1, Compound 6, and Compound 3 Against sPLA2-IIA (5.1 mg/mL Batch)

Thermal shift assays (TSA) were performed using the QuantStudio 6 Pro Real-Time PCR System™ (Applied) and QuantStudio™ software. The final reaction volume was 10.5 μl and contained PTS Buffer, PTS Dye (1× final concentration) and 0.5 mg/mL (final concentration) sPLA2-IIA purified protein, in the absence and presence of 50 μM (final concentration) of ligand (LY315920, Compound 1, Compound 6, and Compound 3). A control with no protein was also performed for all the samples. A continuous temperature increases from 20.0° C. to 99.0° C. was scanned every 0.1° C. in a ramp increment of 0.1° C. per second. Samples were run in duplicate. Melt temperatures were measured using the Protein Thermal Shift Analysis software (Thermo Fisher).

Run TSA for Batches of sPLA₂-IIA to Compare 0.5 mg/mL and 1.0 mg/mL

Thermal shift assay (TSA) optimization was performed using the QuantStudio 6 Pro Real-Time PCR System (Applied Biosystems). The final reaction volume was 10 μl and contained MQ H2O, PTS Dye (1× final concentration) and 0.5 mg/mL or 1.0 mg/mL (final concentration) sPLA2-IIA protein. A control with no protein was also performed for all the samples. A continuous temperature increases from 20.0° C. to 99.0° C. was scanned every 0.1° C. in a ramp increment of 0.05° C. per second. Samples were run in duplicate. Melt temperatures were measured using the Protein Thermal Shift Analysis software (Thermo Fisher, FIG. 4)

Run TSA for sPLA2-X

Thermal shift assay optimization was performed using the QuantStudio 6 Pro Real-Time PCR System (Applied Biosystems). The final reaction volume was 10 μl and contained MilliQ H2O, PTS Dye (IX final concentration) (ThermoFisher), and 2.3 mg/mL (final concentration) sPLA2-X protein. A control with no protein was also performed for all the samples. A continuous temperature increases from 20.0° C. to 99.0° C. was scanned every 0.1° C. in a ramp increment of 0.01° C. per second Samples were run in duplicate. Melt temperatures were measured using the Protein Thermal Shift Analysis software (Thermo Fisher)

Screening Compounds for Inhibition of sPLA₂-X, sPLA₂-V, and sPLA₂-IIA Enzyme Activity

Inhibition assays for sPLA2 groups IIA, V, and X enzyme activity were performed in 384-well plates using recombinant human enzymes; 1,2-bis(Heptanoylthio)-1,2-dideoxy-sn-glycero-3-phosphorylcholine substrate (thio-PC); 5,5′-dithio-bis-(2-nitrobenzoic acid; DTNB) detection reagent; and a range of test compound concentrations spanning 5.5 logs. These sPLA2 enzymes hydrolyze the sn-2 acyl chains of glycerophospholipids, resulting in the release of a free fatty acid and a lysophospholipid. In the case of the thio-PC substrate, this enzymatic hydrolysis results in a free sulfhydryl group remaining on the lysophospholipid. This free sulfhydryl reacts with DTNB to produce the chromogenic product 5-nitro-2-thiobenzoic acid (TNB) which can be quantitated by absorbance at 410 nm. The amount of TNB produced reflects the hydrolytic activity of the enzyme.

The recombinant human sPLA2 groups IIA, V, and X enzymes were expressed in E. coli and purified to >90% purity by standard biochemical methods.

Procedure of Enzyme Inhibition for sPLA₂-X
1. Seven test compounds plus one positive control compound (LY-320915; Varespladib) are assayed on one 384-well assay plate.
2. Dilute the test compounds in 100% DMSO by half-log steps in a 96-well dilution plate using columns 1-11 (one compound per row). Column 12 contains DMSO alone with no compound.
3. Prepare a 3.5 mM thio-PC substrate solution by drying down 7.2 mg of stock thio-PC in ethanol under a gentle stream of N₂ gas. When dry, add 4 mL assay buffer (25 mM Tris, pH7.4; 100 mM KCl; 10 mM CaCl₂; 0.1% Triton X-100) and warm in a 37° C. water bath for 20 minutes with intermittent vortexing.
4. Prepare an “enzyme containing” reaction mix with the following components: 20.4 mL assay buffer; 2 mL DTNB (20 mM in 800 mM Tris, pH8.0); and 11.8 mg recombinant human sPLA2-X enzyme (>95% pure) diluted to a total of 0.8 mL assay buffer.
5. Prepare a “no enzyme” reaction mix with the following components: 477 μL assay buffer and 45 μL DTNB.
6. Dispense 58 μL “no enzyme” reaction mix into each of 8 wells (columns 23 and 24, rows M-P).
7. Dispense 58 μL “enzyme containing” reaction mix into all remaining wells on the plate.
8. From the 96-well compound dilution plate, add 2 μL from each well for each compound to each of 4 wells with “enzyme containing” reaction mix. Columns 23 and 24 on the assay plate will contain only DMSO (vehicle control). Do not add anything to the “no enzyme wells (columns 23 and 24, rows M-P). After compound addition, mix the contents of each well to ensure that the DMSO is evenly distributed in the well.
9. Cover the plate and incubate in the dark at room temperature for 15 minutes.
10. Add 10 μL of thio-PC substrate to all wells. The final concentration of thio-PC in the reactions is 0.5 mM.
11. Cover the plate and incubate in the dark at room temperature for 30 minutes.
12. Measure the absorbance of all wells at 410 nm.
13. Determine the mean absorbance of the “no enzyme” wells. Subtract this value from the absorbance values determined for all other wells (these are now background corrected data).
14. Calculate the IC₅₀ for LY-315920 and for the test compounds from the background corrected data using GraphPad Prism. The IC₅₀ value for LY-315920 positive control should be approximately 10 nM.
   Procedure of Enzyme Inhibition for sPLA2-V
1. Seven test compounds plus one positive control compound (LY-320915; Varespladib) are assayed on one 384-well assay plate.
2. Dilute the test compounds in 100% DMSO by half-log steps in a 96-well dilution plate using columns 1-11. Column 12 contains DMSO alone with no compound.
3. Prepare a 3.5 mM thio-PC substrate solution by drying down 7.2 mg of stock thio-PC in ethanol under a gentle stream of N₂ gas. When dry, add 4 mL assay buffer (25 mM Tris, pH7.4; 100 mM KCl; 10 mM CaCl₂; 0.1% Triton X-100) and warm in a 37° C. water bath for 20 minutes with intermittent vortexing.
4. Prepare an “enzyme containing” reaction mix with the following components: 20.4 mL assay buffer; 2 mL DTNB (20 mM in 800 mM Tris, pH8.0); and 12 mg recombinant human sPLA2-V enzyme (>95% pure) diluted to a total of 0.8 mL assay buffer.
5. Prepare a “no enzyme” reaction mix with the following components: 477 μL assay buffer and 45 mL DTNB.
6. Dispense 58 μL “no enzyme” reaction mix into each of 8 wells (columns 23 and 24, rows M-P).
7. Dispense 58 μL “enzyme containing” reaction mix into all remaining wells on the plate.
8. From the 96-well compound dilution plate, add 2 μL from each well for each compound to each of 4 wells with “enzyme containing” reaction mix. Columns 23 and 24 on the assay plate will contain only DMSO (vehicle control). Do not add anything to the “no enzyme wells (columns 23 and 24, rows M-P). After compound addition, mix the contents of each well to ensure that the DMSO is evenly distributed in the well.
9. Cover the plate and incubate in the dark at room temperature for 15 minutes.
10. Add 10 μL of thio-PC substrate to all wells. The final concentration of thio-PC in the reactions is 0.5 mM.
11. Cover the plate and incubate in the dark at room temperature for 30 minutes.
12. Measure the absorbance of all wells at 410 nm.
13. Determine the mean absorbance of the “no enzyme” wells. Subtract this value from the absorbance values determined for all other wells (these are now background corrected data).
14. Calculate the IC₅₀ for LY-315920 and for the test compounds from the background corrected data using GraphPad Prism. The IC₅₀ value for LY-315920 positive control should be approximately 100 nM.
   Procedure of Enzyme Inhibition for sPLA2-IIA
1. Seven test compounds plus one positive control compound (LY-320915; Varespladib) are assayed on one 384-well assay plate.
2. Dilute the test compounds in 100% DMSO by half-log steps in a 96-well dilution plate using columns 1-11. Column 12 contains DMSO alone with no compound.
3. Prepare an 8 mM thio-PC substrate solution by drying down 28.8 mg of stock thio-PC in ethanol under a gentle stream of N₂ gas. When dry, add 7 mL assay buffer (25 mM Tris, pH7.4; 100 mM KCl; 10 mM CaCl₂; 0.1% Triton X-100) and warm in a 37° C. water bath for 20 minutes with intermittent vortexing.
4. Prepare an “enzyme containing” reaction mix with the following components: 17.4 mL assay buffer; 2 mL DTNB (20 mM in 800 mM Tris, pH8.0); and 180 mg recombinant human sPLA2-IIA enzyme (>90% pure) diluted to a total of 0.8 mL assay buffer.
5. Prepare a “no enzyme” reaction mix with the following components: 410 μL assay buffer and 45 μL DTNB.
6. Dispense 50.5 μL “no enzyme” reaction mix into each of 8 wells (columns 23 and 24, rows M-P).
7. Dispense 50.5 μL “enzyme containing” reaction mix into all remaining wells on the plate.
8. From the 96-well compound dilution plate, add 2 μL from each well for each compound to each of 4 wells with “enzyme containing” reaction mix. Columns 23 and 24 on the assay plate will contain only DMSO (vehicle control). Do not add anything to the “no enzyme wells (columns 23 and 24, rows M-P). After compound addition, mix the contents of each well to ensure that the DMSO is evenly distributed in the well.
9. Cover the plate and incubate in the dark at room temperature for 15 minutes.
10. Add 17.5 μL of thio-PC substrate to all wells. The final concentration of thio-PC in the reactions is 2 mM.
11. Cover the plate and incubate in the dark at room temperature for 30 minutes.
12. Measure the absorbance of all wells at 410 nm.
13. Determine the mean absorbance of the “no enzyme” wells. Subtract this value from the absorbance values determined for all other wells (these are now background corrected data).
14. Calculate the IC₅₀ for LY-315920 and for the test compounds from the background corrected data using GraphPad Prism. The IC₅₀ value for LY-315920 positive control should be approximately 120 nM.

Table 1: data for sPLA2-X, sPLA2-IIA, and sPLA2-V, as determined by the procedures described above. For sPLA2-X, **** indicates an IC₅₀ (nM) of ≤20; *** indicates an IC₅₀ (nM) of >20 and ≤50; ** indicates an IC₅₀ (nM) of >50 and ≤200; and * indicates an IC₅₀ (nM) of >200. For sPLA2-IIA, xxxx indicates an IC₅₀ (nM) of ≤200; xxx indicates an IC₅₀ (nM) of >200 and 500; xx indicates an IC₅₀ (nM) of >500 and 1000; and x indicates an IC₅₀ (nM) of >1000. For sPLA2-V, ++++ indicates an IC₅₀ (nM) of 500; +++ indicates an IC₅₀ (nM) of >500 and ≤1500; ++ indicates an IC₅₀ (nM) of >1500 and ≤5000; and + indicates an IC₅₀ (nM) of >5000.

| | sPLA2 Type X | sPLA2 Type IIa | sPLA2 Type V |
|---|---|---|---|
| Compound # | Inhibition (IC₅₀; nM) | Inhibition (IC₅₀; nM) | Inhibition (IC₅₀; nM) |
| LY-315920 | 12 | 137 | 124 |
| (Control) | | | |
| Compound 1 | ** | x | + |
| Compound 6 | * | x | + |
| Compound 3 | **** | xx | ++ |
| Compound la | ** | x | + |
| Compound 1b | * | x | + |
| Compound 2 | **** | xxx | +++ |
| Compound 4 | **** | xxx | ++++ |
| Compound 5 | *** | xxx | ++ |
| Compound 7 | * | x | + |
| Compound 8 | ** | x | + |
| Compound 9 | * | x | + |
| Compound 10 | *** | xx | +++ |
| Compound 11 | ** | x | + |
| Compound 12 | ** | x | + |
| Compound 13 | * | x | + |
| Compound 14 | * | x | + |
| Compound 15 | *** | x | + |
| Compound 15a | * | x | + |
| Compound 15b | *** | x | + |
| Compound 16 | ** | x | + |
| Compound 17 | * | x | + |
| Compound 18 | **** | x | ++ |
| Compound 19 | * | x | + |
| Compound 20 | * | x | + |
| Compound 21 | * | x | + |
| Compound 22 | **** | x | ++ |
| Compound 23 | * | x | + |
| Compound 24 | * | x | + |
| Compound 25 | * | x | + |
| Compound 26 | * | x | + |
| Compound 27 | * | x | + |
| Compound 28 | **** | x | + |
| Compound 28a | **** | x | + |
| Compound 28b | * | x | + |
| Compound 28c | *** | xxx | ++ |
| Compound 28d | ** | x | ++ |
| Compound 28e | **** | xxxx | + |
| Compound 29 | ** | xx | ++ |
| Compound 30 | **** | x | ++ |
| Compound 31 | **** | x | ++++ |
| Compound 32 | * | x | + |
| Compound 33 | * | x | + |
| Compound 34 | *** | x | ++ |
| Compound 35 | * | x | + |
| Compound 36 | **** | x | +++ |
| Compound 37 | * | x | + |
| Compound 38a | **** | xxx | ++++ |
| Compound 38b | *** | x | + |
| Compound 39 | * | x | + |
| Compound 40 | * | x | + |
| Compound 41 | * | x | + |
| Compound 42 | * | x | + |
| Compound 43 | * | x | + |
| Compound 44 | **** | x | + |
| Compound 45 | *** | x | + |
| Compound 46 | ** | x | + |
| Compound 47 | * | x | + |
| Compound 48 | * | x | + |
| Compound 49 | * | x | + |
| Compound 50 | *** | x | + |
| Compound 51 | * | x | + |
| Compound 52 | ** | x | ++ |
| Compound 53 | ** | x | + |
| Compound 54 | ** | x | + |
| Compound 55 | * | x | + |
| Compound 56 | **** | x | + |
| Compound 57 | ** | x | + |
| Compound 58 | * | x | + |
| Compound 59 | **** | x | + |
| Compound 60 | ND | ND | ND |
| Compound 61 | ND | ND | ND |
| Compound 62 | *** | x | ++ |
| Compound 63 | ND | ND | ND |
| Compound 64 | ND | ND | ND |
| Compound 65 | ND | ND | ND |
| Compound 66 | ND | ND | ND |
| Compound 67 | ND | ND | ND |
| Compound 68 | ** | x | + |
| Compound 69 | * | x | + |
| Compound 70 | **** | x | +++ |

ND = No Data

### Example 78: Determining the Affinity of Compound 5 for sPLA2-X

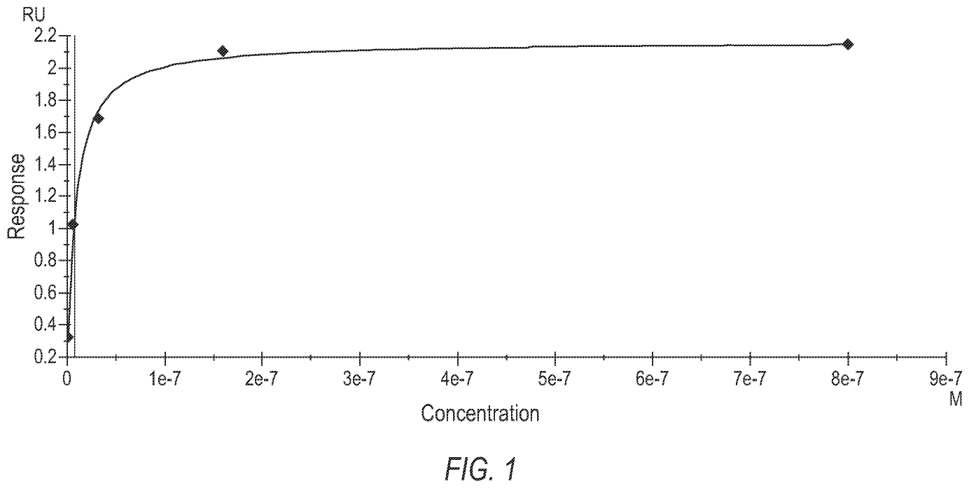

SPLA2-X protein was captured cross-linked on flow cell 2, 3, and 4 with activated NTA surface (NTA Series S Sensorchip (Cytiva)) at 0.01 mg/mL with a flow rate of 5 L/min for 60 sec. Single cycle kinetic binding analysis was performed using a 50 mM stock solution of Compound 5 (prepared in DMSO) diluted 1:5 in running buffer (1×HIBS-EP+buffer (Cytiva)+10 mM CaCl₂+2% o DMSO) from 800 to 1.2 nMV. Flow cell 1 was used as a reference surface. DMSO solvent correction was included. A 1:1 fit was used for the kinetics and steady state affinity analysis. The affinity of Compound 5 for sPLA2-X was measured as: K_{D} (M)=8.02 nM, Rmax=2.13 (FIG. 1).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims.

1-121. (canceled)

122. A compound of Formula IIIf-1, IIIf-2, IIIf-3, IIIf-4, IIIf-5, IIIf-6, or IIIf-7: 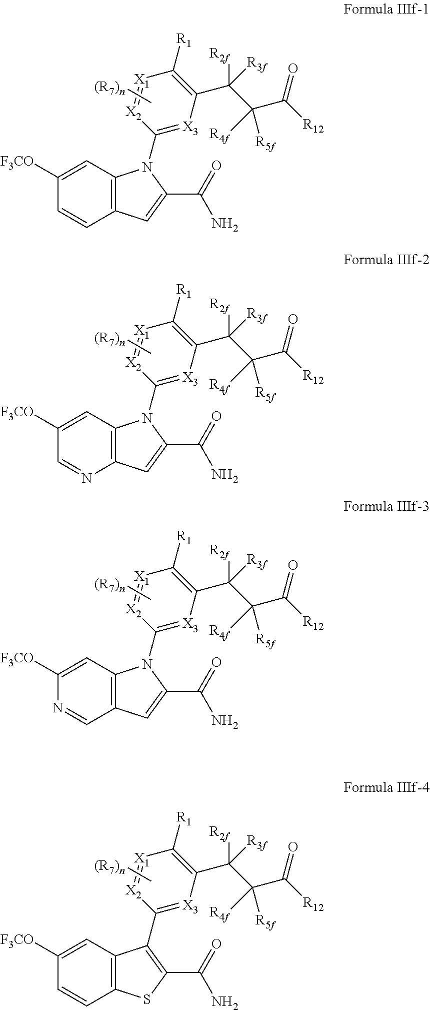 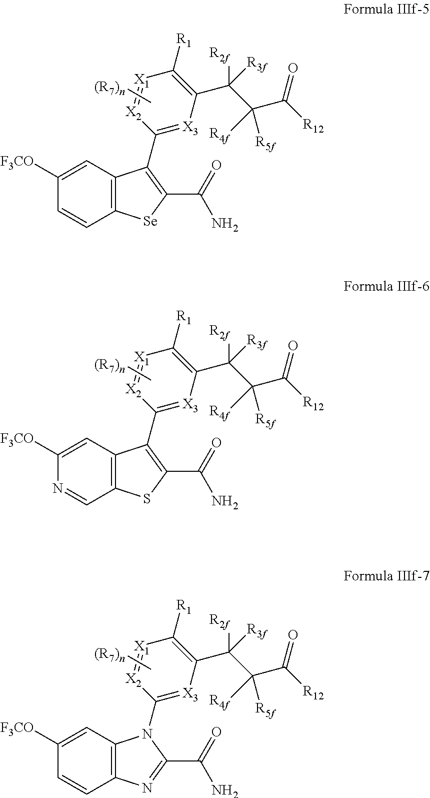
or a pharmaceutically acceptable salt thereof, wherein
X₁-X₃ are each independently N or CH;
R₁ is H or R₇;
R_{2f} is selected from hydrogen CN, OH, NH₂, halo, and C₁₋₆ alkyl;
R_{5f} is selected from hydrogen, CN, OH, NH₂, halo, C₁₋₆ alkyl, and OC₁₋₆ alkyl;
R_{3f} and R_{4f}, together with the carbon atom to which they are attached, form a 3-6 membered cyclyl or a 3-6 membered heterocyclyl, wherein each cyclyl and heterocyclyl are optionally substituted with one or more R₁₀ substituents.
each R₇ is independently R₁₀;
each R₁₀ is selected from oxo, CN, OR₁₁, N(R₁₁)₂, halo, C(O)H, C(O)R₁₁, C(O)OR₁, C(O)N(R₁)₂, (C₁₋₆ alkyl)-C(O)R₁₁, (C₁₋₆ alkyl)-C(O)OR₁₁, (C₁₋₆ alkyl)-C(O)N(R₁₁)₂, N(R₁₁)C(O)R₁₁, N(R₁₁)C(O)OR₁₁, N(R₁₁)C(O)NHR₁₁, N(Ri)-(CH₂)—C(O)R₁₁, N(R₁₁)—(CH₂)—C(O)OR₁₁, N(R₁₁)—(CH₂)—C(O)NHR₁₁, N(R₁₁)SO₂C₁₋₆ alkyl, OSO₂C₁₋₆ alkyl, SO₂C₁₋₆ alkyl, S(O)₂N(R₁₁)₂, C₁₋₆ alkyl, a phenyl, a 3-6 membered cyclyl, a 3-6 membered heterocyclyl, or a 5 or 6 membered heteroaryl, wherein each alkyl, phenyl, cyclyl, heterocyclyl, or heteroaryl are each independently and optionally substituted with one or more R₁₁ substituents;
each R₁₁ is independently selected from hydrogen, oxo, CN, OH, NH₂, halo, OC₁₋₆ alkyl, OC₁₋₆ haloalkyl, C₁₋₆ alkyl, and C₁₋₆ haloalkyl;
each R₁₂ is independently selected from OH, OC₁₋₆ alkyl, O-phenyl, NH₂, NH(C₁₋₆ alkyl), and N(C₁₋₆ alkyl)₂, wherein each alkyl and phenyl are optionally and independently substituted with one to three R₁₀ substituents; and
n is an integer selected from 0, 1, 2, and 3.

123. The compound or pharmaceutically acceptable salt thereof according to claim 122, wherein R_{3f} and R_{4f}, together with the carbon atom to which they are attached, form a 3-6 membered cyclyl optionally substituted with one or more C₁₋₆ alkyl substituents.

124. The compound or pharmaceutically acceptable salt thereof according to claim 123, wherein R_{3f} and R_{4f}, together with the carbon atom to which they are attached, form a cyclopropyl or cyclobutyl moiety, which is optionally substituted with one or more C₁₋₆ alkyl substituents.

125. The compound or pharmaceutically acceptable salt thereof according to claim 122, wherein R₁ is H.

126. The compound or pharmaceutically acceptable salt thereof according to claim 122, wherein each R₇ is independently selected from CN, OC₁₋₆ alkyl, halo, and C₁₋₆ alkyl.

127. The compound or pharmaceutically acceptable salt thereof according to claim 126, wherein each R₇ is independently selected from halo and C₁₋₆ alkyl, and n is 0 or 1.

128. The compound or pharmaceutically acceptable salt thereof according to claim 127, wherein n is 0.

129. The compound or pharmaceutically acceptable salt thereof according to claim 122, wherein the moiety

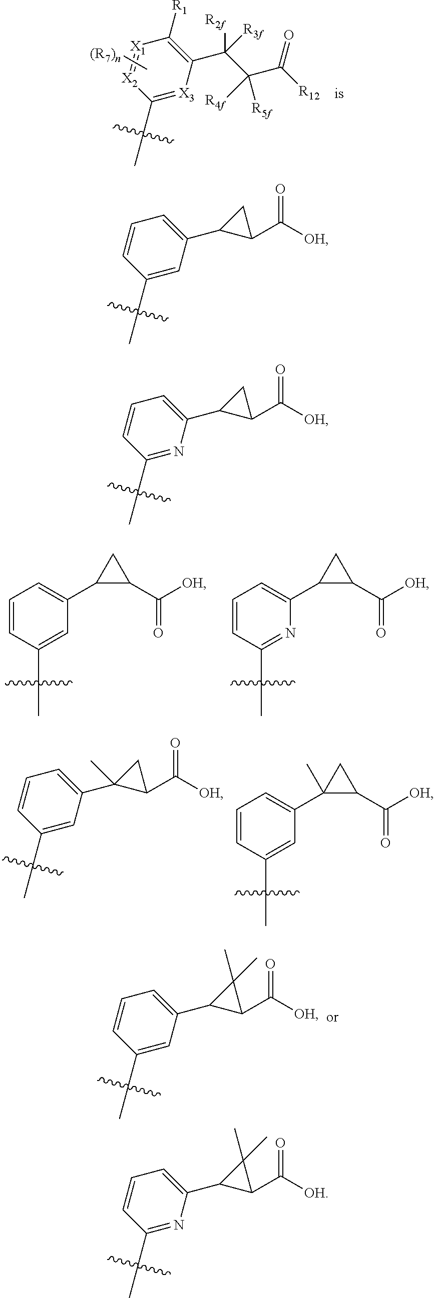

130. The compound or pharmaceutically acceptable salt thereof according to claim 122, wherein the compound is selected from:

| Compound | Name | Structure |
|---|---|---|
| 15 | trans-(rac)-2-(3-(2-carbamoyl-6- (trifluoromethoxy)-1H-indol-1- yl)phenyl)cyclopropane-1-carboxylic acid | 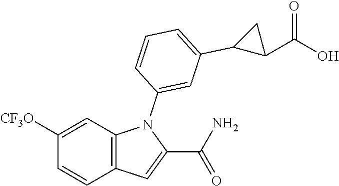 |
| 15b | trans-(−)-2-(3-(2-carbamoyl-6- (trifluoromethoxy)-1H-indol-1- yl)phenyl)cyclopropane-1-carboxylic acid | 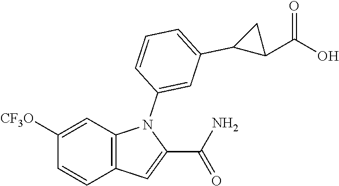 |
| 28 | trans-(rac)-2-(6-(2-carbamoyl-6- (trifluoromethoxy)-1H-indol-1- yl)pyridin-2-yl)cyclopropane-1- carboxylic acid | 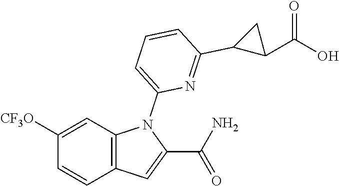 |
| 28a | trans-(−)-2-(6-(2-carbamoyl-6- (trifluoromethoxy)-1H-indol-1- yl)pyridin-2-yl)cyclopropane-1- carboxylic acid | 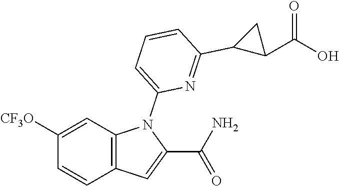 |
| 28b | trans-(+)-2-(6-(2-carbamoyl-6- (trifluoromethoxy)-1H-indol-1- yl)pyridin-2-yl)cyclopropane-1- carboxylic acid | 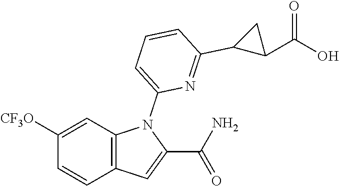 |
| 28c | cis-(rac)-2-(6-(2-carbamoyl-6- (trifluoromethoxy)-1H-indol-1- yl)pyridin-2-yl)cyclopropane-1- carboxylic acid | 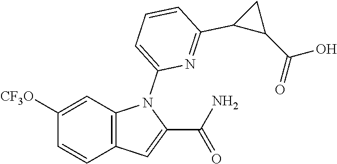 |
| 28d | cis-(−)-2-(6-(2-carbamoyl-6- (trifluoromethoxy)-1H-indol-1- yl)pyridin-2-yl)cyclopropane-1- carboxylic acid | 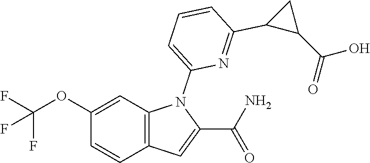 [SMILES: NC(=O)c1cc2ccc(OC(F)(F)F)cc2n1-c1cccc(C2CC2C(=O)O)n1] |
| 28e | cis-(+)-2-(6-(2-carbamoy1-6- (trifluoromethoxy)-1H-indol-1- yl)pyridin-2-yl)cyclopropane-1- carboxylic acid | 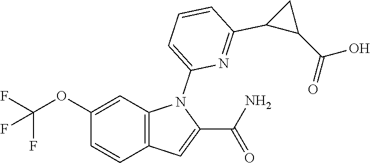 [SMILES: NC(=O)c1cc2ccc(OC(F)(F)F)cc2n1-c1cccc(C2CC2C(=O)O)n1] |
| 38b | trans-2-(3-(2-carbamoyl-6- (trifluoromethoxy)-1H-indol-1- yl)phenyl)-2-methylcyclopropane-1- carboxylic acid | 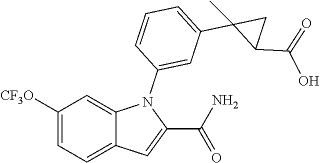 |
| 51 | trans-2-(6-(2-carbamoyl-6-methoxy- 1H-pyrrolo[3,2-b]pyridin-1-yl)pyridin- 2-yl)cyclopropane-1-carboxylic acid | 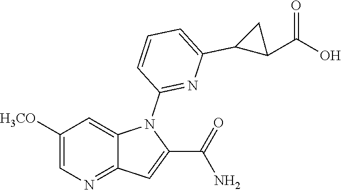 [SMILES: COc1cnc2cc(C(N)=O)n(-c3cccc(C4CC4C(=O)O)n3)c2c1] |
| 56 | trans-2-(6-(2-carbamoyl-5- (trifluoromethoxy)benzo[b]thiophen-3- yl)pyridin-2-yl)cyclopropane-1- carboxylic acid | 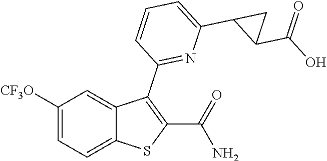 |
| 57 | trans-2-(6-(2-carbamoyl-5- (trifluoromethoxy)benzo[b]selenophen- 3-yl)pyridin-2-yl)cyclopropane-1- carboxylic acid | 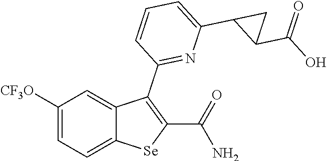 |
| 58 | trans-2-(6-(2-carbamoyl-6- (trifluoromethoxy)-1H- benzo[d]imidazol-1-yl)pyridin-2- yl)cyclopropane-1-carboxylic acid | 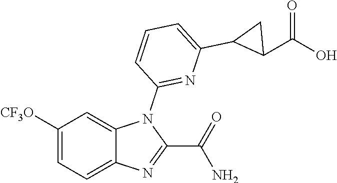 |
| 65 | trans-2-(6-(2-carbamoylthieno[2,3- c]pyridin-3-yl)pyridin-2- yl)cyclopropane-1-carboxylic acid | 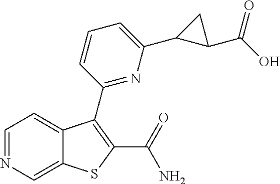 [SMILES: NC(=O)c1sc2cnccc2c1-c1cccc(C2CC2C(=O)O)n1] |
| 66 | trans-2-(6-(2-carbamoyl-6-methoxy- 1H-pyrrolo[3,2-c]pyridin-1-yl)pyridin- 2-yl)cyclopropane-1-carboxylic acid | 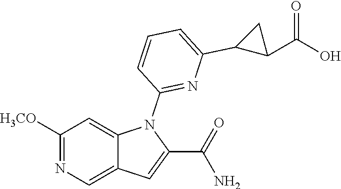 [SMILES: COc1cc2c(cn1)cc(C(N)=O)n2-c1cccc(C2CC2C(=O)O)n1] |

131. The compound or pharmaceutically acceptable salt thereof according to claim 130, wherein the compound is selected from:

| Compound | Name | Structure |
|---|---|---|
| 28a | trans-(−)-2-(6-(2-carbamoyl-6- (trifluoromethoxy)-1H-indol-1- yl)pyridin-2-yl)cyclopropane-1- carboxylic acid | 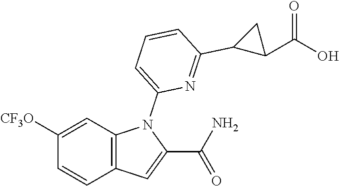 |
| 28b | trans-(+)-2-(6-(2-carbamoyl-6- (trifluoromethoxy)-1H-indol-1- yl)pyridin-2-yl)cyclopropane-1- carboxylic acid | 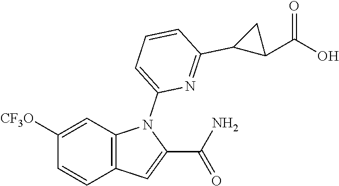 |
| 28d | cis-(−)-2-(6-(2-carbamoyl-6- (trifluoromethoxy)-1H-indol-1- yl)pyridin-2-yl)cyclopropane-1- carboxylic acid | 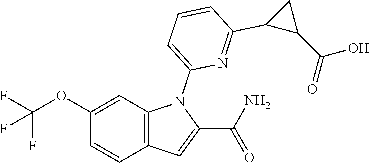 [SMILES: NC(=O)c1cc2ccc(OC(F)(F)F)cc2n1-c1cccc(C2CC2C(=O)O)n1] |
| 28e | cis-(+)-2-(6-(2-carbamoyl-6- (trifluoromethoxy)-1H-indol-1- yl)pyridin-2-yl)cyclopropane-1- carboxylic acid | 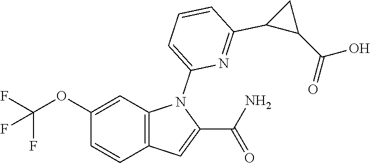 [SMILES: NC(=O)c1cc2ccc(OC(F)(F)F)cc2n1-c1cccc(C2CC2C(=O)O)n1] |

132. The compound or pharmaceutically acceptable salt thereof according to claim 131, wherein the compound is selected from:

| Compound | Name | Structure |
|---|---|---|
| 28a | trans-(−)-2-(6-(2-carbamoyl-6- (trifluoromethoxy)-1H-indol-1- yl)pyridin-2-yl)cyclopropane-1- carboxylic acid | 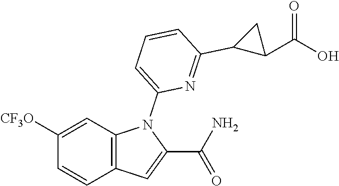 |

133. A compound selected from

| Compound | Name | Structure |
|---|---|---|
| 18 | 3-(6-(2-carbamoy1-6- (trifluoromethoxy)-1H-indol-1- yl)pyridin-2-yl)propanoic acid | 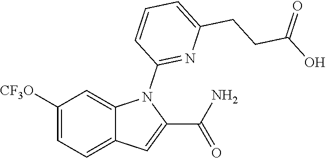 |
| 22 | 3-(3-(2-carbamoyl-6- (trifluoromethoxy)-1H-indol-1- yl)phenyl)-3-methylbutanoic acid | 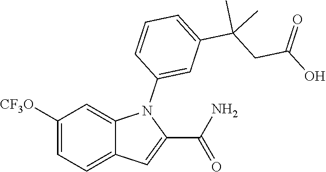 |
| 30 | 2-(1-(3-(2-carbamoyl-6- (trifluoromethoxy)-1H-indol-1- yl)phenyl)cyclobutyl)acetic acid | 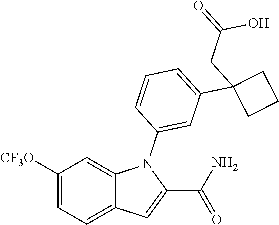 |
| 44 | 2-((6-(2-carbamoy1-6- (trifluoromethoxy)-1H-indol-1- yl)pyridin-2-yl)thio)acetic acid | 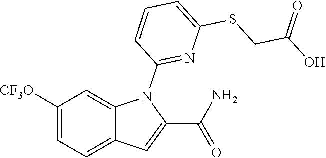 |
| 45 | 2-((6-(2-carbamoyl-6- (trifluoromethoxy)-1H-indol-1- yl)pyridin-2-yl)sulfonyl)acetic acid | 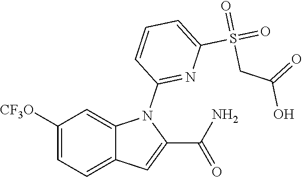 |
| 50 | (1-(6-(2-carbamoy1-6- (trifluoromethoxy)-1H-indol-1- yl)pyridin-2-yl)cyclobutyl)glycine | 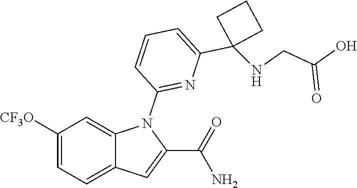 |
| 59 | 2-(1-(6-(2-carbamoyl-6- (trifluoromethoxy)-1H-indol-1- yl)pyridin-2-yl)cyclopropyl)acetic acid | 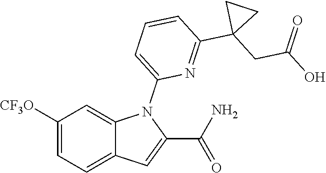 |
| 62 | 3-(6-(2-carbamoyl-6- (trifluoromethoxy)-1H-indol-1- yl)pyridin-2-yl)-3-methylbutanoic acid | 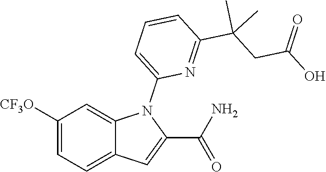 |

or a pharmaceutically acceptable salt thereof.

134. A pharmaceutical composition comprising a compound or pharmaceutically acceptable salt thereof according to claim 122, and a pharmaceutically acceptable carrier.

135. An in vitro method of inhibiting an sPLA2-X enzyme, said method comprising contacting the enzyme with a compound or pharmaceutically acceptable salt thereof according to claim 122, wherein the compound or pharmaceutically acceptable salt thereof is a selective inhibitor of sPLA2-X over other sPLA2 enzymes.

136. The in vitro method according to claim 135, wherein the other sPLA2 enzymes are selected from sPLA2-IIE, sPLA2-IIA, and sPLA2-V enzymes.

137. A method of treating a disease mediated by an sPLA2-X enzyme in a subject, said method comprising administering to the subject a therapeutically effective amount of a composition comprising a compound or pharmaceutically acceptable salt thereof according to claim 122.

138. The method according to claim 137, wherein the disease is selected from cancer, atherosclerosis, or cardiovascular disease.

139. The method according to claim 138, wherein the cancer is selected from multiple myeloma (MM), diffuse large B cell lymphoma (DLBCL), B cell lymphoma or non-small cell lung cancer.

140. The method according to claim 139, wherein the multiple myeloma (MM) and/or diffuse large B cell lymphoma (DLBCL) is recurring, refractory, or relapsing DLBCL and/or MM.

141. The method according to claim 139, wherein the cancer is selected from B cell lymphoma or non-small cell lung cancer.
